(19) United States
(12) Patent Application Publication
(10) Pub. No.: US 2007/0072897 A1
(43) Pub. Date: Mar. 29, 2007

(54) Phenylaminopropanol derivatives and methods of their use

(75) Inventors: Paige Erin Mahaney, Pottstown, PA (US); Gavin David Heffernan, Florence, NJ (US); Richard Dale Coghlan, Phoenixville, PA (US); Stephen Todd Cohn, Spring, TX (US); Callain Younghee Kim, Collegeville, PA (US); Douglas John Jenkins, Collegeville, PA (US); Michael Anthony Marella, Limerick, PA (US); Casey Cameron McComas, Phoenixville, PA (US); Joseph Peter Sabatucci, Collegeville, PA (US); Eugene Anthony Terefenko, Center Valley, PA (US); Eugene John Trybulski, Huntington Valley, PA (US); An Thien Vu, Pottstown, PA (US); Fei Ye, Audubon, PA (US); Puwen Zhang, Audubon, PA (US)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Wyeth, Madison, NJ (US)

(21) Appl. No.: 11/529,441

(22) Filed: Sep. 27, 2006

Related U.S. Application Data
(60) Provisional application No. 60/721,676, filed on Sep. 29, 2005.

(51) Int. Cl. A61K 31/4745; A61K 31/4184; A61K 31/407; A61K 31/404; A61K 31/4439; C07D 403/02; C07D 471/02

(52) U.S. Cl. 514/300; 514/409; 514/418; 514/337; 546/113; 546/277.4; 514/394; 548/307.4; 548/407; 548/484

(57) ABSTRACT

The present invention is directed to phenylaminopropanol derivatives of formulae I, II, and III: 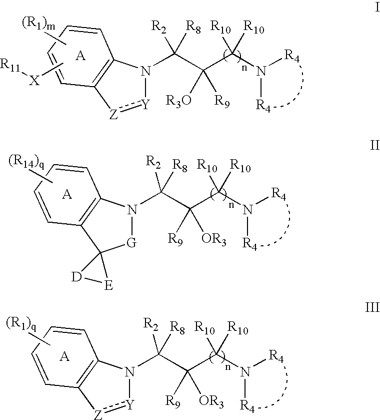 or a pharmaceutically acceptable salt thereof, compositions containing these derivatives, and methods of their use for the prevention and treatment of conditions ameliorated by monoamine reuptake including, inter alia, vasomotor symptoms (VMS), sexual dysfunction, gastrointestinal and genitourinary disorders, chronic fatigue syndrome, fibromyalgia syndrome, nervous system disorders, and combinations thereof, particularly those conditions selected from the group consisting of major depressive disorder, vasomotor symptoms, stress and urge urinary incontinence, fibromyalgia, pain, diabetic neuropathy, schizophrenia, and combinations thereof.

### CROSS REFERENCE TO RELATED APPLICATIONS

This application claims priority to U.S. Application No. 60/721,676 filed Sep. 29, 2005, the entire disclosure of which is incorporated herein by reference.

### FIELD OF THE INVENTION

The present invention relates to phenylaminopropanol derivatives, compositions containing these derivatives, and methods of their use for the prevention and treatment of conditions ameliorated by monoamine reuptake including, inter alia, vasomotor symptoms (VMS), sexual dysfunction, gastrointestinal and genitourinary disorders, chronic fatigue syndrome, fibromylagia syndrome, nervous system disorders, and combinations thereof, particularly those conditions selected from the group consisting of major depressive disorder, vasomotor symptoms, stress and urge urinary incontinence, fibromyalgia, pain, diabetic neuropathy, schizophrenia, and combinations thereof.

### BACKGROUND OF THE INVENTION

Vasomotor symptoms (VMS), referred to as hot flushes and night sweats, are the most common symptoms associated with menopause, occurring in 60% to 80% of all women following natural or surgically-induced menopause. VMS are likely to be an adaptive response of the central nervous system (CNS) to declining sex steroids. To date, the most effective therapies for VMS are hormone-based treatments, including estrogens and/or some progestins. Hormonal treatments are very effective at alleviating VMS, but they are not appropriate for all women. It is well recognized that VMS are caused by fluctuations of sex steroid levels and can be disruptive and disabling in both males and females. A hot flush can last up to thirty minutes and vary in their frequency from several times a week to multiple occurrences per day. The patient experiences a hot flash as a sudden feeling of heat that spreads quickly from the face to the chest and back and then over the rest of the body. It is usually accompanied by outbreaks of profuse sweating. It may sometimes occur several times an hour, and it often occurs at night. Hot flushes and outbreaks of sweats occurring during the night can cause sleep deprivation. Psychological and emotional symptoms observed, such as nervousness, fatigue, irritability, insomnia, depression, memory loss, headache, anxiety, nervousness or inability to concentrate are considered to be caused by the sleep deprivation following hot flush and night sweats (Kramer et al., In: Murphy et al., 3^{rd} Int'l Symposium on Recent Advances in Urological Cancer Diagnosis and Treatment—Proceedings, Paris, France: SCI: 3-7 (1992)).

Hot flushes may be even more severe in women treated for breast cancer for several reasons: 1) many survivors of breast cancer are given tamoxifen, the most prevalent side effect of which is hot flush, 2) many women treated for breast cancer undergo premature menopause from chemotherapy, 3) women with a history of breast cancer have generally been denied estrogen therapy because of concerns about potential recurrence of breast cancer (Loprinzi, et al., Lancet, 2000, 356(9247): 2059-2063).

Men also experience hot flushes following steroid hormone (androgen) withdrawal. This is true in cases of age-associated androgen decline (Katovich, et al., Proceedings of the Society for Experimental Biology & Medicine, 1990, 193(2): 129-35) as well as in extreme cases of hormone deprivation associated with treatments for prostate cancer (Berendsen, et al., European Journal of Pharmacology, 2001, 419(1): 47-54. As many as one-third of these patients will experience persistent and frequent symptoms severe enough to cause significant discomfort and inconvenience.

The precise mechanism of these symptoms is unknown but generally is thought to represent disturbances to normal homeostatic mechanisms controlling thermoregulation and vasomotor activity (Kronenberg, et al., “Thermoregulatory Physiology of Menopausal Hot Flashes: A Review,” Can. J. Physiol. Pharmacol., 1987, 65:1312-1324).

The fact that estrogen treatment (e.g., estrogen replacement therapy) relieves the symptoms establishes the link between these symptoms and an estrogen deficiency. For example, the menopausal stage of life is associated with a wide range of other acute symptoms as described above and these symptoms are generally estrogen responsive.

It has been suggested that estrogens may stimulate the activity of both the norepinephrine (NE) and/or serotonin (5-HT) systems (J. Pharmacology & Experimental Therapeutics, 1986, 236(3) 646-652). It is hypothesized that estrogens modulate NE and 5-HT levels providing homeostasis in the thermoregulatory center of the hypothalamus. The descending pathways from the hypothalamus via brainstem/spinal cord and the adrenals to the skin are involved in maintaining normal skin temperature. The action of NE and 5-HT reuptake inhibitors is known to impinge on both the CNS and peripheral nervous system (PNS). The pathophysiology of VMS is mediated by both central and peripheral mechanisms and, therefore, the interplay between the CNS and PNS may account for the efficacy of dual acting SRI/NRIs in the treatment of thermoregulatory dysfunction. In fact, the physiological aspects and the CNS/PNS involvement in VMS may account for the lower doses proposed to treat VMS (Loprinzi, et al., Lancet, 2000, 356:2059-2063; Stearns et al., JAMA, 2003, 289:2827-2834) compared to doses used to treat the behavioral aspects of depression. The interplay of the CNS/PNS in the pathophysiology of VMS and the presented data within this document were used to support the claims that the norepinephrine system could be targeted to treat VMS.

It has been reported that serotonin 2A (5-HT_{2A}) receptors play a role in temperature regulation (Berendsen, Maturitas, 2000, 36, 155). A low blood estrogen level has been shown to correlate with a high concentration of the 5-HT_{2A} receptor subtype on blood platelets (Biegon, Effects of steroid hormones on the serotonergic system. In: Whitaker-Azmitia, Peroutka editors. The Neuropharmacology of Serotonin. 1990, 427-34) and an upregulation of central 5-HT_{2A} receptors (Fink et al., Nature, 1996, 383, 306). The 5-HT₂ and 5-HT₃ receptor antagonist mirtazapine, was reported to be effective in reducing the frequency and intensity of hot flushes (Waldinger, et al., Maturitas, 2000, 36, 165). The 5-HT₂ receptor antagonist mianserin was also shown to be effective in treating hot flushes (Takagi, et al., Sanfujinka No Sekai (World Obstet Gynecol) 1986, 36, 853). The combination of a norepinephrine reuptake inhibitor with a 5-HT_{2A} receptor antagonist has also been reported to result in enhanced activity in animal models of thermoregulatory dysfunction (Deecher, et al., WO 2004/035036).

Although VMS are most commonly treated by hormone therapy (orally, transdermally, or via an implant), some patients cannot tolerate estrogen treatment (Berendsen, Maturitas, 2000, 36(3): 155-164, Fink et al., Nature, 1996, 383(6598): 306). In addition, hormone replacement therapy is usually not recommended for women or men with or at risk for hormonally sensitive cancers (e.g. breast or prostate cancer). Thus, non-hormonal therapies (e.g. fluoxetine, paroxetine [SRIs] and clonidine) are being evaluated clinically. WO9944601 discloses a method for decreasing hot flushes in a human female by administering fluoxetine. Other options have been studied for the treatment of hot flashes, including steroids, alpha-adrenergic agonists, and beta-blockers, with varying degree of success (Waldinger et al., Maturitas, 2000, 36(3): 165-168).

It has been reported that α₂-adrenergic receptors play a role in thermoregulatory dysfunctions (Freedman et al., Fertility & Sterility, 2000, 74(1): 20-3). These receptors are located both pre- and post-synaptically and mediate an inhibitory role in the central and peripheral nervous system. There are four distinct subtypes of the adrenergic_{α2} receptors, i.e., are α_{2A}, α_{2B}, α_{2C} and α_{2D} (Mackinnon et al., TIPS, 1994, 15: 119; French, Pharmacol. Ther., 1995, 68: 175). It has been reported that a non-select α₂-adrenoceptor antagonist, yohimbine, induces a flush and an α₂-adrenergic receptor agonist, clonidine, alleviates the yohimbine effect (Katovich, et al., Proceedings of the Society for Experimental Biology & Medicine, 1990, 193(2): 129-35, Freedman et al., Fertility & Sterility, 2000, 74(1): 20-3). Clonidine has been used to treat hot flush. However, using such treatment is associated with a number of undesired side effects caused by high doses necessary to abate hot flash described herein and known in the related arts.

Given the complex multifaceted nature of thermoregulation and the interplay between the CNS and PNS in maintaining thermoregulatory homeostasis, multiple therapies and approaches can be developed to target vasomotor symptoms. The present invention focuses on novel compounds and compositions containing these compounds directed to these and other important uses.

### SUMMARY OF THE INVENTION

The present invention is directed to phenylaminopropanol derivatives, compositions containing these derivatives, and methods of their use for the prevention and treatment of conditions ameliorated by monoamine reuptake including, inter alia, vasomotor symptoms (VMS), sexual dysfunction, gastrointestinal and genitourinary disorders, chronic fatigue syndrome, fibromylagia syndrome, nervous system disorders, and combinations thereof, particularly those conditions selected from the group consisting of major depressive disorder, vasomotor symptoms, stress and urge urinary incontinence, fibromyalgia, pain, diabetic neuropathy, schizophrenia, and combinations thereof.

In one embodiment, the invention is directed to compounds of formula I:

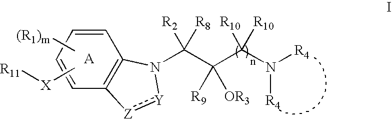

or a pharmaceutically acceptable salt thereof;

wherein:

the dotted line between Y and Z represents an optional second bond;

the dotted line between the two R₄ groups represents an optional heterocyclic ring of 4 to 6 ring atoms that may be formed between the two R₄ groups, together with the nitrogen through which they are attached;

X is —(C(R₁₂)₂)ₒ—, —O(C(R₁₂)₂)ₒ—, —(C(R₁₂)₂)ₒO—, —S(O)ₚ(C(R₁₂)₂)ₒ—, —(C(R₁₂)₂)ₒS(O)ₚ—, —N(R₁₃)C(O)(C(R₁₂)₂)ₒ—, —(C(R₁₂)₂)ₒC(O)N(R₁₃)—, —C(O)N(R₁₃)(C(R₁₂)₂)ₒ—, —(C(R₁₂)₂)ₒN(R₁₃)C(O)—, —(C(R₁₂)₂)ₒN(R₁₃)S(O)₂—, —S(O)₂N(R₁₃)(C(R₁₂)₂)ₒ—, —N(R₁₃)S(O)₂(C(R₁₂)₂)ₒ—, —(C(R₁₂)₂)ₒS(O)₂N(R₁₃)—, —NR₇(C(R₁₂)₂)ₒ—, —(C(R₁₂)₂)ₒNR₇—, or —C≡C—;

Y is N, C(R₆)₂, CR₆, or C═O;

Z is O, S(O)ₚ, N, NR₇, CR₅, or C(R₅)₂;

R₁ is, independently at each occurrence, alkyl, alkoxy, halo, CF₃, OCF₃, hydroxy, alkanoyloxy, nitro, cyano, alkenyl, alkynyl, alkylsulfoxide, alkylsulfone, alkylsulfonamide, or alkylamido; or two adjacent R₁ also represent methylenedioxy;

R₂ is aryl substituted with 0-3 R₁₄ or heteroaryl substituted with 0-3 R₁₄;

R₃ is H or C₁-C₄ alkyl;

R₄ is, independently at each occurrence, H, C₁-C₄ alkyl, C₃-C₆ cycloalkyl, arylalkyl, heteroarylmethyl, cycloheptylmethyl, cyclohexylmethyl, cyclopentylmethyl, or cyclobutylmethyl, or

both R₄ groups, together with the nitrogen through which they are attached, form a heterocyclic ring of 4 to 6 ring atoms, where one carbon may be optionally replaced with N, O, S, or SO₂, and where any carbon ring atom or additional N atom may be optionally substituted with C₁-C₄ alkyl, F, or CF₃;

R₅ is, independently at each occurrence, H, C₁-C₄ alkyl, aryl substituted with 0-3 R₁₄, heteroaryl substituted with 0-3 R₁₄, or cyano; or when two R₅ are present, they may form a carbocyclic ring of 3-5 carbons;

R₆ is, independently at each occurrence, H, C₁-C₄ alkyl, or cyano;

R₇ is H, C₁-C₆ alkyl, C₃-C₆ cycloalkyl, aryl substituted with 0-3 R₁₄; or heteroaryl substituted with 0-3 R₁₄.

R₈ is H, or C₁-C₄ alkyl;

R₉ is H, or C₁-C₄ alkyl;

R₁₀ is, independently at each occurrence, H, or C₁-C₄ alkyl; or R₁₀ and R₄ together with the nitrogen to which R₄ is attached form a nitrogen-containing ring containing 3-6 carbon atoms;

R₁₁ is aryl substituted with 0-3 R₁ or heteroaryl substituted with 0-3 R₁;

R₁₂ is, independently at each occurrence, H, C₁-C₄ alkyl;

R₁₃ is H or C₁-C₄ alkyl;

R₁₄ is, independently at each occurrence, alkyl, alkoxy, halo, CF₃, OCF₃, arylalkyloxy substituted with 0-3 R₁, aryloxy substituted with 0-3 R₁, aryl substituted with 0-3 R₁, heteroaryl substituted with 0-3 R₁, hydroxy, alkanoyloxy, nitro, cyano, alkenyl, alkynyl, alkylsulfoxide, phenylsulfoxide substituted with 0-3 R₁, alkylsulfone, phenylsulfone substituted with 0-3 R₁, alkylsulfonamide, phenylsulfonamide substituted with 0-3 R₁, heteroaryloxy substituted with 0-3 R₁, heteroarylmethyloxy substituted with 0-3 R₁, alkylamido, or arylamido substituted with 0-3 R₁; or two adjacent R₁ also represent methylenedioxy;

m is an integer from 0 to 3;

n is an integer from 1 to 2;

o is an integer from 0 to 3; and

p is an integer from 0 to 2;

wherein 1-3 carbon atoms in ring A may optionally be replaced with N.

In another embodiment, the invention is directed to compounds of formula II:

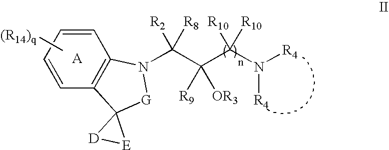

or a pharmaceutically acceptable salt thereof;

wherein:

D and E, together with the carbon atom through which they are attached, form a carbocyclic ring of 6 to 8 atoms or a heterocyclic ring of 5 to 8 atoms containing 1 to 2 heteroatoms selected from O, S(O)ₚ, and NR₇, where any carbon ring atom may be optionally substituted with C₁-C₄ alkyl, F or CF₃;

the dotted line between the two R₄ groups represents an optional heterocyclic ring of 4 to 6 ring atoms that may be formed between the two R₄ groups, together with the nitrogen through which they are attached;

G is NR₇, C(R₆)₂, or C═O;

R₁ is, independently at each occurrence, alkyl, alkoxy, halo, CF₃, OCF₃, hydroxy, alkanoyloxy, nitro, cyano, alkenyl, alkynyl, alkylsulfoxide, alkylsulfone, alkylsulfonamide, or alkylamido; or two adjacent R₁ also represent methylenedioxy;

R₂ is aryl substituted with 0-3 R₁₄ or heteroaryl substituted with 0-3 R₁₄;

R₃ is H or C₁-C₄ alkyl;

R₄ is, independently at each occurrence, H, C₁-C₄ alkyl, C₃-C₆ cycloalkyl, arylalkyl, heteroarylmethyl, cycloheptylmethyl, cyclohexylmethyl, cyclopentylmethyl, or cyclobutylmethyl, or

both R₄ groups, together with the nitrogen through which they are attached, form a heterocyclic ring of 4 to 6 ring atoms, where one carbon may be optionally replaced with N, O, S, or SO₂, and where any carbon ring atom or additional N atom may be optionally substituted with C₁-C₄ alkyl, F, or CF₃;

R₆ is, independently at each occurrence, H, C₁-C₄ alkyl, or cyano;

R₇ is H, C₁-C₆ alkyl, C₃-C₆ cycloalkyl, aryl substituted with 0-3 R₁₄; or heteroaryl substituted with 0-3 R₁₄.

R₈ is H, or C₁-C₄ alkyl;

R₉ is H, or C₁-C₄ alkyl;

R₁₀ is, independently at each occurrence, H, or C₁-C₄ alkyl; or R₁₀ and R₄ together with the nitrogen to which R₄ is attached form a nitrogen-containing ring containing 3-6 carbon atoms;

R₁₄ is, independently at each occurrence, alkyl, alkoxy, halo, CF₃, OCF₃, arylalkyloxy substituted with 0-3 R₁, aryloxy substituted with 0-3 R₁, aryl substituted with 0-3 R₁, heteroaryl substituted with 0-3 R₁, hydroxy, alkanoyloxy, nitro, cyano, alkenyl, alkynyl, alkylsulfoxide, phenylsulfoxide substituted with 0-3 R₁, alkylsulfone, phenylsulfone substituted with 0-3 R₁, alkylsulfonamide, phenylsulfonamide substituted with 0-3 R₁, heteroaryloxy substituted with 0-3 R₁, heteroarylmethyloxy substituted with 0-3 R₁, alkylamido, or arylamido substituted with 0-3 R₁; or two adjacent R₁ also represent methylenedioxy;

n is an integer from 1 to 2;

p is an integer from 0 to 2; and

q is an integer from 0 to 4;

wherein 1-3 carbon atoms in ring A may optionally be replaced with N.

In yet another embodiment, the invention is directed to compounds of formula III:

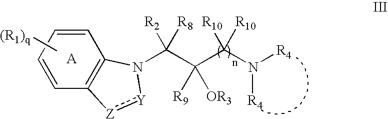

or a pharmaceutically acceptable salt thereof;

wherein:

the dotted line between Y and Z represents an optional second bond;

the dotted line between the two R₄ groups represents an optional heterocyclic ring of 4 to 6 ring atoms that may be formed between the two R₄ groups, together with the nitrogen through which they are attached;

Y is N, C(R₆)₂, CR₆, or C═O;

Z is O, S(O)ₚ, N, NR₇, CR₅, or C(R₅)₂;

R₁ is, independently at each occurrence, alkyl, alkoxy, halo, CF₃, OCF₃, hydroxy, alkanoyloxy, nitro, cyano, alkenyl, alkynyl, alkylsulfoxide, alkylsulfone, alkylsulfonamide, or alkylamido; or two adjacent R₁ also represent methylenedioxy;

R₂ is aryl substituted with 0-3 R₁₄ or heteroaryl substituted with 0-3 R₁₄;

R₃ is H or C₁-C₄ alkyl;

R₄ is, independently at each occurrence, H, C₁-C₄ alkyl, C₃-C₆ cycloalkyl, arylalkyl, heteroarylmethyl, cycloheptylmethyl, cyclohexylmethyl, cyclopentylmethyl, or cyclobutylmethyl, or

both R₄ groups, together with the nitrogen through which they are attached, form a heterocyclic ring of 4 to 6 ring atoms, where one carbon may be optionally replaced with N, O, S, or SO₂, and where any carbon ring atom or additional N atom may be optionally substituted with C₁-C₄ alkyl, F, or CF₃;

R₅ is, independently at each occurrence, H, C₁-C₄ alkyl, aryl substituted with 0-3 R₁₄, heteroaryl substituted with 0-3 R₁₄, or cyano; or when two R₅ are present, they may form a carbocyclic ring of 3-5 carbons;

R₆ is, independently at each occurrence, H, C₁-C₄ alkyl, or cyano;

R₇ is H, C₁-C₆ alkyl, C₃-C₆ cycloalkyl, aryl substituted with 0-3 R₁₄, or heteroaryl substituted with 0-3 R₁₄;

R₈ is H, or C₁-C₄ alkyl;

R₉ is H, or C₁-C₄ alkyl;

R₁₀ is, independently at each occurrence, H, or C₁-C₄ alkyl; or R₁₀ and R₄ together with the nitrogen to which R₄ is attached form a nitrogen-containing ring containing 3-6 carbon atoms;

R₁₄ is, independently at each occurrence, alkyl, alkoxy, halo, CF₃, OCF₃, arylalkyloxy substituted with 0-3 R₁, aryloxy substituted with 0-3 R₁, aryl substituted with 0-3 R₁, heteroaryl substituted with 0-3 R₁, hydroxy, alkanoyloxy, nitro, cyano, alkenyl, alkynyl, alkylsulfoxide, phenylsulfoxide substituted with 0-3 R₁, alkylsulfone, phenylsulfone substituted with 0-3 R₁, alkylsulfonamide, phenylsulfonamide substituted with 0-3 R₁, heteroaryloxy substituted with 0-3 R₁, heteroarylmethyloxy substituted with 0-3 R₁, alkylamido, or arylamido substituted with 0-3 R₁; or two adjacent R₁ also represent methylenedioxy;

n is an integer from 1 to 2; and

q is an integer from 0 to 4;

wherein 1-3 carbon atoms in ring A may optionally be replaced with N.

In yet other embodiments, the present invention is directed to compositions, comprising:
a. at least one compound of formula I, II, or III, or a pharmaceutically acceptable salt thereof; and
b. at least one pharmaceutically acceptable carrier.

In another embodiment, the present invention is directed to methods for treating or preventing a condition ameliorated by monoamine reuptake in a subject in need thereof, comprising the step of:
administering to said subject an effective amount of a compound of formula I, II, III, or pharmaceutically acceptable salt thereof.
   The conditions ameliorated by monoamine reuptake include those selected from the group consisting of vasomotor symptoms, sexual dysfunction, gastrointestinal and genitourinary disorders, chronic fatigue syndrome, fibromylagia syndrome, nervous system disorders, and combinations thereof, particularly those conditions selected from the group consisting of major depressive disorder, vasomotor symptoms, stress and urge urinary incontinence, fibromyalgia, pain, diabetic neuropathy, and combinations thereof.

In another embodiment, the present invention is directed to methods for treating or preventing vasomotor symptoms in a subject in need thereof, comprising the step of:
administering to said subject an effective amount of a compound of formula I, II, III, or pharmaceutically acceptable salt thereof.

In yet another embodiment, the present invention is directed to methods for treating or preventing a depression disorder in a subject in need thereof, comprising the step of:
administering to said subject an effective amount of a compound of formula I, II, III, or pharmaceutically acceptable salt thereof.

In yet other embodiments, the present invention is directed to methods for treating or preventing sexual dysfunction in a subject in need thereof, comprising the step of:
administering to said subject an effective amount of a compound of formula I, II, III, or pharmaceutically acceptable salt thereof.

In further embodiments, the present invention is directed to methods for treating or preventing pain in a subject in need thereof, comprising the step of:
administering to said subject an effective amount of a compound of formula I, II, III, or pharmaceutically acceptable salt thereof.

In another embodiment, the present invention is directed to methods for treating or preventing gastrointestinal or genitourinary disorder, particularly stress incontinence or urge urinary incontinence, in a subject in need thereof, comprising the step of:
administering to said subject an effective amount of a compound of formula I, II, III, or pharmaceutically acceptable salt thereof.

In another embodiment, the present invention is directed to methods for treating or preventing chronic fatigue syndrome in a subject in need thereof, comprising the step of:
administering to said subject an effective amount of a compound of formula I, II, III, or pharmaceutically acceptable salt thereof.

In another embodiment, the present invention is directed to methods for treating or preventing fibromylagia syndrome in a subject in need thereof, comprising the step of:
administering to said subject an effective amount of a compound of formula I, II, III, or pharmaceutically acceptable salt thereof.

In another embodiment, the present invention is directed to methods for treating or preventing schizophrenia in a subject in need thereof, comprising the step of:
administering to said subject an effective amount of a compound of formula I or II, or pharmaceutically acceptable salt thereof.

### BRIEF DESCRIPTION OF THE DRAWINGS

The invention can be more fully understood from the following detailed description and the accompanying drawings that form a part of this application.

FIG. 1 is an overview of estrogen action on norepinephrine/serotonin mediated thermoregulation.

FIG. 2 is a schematic representation of the interactions of norepinephrine and serotonin and their respective receptors (5-HT₂ₐ, α₁ and α₂-adrenergic).

### DETAILED DESCRIPTION OF THE INVENTION

The present invention is directed to phenylaminopropanol derivatives, compositions containing these derivatives, and methods of their use for the prevention and treatment of conditions ameliorated by monoamine reuptake including, inter alia, vasomotor symptoms (VMS), sexual dysfunction, gastrointestinal and genitourinary disorders, chronic fatigue syndrome, fibromylagia syndrome, nervous system disorders, and combinations thereof, particularly those conditions selected from the group consisting of major depressive disorder, vasomotor symptoms, stress and urge urinary incontinence, fibromyalgia, pain, diabetic neuropathy, and combinations thereof.

The following definitions are provided for the full understanding of terms and abbreviations used in this specification.

As used herein and in the appended claims, the singular forms “a,” “an,” and “the” include the plural reference unless the context clearly indicates otherwise. Thus, for example, a reference to “an antagonist” includes a plurality of such antagonists, and a reference to “a compound” is a reference to one or more compounds and equivalents thereof known to those skilled in the art, and so forth.

The abbreviations in the specification correspond to units of measure, techniques, properties, or compounds as follows: “min” means minutes, “h” means hour(s), “μL” means microliter(s), “mL” means milliliter(s), “mM” means millimolar, “M” means molar, “mmole” means millimole(s), “cm” means centimeters, “SEM” means standard error of the mean and “IU” means International Units. “Δ° C.” and Δ “ED₅₀ value” means dose which results in 50% alleviation of the observed condition or effect (50% mean maximum endpoint).

“Norepinephrine transporter” is abbreviated NET.

“Human norepinephrine transporter” is abbreviated hNET.

“Serotonin transporter” is abbreviated SERT.

“Human serotonin transporter” is abbreviated hSERT.

“Norepinephrine reuptake inhibitor” is abbreviated NRI.

“Selective norepinephrine reuptake inhibitor” is abbreviated SNRI.

“Serotonin reuptake inhibitor” is abbreviated SRI.

“Selective serotonin reuptake inhibitor” is abbreviated SSRI.

“Norepinephrine” is abbreviated NE.

“Serotonin” is abbreviated 5-HT.

“Subcutaneous” is abbreviated sc.

“Intraperitoneal” is abbreviated ip.

“Oral” is abbreviated po.

In the context of this disclosure, a number of terms shall be utilized. The term “treatment” as used herein includes preventative (e.g., prophylactic), curative or palliative treatment and “treating” as used herein also includes preventative, curative and palliative treatment.

The term “effective amount,” as used herein, refers to an amount effective, at dosages, and for periods of time necessary, to achieve the desired result with respect to prevention or treatment of vasomotor symptoms, depression disorders, sexual dysfunction, or pain. In particular, with respect to vasomotor symptoms, “effective amount” refers to the amount of compound or composition of compounds that would increase norepinephrine levels to compensate in part or total for the lack of steroid availability in subjects subject afflicted with a vasomotor symptom. Varying hormone levels will influence the amount of compound required in the present invention. For example, the pre-menopausal state may require a lower level of compound due to higher hormone levels than the peri-menopausal state.

It will be appreciated that the effective amount of components of the present invention will vary from patient to patient not only with the particular compound, component or composition selected, the route of administration, and the ability of the components (alone or in combination with one or more combination drugs) to elicit a desired response in the individual, but also with factors such as the disease state or severity of the condition to be alleviated, hormone levels, age, sex, weight of the individual, the state of being of the patient, and the severity of the pathological condition being treated, concurrent medication or special diets then being followed by the particular patient, and other factors which those skilled in the art will recognize, with the appropriate dosage ultimately being at the discretion of the attendant physician. Dosage regimens may be adjusted to provide the improved therapeutic response. An effective amount is also one in which any toxic or detrimental effects of the components are outweighed by the therapeutically beneficial effects.

Preferably, the compounds of the present invention are administered at a dosage and for a time such that the number of hot flushes is reduced as compared to the number of hot flushes prior to the start of treatment. Such treatment can also be beneficial to reduce the overall severity or intensity distribution of any hot flushes still experienced, as compared to the severity of hot flushes prior to the start of the treatment. With respect to depression disorders, sexual dysfunction, and pain, the compounds of the present invention are administered at a dosage and for a time such that there is the prevention, alleviation, or elimination of the symptom or condition.

For example, for an afflicted patient, compounds of formula I, or a pharmaceutically acceptable salt thereof, may be administered, preferably, at a dosage of from about 0.1 mg/day to about 500 mg/day, dosed one or two times daily, more preferably from about 1 mg/day to about 200 mg/day and most preferably from about 1 mg/day to 100 mg/day for a time sufficient to reduce and/or substantially eliminate the number and/or severity of hot flushes or symptom or condition of the depression disorder, sexual dysfunction, or pain.

The terms “component”, “drug” or “pharmacologically active agent” or “active agent” or “medicament” are used interchangeably herein to refer to a compound or compounds or composition of matter which, when administered to an organism (human or animal) induces a desired pharmacologic and/or physiologic effect by local and/or systemic action.

The term “modulation” refers to the capacity to either enhance or inhibit a functional property of a biological activity or process, for example, receptor binding or signaling activity. Such enhancement or inhibition may be contingent on the occurrence of a specific event, such as activation of a signal transduction pathway and/or may be manifest only in particular cell types. The modulator is intended to comprise any compound, e.g., antibody, small molecule, peptide, oligopeptide, polypeptide, or protein, preferably small molecule, or peptide.

As used herein, the term “inhibitor” refers to any agent that inhibits, suppresses, represses, or decreases a specific activity, such as serotonin reuptake activity or the norepinephrine reuptake activity, e.g., antibody, small molecule, peptide, oligopeptide, polypeptide, or protein, preferably small molecule or peptide, that exhibits a partial, complete, competitive and/or inhibitory effect on mammalian, preferably the human norepinephrine reuptake or both serotonin reuptake and the norepinephrine reuptake, thus diminishing or blocking, preferably diminishing, some or all of the biological effects of endogenous norepinephrine reuptake or of both serotonin reuptake and the norepinephrine reuptake.

Within the present invention, the compounds of formula I may be prepared in the form of pharmaceutically acceptable salts. As used herein, the term “pharmaceutically acceptable salts” refers to salts prepared from pharmaceutically acceptable non-toxic acids, including inorganic salts, and organic salts. Suitable non-organic salts include inorganic and organic acids such as acetic, benzenesulfonic, benzoic, camphorsulfonic, citric, ethenesulfonic, fumaric, gluconic, glutamic, hydrobromic, hydrochloric, isethionic, lactic, malic, maleic, mandelic, methanesulfonic, mucic, nitric, pamoic, pantothenic, phosphoric, succinic, sulfuric, tartaric acid, p-toluenesulfonic and the like. Particularly preferred are hydrochloric, hydrobromic, phosphoric, and sulfuric acids, and most preferably is the hydrochloride salt.

“Administering,” as used herein, means either directly administering a compound or composition of the present invention, or administering a prodrug, derivative or analog which will form an equivalent amount of the active compound or substance within the body.

The term “subject” or “patient” refers to an animal including the human species that is treatable with the compositions, and/or methods of the present invention. The term “subject” or “subjects” is intended to refer to both the male and female gender unless one gender is specifically indicated. Accordingly, the term “patient” comprises any mammal which may benefit from treatment or prevention of vasomotor symptoms, depression disorders, sexual dysfunction, or pain, such as a human, especially if the mammal is female, either in the pre-menopausal, peri-menopausal, or post-menopausal period. Furthermore, the term patient includes female animals including humans and, among humans, not only women of advanced age who have passed through menopause but also women who have undergone hysterectomy or for some other reason have suppressed estrogen production, such as those who have undergone long-term administration of corticosteroids, suffer from Cushing's syndrome or have gonadal dysgenesis. However, the term “patient” is not intended to be limited to a woman.

The terms “premature menopause” or “artificial menopause” refer to ovarian failure of unknown cause that may occur before age 40. It may be associated with smoking, living at high altitude, or poor nutritional status. Artificial menopause may result from oophorectomy, chemotherapy, radiation of the pelvis, or any process that impairs ovarian blood supply.

The term “pre-menopausal” means before the menopause, the term “peri-menopausal” means during the menopause and the term “post-menopausal” means after the menopause. “Ovariectomy” means removal of an ovary or ovaries and can be effected according to Merchenthaler et al., Maturitas, 1998, 30(3): 307-316.

“Side effect” refers to a consequence other than the one(s) for which an agent or measure is used, as the adverse effects produced by a drug, especially on a tissue or organ system other then the one sought to be benefited by its administration. In the case, for example, of high doses of NRIs or NRI/SRI compounds alone, the term “side effect” may refer to such conditions as, for example, vomiting, nausea, sweating, and flushes (Janowsky, et al., Journal of Clinical Psychiatry, 1984, 45(10 Pt 2): 3-9).

“Alkyl,” as used herein, refers to an optionally substituted, saturated straight, branched, or cyclic hydrocarbon having from about 1 to about 20 carbon atoms (and all combinations and subcombinations of ranges and specific numbers of carbon atoms therein), with from about 1 to about 8 carbon atoms being preferred, and with from about 1 to about 4 carbon atoms, herein referred to as “lower alkyl”, being more preferred. Alkyl groups include, but are not limited to, methyl, ethyl, n-propyl, isopropyl, n-butyl, isobutyl, t-butyl, n-pentyl, cyclopentyl, isopentyl, neopentyl, n-hexyl, isohexyl, cyclohexyl, cyclooctyl, adamantyl, 3-methylpentyl, 2,2-dimethylbutyl, and 2,3-dimethylbutyl.

“Heteroalkyl,” as used herein, refers to a substituent of the general formula (alkyl-X)ₙ-alkyl-, where each “alkyl” is independently as defined above, “X” is a sulfur, oxygen, or N heteroatom-containing moiety, and n is 1-4, preferably one. Heteroalkyl groups include, but are not limited to, methoxymethyl, ethoxyethyl, methoxyethyl, methylsulfanylmethyl, ethylsulfanylethyl, methylsulfanylethyl, methylaminoethyl, ethylaminoethyl, and methylaminoethyl.

“Alkenyl,” as used herein, refers to an alkyl group of at least two carbon atoms having one or more double bonds, wherein alkyl is as defined herein. Alkenyl groups can be optionally substituted.

“Alkynyl,” as used herein, refers to an alkyl group of at least two carbon atoms having one or more triple bonds, wherein alkyl is as defined herein. Alkynyl groups can be optionally substituted.

“Aryl” as used herein, refers to an optionally substituted, mono-, di-, tri-, or other multicyclic aromatic ring system having from about 5 to about 50 carbon atoms (and all combinations and subcombinations of ranges and specific numbers of carbon atoms therein), with from about 6 to about 10 carbons being preferred. Non-limiting examples include, for example, phenyl, naphthyl, anthracenyl, and phenanthrenyl.

“Heteroaryl,” as used herein, refers to an optionally substituted, mono-, di-, tri-, or other multicyclic aromatic ring system that includes at least one, and preferably from 1 to about 4 heteroatom ring members selected from sulfur, oxygen and nitrogen. Heteroaryl groups can have, for example, from about 3 to about 50 carbon atoms (and all combinations and subcombinations of ranges and specific numbers of carbon atoms therein), with from about 4 to about 10 carbons being preferred. Non-limiting examples of heteroaryl groups include, for example, pyrryl, furyl, pyridyl, 1,2,4-thiadiazolyl, pyrimidyl, thienyl, isothiazolyl, imidazolyl, tetrazolyl, pyrazinyl, pyrimidyl, quinolyl, isoquinolyl, thiophenyl, benzothienyl, isobenzofuryl, pyrazolyl, indolyl, purinyl, carbazolyl, benzimidazolyl, and isoxazolyl.

“Heterocyclic ring,” as used herein, refers to a stable 5- to 7-membered monocyclic or bicyclic or 7- to 10-membered bicyclic heterocyclic ring that is saturated, partially unsaturated or unsaturated (aromatic), and which contains carbon atoms and from 1 to 4 heteroatoms independently selected from the group consisting of N, O and S and including any bicyclic group in which any of the above defined heterocyclic rings is fused to a benzene ring. The nitrogen and sulfur heteroatoms may optionally be oxidized. The heterocyclic ring may be attached to its pendant group at any heteroatom or carbon atom that results in a stable structure. The heterocyclic rings described herein may be substituted on carbon or on a nitrogen atom if the resulting compound is stable. If specifically noted, a nitrogen atom in the heterocycle may optionally be quaternized. It is preferred that when the total number of S and O atoms in the heterocycle exceeds one, then these heteroatoms are not adjacent to one another. It is preferred that the total number of S and O atoms in the heterocycle is not more than one. Examples of heterocycles include, but are not limited to, 1H-indazole, 2-pyrrolidonyl, 2H,6H-1,5,2-dithiazinyl, 2H-pyrrolyl, 3H-indolyl, 4-piperidonyl, 4aH-carbazole, 4H-quinolizinyl, 6H-1,2,5-thiadiazinyl, acridinyl, azocinyl, benzimidazolyl, benzofuranyl, benzothiofuranyl, benzothiophenyl, benzoxazolyl, benzthiazolyl, benztriazolyl, benztetrazolyl, benzisoxazolyl, benzisothiazolyl, benzimidazalonyl, carbazolyl, 4H-carbazolyl, α-, β-, or γ-carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, indolenyl, indolinyl, indolizinyl, indolyl, isobenzofuranyl, isochromanyl, isoindazolyl, isoindolinyl, isoindolyl, isoquinolinyl, isothiazolyl, isoxazolyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolidinylpyrimidinyl, phenanthridinyl, phenanthrolinyl, phenoxazinyl, phenazinyl, phenothiazinyl, phenoxathiinyl, phenoxazinyl, phthalazinyl, piperazinyl, piperidinyl, pteridinyl, piperidonyl, 4-piperidonyl, pteridinyl, purinyl, pyranyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, 4H-quinolizinyl, quinoxalinyl, quinuclidinyl, carbolinyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, xanthenyl. Preferred heterocycles include, but are not limited to, pyridinyl, furanyl, thienyl, pyrrolyl, pyrazolyl, imidazolyl, indolyl, benzimidazolyl, 1H-indazolyl, oxazolidinyl, benzotriazolyl, benzisoxazolyl, oxindolyl, benzoxazolinyl, or isatinyl. Also included are fused ring and spiro compounds containing, for example, the above heterocycles.

“Alkoxy,” as used herein, refers to the group R—O— where R is an alkyl group as defined herein.

“Aryloxy,” as used herein, refers to the group R—O— where R is an aryl group, as defined herein.

“Heteroaryloxy,” as used herein, refers to the group R—O— where R is a heteroaryl group, as defined herein.

“Heteroarylmethyl” as used herein, refers to the group R—CH₂— where R is a heteroaryl group, as defined herein.

“Heteroarylmethoxy,” as used herein, refers to the group R—CH₂—O— where R is a heteroaryl group, as defined herein.

“Arylalkoxy,” as used herein, refers to the group R_{z}—Rₓ—O— where R_{z} is an aryl group and Rₓ is an alkyl group, as defined herein.

“Alkanoyloxy,” as used herein, refers to the group R—C(═O)—O— where R is an alkyl group of 1 to 5 carbon atoms.

“Arylalkyl” as used herein, refers to the group R_{z}—R_{y}— where R_{z} is an aryl group, as defined herein, and where R_{y} is an alkyl group, as defined herein.

“Alkylsulfoxide,” as used herein, refers to as used herein, refers to —S(═O)—R, where R is alkyl, as defined above.

“Alkylsulfone,” as used herein, refers to —S(═O)₂—R, where R is alkyl, as defined above.

“Arylsulfoxide,” as used herein, refers to as used herein, refers to —S(═O)—R, where R is aryl, as defined above.

“Arylsulfone,” as used herein, refers to —S(═O)₂—R, where R is aryl, as defined above.

“Alkylsulfonamide,” as used herein, refers to —NR—S(═O)₂—R, where each R is independently, alkyl, as defined above or the NR part may also be NH.

“Arylsulfonamide,” as used herein, refers to —NR—S(═O)₂—R, where each R is independently, aryl, as defined above or the NR part may also be NH (provided that the other R is aryl).

“Heteroarylmethoxy,” as used herein, refers to —OCH₂—R, where R is heteroaryl, as defined above.

“Alkylamido,” as used herein, refers to —NR—C(═O)—R, where each R is independently, alkyl, as defined above, or the NR part may also be NH.

“Arylamido,” as used herein, refers to —NR_{y}—C(═O)—R_{z}, where R_{y} and R_{z} are H or aryl (provided that at least one of R_{y} and R_{z} is aryl), as defined above.

“Halo,” as used herein, refers to chloro, bromo, fluoro, and iodo.

When any variable occurs more than one time in any constituent or any formula, its definition in each occurrence is independent of its definition at every other occurrence. Combinations of substituents and/or variables and/or replacements atoms or groups are permissible only if such combinations result in a stable compound.

In one embodiment, the invention is directed to compounds of A compound of formula I:

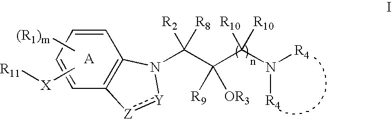

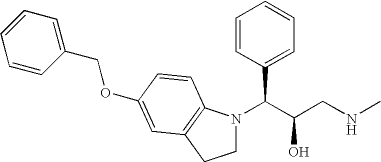

or a pharmaceutically acceptable salt thereof;

wherein:

the dotted line between Y and Z represents an optional second bond;

the dotted line between the two R₄ groups represents an optional heterocyclic ring of 4 to 6 ring atoms that may be formed between the two R₄ groups, together with the nitrogen through which they are attached;

X is —(C(R₁₂)₂)ₒ—, —O(C(R₁₂)₂)ₒ—, —(C(R₁₂)₂)ₒO—, —S(O)ₚ(C(R₁₂)₂)ₒ—, —(C(R₁₂)₂)ₒS(O)ₚ—, —N(R₁₃)C(O)(C(R₁₂)₂)ₒ—, —(C(R₁₂)₂)ₒC(O)N(R₁₃)—, —C(O)N(R₁₃)(C(R₁₂)₂)ₒ—, —(C(R₁₂)₂)ₒN(R₁₃)C(O)—, —(C(R₁₂)₂)ₒN(R₁₃)S(O)₂—, —S(O)₂N(R₁₃)(C(R₁₂)₂)ₒ—, —N(R₁₃)S(O)₂(C(R₁₂)₂)ₒ—, —(C(R₁₂)₂)ₒS(O)₂N(R₁₃)—, —NR₇(C(R₁₂)₂)ₒ—, —(C(R₁₂)₂)ₒNR₇—, or —C≡C—;

Y is N, C(R₆)₂, CR₆, or C═O;

Z is O, S(O)ₚ, N, NR₇, CR₅, or C(R₅)₂;

R₁ is, independently at each occurrence, alkyl, alkoxy, halo, CF₃, OCF₃, hydroxy, alkanoyloxy, nitro, cyano, alkenyl, alkynyl, alkylsulfoxide, alkylsulfone, alkylsulfonamide, or alkylamido; or two adjacent R₁ also represent methylenedioxy;

R₂ is aryl substituted with 0-3 R₁₄ or heteroaryl substituted with 0-3 R₁₄;

R₃ is H or C₁-C₄ alkyl;

R₄ is, independently at each occurrence, H, C₁-C₄ alkyl, C₃-C₆ cycloalkyl, arylalkyl, heteroarylmethyl, cycloheptylmethyl, cyclohexylmethyl, cyclopentylmethyl, or cyclobutylmethyl, or

both R₄ groups, together with the nitrogen through which they are attached, form a heterocyclic ring of 4 to 6 ring atoms, where one carbon may be optionally replaced with N, O, S, or SO₂, and where any carbon ring atom or additional N atom may be optionally substituted with C₁-C₄ alkyl, F, or CF₃;

R₅ is, independently at each occurrence, H, C₁-C₄ alkyl, aryl substituted with 0-3 R₁₄, heteroaryl substituted with 0-3 R₁₄, or cyano; or when two R₅ are present, they may form a carbocyclic ring of 3-5 carbons;

R₆ is, independently at each occurrence, H, C₁-C₄ alkyl, or cyano;

R₇ is H, C₁-C₆ alkyl, C₃-C₆ cycloalkyl, aryl substituted with 0-3 R₁₄; or heteroaryl substituted with 0-3 R₁₄.

R₈ is H, or C₁-C₄ alkyl;

R₉ is H, or C₁-C₄ alkyl;

R₁₀ is, independently at each occurrence, H, or C₁-C₄ alkyl; or R₁₀ and R₄ together with the nitrogen to which R₄ is attached form a nitrogen-containing ring containing 3-6 carbon atoms;

R₁₁ is aryl substituted with 0-3 R₁ or heteroaryl substituted with 0-3 R₁;

R₁₂ is, independently at each occurrence, H, C₁-C₄ alkyl;

R₁₃ is H or C₁-C₄ alkyl;

R₁₄ is, independently at each occurrence, alkyl, alkoxy, halo, CF₃, OCF₃, arylalkyloxy substituted with 0-3 R₁, aryloxy substituted with 0-3 R₁, aryl substituted with 0-3 R₁, heteroaryl substituted with 0-3 R₁, hydroxy, alkanoyloxy, nitro, cyano, alkenyl, alkynyl, alkylsulfoxide, phenylsulfoxide substituted with 0-3 R₁, alkylsulfone, phenylsulfone substituted with 0-3 R₁, alkylsulfonamide, phenylsulfonamide substituted with 0-3 R₁, heteroaryloxy substituted with 0-3 R₁, heteroarylmethyloxy substituted with 0-3 R₁, alkylamido, or arylamido substituted with 0-3 R₁; or two adjacent R₁ also represent methylenedioxy;

m is an integer from 0 to 3;

n is an integer from 1 to 2;

o is an integer from 0 to 3; and

p is an integer from 0 to 2;

wherein 1-3 carbon atoms in ring A may optionally be replaced with N.

In preferred embodiments of the compound of formula I,

the dotted line between Y and Z represents a second bond;

Y is CR₆;

Z is CR₅.

In preferred embodiments of the compound of formula I,

the bond between Y and Z is a single bond;

Y is C(R₆)₂; and

Z is C(R₅)₂.

In preferred embodiments of the compound of formula I,

the bond between Y and Z is a single bond;

Y is C═O; and

Z is C(R₅)₂.

In preferred embodiments of the compound of formula I,

the bond between Y and Z is a single bond;

Y is C═O; and

Z is NR₇.

In preferred embodiments of the compound of formula I, X is —(C(R₁₂)₂)ₒ—, —(C(R₁₂)₂)ₒO—, or —C≡C—.

In preferred embodiments of the compound of formula I, Y is C(R₆)₂, CR₆, or C═O.

In preferred embodiments of the compound of formula I, Z is CR₅ or C(R₅)₂.

In preferred embodiments of the compound of formula I, R₁ is, independently at each occurrence, alkyl, alkoxy, halo, CF₃, OCF₃, hydroxy, alkanoyloxy, nitro, or cyano.

In preferred embodiments of the compound of formula I, R₂ is aryl substituted with 0-2 R₁₄, especially, R₂ is phenyl, fluorophenyl, or difluorophenyl.

In preferred embodiments of the compound of formula I, R₃ is H.

In preferred embodiments of the compound of formula I, R₄ is H or methyl.

In preferred embodiments of the compound of formula I, R₅ is, independently at each occurrence, H, C₁-C₄ alkyl, aryl substituted with 0-3 R₁₄, especially H, methyl, ethyl, n-propyl, isopropyl, aryl substituted with alkoxy, aryl substituted with aryloxy or phenyl substituted with 1-2 halo.

In preferred embodiments of the compound of formula I, R₆ is, independently at each occurrence, H, methyl, ethyl, n-propyl, or isopropyl.

In preferred embodiments of the compound of formula I, R₇ is H, C₁-C₆ alkyl, or aryl substituted with 0-3 R₁₄.

In preferred embodiments of the compound of formula I, R₈ is H.

In preferred embodiments of the compound of formula I, R₉ is H.

In preferred embodiments of the compound of formula I, R₁₀ is H.

In preferred embodiments of the compound of formula I, R₁₁ is aryl substituted with 0-3 R₁, especially R₁₁ is aryl substituted with 0-2 R₁, and more especially, phenyl, or aryl substituted with 1-2 halo or alkoxy.

In preferred embodiments of the compound of formula I, n is 1.

In preferred embodiments of the compound of formula I, none of the carbon atoms in ring A are replaced with N.

In preferred embodiments of the compound of formula I,

the dotted line between Y and Z represents a second bond;

Y is CR₆;

Z is CR₅;

X is —(C(R₁₂)₂)ₒ—, —(C(R₁₂)₂)ₒO—, or —C≡C—;

R₁ is, independently at each occurrence, alkyl, alkoxy, halo, CF₃, OCF₃, hydroxy or cyano;

R₂ is aryl substituted with 0-3 R₁₄ or heteroaryl substituted with 0-3 R₁₄;

R₃ is H;

R₄ is, independently at each occurrence, H or methyl;

R₅ is, independently at each occurrence, H, methyl or aryl substituted with 0-3 R₁₄;

R₆ is H;

R₈ is H;

R₉ is H;

R₁₀ is H;

R₁₁ is aryl substituted with 0-3 R₁ or heteroaryl substituted with 0-3 R₁;

R₁₂ is, independently at each occurrence, H or C₁-C₄ alkyl;

R₁₄ is, independently at each occurrence, alkyl, alkoxy, halo, CF₃, OCF₃, hydroxy or cyano;

m is an integer from 0 to 2;

n is 1; and

o is an integer from 0 to 3;

wherein none of the carbon atoms in ring A are replaced with N.

In preferred embodiments of the compound of formula I,

the bond between Y and Z is a single bond;

Y is C(R₆)₂;

Z is C(R₅)₂;

X is —(C(R₁₂)₂)ₒ—, —(C(R₁₂)₂)ₒO—, or —C≡C—;

R₁ is, independently at each occurrence, alkyl, alkoxy, halo, CF₃, OCF₃, hydroxy or cyano;

R₂ is aryl substituted with 0-3 R₁₄ or heteroaryl substituted with 0-3 R₁₄;

R₃ is H;

R₄ is, independently at each occurrence, H or methyl;

R₅ is, independently at each occurrence, H, C₁-C₄ alkyl or aryl substituted with 0-3 R₁₄;

R₆ is independently at each occurrence, H or C₁-C₄ alkyl;

R₈ is H;

R₉ is H;

R₁₀ is H;

R₁₁ is aryl substituted with 0-3 R₁ or heteroaryl substituted with 0-3 R₁;

R₁₂ is, independently at each occurrence, H or C₁-C₄ alkyl;

R₁₄ is, independently at each occurrence, alkyl, alkoxy, halo, CF₃, OCF₃, hydroxy or cyano;

m is an integer from 0 to 2;

n is 1; and

o is an integer from 0 to 3;

wherein none of the carbon atoms in ring A are replaced with N.

In preferred embodiments of the compound of formula I,

the bond between Y and Z is a single bond;

Y is C═O;

Z is C(R₅)₂;

X is —(C(R₁₂)₂)ₒ—, —(C(R₁₂)₂)ₒO—, or —C≡C—;

R₁ is, independently at each occurrence, alkyl, alkoxy, halo, CF₃, OCF₃, hydroxy or cyano;

R₂ is aryl substituted with 0-3 R₁₄ or heteroaryl substituted with 0-3 R₁₄;

R₃ is H;

R₄ is, independently at each occurrence, H or methyl;

R₅ is, independently at each occurrence, H, or C₁-C₄ alkyl;

R₈ is H;

R₉ is H;

R₁₀ is H;

R₁₁ is aryl substituted with 0-3 R₁ or heteroaryl substituted with 0-3 R₁;

R₁₂ is, independently at each occurrence, H or C₁-C₄ alkyl;

R₁₄ is, independently at each occurrence, alkyl, alkoxy, halo, CF₃, OCF₃, hydroxy or cyano;

m is an integer from 0 to 2;

n is 1; and

o is an integer from 0 to 3;

wherein none of the carbon atoms in ring A are replaced with N.

In preferred embodiments of the compound of formula I,

the bond between Y and Z is a single bond;

Y is C═O;

Z is NR₇;

X is —(C(R₁₂)₂)ₒ—, —(C(R₁₂)₂)ₒO—, or —C≡C—;

R₁ is, independently at each occurrence, alkyl, alkoxy, halo, CF₃, OCF₃, hydroxy or cyano;

R₂ is aryl substituted with 0-3 R₁₄ or heteroaryl substituted with 0-3 R₁₄;

R₃ is H;

R₄ is, independently at each occurrence, H or methyl;

R₇ is C₁-C₆ alkyl, C₃-C₆ cycloalkyl, aryl substituted with 0-3 R₁₄ or heteroaryl substituted with 0-3 R₁₄;

R₈ is H;

R₉ is H;

R₁₀ is H;

R₁₁ is aryl substituted with 0-3 R₁ or heteroaryl substituted with 0-3 R₁;

R₁₂ is, independently at each occurrence, H or C₁-C₄ alkyl;

R₁₄ is, independently at each occurrence, alkyl, alkoxy, halo, CF₃, OCF₃, hydroxy or cyano;

m is an integer from 0 to 2;

n is 1; and

o is an integer from 0 to 3;

wherein none of the carbon atoms in ring A are replaced with N.

In another embodiment, the invention is directed to compounds of formula II:

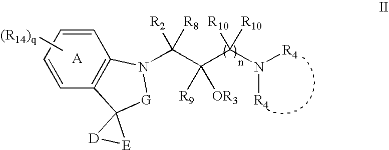

or a pharmaceutically acceptable salt thereof;

wherein:

D and E, together with the carbon atom through which they are attached, form a carbocyclic ring of 6 to 8 atoms or a heterocyclic ring of 5 to 8 atoms containing 1 to 2 heteroatoms selected from O, S(O)ₚ, and NR₇, where any carbon ring atom may be optionally substituted with C₁-C₄ alkyl, F or CF₃;

the dotted line between the two R₄ groups represents an optional heterocyclic ring of 4 to 6 ring atoms that may be formed between the two R₄ groups, together with the nitrogen through which they are attached;

G is NR₇, C(R₆)₂, or C═O;

R₁ is, independently at each occurrence, alkyl, alkoxy, halo, CF₃, OCF₃, hydroxy, alkanoyloxy, nitro, cyano, alkenyl, alkynyl, alkylsulfoxide, alkylsulfone, alkylsulfonamide, or alkylamido; or two adjacent R₁ also represent methylenedioxy;

R₂ is aryl substituted with 0-3 R₁₄ or heteroaryl substituted with 0-3 R₁₄;

R₃ is H or C₁-C₄ alkyl;

R₄ is, independently at each occurrence, H, C₁-C₄ alkyl, C₃-C₆ cycloalkyl, arylalkyl, heteroarylmethyl, cycloheptylmethyl, cyclohexylmethyl, cyclopentylmethyl, or cyclobutylmethyl, or

both R₄ groups, together with the nitrogen through which they are attached, form a heterocyclic ring of 4 to 6 ring atoms, where one carbon may be optionally replaced with N, O, S, or SO₂, and where any carbon ring atom or additional N atom may be optionally substituted with C₁-C₄ alkyl, F, or CF₃;

R₆ is, independently at each occurrence, H, C₁-C₄ alkyl, or cyano;

R₇ is H, C₁-C₆ alkyl, C₃-C₆ cycloalkyl, aryl substituted with 0-3 R₁₄; or heteroaryl substituted with 0-3 R₁₄.

R₈ is H, or C₁-C₄ alkyl;

R₉ is H, or C₁-C₄ alkyl;

R₁₀ is, independently at each occurrence, H, or C₁-C₄ alkyl; or R₁₀ and R₄ together with the nitrogen to which R₄ is attached form a nitrogen-containing ring containing 3-6 carbon atoms;

R₁₄ is, independently at each occurrence, alkyl, alkoxy, halo, CF₃, OCF₃, arylalkyloxy substituted with 0-3 R₁, aryloxy substituted with 0-3 R₁, aryl substituted with 0-3 R₁, heteroaryl substituted with 0-3 R₁, hydroxy, alkanoyloxy, nitro, cyano, alkenyl, alkynyl, alkylsulfoxide, phenylsulfoxide substituted with 0-3 R₁, alkylsulfone, phenylsulfone substituted with 0-3 R₁, alkylsulfonamide, phenylsulfonamide substituted with 0-3 R₁, heteroaryloxy substituted with 0-3 R₁, heteroarylmethyloxy substituted with 0-3 R₁, alkylamido, or arylamido substituted with 0-3 R₁; or two adjacent R₁ also represent methylenedioxy;

n is an integer from 1 to 2;

p is an integer from 0 to 2; and

q is an integer from 0 to 4;

wherein 1-3 carbon atoms in ring A may optionally be replaced with N.

In preferred embodiments of the compound of formula II, G is C═O.

In preferred embodiments of the compound of formula II, G is C(R₆)₂.

In preferred embodiments of the compound of formula II, R₁ is, independently at each occurrence, alkyl, alkoxy, halo, CF₃, OCF₃, hydroxy, alkanoyloxy, nitro, or cyano.

In preferred embodiments of the compound of formula II, R₂ is aryl substituted with 0-2 R₁₄.

In preferred embodiments of the compound of formula II, R₂ is phenyl, fluorophenyl, or difluorophenyl.

In preferred embodiments of the compound of formula II, R₃ is H.

In preferred embodiments of the compound of formula II, R₄ is H or methyl.

In preferred embodiments of the compound of formula II, R₆ is, independently at each occurrence, H, methyl, ethyl, n-propyl, or isopropyl.

In preferred embodiments of the compound of formula II, R₇ is H, C₁-C₆ alkyl, or aryl substituted with 0-3 R₁₄.

In preferred embodiments of the compound of formula II, R₈ is H.

In preferred embodiments of the compound of formula II, R₉ is H.

In preferred embodiments of the compound of formula II, R₁₀ is H.

In preferred embodiments of the compound of formula II, R₁₄ is, independently at each occurrence, alkyl, alkoxy, halo, CF₃, OCF₃, hydroxy, alkanoyloxy, nitro, or cyano.

In preferred embodiments of the compound of formula II, n is 1.

In preferred embodiments of the compound of formula II, p is 0 or 1.

In preferred embodiments of the compound of formula II, none of the carbon atoms in ring A are replaced with N.

In preferred embodiments of the compound of formula II,

D and E, together with the carbon atom through which they are attached, form a carbocyclic ring of 6 to 7 atoms;

G is C(R₆)₂;

R₂ is aryl substituted with 0-3 R₁₄ or heteroaryl substituted with 0-3 R₁₄;

R₃ is H;

R₄ is, independently at each occurrence, H or methyl;

R₆ is, independently at each occurrence, H or C₁-C₄ alkyl;

R₈ is H;

R₉ is H;

R₁₀ is H;

R₁₄ is, independently at each occurrence, alkyl, alkoxy, halo, CF₃, OCF₃, hydroxy or cyano;

n is 1; and

q is an integer from 0 to 3;

wherein none of the carbon atoms in ring A are replaced with N.

In preferred embodiments of the compound of formula II,

D and E, together with the carbon atom through which they are attached, form a carbocyclic ring of 6 to 7 atoms;

G is C═O;

R₂ is aryl substituted with 0-3 R₁₄ or heteroaryl substituted with 0-3 R₁₄;

R₃ is H;

R₄ is, independently at each occurrence, H or methyl;

R₈ is H;

R₉ is H;

R₁₀ is H;

R₁₄ is, independently at each occurrence, alkyl, alkoxy, halo, CF₃, OCF₃, hydroxy or cyano;

n is 1;

q is an integer from 0 to 3;

wherein none of the carbon atoms in ring A are replaced with N.

In yet another embodiment, the invention is directed to compounds of formula III:

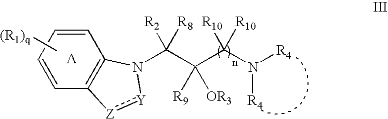

or a pharmaceutically acceptable salt thereof;

wherein:

the dotted line between Y and Z represents an optional second bond;

the dotted line between the two R₄ groups represents an optional heterocyclic ring of 4 to 6 ring atoms that may be formed between the two R₄ groups, together with the nitrogen through which they are attached;

Y is N, C(R₆)₂, CR₆, or C═O;

Z is O, S(O)ₚ, N, NR₇, CR₅, or C(R₅)₂;

R₁ is, independently at each occurrence, alkyl, alkoxy, halo, CF₃, OCF₃, hydroxy, alkanoyloxy, nitro, cyano, alkenyl, alkynyl, alkylsulfoxide, alkylsulfone, alkylsulfonamide, or alkylamido; or two adjacent R₁ also represent methylenedioxy;

R₂ is aryl substituted with 0-3 R₁₄ or heteroaryl substituted with 0-3 R₁₄;

R₃ is H or C₁-C₄ alkyl;

R₄ is, independently at each occurrence, H, C₁-C₄ alkyl, C₃-C₆ cycloalkyl, arylalkyl, heteroarylmethyl, cycloheptylmethyl, cyclohexylmethyl, cyclopentylmethyl, or cyclobutylmethyl, or

both R₄ groups, together with the nitrogen through which they are attached, form a heterocyclic ring of 4 to 6 ring atoms, where one carbon may be optionally replaced with N, O, S, or SO₂, and where any carbon ring atom or additional N atom may be optionally substituted with C₁-C₄ alkyl, F, or CF₃;

R₅ is, independently at each occurrence, H, C₁-C₄ alkyl, aryl substituted with 0-3 R₁₄, heteroaryl substituted with 0-3 R₁₄, or cyano; or when two R₅ are present, they may form a carbocyclic ring of 3-5 carbons;

R₆ is, independently at each occurrence, H, C₁-C₄ alkyl, or cyano;

R₇ is H, C₁-C₆ alkyl, C₃-C₆ cycloalkyl, aryl substituted with 0-3 R₁₄, or heteroaryl substituted with 0-3 R₁₄;

R₈ is H, or C₁-C₄ alkyl;

R₉ is H, or C₁-C₄ alkyl;

R₁₀ is, independently at each occurrence, H, or C₁-C₄ alkyl; or R₁₀ and R₄ together with the nitrogen to which R₄ is attached form a nitrogen-containing ring containing 3-6 carbon atoms;

R₁₄ is, independently at each occurrence, alkyl, alkoxy, halo, CF₃, OCF₃, arylalkyloxy substituted with 0-3 R₁, aryloxy substituted with 0-3 R₁, aryl substituted with 0-3 R₁, heteroaryl substituted with 0-3 R₁, hydroxy, alkanoyloxy, nitro, cyano, alkenyl, alkynyl, alkylsulfoxide, phenylsulfoxide substituted with 0-3 R₁, alkylsulfone, phenylsulfone substituted with 0-3 R₁, alkylsulfonamide, phenylsulfonamide substituted with 0-3 R₁, heteroaryloxy substituted with 0-3 R₁, heteroarylmethyloxy substituted with 0-3 R₁, alkylamido, or arylamido substituted with 0-3 R₁; or two adjacent R₁ also represent methylenedioxy;

n is an integer from 1 to 2; and

q is an integer from 0 to 4;

wherein 1-3 carbon atoms in ring A may optionally be replaced with N.

In preferred embodiments of the compound of formula III,

the dotted line between Y and Z represents a second bond.

Y is CR₆; and

Z is CR₅.

In preferred embodiments of the compound of formula III,

the bond between Y and Z is a single bond;

Y is C(R₆)₂; and

Z is C(R₅)₂.

In preferred embodiments of the compound of formula III,

the bond between Y and Z is a single bond;

Y is C═O;

Z is C(R₅)₂.

In preferred embodiments of the compound of formula III,

the bond between Y and Z is a single bond;

Y is C═O;

Z is NR₇.

In preferred embodiments of the compound of formula III, Y is C(R₆)₂, CR₆, or C═O.

In preferred embodiments of the compound of formula III, Z is CR₅ or C(R₅)₂.

In preferred embodiments of the compound of formula III, R₁ is, independently at each occurrence, alkyl, alkoxy, halo, CF₃, OCF₃, hydroxy, alkanoyloxy, nitro, or cyano.

In preferred embodiments of the compound of formula III, R₂ is aryl substituted with 0-2 R₁₄.

In preferred embodiments of the compound of formula III, R₂ is phenyl, fluorophenyl, or difluorophenyl.

In preferred embodiments of the compound of formula III, R₃ is H.

In preferred embodiments of the compound of formula III, R₄ is H or methyl.

In preferred embodiments of the compound of formula III, R₅ is, independently at each occurrence, H, C₁-C₄ alkyl, aryl substituted with 0-3 R₁₄.

In preferred embodiments of the compound of formula III, R₅ is, independently at each occurrence, H, methyl, ethyl, n-propyl, isopropyl, aryl substituted with alkoxy, aryl substituted with aryloxy or phenyl substituted with 1-2 halo.

In preferred embodiments of the compound of formula III, R₆ is, independently at each occurrence, H, methyl, ethyl, n-propyl, or isopropyl.

In preferred embodiments of the compound of formula III, R₇ is H, C₁-C₆ alkyl, or aryl substituted with 0-3 R₁₄.

In preferred embodiments of the compound of formula III, R₈ is H.

In preferred embodiments of the compound of formula III, R₉ is H.

In preferred embodiments of the compound of formula III, R₁₀ is H.

In preferred embodiments of the compound of formula III, n is 1.

In preferred embodiments of the compound of formula III, q is an integer from 0 to 2.

In preferred embodiments of the compound of formula III, none of the carbon atoms in ring A are replaced with N.

In preferred embodiments of the compound of formula III, the dotted line between Y and Z represents a second bond;

Y is CR₆;

Z is CR₅;

R₁ is, independently at each occurrence, alkyl, alkoxy, halo, CF₃, OCF₃, hydroxy or cyano;

R₂ is aryl substituted with 0-3 R₁₄ or heteroaryl substituted with 0-3 R₁₄;

R₃ is H;

R₄ is, independently at each occurrence, H or methyl;

R₅ is, independently at each occurrence, H, methyl or aryl substituted with 0-3 R₁₄;

R₆ is H;

R₈ is H;

R₉ is H;

R₁₀ is H;

R₁₄ is, independently at each occurrence, alkyl, alkoxy, halo, CF₃, OCF₃, hydroxy or cyano;

n is 1; and

q is an integer from 0 to 3;

wherein none of the carbon atoms in ring A are replaced with N.

In preferred embodiments of the compound of formula III,

the bond between Y and Z is a single bond;

Y is C(R₆)₂;

Z is C(R₅)₂;

R₁ is, independently at each occurrence, alkyl, alkoxy, halo, CF₃, OCF₃, hydroxy or cyano;

R₂ is aryl substituted with 0-3 R₁₄ or heteroaryl substituted with 0-3 R₁₄;

R₃ is H;

R₄ is, independently at each occurrence, H or methyl;

R₅ is, independently at each occurrence, H, C₁-C₄ alkyl or aryl substituted with 0-3 R₁₄;

R₆ is independently at each occurrence, H or C₁-C₄ alkyl;

R₈ is H;

R₉ is H;

R₁₀ is H;

R₁₄ is, independently at each occurrence, alkyl, alkoxy, halo, CF₃, OCF₃, hydroxy or cyano;

n is 1; and

q is an integer from 0 to 3;

wherein none of the carbon atoms in ring A are replaced with N.

In preferred embodiments of the compound of formula III,

the bond between Y and Z is a single bond;

Y is C═O;

Z is C(R₅)₂;

R₁ is, independently at each occurrence, alkyl, alkoxy, halo, CF₃, OCF₃, hydroxy or cyano;

R₂ is aryl substituted with 0-3 R₁₄ or heteroaryl substituted with 0-3 R₁₄;

R₃ is H;

R₄ is, independently at each occurrence, H or methyl;

R₅ is, independently at each occurrence, H, or C₁-C₄ alkyl;

R₈ is H;

R₉ is H;

R₁₀ is H;

R₁₄ is, independently at each occurrence, alkyl, alkoxy, halo, CF₃, OCF₃, hydroxy or cyano;

n is 1; and

q is an integer from 0 to 3;

wherein none of the carbon atoms in ring A are replaced with N.

In preferred embodiments of the compound of formula III,

the bond between Y and Z is a single bond;

Y is C═O;

Z is NR₇;

R₁ is, independently at each occurrence, alkyl, alkoxy, halo, CF₃, OCF₃, hydroxy or cyano;

R₂ is aryl substituted with 0-3 R₁₄ or heteroaryl substituted with 0-3 R₁₄;

R₃ is H;

R₄ is, independently at each occurrence, H or methyl;

R₇ is C₁-C₆ alkyl, C₃-C₆ cycloalkyl, aryl substituted with 0-3 R₁₄ or heteroaryl substituted with 0-3 R₁₄;

R₈ is H;

R₉ is H;

R₁₀ is H;

R₁₄ is, independently at each occurrence, alkyl, alkoxy, halo, CF₃, OCF₃, hydroxy or cyano;

n is 1; and

q is an integer from 0 to 3;

wherein none of the carbon atoms in ring A are replaced with N.

Preferred compounds of the invention include, but are not limited to:
1-[5-(benzyloxy)-1H-indol-1-yl]-3-(methylamino)-1-phenylpropan-2-ol;
1-[4-(benzyloxy)-1H-indol-1-yl]-3-(methylamino)-1-phenylpropan-2-ol;
1-[6-(benzyloxy)-1H-indol-1-yl]-3-(methylamino)-1-phenylpropan-2-ol;
1-[7-(benzyloxy)-1H-indol-1-yl]-3-(methylamino)-1-phenylpropan-2-ol;
1-{5-[(2-methoxybenzyl)oxy]-1H-indol-1-yl}-3-(methylamino)-1-phenylpropan-2-ol;
1-{5-[(3-methoxybenzyl)oxy]-1H-indol-1-yl}-3-(methylamino)-1-phenylpropan-2-ol;
1-{5-[(4-methoxybenzyl)oxy]-1H-indol-1-yl}-3-(methylamino)-1-phenylpropan-2-ol;
1-{5-[(2-chlorobenzyl)oxy]-1H-indol-1-yl}-3-(methylamino)-1-phenylpropan-2-ol;
1-{5-[(3-chlorobenzyl)oxy]-1H-indol-1-yl}-3-(methylamino)-1-phenylpropan-2-ol;
1-{5-[(4-chlorobenzyl)oxy]-1H-indol-1-yl}-3-(methylamino)-1-phenylpropan-2-ol;
1-{5-[(2-fluorobenzyl)oxy]-1H-indol-1-yl}-3-(methylamino)-1-phenylpropan-2-ol;
1-{5-[(3-fluorobenzyl)oxy]-1H-indol-1-yl}-3-(methylamino)-1-phenylpropan-2-ol;
1-{5-[(4-fluorobenzyl)oxy]-1H-indol-1-yl}-3-(methylamino)-1-phenylpropan-2-ol;
3-(methylamino)-1-{5-[(2-methylbenzyl)oxy]-1H-indol-1-yl}-1-phenylpropan-2-ol;
3-(methylamino)-1-{5-[(3-methylbenzyl)oxy]-1H-indol-1-yl}-1-phenylpropan-2-ol;
3-(methylamino)-1-{5-[(4-methylbenzyl)oxy]-1H-indol-1-yl}-1-phenylpropan-2-ol;
3-(methylamino)-1-phenyl-1-[5-(1-phenylethoxy)-1H-indol-1-yl]propan-2-ol;
3-(methylamino)-1-phenyl-1-[5-(2-phenylethoxy)-1H-indol-1-yl]propan-2-ol;
3-(methylamino)-1-(5-phenoxy-1H-indol-1-yl)-1-phenylpropan-2-ol;
3-(methylamino)-1-(4-phenoxy-1H-indol-1-yl)-1-phenylpropan-2-ol;
3-(methylamino)-1-phenyl-1-(4-phenyl-1H-indol-1-yl)propan-2-ol;
3-(methylamino)-1-phenyl-1-(6-phenyl-1H-indol-1-yl)propan-2-ol;
3-(methylamino)-1-phenyl-1-(7-phenyl-1H-indol-1-yl)propan-2-ol;
1-[5-(benzyloxy)-1H-indol-1-yl]-1-(3-fluorophenyl)-3-(methylamino)propan-2-ol;
1-[5-(benzyloxy)-2,3-dihydro-1H-indol-1-yl]-1-(3-fluorophenyl)-3-(methylamino)propan-2-ol;
1-[5-(benzyloxy)-2,3-dihydro-1H-indol-1-yl]-3-(methylamino)-1-phenylpropan-2-ol;
5′-chloro-1′-[2-hydroxy-3-(methylamino)-1-phenylpropyl]spiro[cyclohexane-1,3′-indol]-2′(1′H)-one;
6′-chloro-1′-[(2-hydroxy-3-(methylamino)-1-phenylpropyl]spiro[cyclohexane-1,3′-indol]-2′(1′H)-one;
6′-fluoro-1′-[2-hydroxy-3-(methylamino)-1-phenylpropyl]spiro[cyclohexane-1,3′-indol]-2′(1′H)-one;
5′-fluoro-1′-[2-hydroxy-3-(methylamino)-1-phenylpropyl]spiro[cyclohexane-1,3′-indol]-2′(1′H)-one;
7′-chloro-1′-[2-hydroxy-3-(methylamino)-1-phenylpropyl]spiro[cyclohexane-1,3′-indol]-2′(1′H)-one;
6′-fluoro-1′-[1-(3-fluorophenyl)-2-hydroxy-3-(methylamino)propyl]spiro[cyclohexane-1,3′-indol]-2′(1′H)-one;
3-(methylamino)-1-phenyl-1-spiro[cyclohexane-1,3′-indol]-1′(2′H)-ylpropan-2-ol;
1-(3-fluorophenyl)-3-(methylamino)-1-{3-[2-(trifluoromethoxy)phenyl]-1H-indol-1-yl}propan-2-ol;
1-(3-fluorophenyl)-1-[3-(2-isopropoxyphenyl)-1H-indol-1-yl]-3-(methylamino)propan-2-ol;
1-(3-fluorophenyl)-1-[3-(4-fluorophenyl)-1H-indol-1-yl]-3-(methylamino)propan-2-ol;
1-(3-fluorophenyl)-3-(methylamino)-1-[3-(2-phenoxyphenyl)-1H-indol-1-yl]propan-2-ol;
1-[3-(2,4-difluorophenyl)-1H-indol-1-yl]-1-(3-fluorophenyl)-3-(methylamino)propan-2-ol;
1-[3-(2,5-difluorophenyl)-1H-indol-1-yl]-1-(3-fluorophenyl)-3-(methylamino)propan-2-ol;
1-[3-(2,3-dimethoxyphenyl)-1H-indol-1-yl]-1-(3-fluorophenyl)-3-(methylamino)propan-2-ol;
1-[3-(2,4-dichlorophenyl)-1H-indol-1-yl]-1-(3-fluorophenyl)-3-(methylamino)propan-2-ol;
1-[3-(2-ethoxyphenyl)-1H-indol-1-yl]-1-(3-fluorophenyl)-3-(methylamino)propan-2-ol;
1-(7-chloro-5-methoxy-1H-pyrrolo[2,3-c]pyridin-1-yl)-1-(3-fluorophenyl)-3-(methylamino)propan-2-ol;
1-(7-chloro-5-methyl-1H-pyrrolo[2,3-c]pyridin-1-yl)-3-(methylamino)-1-phenylpropan-2-ol;
1-(5-methoxy-1H-pyrrolo[2,3-c]pyridin-1-yl)-3-(methylamino)-1-phenylpropan-2-ol;
1-(3-fluorophenyl)-1-(5-methoxy-1H-pyrrolo[2,3-c]pyridin-1-yl)-3-(methylamino)propan-2-ol;
3-(methylamino)-1-(5-methyl-1H-pyrrolo[2,3-c]pyridin-1-yl)-1-phenylpropan-2-ol;
1-(3-fluorophenyl)-3-(methylamino)-1-(5-methyl-1H-pyrrolo[2,3-c]pyridin-1-yl)propan-2-ol;
3-(methylamino)-1-(7-methyl-1H-pyrrolo[2,3-c]pyridin-1-yl)-1-phenylpropan-2-ol;
1-(3-fluorophenyl)-3-(methylamino)-1-(7-methyl-1H-pyrrolo[2,3-c]pyridin-1-yl)propan-2-ol;
1-(3,3-diethyl-2,3-dihydro-1H-indol-1-yl)-1-(3-fluorophenyl)-3-(methylamino)propan-2-ol;
1-(6-fluoro-3,3-dimethyl-2,3-dihydro-1H-indol-1-yl)-1-(3-fluorophenyl)-3-(methylamino)propan-2-ol;
1-(4-benzyl-3,4-dihydroquinoxalin-1(2H)-yl)-1-(3-fluorophenyl)-3-(methylamino)propan-2-ol;
1-(5-fluoro-3,3-dimethyl-2,3-dihydro-1H-indol-1-yl)-1-(3-fluorophenyl)-3-(methylamino)propan-2-ol;
1-(3-fluorophenyl)-3-(methylamino)-1-[(3S)-3-methyl-2,3-dihydro-1H-indol-1-yl]propan-2-ol;
1-(3-fluorophenyl)-3-(methylamino)-1-[(3R)-3-methyl-2,3-dihydro-1H-indol-1-yl]propan-2-ol;
1-(3-fluorophenyl)-1-(3-isopropyl-2,3-dihydro-1H-indol-1-yl)-3-(methylamino)propan-2-ol;
1-(3-ethyl-2,3-dihydro-1H-indol-1-yl)-1-(3-fluorophenyl)-3-(methylamino)propan-2-ol;
1-(3-ethyl-2,3-dihydro-1H-indol-1-yl)-3-(methylamino)-1-phenylpropan-2-ol;
1-(3-isopropyl-2,3-dihydro-1H-indol-1-yl)-3-(methylamino)-1-phenylpropan-2-ol;
3-amino-1-(3,5-difluorophenyl)-1-(3,3-dimethyl-2,3-dihydro-1H-indol-1-yl)propan-2-ol;
1-[1-(3,5-difluorophenyl)-2-hydroxy-3-(methylamino)propyl]-7-fluoro-3,3-dimethyl-1,3-dihydro-2H-indol-2-one;
5,7-difluoro-1-[1-(3-fluorophenyl)-2-hydroxy-3-(methylamino)propyl]-3,3-dimethyl-1,3-dihydro-2H-indol-2-one;
1-[1-(3,5-difluorophenyl)-2-hydroxy-3-(methylamino)propyl]-3,3-dimethyl-1,3-dihydro-2H-indol-2-one;
1-[2-hydroxy-3-(methylamino)-1-phenylpropyl]-1H-indol-5-ol;
1-[1-(3-fluorophenyl)-2-hydroxy-3-(methylamino)propyl]-1H-indol-5-ol:
5′-(benzyloxy)-1′-[2-hydroxy-3-(methylamino)-1-phenylpropyl]spiro[cyclohexane-1,3′-indol]-2′(1′H)-one;
5-(benzyloxy)-1-[2-hydroxy-3-(methylamino)-1-phenylpropyl]-3,3-dimethyl-1,3-dihydro-2H-indol-2-one;
1-[1-(3-chlorophenyl)-2-hydroxy-3-(methylamino)propyl]-7-fluoro-3,3-dimethyl-1,3-dihydro-2H-indol-2-one;
1-(3-chloro-5-fluorophenyl)-1-(1H-indol-1-yl)-3-(methylamino)propan-2-ol;
3-chloro-N-{1-[2-hydroxy-3-(methylamino)-1-phenylpropyl]-1H-indol-5-yl}-4-methylbenzamide;
3-chloro-N-{1-[2-hydroxy-3-(methylamino)-1-phenylpropyl]-2,3-dihydro-1H-indol-5-yl}benzamide;
3-chloro-N-{1-[2-hydroxy-3-(methylamino)-1-phenylpropyl]-1H-indol-5-yl}benzamide;
N-{1-[2-hydroxy-3-(methylamino)-1-phenylpropyl]-2,3-dihydro-1H-indol-5-yl}benzamide;
N-{1-[2-hydroxy-3-(methylamino)-1-phenylpropyl]-1H-indol-5-yl}benzamide;
N-{1-[2-hydroxy-3-(methylamino)-1-phenylpropyl]-2,3-dihydro-1H-indol-5-yl}cyclohexanecarboxamide;
N-{1-[2-hydroxy-3-(methylamino)-1-phenylpropyl]-1H-indol-5-yl}cyclohexanecarboxamide;

N-(3-chlorophenyl)-1-[2-hydroxy-3-(methylamino)-1-phenylpropyl]indoline-5-carboxamide;
N-(3-chlorophenyl)-1-[2-hydroxy-3-(methylamino)-1-phenylpropyl]-1H-indole-5-carboxamide;
3-(methylamino)-1-(6-phenoxy-1H-indol-1-yl)-1-phenylpropan-2-ol;
3-(methylamino)-1-(7-phenoxy-1H-indol-1-yl)-1-phenylpropan-2-ol;
3-amino-1-[5-(benzyloxy)-1H-indol-1-yl]-1-phenylpropan-2-ol;
1-[5-(benzyloxy)-1H-indol-1-yl]-3-(ethylamino)-1-phenylpropan-2-ol;
1-[5-(benzyloxy)-1H-indol-1-yl]-1-phenyl-3-(propylamino)propan-2-ol;
1-[5-(benzyloxy)-1H-indol-1-yl]-3-(isopropylamino)-1-phenylpropan-2-ol;
1-[5-(benzyloxy)-1H-indol-1-yl]-3-(dimethylamino)-1-phenylpropan-2-ol;
1-[5-(benzyloxy)-1H-indol-1-yl]-3-[ethyl(methyl)amino]-1-phenylpropan-2-ol;
1-[5-(benzyloxy)-1H-indol-1-yl]-3-(diethylamino)-1-phenylpropan-2-ol;
1-[5-(benzyloxy)-1H-indol-1-yl]-1-phenyl-3-pyrrolidin-1-ylpropan-2-ol;
1-[5-(benzyloxy)-1H-indol-1-yl]-1-phenyl-3-piperidin-1-ylpropan-2-ol;
1-[5-(benzyloxy)-1H-indol-1-yl]-3-(4-methylpiperazin-1-yl)-1-phenylpropan-2-ol hydrochloride
3-(methylamino)-1-phenyl-1-[5-(pyridin-2-ylmethoxy)-1H-indol-1-yl]propan-2-ol;
3-(methylamino)-1-phenyl-1-[5-(phenylethynyl)-1H-indol-1-yl]propan-2-ol;
3-(methylamino)-1-phenyl-1-[5-(2-phenylethyl)-1H-indol-1-yl]propan-2-ol;
1′-[3-amino-2-hydroxy-1-phenylpropyl]-6′-fluorospiro[cyclohexane-1,3′-indol]-2′(1′H)-one;
1′-[3-(ethylamino)-2-hydroxy-1-phenylpropyl]-6′-fluorospiro[cyclohexane-1,3′-indol]-2′(1′H)-one;
6′-fluoro-1′-[2-hydroxy-3-(isopropylamino)-1-phenylpropyl]spiro[cyclohexane-1,3′-indol]-2′(1′H)-one;
6′-fluoro-1′-[2-hydroxy-1-phenyl-3-(propylamino)propyl]spiro[cyclohexane-1,3′-indol]-2′(1′H)-one;
1′-[3-amino-2-hydroxy-1-phenylpropyl]-5′-fluorospiro[cyclohexane-1,3′-indol]-2′(1′H)-one;
1′-[3-(ethylamino)-2-hydroxy-1-phenylpropyl]-5′-fluorospiro[cyclohexane-1,3′-indol]-2′(1′H)-one;
5′-fluoro-1′-[2-hydroxy-3-(isopropylamino)-1-phenylpropyl]spiro[cyclohexane-1,3′-indol]-2′(1′H)-one;
5′-fluoro-1′-[2-hydroxy-1-phenyl-3-(propylamino)propyl]spiro[cyclohexane-1,3′-indol]-2′(1′H)-one;
1′-[3-(dimethylamino)-2-hydroxy-1-phenylpropyl]-5′-fluorospiro[cyclohexane-1,3′-indol]-2′(1′H)-one;
5′-fluoro-1′-[2-hydroxy-3-morpholin-4-yl-1-phenylpropyl]spiro[cyclohexane-1,3′-indol]-2′(1′H)-one;
1′-[2-hydroxy-3-(methylamino)-1-phenylpropyl]-5′-methoxyspiro[cyclohexane-1,3′-indol]-2′(1′H)-one;
1′-[2-hydroxy-3-(methylamino)-1-phenylpropyl]-6′-methoxyspiro[cyclohexane-1,3′-indol]-2′(1′H)-one;
1′-[2-hydroxy-3-(methylamino)-1-phenylpropyl]-2′-oxo-1′,2′-dihydrospiro[cyclohexane-1,3′-indole]-5′-carbonitrile;
1′-[2-hydroxy-3-(methylamino)-1-phenylpropyl]-2′-oxo-1′,2′-dihydrospiro[cyclohexane-1,3′-indole]-6′-carbonitrile;
4′,5′-difluoro-1′-[2-hydroxy-3-(methylamino)-1-phenylpropyl]spiro[cyclohexane-1,3′-indol]-2′(1′H)-one;
7′-fluoro-1′-[1-(3-fluorophenyl)-2-hydroxy-3-(methylamino)propyl]spiro[cyclohexane-1,3′-indol]-2′(1′H)-one;
1′-[1-(3-chlorophenyl)-2-hydroxy-3-(methylamino)propyl]-6′-fluorospiro[cyclohexane-1,3′-indol]-2′(1′H)-one;
1-[1-(3-chloro-5-fluorophenyl)-2-hydroxy-3-(methylamino)propyl]-7-fluoro-3,3-dimethyl-1,3-dihydro-2H-indol-2-one;
1-(3-chloro-5-fluorophenyl)-1-(2,3-dihydro-1H-indol-1-yl)-3-(methylamino)propan-2-ol;
1-(3-chloro-5-fluorophenyl)-1-(7-fluoro-3,3-dimethyl-2,3-dihydro-1H-indol-1-yl)-3-(methylamino)propan-2-ol;
1-(3-chloro-5-fluorophenyl)-1-(3,3-dimethyl-2,3-dihydro-1H-indol-1-yl)-3-(methylamino)propan-2-ol;
7′-fluoro-1′-[1-(3-fluorophenyl)-2-hydroxy-3-(methylamino)propyl]spiro[cyclobutane-1,3′-indol]-2′(1′H)-one;
7′-fluoro-1′-[1-(3-fluorophenyl)-2-hydroxy-3-(methylamino)propyl]spiro[cyclopentane-1,3′-indol]-2′(1′H)-one;
6-fluoro-1-[1-(3-fluorophenyl)-2-hydroxy-3-(methylamino)propyl]-3,3-dimethyl-1,3-dihydro-2H-indol-2-one;
1-(7-fluoro-2,3-dihydro-1H-indol-1-yl)-3-(methylamino)-1-phenylpropan-2-ol;
4-fluoro-3-[1-(3-fluorophenyl)-2-hydroxy-3-(methylamino)propyl]-1-phenyl-1,3-dihydro-2H-benzimidazol-2-one;
4-fluoro-1-(3-fluorophenyl)-3-[1-(3-fluorophenyl)-2-hydroxy-3-(methylamino)propyl]-1,3-dihydro-2H-benzimidazol-2-one;
1-[3-amino-1-(3,5-difluorophenyl)-2-hydroxypropyl]-7-fluoro-3,3-dimethyl-1,3-dihydro-2H-indol-2-one; and

pharmaceutically acceptable salts thereof, especially hydrochloride salt.

Especially preferred compounds of the invention include, but are not limited to:
(1S,2R)-1-[5-(benzyloxy)-1H-indol-1-yl]-3-(methylamino)-1-phenylpropan-2-ol;
(1S,2R)-1-[4-(benzyloxy)-1H-indol-1-yl]-3-(methylamino)-1-phenylpropan-2-ol;
(1S,2R)-1-[6-(benzyloxy)-1H-indol-1-yl]-3-(methylamino)-1-phenylpropan-2-ol;
(1S,2R)-1-[7-(benzyloxy)-1H-indol-1-yl]-3-(methylamino)-1-phenylpropan-2-ol;
(1S,2R)-1-{5-[(2-methoxybenzyl)oxy]-1H-indol-1-yl}-3-(methylamino)-1-phenylpropan-2-ol;
(1S,2R)-1-{5-[(3-methoxybenzyl)oxy]-1H-indol-1-yl}-3-(methylamino)-1-phenylpropan-2-ol;
(1S,2R)-1-{5-[(4-methoxybenzyl)oxy]-1H-indol-1-yl}-3-(methylamino)-1-phenylpropan-2-ol;
(1S,2R)-1-{5-[(2-chlorobenzyl)oxy]-1H-indol-1-yl}-3-(methylamino)-1-phenylpropan-2-ol;
(1S,2R)-1-{5-[(3-chlorobenzyl)oxy]-1H-indol-1-yl}-3-(methylamino)-1-phenylpropan-2-ol;
(1S,2R)-1-{5-[(4-chlorobenzyl)oxy]-1H-indol-1-yl}-3-(methylamino)-1-phenylpropan-2-ol;
(1S,2R)-1-{5-[(2-fluorobenzyl)oxy]-1H-indol-1-yl}-3-(methylamino)-1-phenylpropan-2-ol;
(1S,2R)-1-{5-[(3-fluorobenzyl)oxy]-1H-indol-1-yl}-3-(methylamino)-1-phenylpropan-2-ol;
(1S,2R)-1-{5-[(4-fluorobenzyl)oxy]-1H-indol-1-yl}-3-(methylamino)-1-phenylpropan-2-ol;
(1S,2R)-3-(methylamino)-1-{5-[(2-methylbenzyl)oxy]-1H-indol-1-yl}-1-phenylpropan-2-ol;
(1S,2R)-3-(methylamino)-1-{5-[(3-methylbenzyl)oxy]-1H-indol-1-yl}-1-phenylpropan-2-ol;
(1S,2R)-3-(methylamino)-1-{5-[(4-methylbenzyl)oxy]-1H-indol-1-yl}-1-phenylpropan-2-ol;
(1S,2R)-3-(methylamino)-1-phenyl-1-[5-(1-phenylethoxy)-1H-indol-1-yl]propan-2-ol;
(1S,2R)-3-(methylamino)-1-phenyl-1-[5-(2-phenylethoxy)-1H-indol-1-yl]propan-2-ol;
(1S,2R)-3-(methylamino)-1-(5-phenoxy-1H-indol-1-yl)-1-phenylpropan-2-ol;
(1S,2R)-3-(methylamino)-1-(4-phenoxy-1H-indol-1-yl)-1-phenylpropan-2-ol;
(1S,2R)-3-(methylamino)-1-phenyl-1-(4-phenyl-1H-indol-1-yl)propan-2-ol;
(1S,2R)-3-(methylamino)-1-phenyl-1-(6-phenyl-1H-indol-1-yl)propan-2-ol;
(1S,2R)-3-(methylamino)-1-phenyl-1-(7-phenyl-1H-indol-1-yl)propan-2-ol;
(1S,2R)-1-[5-(benzyloxy)-1H-indol-1-yl]-1-(3-fluorophenyl)-3-(methylamino)propan-2-ol;
(1S,2R)-1-[5-(benzyloxy)-2,3-dihydro-1H-indol-1-yl]-1-(3-fluorophenyl)-3-(methylamino)propan-2-ol;
(1S,2R)-1-[5-(benzyloxy)-2,3-dihydro-1H-indol-1-yl]-3-(methylamino)-1-phenylpropan-2-ol;
5′-chloro-1′-[(1S,2R)-2-hydroxy-3-(methylamino)-1-phenylpropyl]spiro[cyclohexane-1,3′-indol]-2′(1′H)-one;
6′-chloro-1′-[(1S,2R)-2-hydroxy-3-(methylamino)-1-phenylpropyl]spiro[cyclohexane-1,3′-indol]-2′(1′H)-one;
6′-fluoro-1′-[(1S,2R)-2-hydroxy-3-(methylamino)-1-phenylpropyl]spiro[cyclohexane-1,3′-indol]-2′(1′H)-one;
5′-fluoro-1′-[(1S,2R)-2-hydroxy-3-(methylamino)-1-phenylpropyl]spiro[cyclohexane-1,3′-indol]-2′(1′H)-one;
7′-chloro-1′-[(1S,2R)-2-hydroxy-3-(methylamino)-1-phenylpropyl]spiro[cyclohexane-1,3′-indol]-2′(1′H)-one;
6′-fluoro-1′-[(1S,2R)-1-(3-fluorophenyl)-2-hydroxy-3-(methylamino)propyl]spiro[cyclohexane-1,3′-indol]-2′(1′H)-one;
(1S,2R)-3-(methylamino)-1-phenyl-1-spiro[cyclohexane-1,3′-indol]-1′(2′H)-ylpropan-2-ol;
(1S,2R)-1-(3-fluorophenyl)-3-(methylamino)-1-{3-[2-(trifluoromethoxy)phenyl]-1H-indol-1-yl}propan-2-ol;
(1S,2R)-1-(3-fluorophenyl)-1-[3-(2-isopropoxyphenyl)-1H-indol-1-yl]-3-(methylamino)propan-2-ol;
(1S,2R)-1-(3-fluorophenyl)-1-[3-(4-fluorophenyl)-1H-indol-1-yl]-3-(methylamino)propan-2-ol;
(1S,2R)-1-(3-fluorophenyl)-3-(methylamino)-1-[3-(2-phenoxyphenyl)-1H-indol-1-yl]propan-2-ol;
(1S,2R)-1-[3-(2,4-difluorophenyl)-1H-indol-1-yl]-1-(3-fluorophenyl)-3-(methylamino)propan-2-ol;
(1S,2R)-1-[3-(2,5-difluorophenyl)-1H-indol-1-yl]-1-(3-fluorophenyl)-3-(methylamino)propan-2-ol;
(1S,2R)-1-[3-(2,3-dimethoxyphenyl)-1H-indol-1-yl]-1-(3-fluorophenyl)-3-(methylamino)propan-2-ol;
(1S,2R)-1-[3-(2,4-dichlorophenyl)-1H-indol-1-yl]-1-(3-fluorophenyl)-3-(methylamino)propan-2-ol;
(1S,2R)-1-[3-(2-ethoxyphenyl)-1H-indol-1-yl]-1-(3-fluorophenyl)-3-(methylamino)propan-2-ol;
(1S,2R)-1-(7-chloro-5-methoxy-1H-pyrrolo[2,3-c]pyridin-1-yl)-1-(3-fluorophenyl)-3-(methylamino)propan-2-ol;
(1S,2R)-1-(7-chloro-5-methyl-1H-pyrrolo[2,3-c]pyridin-1-yl)-3-(methylamino)-1-phenylpropan-2-ol;
(1S ,2R)-1-(5-methoxy-1H-pyrrolo[2,3-c]pyridin-1-yl)-3-(methylamino)-1-phenylpropan-2-ol;
(1S,2R)-1-(3-fluorophenyl)-1-(5-methoxy-1H-pyrrolo[2,3-c]pyridin-1-yl)-3-(methylamino)propan-2-ol;
(1S,2R)-3-(methylamino)-1-(5-methyl-1H-pyrrolo[2,3-c]pyridin-1-yl)-1-phenylpropan-2-ol;
(1S,2R)-1-(3-fluorophenyl)-3-(methylamino)-1-(5-methyl-1H-pyrrolo[2,3-c]pyridin-1-yl)propan-2-ol;
(1S,2R)-3-(methylamino)-1-(7-methyl-1H-pyrrolo[2,3-c]pyridin-1-yl)-1-phenylpropan-2-ol;
(1S,2R)-1-(3-fluorophenyl)-3-(methylamino)-1-(7-methyl-1H-pyrrolo[2,3-c]pyridin-1-yl)propan-2-ol;
(1S,2R)-1-(3,3-diethyl-2,3-dihydro-1H-indol-1-yl)-1-(3-fluorophenyl)-3-(methylamino)propan-2-ol;
(1S,2R)-1-(6-fluoro-3,3-dimethyl-2,3-dihydro-1H-indol-1-yl)-1-(3-fluorophenyl)-3-(methylamino)propan-2-ol;
(1S,2R)-1-(4-benzyl-3,4-dihydroquinoxalin-1(2H)-yl)-1-(3-fluorophenyl)-3-(methylamino)propan-2-ol;
(1S,2R)-1-(5-fluoro-3,3-dimethyl-2,3-dihydro-1H-indol-1-yl)-1-(3-fluorophenyl)-3-(methylamino)propan-2-ol;
(1S,2R)-1-(3-fluorophenyl)-3-(methylamino)-1-[(3S)-3-methyl-2,3-dihydro-1H-indol-1-yl]propan-2-ol;
(1S,2R)-1-(3-fluorophenyl)-3-(methylamino)-1-[(3R)-3-methyl-2,3-dihydro-1H-indol-1-yl]propan-2-ol;
(1S,2R)-1-(3-fluorophenyl)-1-(3-isopropyl-2,3-dihydro-1H-indol-1-yl)-3-(methylamino)propan-2-ol;
(1S,2R)-1-(3-ethyl-2,3-dihydro-1H-indol-1-yl)-1-(3-fluorophenyl)-3-(methylamino)propan-2-ol;
(1S,2R)-1-(3-ethyl-2,3-dihydro-1H-indol-1-yl)-3-(methylamino)-1-phenylpropan-2-ol;
(1S,2R)-1-(3-isopropyl-2,3-dihydro-1H-indol-1-yl)-3-(methylamino)-1-phenylpropan-2-ol;
(1S,2R)-3-amino-1-(3,5-difluorophenyl)-1-(3,3-dimethyl-2,3-dihydro-1H-indol-1-yl)propan-2-ol;
1-[(1S,2R)-1-(3,5-difluorophenyl)-2-hydroxy-3-(methylamino)propyl]-7-fluoro-3,3-dimethyl-1,3-dihydro-2H-indol-2-one;
5,7-difluoro-1-[(1S,2R)-1-(3-fluorophenyl)-2-hydroxy-3-(methylamino)propyl]-3,3-dimethyl-1,3-dihydro-2H-indol-2-one;
1-[(1S,2R)-1-(3,5-difluorophenyl)-2-hydroxy-3-(methylamino)propyl]-3,3-dimethyl-1,3-dihydro-2H-indol-2-one;
1-[(1S,2R)-2-hydroxy-3-(methylamino)-1-phenylpropyl]-1H-indol-5-ol;
1-[(1S,2R)-1-(3-fluorophenyl)-2-hydroxy-3-(methylamino)propyl]-1H-indol-5-ol:
5′-(benzyloxy)-1′-[(1S,2R)-2-hydroxy-3-(methylamino)-1-phenylpropyl]spiro[cyclohexane-1,3′-indol]-2′(1′H)-one;
5-(benzyloxy)-1-[(1S,2R)-2-hydroxy-3-(methylamino)-1-phenylpropyl]-3,3-dimethyl-1,3-dihydro-2H-indol-2-one;
1-[(1S,2R)-1-(3-chlorophenyl)-2-hydroxy-3-(methylamino)propyl]-7-fluoro-3,3-dimethyl-1,3-dihydro-2H-indol-2-one;
(1S,2R)-1-(3-chloro-5-fluorophenyl)-1-(1H-indol-1-yl)-3-(methylamino)propan-2-ol;
3-chloro-N-{1-[(1S,2R)-2-hydroxy-3-(methylamino)-1-phenylpropyl]-1H-indol-5-yl}-4-methylbenzamide;
3-chloro-N-{1-[(1S,2R)-2-hydroxy-3-(methylamino)-1-phenylpropyl]-2,3-dihydro-1H-indol-5-yl}benzamide;
3-chloro-N-{1-[(1S,2R)-2-hydroxy-3-(methylamino)-1-phenylpropyl]-1H-indol-5-yl}benzamide;
N-{1-[(1S,2R)-2-hydroxy-3-(methylamino)-1-phenylpropyl]-2,3-dihydro-1H-indol-5-yl}benzamide;
N-{1-[(1S,2R)-2-hydroxy-3-(methylamino)-1-phenylpropyl]-1H-indol-5-yl}benzamide;
N-{1-[(1S,2R)-2-hydroxy-3-(methylamino)-1-phenylpropyl]-2,3-dihydro-1H-indol-5-yl}cyclohexanecarboxamide;
N-{1-[(1S,2R)-2-hydroxy-3-(methylamino)-1-phenylpropyl]-1H-indol-5-yl}cyclohexanecarboxamide;
N-(3-chlorophenyl)-1-[(1S,2R)-2-hydroxy-3-(methylamino)-1-phenylpropyl]indoline-5-carboxamide;
N-(3-chlorophenyl)-1-[(1S,2R)-2-hydroxy-3-(methylamino)-1-phenylpropyl]-1H-indole-5-carboxamide;
(1S,2R)-3-(methylamino)-1-(6-phenoxy-1H-indol-1-yl)-1-phenylpropan-2-ol;
(1S,2R)-3-(methylamino)-1-(7-phenoxy-1H-indol-1-yl)-1-phenylpropan-2-ol;
(1S,2R)-3-amino-1-[5-(benzyloxy)-1H-indol-1-yl]-1-phenylpropan-2-ol;
(1S,2R)-1-[5-(benzyloxy)-1H-indol-1-yl]-3-(ethylamino)-1-phenylpropan-2-ol;
(1S,2R)-1-[5-(benzyloxy)-1H-indol-1-yl]-1-phenyl-3-(propylamino)propan-2-ol;
(1S,2R)-1-[5-(benzyloxy)-1H-indol-1-yl]-3-(isopropylamino)-1-phenylpropan-2-ol;
(1S,2R)-1-[5-(benzyloxy)-1H-indol-1-yl]-3-(dimethylamino)-1-phenylpropan-2-ol;
(1S,2R)-1-[5-(benzyloxy)-1H-indol-1-yl]-3-[ethyl(methyl)amino]-1-phenylpropan-2-ol;
(1S,2R)-1-[5-(benzyloxy)-1H-indol-1-yl]-3-(diethylamino)-1-phenylpropan-2-ol;
(1S,2R)-1-[5-(benzyloxy)-1H-indol-1-yl]-1-phenyl-3-pyrrolidin-1-ylpropan-2-ol;
(1S,2R)-1-[5-(benzyloxy)-1H-indol-1-yl]-1-phenyl-3-piperidin-1-ylpropan-2-ol;
(1S,2R)-1-[5-(benzyloxy)-1H-indol-1-yl]-3-(4-methylpiperazin-1-yl)-1-phenylpropan-2-ol hydrochloride
(1S,2R)-3-(methylamino)-1-phenyl-1-[5-(pyridin-2-ylmethoxy)-1H-indol-1-yl]propan-2-ol;
(1S,2R)-3-(methylamino)-1-phenyl-1-[5-(phenylethynyl)-1H-indol-1-yl]propan-2-ol;
(1S,2R)-3-(methylamino)-1-phenyl-1-[5-(2-phenylethyl)-1H-indol-1-yl]propan-2-ol;
1′-[(1S,2R)-3-amino-2-hydroxy-1-phenylpropyl]-6′-fluorospiro[cyclohexane-1,3′-indol]-2′(1′H)-one;
1′-[(1S,2R)-3-(ethylamino)-2-hydroxy-1-phenylpropyl]-6′-fluorospiro[cyclohexane-1,3′-indol]-2′(1′H)-one;
6′-fluoro-1′-[(1S,2R)-2-hydroxy-3-(isopropylamino)-1-phenylpropyl]spiro[cyclohexane-1,3′-indol]-2′(1′H)-one;
6′-fluoro-1′-[(1S,2R)-2-hydroxy-1-phenyl-3-(propylamino)propyl]spiro[cyclohexane-1,3′-indol]-2′(1′H)-one;
1′-[(1S,2R)-3-amino-2-hydroxy-1-phenylpropyl]-5′-fluorospiro[cyclohexane-1,3′-indol]-2′(1′H)-one;
1′-[(1S,2R)-3-(ethylamino)-2-hydroxy-1-phenylpropyl]-5′-fluorospiro[cyclohexane-1,3′-indol]-2′(1′H)-one;
5′-fluoro-1′-[(1S,2R)-2-hydroxy-3-(isopropylamino)-1-phenylpropyl]spiro[cyclohexane-1,3′-indol]-2′(1′H)-one;
5′-fluoro-1′-[(1S,2R)-2-hydroxy-1-phenyl-3-(propylamino)propyl]spiro[cyclohexane-1,3′-indol]-2′(1′H)-one;
1′-[(1S,2R)-3-(dimethylamino)-2-hydroxy-1-phenylpropyl]-5′-fluorospiro[cyclohexane-1,3′-indol]-2′(1′H)-one;
5′-fluoro-1′-[(1S,2R)-2-hydroxy-3-morpholin-4-yl-1-phenylpropyl]spiro[cyclohexane-1,3′-indol]-2′(1′H)-one;
1′-[(1S,2R)-2-hydroxy-3-(methylamino)-1-phenylpropyl]-5′-methoxyspiro[cyclohexane-1,3′-indol]-2′(1′H)-one;
1′-[(1S,2R)-2-hydroxy-3-(methylamino)-1-phenylpropyl]-6′-methoxyspiro[cyclohexane-1,3′-indol]-2′(1′H)-one;
1′-[(1S,2R)-2-hydroxy-3-(methylamino)-1-phenylpropyl]-2′-oxo-1′,2′-dihydrospiro[cyclohexane-1,3′-indole]-5′-carbonitrile;
1′-[(1S,2R)-2-hydroxy-3-(methylamino)-1-phenylpropyl]-2′-oxo-1′,2′-dihydrospiro[cyclohexane-1,3′-indole]-6′-carbonitrile;
4′,5′-difluoro-1′-[(1S,2R)-2-hydroxy-3-(methylamino)-1-phenylpropyl]spiro[cyclohexane-1,3′-indol]-2′(1′H)-one;
7′-fluoro-1′-[(1S,2R)-1-(3-fluorophenyl)-2-hydroxy-3-(methylamino)propyl]spiro[cyclohexane-1,3′-indol]-2′(1′H)-one;
1′-[(1S,2R)-1-(3-chlorophenyl)-2-hydroxy-3-(methylamino)propyl]-6′-fluorospiro[cyclohexane-1,3′-indol]-2′(1′H)-one;
1-[(1S,2R)-1-(3-chloro-5-fluorophenyl)-2-hydroxy-3-(methylamino)propyl]-7-fluoro-3,3-dimethyl-1,3-dihydro-2H-indol-2-one;
(1S,2R)-1-(3-chloro-5-fluorophenyl)-1-(2,3-dihydro-1H-indol-1-yl)-3-(methylamino)propan-2-ol;
(1S,2R)-1-(3-chloro-5-fluorophenyl)-1-(7-fluoro-3,3-dimethyl-2,3-dihydro-1H-indol-1-yl)-3-(methylamino)propan-2-ol;
(1S,2R)-1-(3-chloro-5-fluorophenyl)-1-(3,3-dimethyl-2,3-dihydro-1H-indol-1-yl)-3-(methylamino)propan-2-ol;
7′-fluoro-1′-[(1S,2R)-1-(3-fluorophenyl)-2-hydroxy-3-(methylamino)propyl]spiro[cyclobutane-1,3′-indol]-2′(1′H)-one;
7′-fluoro-1′-[(1S,2R)-1-(3-fluorophenyl)-2-hydroxy-3-(methylamino)propyl]spiro[cyclopentane-1,3′-indol]-2′(1′H)-one;
6-fluoro-1-[(1S,2R)-1-(3-fluorophenyl)-2-hydroxy-3-(methylamino)propyl]-3,3-dimethyl-1,3-dihydro-2H-indol-2-one;
(1S,2R)-1-(7-fluoro-2,3-dihydro-1H-indol-1-yl)-3-(methylamino)-1-phenylpropan-2-ol;
4-fluoro-3-[(1S,2R)-1-(3-fluorophenyl)-2-hydroxy-3-(methylamino)propyl]-1-phenyl-1,3-dihydro-2H-benzimidazol-2-one;
4-fluoro-1-(3-fluorophenyl)-3-[(1S,2R)-1-(3-fluorophenyl)-2-hydroxy-3-(methylamino)propyl]-1,3-dihydro-2H-benzimidazol-2-one;
1-[(1S,2R)-3-amino-1-(3,5-difluorophenyl)-2-hydroxypropyl]-7-fluoro-3,3-dimethyl-1,3-dihydro-2H-indol-2-one; and

pharmaceutically acceptable salts thereof, especially hydrochloride salt.

Some of the compounds of the present invention may contain chiral centers and such compounds may exist in the form of stereoisomers (i.e. enantiomers). The present invention includes all such stereoisomers and any mixtures thereof including racemic mixtures. Racemic mixtures of the stereoisomers as well as the substantially pure stereoisomers are within the scope of the invention. The term “substantially pure,” as used herein, refers to at least about 90 mole %, more preferably at least about 95 mole %, and most preferably at least about 98 mole % of the desired stereoisomer is present relative to other possible stereoisomers. Preferred enantiomers may be isolated from racemic mixtures by any method known to those skilled in the art, including high performance liquid chromatography (HPLC) and the formation and crystallization of chiral salts or prepared by methods described herein. See, for example, Jacques, et al., Enantiomers, Racemates and Resolutions (Wiley Interscience, New York, 1981); Wilen, S. H., et al., Tetrahedron, 33:2725 (1977); Eliel, E. L. Stereochemistry of Carbon Compounds, (McGraw-Hill, NY, 1962); Wilen, S. H. Tables of Resolving Agents and Optical Resolutions, p. 268 (E. L. Eliel, Ed., University of Notre Dame Press, Notre Dame, Ind. 1972).

The present invention includes prodrugs of the compounds of formula I, II, or III. “Prodrug,” as used herein, means a compound which is convertible in vivo by metabolic means (e.g. by hydrolysis) to a compound of formula I, II, or III. Various forms of prodrugs are known in the art, for example, as discussed in Bundgaard, (ed.), Design of Prodrugs, Elsevier (1985); Widder, et al. (ed.), Methods in Enzymology, vol. 4, Academic Press (1985); Krogsgaard-Larsen, et al., (ed). “Design and Application of Prodrugs,” Textbook of Drug Design and Development, Chapter 5, 113-191 (1991), Bundgaard, et al., Journal of Drug Deliver Reviews, 1992, 8:1-38, Bundgaard, J. of Pharmaceutical Sciences, 1988, 77:285 et seq.; and Higuchi and Stella (eds.) Prodrugs as Novel Drug Delivery Systems, American Chemical Society (1975).

Further, the compounds of formula I, II, or III may exist in unsolvated as well as in solvated forms with pharmaceutically acceptable solvents such as water, ethanol, and the like. In general, the solvated forms are considered equivalent to the unsolvated forms for the purpose of the present invention.

In certain embodiments, the compounds of formula I, II, or III specifically exclude the following compounds:
1-(1H-indol-1-yl)-3-(4-methylpiperazin-1-yl)-1-phenylpropan-2-ol;
1-(5-fluoro-1H-indol-1-yl)-3-(4-methylpiperazin-1-yl)-1-phenylpropan-2-ol;
1-(1H-indol-1-yl)-3-morpholin-4-yl-1-phenylpropan-2-ol;
3-(dimethylamino)-1-(1H-indol-1-yl)-1-phenylpropan-2-ol;
3-(ethylamino)-1-(1H-indol-1-yl)-1-phenylpropan-2-ol;
1-(1H-indol-1-yl)-3-(isopropylamino)-1-phenylpropan-2-ol;
3-(benzylamino)-1-(1H-indol-1-yl)-1-phenylpropan-2-ol;
3-[(cyclohexylmethyl)amino]-1-(1H-indol-1-yl)-1-phenylpropan-2-ol;
3-[(cyclohexylmethyl)amino]-1-(3-methyl-1H-indol-1-yl)-1-phenylpropan-2-ol;
3-(isopropylamino)-1-(3-methyl-1H-indol-1-yl)-1-phenylpropan-2-ol;
1-(1H-indol-1-yl)-3-(methylamino)-1-phenylpropan-2-ol;
3-(ethylamino)-1-(3-methyl-1H-indol-1-yl)-1-phenylpropan-2-ol;
1-(1H-indol-1-yl)-1-phenyl-3-piperazin-1-ylpropan-2-ol di;
1-(1H-indol-1-yl)-1-phenyl-3-[(pyridin-4-ylmethyl)amino]propan-2-ol;
1-(5-chloro-1H-indol-1-yl)-1-phenyl-3-piperidin-1-ylpropan-2-ol;
1-(1H-indol-1-yl)-3-(methylamino)-1-phenylpropan-2-ol;
3-amino-1-(1H-indol-1-yl)-1-phenylpropan-2-ol;
3-(ethylamino)-1-(5-fluoro-1H-indol-1-yl)-1-phenylpropan-2-ol;
3-amino-1-(5-fluoro-1H-indol-1-yl)-1-phenylpropan-2-ol;
1-(5-fluoro-1H-indol-1-yl)-3-(methylamino)-1-phenylpropan-2-ol;
3-(methylamino)-1-(3-methyl-1H-indol-1-yl)-1-phenylpropan-2-ol;
1-(1H-indol-1-yl)-3-(methylamino)-1-phenylpropan-2-ol;
1-(1H-indol-1-yl)-3-(methylamino)-1-phenylpropan-2-ol;
3-amino-1-(3-methyl-1H-indol-1-yl)-1-phenylpropan-2-ol;
3-[ethyl(methyl)amino]-1-(1H-indol-1-yl)-1-phenylpropan-2-ol;
1-(5-chloro-1H-indol-1-yl)-3-(methylamino)-1-phenylpropan-2-ol;
1-(5-chloro-1H-indol-1-yl)-3-(methylamino)-1-phenylpropan-1-ol;
1-[2-hydroxy-3-(methylamino)-1-phenylpropyl]-1H-indole-3-carbonitrile;
1-(1H-indol-1-yl)-3-(methylamino)-1-phenylpropan-2-ol;
1-(1H-indol-1-yl)-3-(methylamino)-1-phenylpropan-2-ol;
3-(methylamino)-1-(3-methyl-1H-indol-1-yl)-1-phenylpropan-2-ol;
1-(3-chlorophenyl)-1-(1H-indol-1-yl)-3-(methylamino)propan-2-ol;
1-(4-chlorophenyl)-1-(1H-indol-1-yl)-3-(methylamino)propan-2-ol;
1-(1H-indol-1-yl)-3-(methylamino)-1-[3-(trifluoromethoxy)phenyl]propan-2-ol;
1-(1H-indol-1-yl)-3-(methylamino)-1-[2-(trifluoromethoxy)phenyl]propan-2-ol;
1-(1H-indol-1-yl)-3-(methylamino)-1-[2-(trifluoromethoxy)phenyl]propan-2-ol;
1-(2-chlorophenyl)-1-(1H-indol-1-yl)-3-(methylamino)propan-2-ol;
1-(1H-indol-1-yl)-3-(methylamino)-1-[4-(trifluoromethoxy)phenyl]propan-2-ol;
1-(1H-indol-1-yl)-3-(methylamino)-1-[4-(trifluoromethoxy)phenyl]propan-2-ol;
1-(1H-indol-1-yl)-3-(methylamino)-1-[4-(trifluoromethoxy)phenyl]propan-2-ol;
4-amino-1-(3-chlorophenyl)-1-(1H-indol-1-yl)butan-2-ol
1-(3-bromophenyl)-1-(1H-indol-1-yl)-3-(methylamino)propan-2-ol;
3-[2-hydroxy-1-(1H-indol-1-yl)-3-(methylamino)propyl]benzonitrile
1-(3-fluorophenyl)-1-(1H-indol-1-yl)-3-(methylamino)propan-2-ol;
1-(3-fluorophenyl)-3-(methylamino)-1-[3-(3-methylphenyl)-1H-indol-1-yl]propan-2-ol;
1-(4-fluorophenyl)-3-(methylamino)-1-(3-methyl-1H-indol-1-yl)propan-2-ol;
1-(2-fluorophenyl)-1-(1H-indol-1-yl)-3-(methylamino)propan-2-ol;
1-(4-fluorophenyl)-1-(1H-indol-1-yl)-3-(methylamino)propan-2-ol;
1-(1H-indol-1-yl)-3-(methylamino)-1-(3-methylphenyl)propan-2-ol;
1-(1H-indol-1-yl)-3-(methylamino)-1-(2-methylphenyl)propan-2-ol;
1-(1H-indol-1-yl)-3-(methylamino)-1-(2-methylphenyl)propan-2-ol;
3-(ethylamino)-1-(3-fluorophenyl)-1-(1H-indol-1-yl)propan-2-ol;
1-(3-fluorophenyl)-1-(1H-indol-1-yl)-3-morpholin-4-ylpropan-2-ol;
1-(3-fluorophenyl)-1-(1H-indol-1-yl)-3-(propylamino)propan-2-ol;
1-(3-fluorophenyl)-1-(1H-indol-1-yl)-3-(4-methylpiperazin-1-yl)propan-2-ol;
1-(1H-indol-1-yl)-3-(methylamino)-1-(4-methylphenyl)propan-2-ol;
1-(2,3-dihydro-1H-indol-1-yl)-1-(3-fluorophenyl)-3-(methylamino)propan-2-ol;
1-(2,3-dihydro-1H-indol-1-yl)-3-(methylamino)-1-phenylpropan-2-ol;
1-(3-fluorophenyl)-3-(methylamino)-1-[3-(2-methylphenyl)-1H-indol-1-yl]propan-2-ol;
1-(3-fluorophenyl)-3-(methylamino)-1-(2-methyl-2,3-dihydro-1H-indol-1-yl)propan-2-ol;
1-(7-fluoro-3,3-dimethyl-2,3-dihydro-1H-indol-1-yl)-1-(3-fluorophenyl)-3-(methylamino)propan-2-ol;
1-(7-fluoro-3,3-dimethyl-2,3-dihydro-1H-indol-1-yl)-3-(methylamino)-1-phenylpropan-2-ol;
3-(methylamino)-1-(7-methyl-2,3-dihydro-1H-indol-1-yl)-1-phenylpropan-2-ol;
1-(3-fluorophenyl)-3-(methylamino)-1-(7-methyl-2,3-dihydro-1H-indol-1-yl)propan-2-ol;
1-(3-fluorophenyl)-3-(methylamino)-1-(5-methyl-2,3-dihydro-1H-indol-1-yl)propan-2-ol;
1-(1H-indol-1-yl)-1-(3-methoxyphenyl)-3-(methylamino)propan-2-ol;
1-(1H-indol-1-yl)-1-(4-methoxyphenyl)-3-(methylamino)propan-2-ol;
3-(methylamino)-1-(2-methyl-1H-indol-1-yl)-1-phenylpropan-2-ol;
1-(1H-benzimidazol-1-yl)-3-(methylamino)-1-phenylpropan-2-ol;
3-(methylamino)-1-(2-methyl-1H-benzimidazol-1-yl)-1-phenylpropan-2-ol;
1-(4-methoxy-1H-indol-1-yl)-3-(methylamino)-1-phenylpropan-2-ol;
1-(5-fluoro-1H-indol-1-yl)-3-(methylamino)-1-phenylpropan-2-ol;
1-(5-methoxy-1H-indol-1-yl)-3-(methylamino)-1-phenylpropan-2-ol;
1-(7-methoxy-1H-indol-1-yl)-3-(methylamino)-1-phenylpropan-2-ol;
1-(4-methoxy-1H-indol-1-yl)-3-(methylamino)-1-phenylpropan-2-ol;
1-(6-methoxy-1H-indol-1-yl)-3-(methylamino)-1-phenylpropan-2-ol;
1-(5-methoxy-1H-indol-1-yl)-3-(methylamino)-1-phenylpropan-2-ol;
1-(3-fluorophenyl)-1-(6-methoxy-1H-indol-1-yl)-3-(methylamino)propan-2-ol;
3-(methylamino)-1-phenyl-1-(1H-pyrrolo[2,3-b]pyridin-1-yl)propan-2-ol;
1-(5-chloro-2,3-dihydro-1H-indol-1-yl)-1-(3-fluorophenyl)-3-(methylamino)propan-2-ol;
3-(methylamino)-1-phenyl-1-(1H-pyrrolo[2,3-c]pyridin-1-yl)propan-2-ol;
1-(5-fluoro-1H-indol-1-yl)-1-(3-fluorophenyl)-3-(methylamino)propan-2-ol;
3-(methylamino)-1-(3-fluorophenyl)-1-(1H-pyrrolo[2,3-c]pyridin-1-yl)propan-2-ol;
1-(5-chloro-2,3-dihydro-1H-indol-1-yl)-3-(methylamino)-1-phenylpropan-2-ol;
3-(methylamino)-1-(6-methyl-1H-indol-1-yl)-1-phenylpropan-2-ol;
3-(methylamino)-1-(7-methyl-1H-indol-1-yl)-1-phenylpropan-2-ol;
1-(3-fluorophenyl)-3-(methylamino)-1-(5-methyl-1H-indol-1-yl)propan-2-ol;
1-(3-fluorophenyl)-3-(methylamino)-1-(7-methyl-1H-indol-1-yl)propan-2-ol;
3-(methylamino)-1-(4-methyl-1H-indol-1-yl)-1-phenylpropan-2-ol;
3-(methylamino)-1-(5-methyl-1H-indol-1-yl)-1-phenylpropan-2-ol;
1-(3-fluorophenyl)-3-(methylamino)-1-(4-methyl-1H-indol-1-yl)propan-2-ol;
1-(3-ethyl-1H-indol-1-yl)-1-(3-fluorophenyl)-3-(methylamino)propan-2-ol;
1-(3-fluorophenyl)-3-(methylamino)-1-(3-phenyl-1H-indol-1-yl)propan-2-ol;
7-fluoro-1-[2hydroxy-3-(methylamino)-1-phenylpropyl]-3,3-dimethyl-1,3-dihydro-2H-indol-2-one;
1-[2hydroxy-3-(methylamino)-1-phenylpropyl]-3,3-dimethyl-1,3-dihydro-2H-indol-2-one;
7-fluoro-1-[1-(3-fluorophenyl)-2-hydroxy-3-(methylamino)propyl]-3,3-dimethyl-1,3-dihydro-2H-indol-2-one;
1-(1H-indol-1-yl)-3-(methylamino)-1-(2-thienyl)propan-2-ol;
1(1H-indol-1-yl)-3-(methylamino)-1-(2-thienyl)propan-2-ol;
1′-[2-hydroxy-3-(methylamino)-1-phenylpropyl]spiro[cyclohexane-1,3′-indol]-2′(1′H)-one;
2-(3-fluorophenyl)-2-(1H-indol-1-yl)-1-[(2S)-pyrrolidin-2-yl]ethanol;
2-(3-fluorophenyl)-2-(1H-indol-1-yl)-1-[pyrrolidin-2-yl]ethanol;
1′-[2hydroxy-3-(methylamino)-1-phenylpropyl]spiro[cyclobutane-1,3′-indol]-2′(1′H)-one;
1′-[2hydroxy-3-(methylamino)-1-phenylpropyl]spiro[cyclopentane-1,3′-indol]-2′(1′H)-one;
1′-[2hydroxy-3-(methylamino)-1-phenylpropyl]spiro[cyclopropane-1,3′-indol]-2′(1′H)-one;
5-fluoro-1-[2hydroxy-3-(methylamino)-1-phenylpropyl]-3,3-dimethyl-1,3-dihydro-2H-indol-2-one;
3-(cyclopropylamino)-1-(3-fluorophenyl)-1-(1H-indol-1-yl)propan-2-ol;
7′-fluoro-1′-[2hydroxy-3-(methylamino)-1-phenylpropyl]spiro[cyclohexane-1,3′-indol]-2′(1′H)-one;
5′-bromo-1′-[2hydroxy-3-(methylamino)-1-phenylpropyl]spiro[cyclohexane-1,3′-indol]-2′(1′H)-one;
1-(3-fluorophenyl)-1-[3-(2-fluorophenyl)-1H-indol-1-yl]-3-(methylamino)propan-2-ol;
1-[3-(3,4-dichlorophenyl)-1H-indol-1-yl]-1-(3-fluorophenyl)-3-(methylamino)propan-2-ol;
1-(3-fluorophenyl)-1-[3-(3-fluorophenyl)-1H-indol-1-yl]-3-(methylamino)propan-2-ol;
1-(5-fluoro-3-methyl-1H-indol-1-yl)-3-(methylamino)-1-phenylpropan-2-ol;
3-amino-1-(5-fluoro-3-methyl-1H-indol-1-yl)-1-phenylpropan-2-ol;
1-(5-chloro-3-methyl-1H-indol-1-yl)-3-(methylamino)-1-phenylpropan-2-ol;
3amino-1-(5-chloro-3-methyl-1H-indol-1-yl)-1-phenylpropan-2-ol;
[3-(5-chloro-3-methyl-1H-indol-1-yl)-2-methoxy-3-phenylpropyl]methylamine;
1-(7-chloro-3-methyl-1H-indol-1-yl)-3-(methylamino)-1-phenylpropan-2-ol;
[3-(5-fluoro-3-methyl-1H-indol-1-yl)-2-methoxy-3-phenylpropyl]methylamine;
1-(4-bromo-1H-indol-1-yl)-3-(methylamino)-1-phenylpropan-2-ol;
1-(4-bromo-1H-indol-1-yl)-1-(3-fluorophenyl)-3-(methylamino)propan-2-ol;
1-(5-bromo-1H-indol-1-yl)-3-(methylamino)-1-phenylpropan-2-ol;
1-(5-bromo-1H-indol-1-yl)-1-(3-fluorophenyl)-3-(methylamino)propan-2-ol;
1-[2-hydroxy-3-(methylamino)-1-phenylpropyl]-1H-indole-4-carbonitrile;
1-(6-bromo-1H-indol-1-yl)-3-(methylamino)-1-phenylpropan-2-ol;
1-[2-hydroxy-3-(methylamino)-1-phenylpropyl]-1H-indole-5-carbonitrile;
1-[1-(3-fluorophenyl)-2-hydroxy-3-(methylamino)propyl]-1H-indole-4-carbonitrile;
1-(6-bromo-1H-indol-1-yl)-1-(3-fluorophenyl)-3-(methylamino)propan-2-ol;
1-(6-fluoro-1H-indol-1-yl)-1-(3-fluorophenyl)-3-(methylamino)propan-2-ol;
3-amino-1-(3-fluorophenyl)-1-(1H-indol-1-yl)propan-2-ol;
1-(7-bromo-1H-indol-1-yl)-1-(3-fluorophenyl)-3-(methylamino)propan-2-ol;
1-(1H-indol-1-yl)-3-(methylamino)-1-[3-(trifluoromethyl)phenyl]propan-2-ol;
1-(3-fluorophenyl)-3-(methylamino)-1-spiro[cyclohexane-1,3′-indol]-1′(2′H)-ylpropan-2-ol;
1-(2,3-dihydro-1H-indol-1-yl)-3-(methylamino)-1-[3-(trifluoromethyl)phenyl]propan-2-ol;
1-(3-fluorophenyl)-1-(1H-indol-1-yl)-3-(methylamino)propan-2-ol;
1-(3,4-difluorophenyl)-1-(1H-indol-1-yl)-3-(methylamino)propan-2-ol;
1-(3-fluorophenyl)-3-(methylamino)-1-(3-methyl-1H-indol-1-yl)propan-2-ol;
1-(4-chloro-1H-indol-1-yl)-3-(methylamino)-1-phenylpropan-2-ol;
1-(6-chloro-1H-indol-1-yl)-3-(methylamino)-1-phenylpropan-2-ol;
1-(7-chloro-1H-indol-1-yl)-3-(methylamino)-1-phenylpropan-2-ol;
1-(7-chloro-1H-indol-1-yl)-1-(3-fluorophenyl)-3-(methylamino)propan-2-ol;
1-(4-chloro-1H-indol-1-yl)-1-(3-fluorophenyl)-3-(methylamino)propan-2-ol;
1-(6-chloro-1H-indol-1-yl)-1-(3-fluorophenyl)-3-(methylamino)propan-2-ol;
1-(5-chloro-1H-indol-1-yl)-3-(methylamino)-1-phenylpropan-2-ol;
1-(5-chloro-1H-indol-1-yl)-1-(3-fluorophenyl)-3-(methylamino)propan-2-ol;
1-(3-isopropyl-1H-indol-1-yl)-3-(methylamino)-1-phenylpropan-2-ol;
1-(3-fluorophenyl)-1-(3-isopropyl-1H-indol-1-yl)-3-(methylamino)propan-2-ol;
1-(3,5-difluorophenyl)-1-(1H-indol-1-yl)-3-(methylamino)propan-2-ol;
1-(3,5-difluorophenyl)-1-(2,3-dihydro-1H-indol-1-yl)-3-(methylamino)propan-2-ol;
4-amino-1-(3-fluorophenyl)-1-(1H-indol-1-yl)butan-2-ol;
1-(3,3-dimethyl-2,3-dihydro-1H-indol-1-yl)-1-(3-fluorophenyl)-3-(methylamino)propan-2-ol;
1-(3,5-difluorophenyl)-1-(3,3-dimethyl-2,3-dihydro-1H-indol-1-yl)-3-(methylamino)propan-2-ol;
1-(3,3-dimethyl-2,3-dihydro-1H-indol-1-yl)-3-(methylamino)-1-phenylpropan-2-ol;
1-(3-fluorophenyl)-3-(methylamino)-1-(3-methyl-2,3-dihydro-1H-indol-1-yl)propan-2-ol;
1-(3-fluorophenyl)-3-(methylamino)-1-spiro[cyclopentane-1,3′-indol]-1′(2′H)-ylpropan-2-ol;
1-(3-fluorophenyl)-1-[3-(4-methoxyphenyl)-1H-indol-1-yl]-3-(methylamino)propan-2-ol;
1-(3-fluorophenyl)-3-(methylamino)-1-[3-(4-methylphenyl)-1H-indol-1-yl]propan-2-ol;
1-[3-(4-tert-butylphenyl)-1H-indol-1-yl]-1-(3-fluorophenyl)-3-(methylamino)propan-2-ol;
1-(3-fluorophenyl)-1-[3-(3-methoxyphenyl)-1H-indol-1-yl]-3-(methylamino)propan-2-ol;
1-(3-fluorophenyl)-3-(methylamino)-1-{3-[4-(trifluoromethyl)phenyl]-1H-indol-1-yl}propan-2-ol;
1-(3,5-difluorophenyl)-1-(6-fluoro-2,3-dihydro-1H-indol-1-yl)-3-(methylamino)propan-2-ol;
1-(3-fluorophenyl)-3-(methylamino)-1-{3-[2-(trifluoromethyl)phenyl]-1H-indol-1-yl}propan-2-ol;
1-(3-fluorophenyl)-1-[3-(2-methoxyphenyl)-1H-indol-1-yl]-3-(methylamino)propan-2-ol;
1-(3-fluorophenyl)-3-(methylamino)-1-{3-[3-(trifluoromethyl)phenyl]-1H-indol-1-yl}propan-2-ol;
3-amino-1-(3-methyl-1H-indol-1-yl)-1-phenylpropan-2-ol;
1-(7-fluoro-3-methyl-1H-indol-1-yl)-3-(methylamino)-1-phenylpropan-2-ol;
3-amino-1-(7-fluoro-3-methyl-1H-indol-1-yl)-1-phenylpropan-2-ol;
1-(7-fluoro-1H-indol-1-yl)-3-(methylamino)-1-phenylpropan-2-ol;
1-(4-fluoro-1H-indol-1-yl)-3-(methylamino)-1-phenylpropan-2-ol;
1-(7-fluoro-1H-indol-1-yl)-1-(3-fluorophenyl)-3-(methylamino)propan-2-ol;
1-(4-fluoro-1H-indol-1-yl)-1-(3-fluorophenyl)-3-(methylamino)propan-2-ol;
1-(3-fluorophenyl)-3-(methylamino)-1-[5-(trifluoromethyl)-1H-indol-1-yl]propan-2-ol;
1-(6-fluoro-1H-indol-1-yl)-3-(methylamino)-1-phenylpropan-2-ol;
3-(methylamino)-1-phenyl-1-[6-(trifluoromethyl)-1H-indol-1-yl]propan-2-ol;
3-(methylamino)-1-phenyl-1-[5-(trifluoromethyl)-1H-indol-1-yl]propan-2-ol;
1-(3-tert-butyl-1H-indol-1-yl)-1-(3-fluorophenyl)-3-(methylamino)propan-2-ol;
1-(1H-indol-1-yl)-2-methyl-3-(methylamino)-1-phenylpropan-2-ol;
3-(1H-indol-1-yl)-1-(methylamino)-3-phenylbutan-2-ol;
1-tert-butyl-3-[2hydroxy-3-(methylamino)-1-phenylpropyl]-1,3-dihydro-2H-benzimidazol-2-one;
1-[2-hydroxy-3-(methylamino)-1-phenylpropyl]-3-propyl-1,3-dihydro-2H-benzimidazol-2-one;
5-bromo-1-[2hydroxy-3-(methylamino)-1-phenylpropyl]-3,3-dimethyl-1,3-dihydro-2H-indol-2-one;
6-fluoro-1-[2hydroxy-3-(methylamino)-1-phenylpropyl]-3,3-dimethyl-1,3-dihydro-2H-indol-2-one;
4-fluoro-1-[2hydroxy-3-(methylamino)-1-phenylpropyl]-3,3-dimethyl-1,3-dihydro-2H-indol-2-one;
1-cyclobutyl-3-[2hydroxy-3-(methylamino)-1-phenylpropyl]-1,3-dihydro-2H-benzimidazol-2-one;
5-fluoro-3-[2hydroxy-3-(methylamino)-1-phenylpropyl]-1-propyl-1,3-dihydro-2H-benzimidazol-2-one;
1-ethyl-3-[1-(3-fluorophenyl)-2-hydroxy-3-(methylamino)propyl]-1,3-dihydro-2H-benzimidazol-2-one;
1-ethyl-3-[2hydroxy-3-(methylamino)-1-phenylpropyl]-1,3-dihydro-2H-benzimidazol-2-one;
4-fluoro-3-[2hydroxy-3-(methylamino)-1-phenylpropyl]-1-isopropyl-1,3-dihydro-2H-benzimidazol-2-one;
1-cyclopentyl-3-[2hydroxy-3-(methylamino)-1-phenylpropyl]-1,3-dihydro-2H-benzimidazol-2-one;
1-[2hydroxy-3-(methylamino)-1-phenylpropyl]-3-isopropyl-1,3-dihydro-2H-benzimidazol-2-one;
3-[3(ethylamino)-2-hydroxy-1-phenylpropyl]-5-fluoro-1-isopropyl-1,3-dihydro-2H-benzimidazol-2-one;
1-[2hydroxy-3-(methylamino)-1-phenylpropyl]-3-methyl-1,3-dihydro-2H-benzimidazol-2-one;
1-ethyl-5-fluoro-3-[2-hydroxy-3-(methylamino)-1-phenylpropyl]-1,3-dihydro-2H-benzimidazol-2-one;
1-ethyl-4-fluoro-3-[2-hydroxy-3-(methylamino)-1-phenylpropyl]-1,3-dihydro-2H-benzimidazol-2-one;
4-fluoro-3-[1-(3-fluorophenyl)-2-hydroxy-3-(methylamino)propyl]-1-isopropyl-1,3-dihydro-2H-benzimidazol-2-one;
1-ethyl-4-fluoro-3-[2hydroxy-3-(methylamino)-1-(3-fluorophenyl)-propyl]-1,3-dihydro-2H-benzimidazol-2-one;
1-[1-(3-fluorophenyl)-2-hydroxy-3-(methylamino)propyl]-3,3-dimethyl-1,3-dihydro-2H-indol-2-one;
1-[3-(2,3-difluorophenyl)-1H-indol-1-yl]-1-(3-fluorophenyl)-3-(methylamino)propan-2-ol;
1-[3-(2-chlorophenyl)-1H-indol-1-yl]-1-(3-fluorophenyl)-3-(methylamino)propan-2-ol;
1-(2,3-dihydro-4H-1,4-benzoxazin-4-yl)-3-(methylamino)-1-phenylpropan-2-ol;
3-(methylamino)-1-(4-methyl-3,4-dihydroquinoxalin-1(2H)-yl)-1-phenylpropan-2-ol;
3-(methylamino)-1-phenyl-1-[4-(2,2,2-trifluoroethyl)-3,4-dihydroquinoxalin-1(2H)-yl]propan-2-ol;
1-(6-chloro-2,3-dihydro-4H-1,4-benzoxazin-4-yl)-1-(3,5-difluorophenyl)-3-(methylamino)propan-2-ol;
1-(3-fluorophenyl)-3-(methylamino)-1-(2-methyl-2,3-dihydro-4H-1,4-benzoxazin-4-yl)propan-2-ol;
1-(6-chloro-2,3-dihydro-4H-1,4-benzoxazin-4-yl)-3-(methylamino)-1-phenylpropan-2-ol;
3-(methylamino)-1-(6-methyl-2,3-dihydro-4H-1,4-benzoxazin-4-yl)-1-phenylpropan-2-ol;
1-(6-chloro-2,3-dihydro-4H-1,4-benzoxazin-4-yl)-3-(methylamino)-1-phenylpropan-2-ol;
1-(6-chloro-2,3-dihydro-4H-1,4-benzoxazin-4-yl)-3-(methylamino)-1-phenylpropan-2-ol;
1-(6-chloro-2,3-dihydro-4H-1,4-benzoxazin-4-yl)-1-(3-fluorophenyl)-3-(methylamino)propan-2-ol;
1-(2,2-dimethyl-2,3-dihydro-4H-1,4-benzoxazin-4-yl)-1-(3-fluorophenyl)-3-(methylamino)propan-2-ol;
1-(2,2-dimethyl-2,3-dihydro-4H-1,4-benzoxazin-4-yl)-3-(methylamino)-1-phenylpropan-2-ol;
1-(2,3-dihydro-4H-1,4-benzothiazin-4-yl)-1-(3-fluorophenyl)-3-(methylamino)propan-2-ol;
1-(3-fluorophenyl)-3-(methylamino)-1-(2-phenyl-2,3-dihydro-4H-1,4-benzoxazin-4-yl)propan-2-ol;
1-(3-fluorophenyl)-3-(methylamino)-1-[2-phenyl-2,3-dihydro-4H-1,4-benzoxazin-4-yl]propan-2-ol;
1-(3-fluorophenyl)-3-(methylamino)-1-[2-phenyl-2,3-dihydro-4H-1,4-benzoxazin-4-yl]propan-2-ol; and

pharmaceutically acceptable salts thereof.

The compounds of the present invention may be prepared in a number of ways well known to those skilled in the art. The compounds can be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale.

As will be readily understood, functional groups present may contain protecting groups during the course of synthesis. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Protecting groups that may be employed in accordance with the present invention may be described in Greene, T. W. and Wuts, P. G. M., Protective Groups in Organic Synthesis 2d. Ed., Wiley & Sons, 1991.

Compounds of the present invention are suitably prepared in accordance with the following general description and specific examples. Variables used are as defined for formula I, unless otherwise noted. The reagents used in the preparation of the compounds of this invention can be either commercially obtained or can be prepared by standard procedures described in the literature.

The compounds of this invention contain chiral centers, providing various stereoisomeric forms such as enantiomeric mixtures as well as optical isomers. The individual optical isomers can be prepared directly through asymmetric and/or stereospecific synthesis or by conventional chiral separation of optical isomers from the enantiomeric mixture.

The compounds of the present invention may be prepared in a number of ways well known to those skilled in the art. The compounds can be synthesized, for example, by the methods described below, or variations thereon as appreciated by the skilled artisan. All processes disclosed in association with the present invention are contemplated to be practiced on any scale, including milligram, gram, multigram, kilogram, multikilogram or commercial industrial scale. Compounds of the present invention are suitably prepared in accordance with the following general description and specific examples. Variables used are as defined for formula I, unless otherwise noted. The reagents used in the preparation of the compounds of this invention can be either commercially obtained or can be prepared by standard procedures described in the literature.

The compounds of this invention contain chiral centers, providing various stereoisomeric forms such as enantiomeric mixtures as well as optical isomers. The individual optical isomers can be prepared directly through asymmetric and/or stereospecific synthesis or by conventional chiral separation of optical isomers from the enantiomeric mixture.

As will be readily understood, functional groups present may contain protecting groups during the course of synthesis. Protecting groups are known per se as chemical functional groups that can be selectively appended to and removed from functionalities, such as hydroxyl groups and carboxyl groups. These groups are present in a chemical compound to render such functionality inert to chemical reaction conditions to which the compound is exposed. Any of a variety of protecting groups may be employed with the present invention. Protecting groups that may be employed in accordance with the present invention may be described in Greene, T. W. and Wuts, P. G. M., Protective Groups in Organic Synthesis 2d. Ed., Wiley & Sons, 1991.

In accordance with this invention, compounds of formula I are produced by the following reaction schemes (Schemes I to IV). Depending on the desired diastereomer, the compounds can be prepared via two different synthetic routes (A and B, Schemes I and II). If it is desired to synthesize compounds of formula I-a, they can be prepared from compounds of formula 1 by selectively converting the primary alcohol into a leaving group and displacing it with a desired amine. (Route A, Scheme I) Any conventional method for the selective conversion of a primary alcohol into a leaving group, and any conventional method for displacing a primary leaving group with an amine can be utilized for this conversion. In accordance with the preferred embodiment of this invention, the diol of formula 1 is treated with para-toluenesulfonyl chloride in pyridine to form the tosylate of formula 2, which is converted to the compound of formula I-a through treatment with an excess of alcoholic amine solution, either at room temperature or heated to about 40° C. to about 80° C. in a sealed tube. Compounds of formula I-a can be converted to a pharmaceutically acceptable salt using any conventional method.

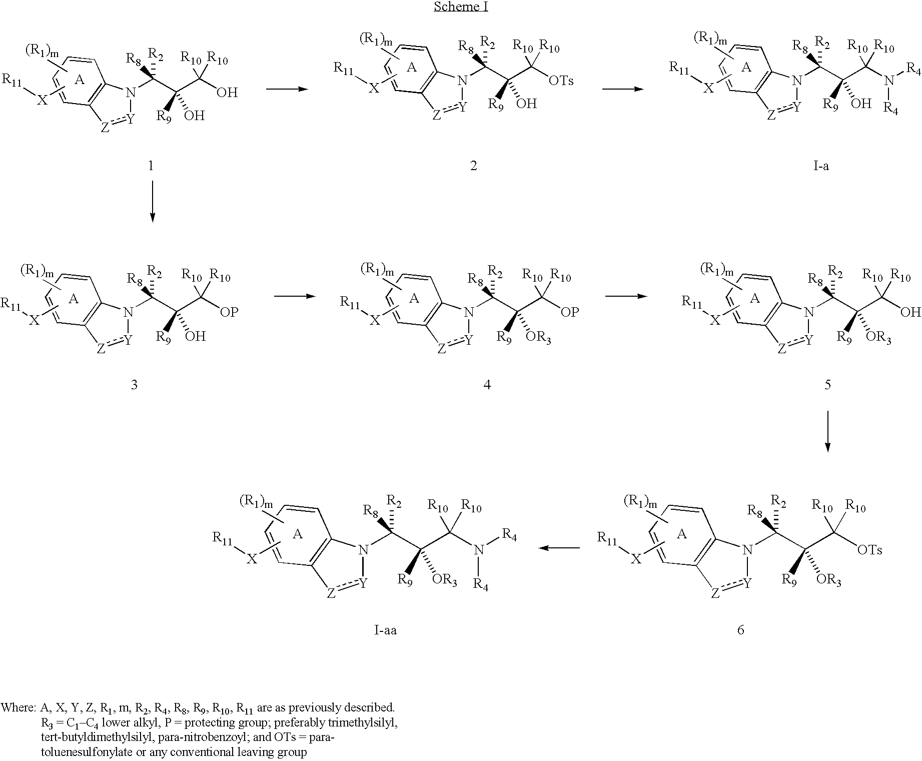

If it is desired to form compounds of formula I-aa, they can be prepared from compounds of formula 1 via selective protection of the primary alcohol, followed by alkylation of the secondary alcohol, and deprotection of the primary alcohol. Any conventional alcohol protecting groups can be utilized for this conversion and any method for the selective protection of a primary alcohol can be employed. According to the preferred embodiment of this invention, the reaction is carried out at low temperature in dichloromethane with trimethylsilyl chloride and triethylamine as base to form compounds of formula 3. Alkylation of the secondary alcohol can be accomplished via any conventional method of alkylating a secondary alcohol found in the literature. According to the preferred embodiment of this invention, compounds of formula 3 are reacted with an alkyl halide using sodium hydride as base to form compounds of formula 4, which can be deprotected to form compounds of formula 5 via any conventional method for deprotection of a primary alcohol. According to the preferred embodiment of this invention, compounds of formula 4 are treated with dilute aqueous hydrochloric acid or trifluoroacetic acid in dichloromethane to form compounds of formula 5. Conversion of the primary alcohol in compounds of formula 5 to complete the synthesis of compounds of formula I-aa can be performed as previously described for the synthesis of compounds of formula I-a. Compounds of formula I-aa can be converted to a pharmaceutically acceptable salt using any conventional method.

Alternatively, compounds of formula 6 can be prepared directly from compounds of formula 2. Any method of alkylating a hydroxyl group in the presence of a tosyl group can be employed for this conversion. In accordance with the preferred embodiment of this invention, compounds of formula 2 are treated with an alkyl trifluoromethanesulfonate, e.g. methyl trifluoromethanesulfonate, in the presence of a hindered base, e.g. 2,6-di-tert-butyl-4-methylpyridine. The reaction can be performed either at room temperature or heated to about 40° C. to about 80° C. Compounds of formula 6 can be converted to compounds of formula I-aa as previously described for the synthesis of compounds of formula I-a. Compounds of formula I-aa can be converted to a pharmaceutically acceptable salt using any conventional method.

If it is desired to form compounds of I-b, they can also be prepared from compounds of formula 1 via Route B (Scheme II). This route involves the selective protection of the primary alcohol followed by conversion of the secondary alcohol to a leaving group. Any conventional method for the selective protection of a primary alcohol, and any conventional method for converting of a secondary alcohol into a leaving group can be utilized for this conversion. In accordance with the preferred embodiment of this invention, compounds of formula 1 are treated with para-nitrobenzoyl chloride in pyridine at low temperature (preferably below about 0° C.) to form compounds of formula 7. Compounds of formula 7 can be converted to a secondary mesylate of formula 8 via reaction with methanesulfonyl chloride in dichloromethane using triethylamine as base. The reaction is preferably carried out at temperatures between about −15° C. and about 10° C. Deprotection of the primary alcohol in compounds of formula 8 allows for the formation of a primary epoxide through an S_{N}2 reaction resulting in an inversion of the stereocenter. Any conventional method for deprotection of a primary alcohol, and any conventional method for epoxide formation onto an alpha leaving group can be employed for this conversion. In accordance with the preferred embodiment of this invention, compounds of formula 8 are treated with an aqueous solution of a suitable base in organic solvent, preferably, aqueous sodium hydroxide in dioxane. The resulting epoxide of formula 9 can be ring-opened regioselectively with an amine to produce the desired aminoalcohol of formula I-b. Any conventional method for the regioselective ring opening of a primary epoxide can be employed for this conversion. In accordance with the preferred embodiment of this invention, compounds of formula 9 are treated with an excess of an alcoholic amine solution in a sealed flask, either at room temperature or heated to about 40° C. to about 90° C. Compounds of formula I-b can be converted into a pharmaceutically acceptable salt using conventional methods.

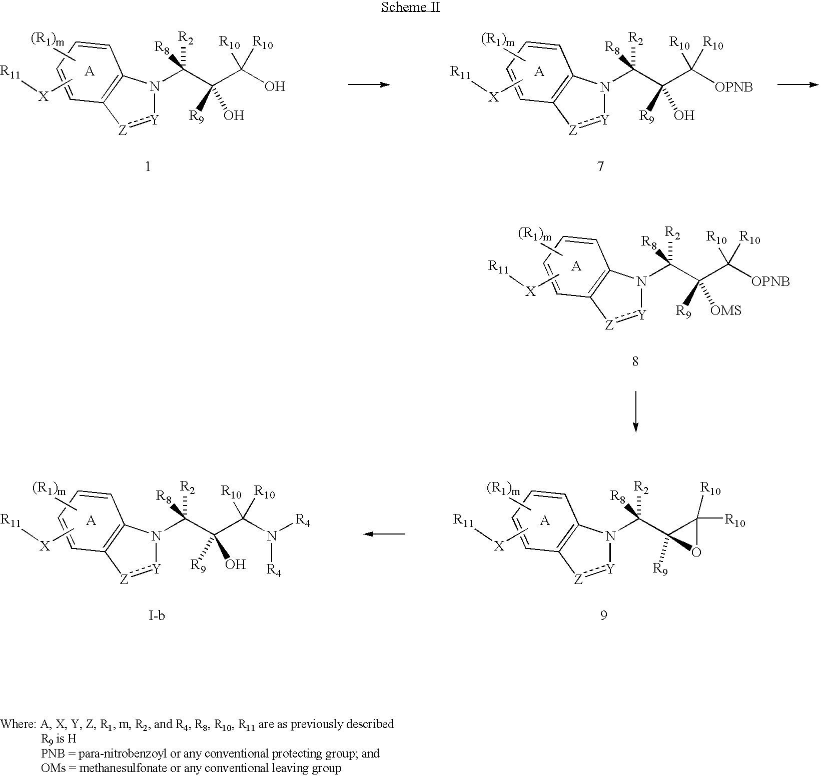

If it is desired to form compounds of formula I-bb, they can be made from compounds of formula I-b via protection of the amine, alkylation of the secondary alcohol and deprotection of the amine (Scheme III). Any conventional method for protection of an amine, alkylation of a secondary alcohol, and deprotection of an amine can be utilized for this conversion. In accordance with the preferred embodiment of this invention, compounds of formula I-b are treated with boc anhydride, where boc=tert-butoxycarbonyl, to form compounds of formula 10 which can be alkylated with an alkyl halide using sodium hydride as base to form compounds of formula 11. Deprotection is accomplished using an acid, preferably trifluoroacetic acid in dichloromethane to form compounds of formula I-bb that can be converted into a pharmaceutically acceptable salt using conventional methods.

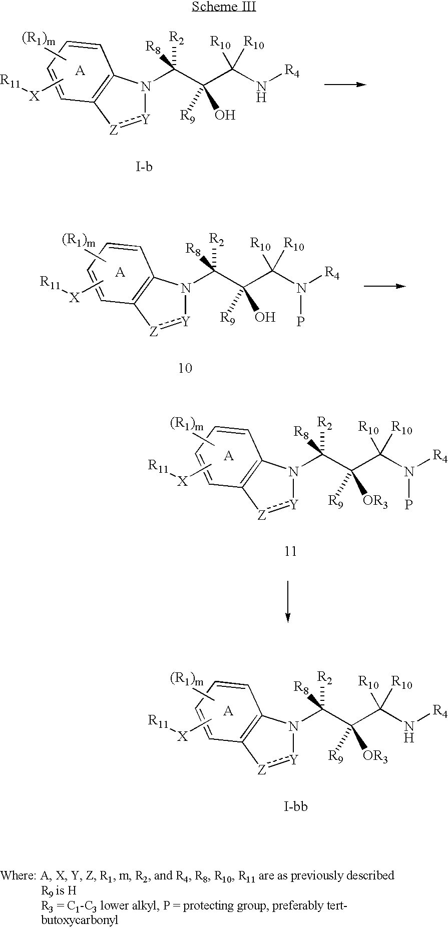

Compounds of formula 1 are formed via a regio- and stereo-selective ring opening of an appropriately substituted epoxide of formula 13 (formed via an epoxidation of an appropriately substituted allylic alcohol 14) with an appropriately substituted compound of formula 12 (Scheme IV). Any conventional method for the regio- and stereo-selective ring opening of an epoxide can be employed for this conversion. In accordance with the preferred embodiment of this invention, compounds of formula 12 are treated with a base, e.g. sodium hydride, sodium tert-butoxide, potassium hydroxide, potassium tert-butoxide or potassium hydroxide, then treated with the epoxide of formula 13. The epoxide of formula 13 can be pre-treated with a Lewis acid, e.g. titanium iso-propoxide, boron-trifluoride, etc. to ensure regio-selective ring-opening. The reaction occurs at room temperature over a duration of about 2 hours to about 72 hours. Alternatively, compounds of formula 12 that are suitably nucleophilic, e.g. indoline, can be heated with the epoxide of formula 13 at temperatures from about 50° C. to about 170° C. to form compounds of formula 1.

Epoxidation of trans-allylic alcohols 14 can be performed either racemically or asymmetrically using methods described in the literature. In accordance with the preferred embodiment of this invention, racemic epoxidation is conducted with either peracetic acid or meta-chloroperbenzoic acid. If it is desired to produce a single enantiomer of compounds of formula I, asymmetric epoxidation of an allylic alcohol can be performed with tert-butylhydroperoxide or cumene hydroperoxide in the presence of the appropriate tartrate ester, titanium (IV) isopropoxide, and molecular sieves. This method is well established in the literature (e.g. K. B. Sharpless, et. al., J. Org. Chem. 1986, 51, 3710). Compounds of formula 12 and the starting allylic alcohols 14 are either available from commercial sources or are accessible through methods well established in the literature.

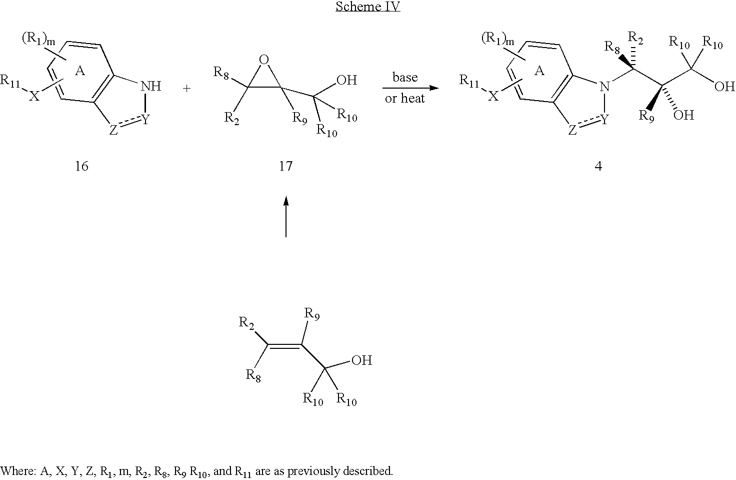

In accordance with this invention, compounds of formula II are produced by the following reaction schemes (Schemes V to VIII). Depending on the desired diastereomer, the compounds can be prepared via two different synthetic routes (A and B, Schemes V and VI). If it is desired to synthesize compounds of formula II-a, they can be prepared from compounds of formula 15 by selectively converting the primary alcohol into a leaving group and displacing it with a desired amine. (Route A, Scheme V) Any conventional method for the selective conversion of a primary alcohol into a leaving group, and any conventional method for displacing a primary leaving group with an amine can be utilized for this conversion. In accordance with the preferred embodiment of this invention, the diol of formula 15 is treated with para-toluenesulfonyl chloride in pyridine to form the tosylate of formula 16, which is converted to the compound of formula II-a through treatment with an excess of alcoholic amine solution, either at room temperature or heated to about 40° C. to about 80° C. in a sealed tube. Compounds of formula II-a can be converted to a pharmaceutically acceptable salt using any conventional method.

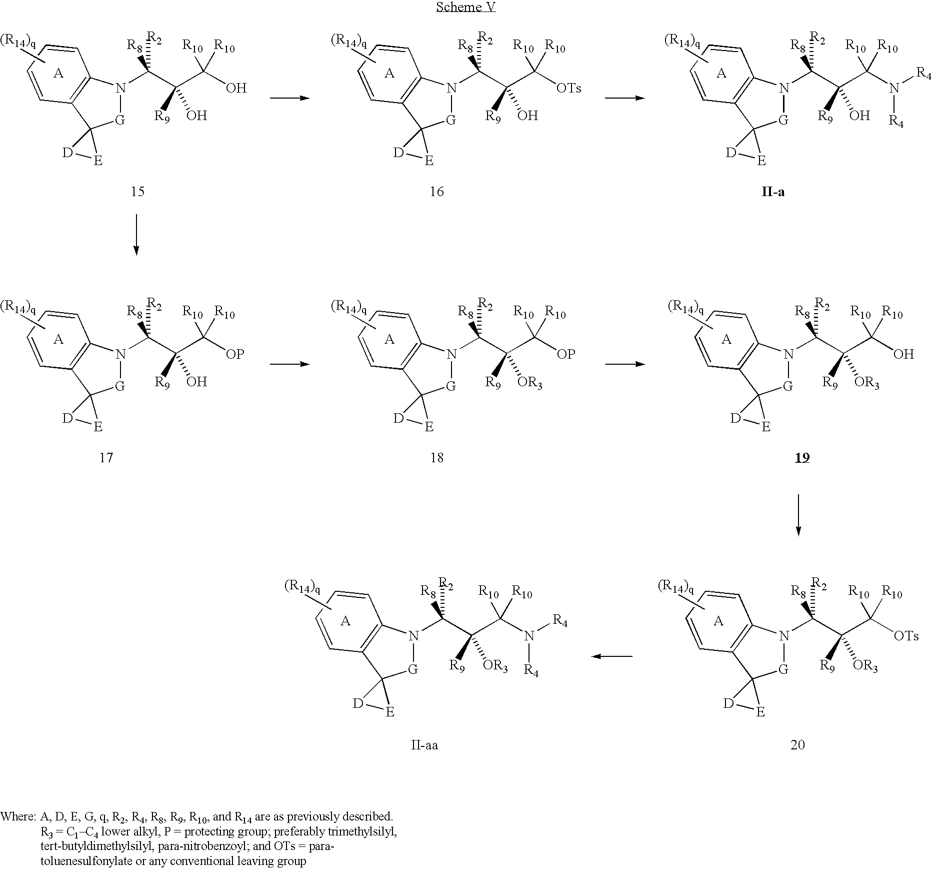

If it is desired to form compounds of formula II-aa, they can be prepared from compounds of formula 15 via selective protection of the primary alcohol, followed by alkylation of the secondary alcohol, and deprotection of the primary alcohol. Any conventional alcohol protecting groups can be utilized for this conversion and any method for the selective protection of a primary alcohol can be employed. According to the preferred embodiment of this invention, the reaction is carried out at low temperature in dichloromethane with trimethylsilyl chloride and triethylamine as base to form compounds of formula 17. Alkylation of the secondary alcohol can be accomplished via any conventional method of alkylating a secondary alcohol found in the literature. According to the preferred embodiment of this invention, compounds of formula 17 are reacted with an alkyl halide using sodium hydride as base to form compounds of formula 18, which can be deprotected to form compounds of formula 19 via any conventional method for deprotection of a primary alcohol. According to the preferred embodiment of this invention, compounds of formula 18 are treated with dilute aqueous hydrochloric acid or trifluoroacetic acid in dichloromethane to form compounds of formula 19. Conversion of the primary alcohol in compounds of formula 19 to complete the synthesis of compounds of formula II-aa can be performed as previously described for the synthesis of compounds of formula II-a. Compounds of formula II-aa can be converted to a pharmaceutically acceptable salt using any conventional method.

Alternatively, compounds of formula 20 can be prepared directly from compounds of formula 16. Any method of alkylating a hydroxyl group in the presence of a tosyl group can be employed for this conversion. In accordance with the preferred embodiment of this invention, compounds of formula 16 are treated with an alkyl trifluoromethanesulfonate, e.g. methyl trifluoromethanesulfonate, in the presence of a hindered base, e.g. 2,6-di-tert-butyl-4-methylpyridine. The reaction can be performed either at room temperature or heated to about 40° C. to about 80° C. Compounds of formula 20 can be converted to compounds of formula II-aa as previously described for the synthesis of compounds of formula II-a. Compounds of formula II-aa can be converted to a pharmaceutically acceptable salt using any conventional method.

If it is desired to form compounds of II-b, they can also be prepared from compounds of formula 15 via Route B (Scheme VI). This route involves the selective protection of the primary alcohol followed by conversion of the secondary alcohol to a leaving group. Any conventional method for the selective protection of a primary alcohol, and any conventional method for converting of a secondary alcohol into a leaving group can be utilized for this conversion. In accordance with the preferred embodiment of this invention, compounds of formula 15 are treated with para-nitrobenzoyl chloride in pyridine at low temperature (preferably below about 0° C.) to form compounds of formula 21. Compounds of formula 21 can be converted to a secondary mesylate of formula 22 via reaction with methanesulfonyl chloride in dichloromethane using triethylamine as base. The reaction is preferably carried out at temperatures between about −15° C. and about 10° C. Deprotection of the primary alcohol in compounds of formula 22 allows for the formation of a primary epoxide through an S_{N}2 reaction resulting in an inversion of the stereocenter. Any conventional method for deprotection of a primary alcohol, and any conventional method for epoxide formation onto an alpha leaving group can be employed for this conversion. In accordance with the preferred embodiment of this invention, compounds of formula 22 are treated with an aqueous solution of a suitable base in organic solvent, preferably, aqueous sodium hydroxide in dioxane. The resulting epoxide of formula 23 can be ring-opened regioselectively with an amine to produce the desired aminoalcohol of formula II-b. Any conventional method for the regioselective ring opening of a primary epoxide can be employed for this conversion. In accordance with the preferred embodiment of this invention, compounds of formula 23 are treated with an excess of an alcoholic amine solution in a sealed flask, either at room temperature or heated to about 40° C. to about 90° C. Compounds of formula II-b can be converted into a pharmaceutically acceptable salt using conventional methods.

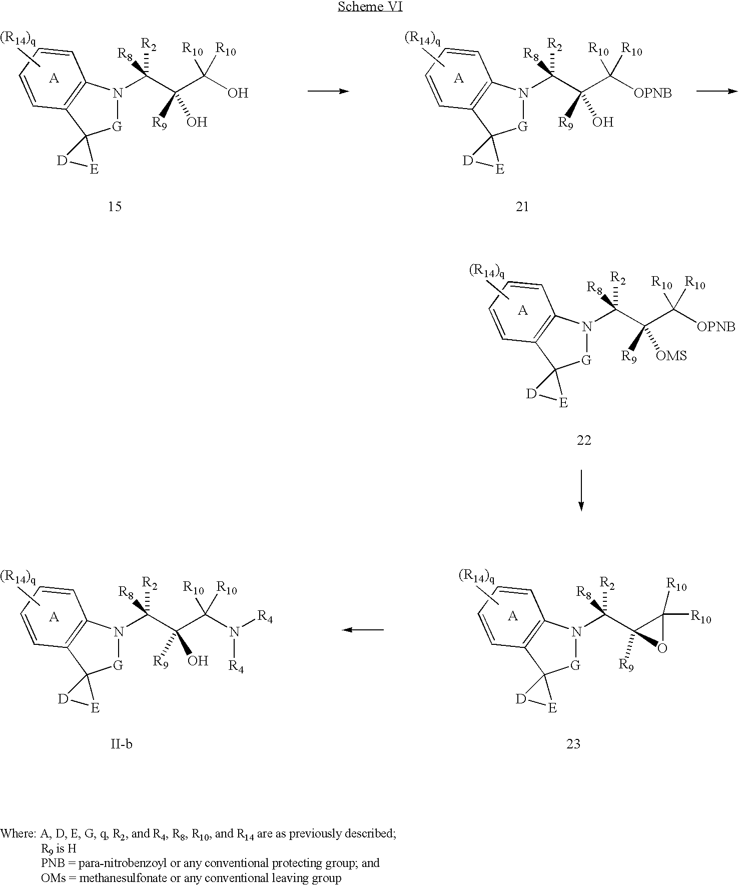

If it is desired to form compounds of formula II-bb, they can be made from compounds of formula II-b via protection of the amine, alkylation of the secondary alcohol and deprotection of the amine (Scheme VII). Any conventional method for protection of an amine, alkylation of a secondary alcohol, and deprotection of an amine can be utilized for this conversion. In accordance with the preferred embodiment of this invention, compounds of formula II-b are treated with boc anhydride, where boc=tert-butoxycarbonyl, to form compounds of formula 24 which can be alkylated with an alkyl halide using sodium hydride as base to form compounds of formula 25. Deprotection is accomplished using an acid, preferably trifluoroacetic acid in dichloromethane to form compounds of formula II-bb that can be converted into a pharmaceutically acceptable salt using conventional methods.

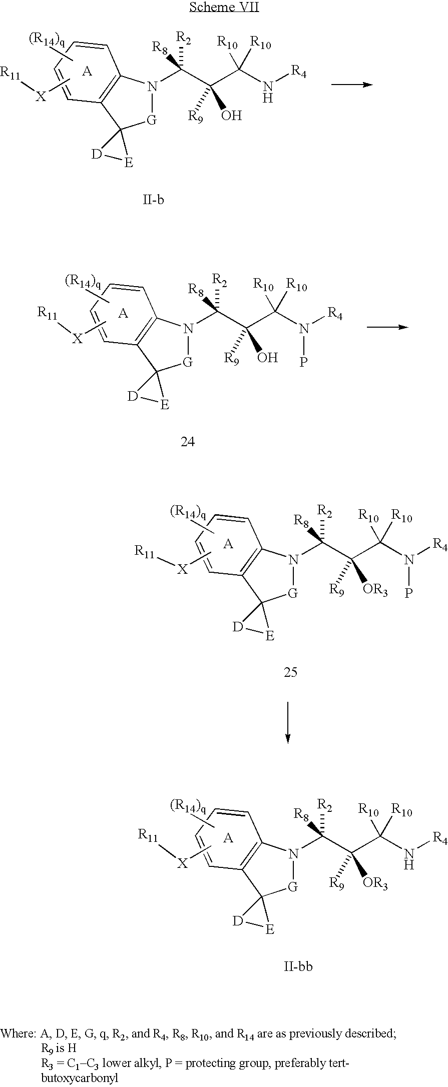

Compounds of formula 15 are formed via a regio- and stereo-selective ring opening of an appropriately substituted epoxide of formula 13 (formed via an epoxidation of an appropriately substituted allylic alcohol 14) with an appropriately substituted compound of formula 26 (Scheme IV). Any conventional method for the regio- and stereo-selective ring opening of an epoxide can be employed for this conversion. In accordance with the preferred embodiment of this invention, compounds of formula 26 are treated with a base, e.g. sodium hydride, sodium tert-butoxide, potassium hydroxide, potassium tert-butoxide or potassium hydroxide, then treated with the epoxide of formula 13. The epoxide of formula 13 can be pre-treated with a Lewis acid, e.g. titanium iso-propoxide, boron-trifluoride, etc. to ensure regio-selective ring-opening. The reaction occurs at room temperature over a duration of about 2 hours to about 72 hours. Alternatively, compounds of formula 26 that are suitably nucleophilic, e.g. indoline, can be heated with the epoxide of formula 13 at temperatures from about 50° C. to about 170° C. to form compounds of formula 15.

Epoxidation of trans-allylic alcohols 14 can be performed either racemically or asymmetrically using methods described in the literature. In accordance with the preferred embodiment of this invention, racemic epoxidation is conducted with either peracetic acid or meta-chloroperbenzoic acid. If it is desired to produce a single enantiomer of compounds of formula II, asymmetric epoxidation of an allylic alcohol can be performed with tert-butylhydroperoxide or cumene hydroperoxide in the presence of the appropriate tartrate ester, titanium (IV) isopropoxide, and molecular sieves. This method is well established in the literature (e.g. K. B. Sharpless, et. al., J. Org. Chem. 1986, 51, 3710). Compounds of formula 26 and the starting allylic alcohols 13 are either available from commercial sources or are accessible through methods well established in the literature.

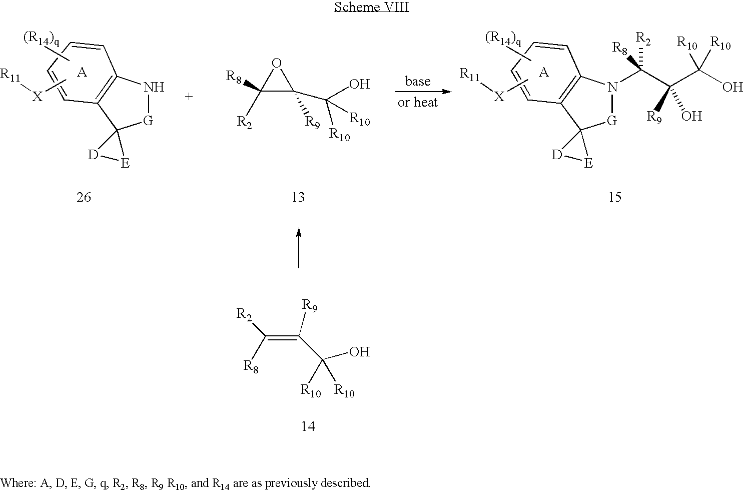

In accordance with this invention, compounds of formula III are produced by the following reaction schemes (Schemes IX to XII). Depending on the desired diastereomer, the compounds can be prepared via two different synthetic routes (A and B, Schemes IX and X). If it is desired to synthesize compounds of formula III-a, they can be prepared from compounds of formula 27 by selectively converting the primary alcohol into a leaving group and displacing it with a desired amine. (Route A, Scheme IX) Any conventional method for the selective conversion of a primary alcohol into a leaving group, and any conventional method for displacing a primary leaving group with an amine can be utilized for this conversion. In accordance with the preferred embodiment of this invention, the diol of formula 27 is treated with para-toluenesulfonyl chloride in pyridine to form the tosylate of formula 28, which is converted to the compound of formula III-a through treatment with an excess of alcoholic amine solution, either at room temperature or heated to about 40° C. to about 80° C. in a sealed tube. Compounds of formula III-a can be converted to a pharmaceutically acceptable salt using any conventional method.

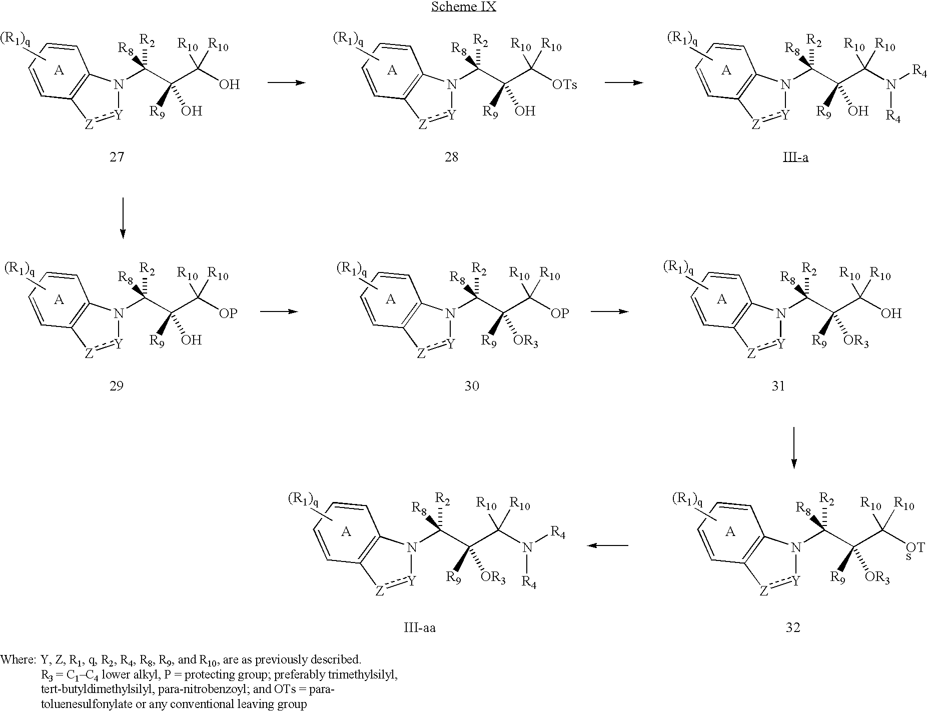

If it is desired to form compounds of formula III-aa, they can be prepared from compounds of formula 27 via selective protection of the primary alcohol, followed by alkylation of the secondary alcohol, and deprotection of the primary alcohol. Any conventional alcohol protecting groups can be utilized for this conversion and any method for the selective protection of a primary alcohol can be employed. According to the preferred embodiment of this invention, the reaction is carried out at low temperature in dichloromethane with trimethylsilyl chloride and triethylamine as base to form compounds of formula 3. Alkylation of the secondary alcohol can be accomplished via any conventional method of alkylating a secondary alcohol found in the literature. According to the preferred embodiment of this invention, compounds of formula 29 are reacted with an alkyl halide using sodium hydride as base to form compounds of formula 30, which can be deprotected to form compounds of formula 31 via any conventional method for deprotection of a primary alcohol. According to the preferred embodiment of this invention, compounds of formula 30 are treated with dilute aqueous hydrochloric acid or trifluoroacetic acid in dichloromethane to form compounds of formula 31. Conversion of the primary alcohol in compounds of formula 31 to complete the synthesis of compounds of formula III-aa can be performed as previously described for the synthesis of compounds of formula III-a. Compounds of formula III-aa can be converted to a pharmaceutically acceptable salt using any conventional method.

Alternatively, compounds of formula 32 can be prepared directly from compounds of formula 28. Any method of alkylating a hydroxyl group in the presence of a tosyl group can be employed for this conversion. In accordance with the preferred embodiment of this invention, compounds of formula 28 are treated with an alkyl trifluoromethanesulfonate, e.g. methyl trifluoromethanesulfonate, in the presence of a hindered base, e.g. 2,6-di-tert-butyl-4-methylpyridine. The reaction can be performed either at room temperature or heated to about 40° C. to about 80° C. Compounds of formula 32 can be converted to compounds of formula III-aa as previously described for the synthesis of compounds of formula III-a. Compounds of formula III-aa can be converted to a pharmaceutically acceptable salt using any conventional method.

If it is desired to form compounds of III-b, they can also be prepared from compounds of formula 27 via Route B (Scheme X). This route involves the selective protection of the primary alcohol followed by conversion of the secondary alcohol to a leaving group. Any conventional method for the selective protection of a primary alcohol, and any conventional method for converting of a secondary alcohol into a leaving group can be utilized for this conversion. In accordance with the preferred embodiment of this invention, compounds of formula 27 are treated with para-nitrobenzoyl chloride in pyridine at low temperature (preferably below about 0° C.) to form compounds of formula 33. Compounds of formula 33 can be converted to a secondary mesylate of formula 34 via reaction with methanesulfonyl chloride in dichloromethane using triethylamine as base. The reaction is preferably carried out at temperatures between about −15° C. and about 10° C. Deprotection of the primary alcohol in compounds of formula 34 allows for the formation of a primary epoxide through an S_{N}2 reaction resulting in an inversion of the stereocenter. Any conventional method for deprotection of a primary alcohol, and any conventional method for epoxide formation onto an alpha leaving group can be employed for this conversion. In accordance with the preferred embodiment of this invention, compounds of formula 34 are treated with an aqueous solution of a suitable base in organic solvent, preferably, aqueous sodium hydroxide in dioxane. The resulting epoxide of formula 35 can be ring-opened regioselectively with an amine to produce the desired aminoalcohol of formula III-b. Any conventional method for the regioselective ring opening of a primary epoxide can be employed for this conversion. In accordance with the preferred embodiment of this invention, compounds of formula 35 are treated with an excess of an alcoholic amine solution in a sealed flask, either at room temperature or heated to about 40° C. to about 90° C. Compounds of formula III-b can be converted into a pharmaceutically acceptable salt using conventional methods.

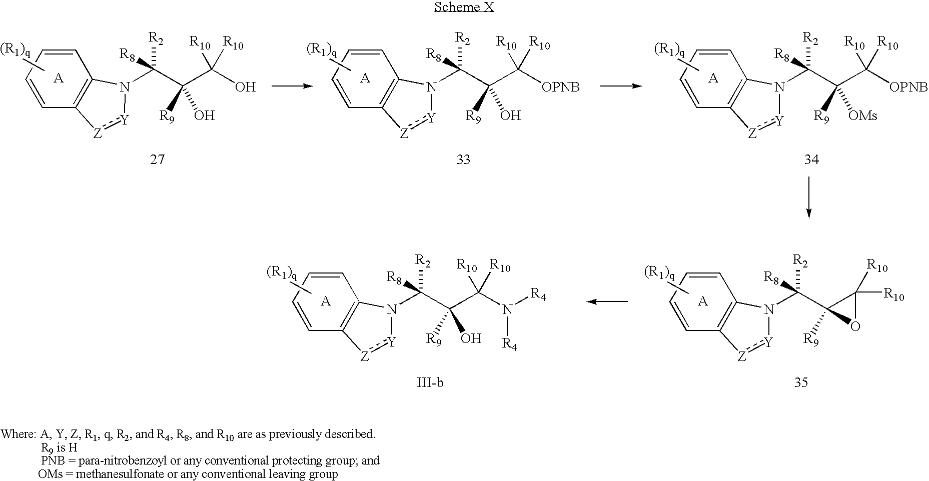

If it is desired to form compounds of formula III-bb, they can be made from compounds of formula III-b via protection of the amine, alkylation of the secondary alcohol and deprotection of the amine (Scheme XI). Any conventional method for protection of an amine, alkylation of a secondary alcohol, and deprotection of an amine can be utilized for this conversion. In accordance with the preferred embodiment of this invention, compounds of formula III-b are treated with boc anhydride, where boc=tert-butoxycarbonyl, to form compounds of formula 36 which can be alkylated with an alkyl halide using sodium hydride as base to form compounds of formula 37. Deprotection is accomplished using an acid, preferably trifluoroacetic acid in dichloromethane to form compounds of formula III-bb that can be converted into a pharmaceutically acceptable salt using conventional methods.

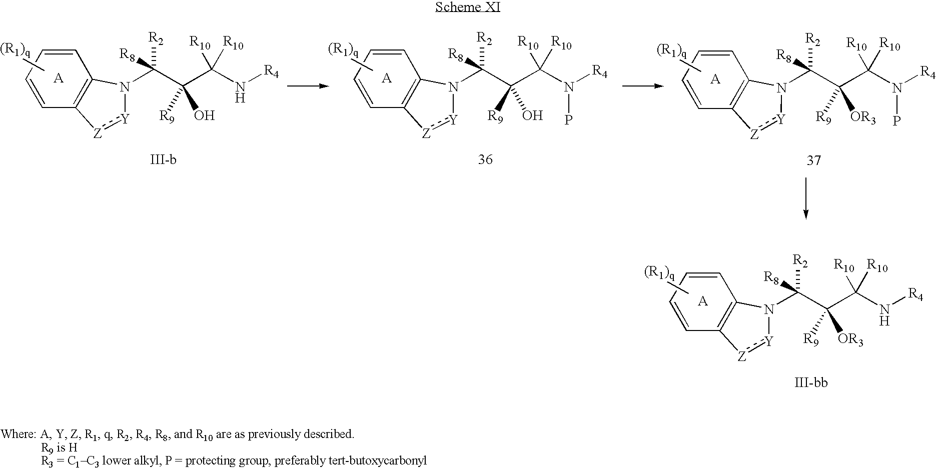

Compounds of formula 27 are formed via a regio- and stereo-selective ring opening of an appropriately substituted epoxide of formula 13 (formed via an epoxidation of an appropriately substituted allylic alcohol 14) with an appropriately substituted compound of formula 38 (Scheme XII). Any conventional method for the regio- and stereo-selective ring opening of an epoxide can be employed for this conversion. In accordance with the preferred embodiment of this invention, compounds of formula 38 are treated with a base, e.g. sodium hydride, sodium tert-butoxide, potassium hydroxide, potassium tert-butoxide or potassium hydroxide, then treated with the epoxide of formula 13. The epoxide of formula 13 can be pre-treated with a Lewis acid, e.g. titanium iso-propoxide, boron-trifluoride, etc. to ensure regio-selective ring-opening. The reaction occurs at room temperature over a duration of about 2 hours to about 72 hours. Alternatively, compounds of formula 38 that are suitably nucleophilic, e.g. indoline, can be heated with the epoxide of formula 13 at temperatures from about 50° C. to about 170° C. to form compounds of formula 27.

Epoxidation of trans-allylic alcohols 14 can be performed either racemically or asymmetrically using methods described in the literature. In accordance with the preferred embodiment of this invention, racemic epoxidation is conducted with either peracetic acid or meta-chloroperbenzoic acid. If it is desired to produce a single enantiomer of compounds of formula I, asymmetric epoxidation of an allylic alcohol can be performed with tert-butylhydroperoxide or cumene hydroperoxide in the presence of the appropriate tartrate ester, titanium (IV) isopropoxide, and molecular sieves. This method is well established in the literature (e.g. K. B. Sharpless, et. al., J. Org. Chem. 1986, 51, 3710). Compounds of formula 38 and the starting allylic alcohols 14 are either available from commercial sources or are accessible through methods well established in the literature.

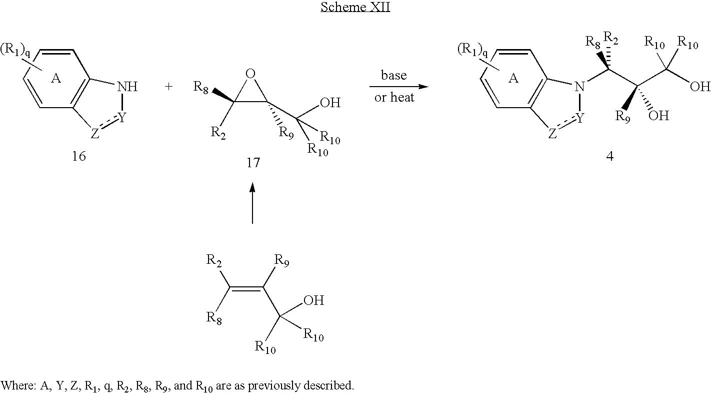

In other embodiments, the invention is directed to pharmaceutical compositions, comprising:
a. at least compound of formula I, II, or III, or pharmaceutically acceptable salt thereof; and
b. at least one pharmaceutically acceptable carrier.

Generally, the compound of formula I, II, or III, or a pharmaceutically acceptable salt thereof, will be present at a level of from about 0.1%, by weight, to about 90% by weight, based on the total weight of the pharmaceutical composition, based on the total weight of the pharmaceutical composition. Preferably, the compound of formula I, II, or III, or a pharmaceutically acceptable salt thereof, will be present at a level of at least about 1%, by weight, based on the total weight of the pharmaceutical composition. More preferably, the compound of formula I, II, or III, or a pharmaceutically acceptable salt thereof, will be present at a level of at least about 5%, by weight, based on the total weight of the pharmaceutical composition. Even more preferably, the norepinephrine reuptake inhibitor or a pharmaceutically acceptable salt thereof will be present at a level of at least about 10%, by weight, based on the total weight of the pharmaceutical composition. Yet even more preferably, the compound of formula I, II, or III, or a pharmaceutically acceptable salt thereof, will be present at a level of at least about 25%, by weight, based on the total weight of the pharmaceutical composition.

Such compositions are prepared in accordance with acceptable pharmaceutical procedures, such as described in Remington's Pharmaceutical Sciences, 17th edition, ed. Alfonoso R. Gennaro, Mack Publishing Company, Easton, Pa. (1985). Pharmaceutically acceptable carriers are those that are compatible with the other ingredients in the formulation and biologically acceptable.

The compounds of this invention may be administered orally or parenterally, neat or in combination with conventional pharmaceutical carriers. Applicable solid carriers can include one or more substances that may also act as flavoring agents, lubricants, solubilizers, suspending agents, fillers, glidants, compression aids, binders or tablet-disintegrating agents or an encapsulating material. In powders, the carrier is a finely divided solid that is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium phosphate, magnesium stearate, talc, sugars, lactose, dextrin, starch, gelatin, cellulose, methyl cellulose, sodium carboxymethyl cellulose, polyvinylpyrrolidine, low melting waxes and ion exchange resins.

Liquid carriers may be used in preparing solutions, suspensions, emulsions, syrups, and elixirs. The active ingredient of this invention can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particularly containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also be an oily ester such as ethyl oleate and isopropyl myristate. Sterile liquid carriers are used in sterile liquid form compositions for parenteral administration.

Liquid pharmaceutical compositions, which are sterile solutions or suspensions, can be administered by, for example, intramuscular, intraperitoneal or subcutaneous injection. Sterile solutions can also be administered intravenously. Oral administration may be either liquid or solid composition form.

Preferably the pharmaceutical composition is in unit dosage form, e.g. as tablets, capsules, powders, solutions, suspensions, emulsions, granules, or suppositories. In such form, the composition is sub-divided in unit dose containing appropriate quantities of the active ingredient; the unit dosage forms can be packaged compositions, for example packeted powders, vials, ampoules, prefilled syringes or sachets containing liquids. The unit dosage form can be, for example, a capsule or tablet itself, or it can be the appropriate number of any such compositions in package form.

In another embodiment of the present invention, the compounds useful in the present invention may be administered to a mammal with one or more other pharmaceutical active agents such as those agents being used to treat any other medical condition present in the mammal. Examples of such pharmaceutical active agents include pain relieving agents, anti-angiogenic agents, anti-neoplastic agents, anti-diabetic agents, anti-infective agents, or gastrointestinal agents, or combinations thereof.

The one or more other pharmaceutical active agents may be administered in a therapeutically effective amount simultaneously (such as individually at the same time, or together in a pharmaceutical composition), and/or successively with one or more compounds of the present invention.

The term “combination therapy” refers to the administration of two or more therapeutic agents or compounds to treat a therapeutic condition or disorder described in the present disclosure, for example hot flush, sweating, thermoregulatory-related condition or disorder, or other. Such administration includes use of each type of therapeutic agent in a concurrent manner. In either case, the treatment regimen will provide beneficial effects of the drug combination in treating the conditions or disorders described herein.

The route of administration may be any route, which effectively transports the active compound of formula I, II, or III, or a pharmaceutically acceptable salt thereof, to the appropriate or desired site of action, such as oral, nasal, pulmonary, transdermal, such as passive or iontophoretic delivery, or parenteral, e.g. rectal, depot, subcutaneous, intravenous, intraurethral, intramuscular, intranasal, ophthalmic solution or an ointment. Furthermore, the administration of compound of formula I, II, or III, or pharmaceutically acceptable salt thereof, with other active ingredients may be concurrent or simultaneous.

It is believed that the present invention described presents a substantial breakthrough in the field of treatment, alleviation, inhibition, and/or prevention of conditions ameliorated by monoamine reuptake including, inter alia, vasomotor symptoms (VMS), sexual dysfunction, gastrointestinal and genitourinary disorders, chronic fatigue syndrome, fibromylagia syndrome, nervous system disorders, and combinations thereof, particularly those conditions selected from the group consisting of major depressive disorder, vasomotor symptoms, stress and urge urinary incontinence, fibromyalgia, pain, diabetic neuropathy, schizophrenia, and combinations thereof.

Accordingly, in one embodiment, the present invention is directed to methods for treating or preventing a condition ameliorated by monoamine reuptake in a subject in need thereof, comprising the step of:

administering to said subject an effective amount of a compound of formula I, II, or III or pharmaceutically acceptable salt thereof.

The conditions ameliorated by monoamine reuptake include those selected from the group consisting of vasomotor symptoms, sexual dysfunction, gastrointestinal and genitourinary disorders, chronic fatigue syndrome, fibromylagia syndrome, nervous system disorders, and combinations thereof, particularly those conditions selected from the group consisting of major depressive disorder, vasomotor symptoms, stress and urge urinary incontinence, fibromyalgia, pain, diabetic neuropathy, and combinations thereof.

“Vasomotor symptoms,” “vasomotor instability symptoms” and “vasomotor disturbances” include, but are not limited to, hot flushes (flashes), insomnia, sleep disturbances, mood disorders, irritability, excessive perspiration, night sweats, fatigue, and the like, caused by, inter alia, thermoregulatory dysfunction.

The term “hot flush” is an art-recognized term that refers to an episodic disturbance in body temperature typically consisting of a sudden skin flushing, usually accompanied by perspiration in a subject.

The term “sexual dysfunction” includes, but is not limited to, condition relating to desire and/or arousal.

As used herein, “gastrointestinal and genitourinary disorders” includes irritable bowel syndrome, symptomatic GERD, hypersensitive esophagus, nonulcer dyspepsia, noncardiac chest pain, biliary dyskinesia, sphincter of Oddi dysfunction, incontinence (i.e., urge incontinence, stress incontinence, genuine stress incontinence, and mixed incontinence) (including the involuntary voiding of feces or urine, and dribbling or leakage or feces or urine which may be due to one or more causes including but not limited to pathology altering sphincter control, loss of cognitive function, overdistention of the bladder, hyperreflexia and/or involuntary urethral relaxation, weakness of the muscles associated with the bladder or. neurologic abnormalities), interstitial cystitis (irritable bladder), and chronic pelvic pain (including, but not limited to vulvodynia, prostatodynia, and proctalgia).

As used herein, “chronic fatigue syndrome” (CFS) is a condition characterized by physiological symptoms selected from weakness, muscle aches and pains, excessive sleep, malaise, fever, sore throat, tender lymph nodes, impaired memory and/or mental concentration, insomnia, disordered sleep, localized tenderness, diffuse pain and fatigue, and combinations thereof.

As used herein, “fibromyalgia syndrome” (FMS) includes FMS and other somatoform disorders, including FMS associated with depression, somatization disorder, conversion disorder, pain disorder, hypochondriasis, body dysmorphic disorder, undifferentiated somatoform disorder, and somatoform NOS. FMS and other somatoform disorders are accompanied by physiological symptoms selected from a generalized heightened perception of sensory stimuli, abnormalities in pain perception in the form of allodynia (pain with innocuous stimulation), abnormalities in pain perception in the form of hyperalgesia (increased sensitivity to painful stimuli), and combinations thereof.

As used herein, “nervous system disorders,” includes addictive disorders (including those due to alcohol, nicotine, and other psychoactive substances) and withdrawal syndrome, age-associated learning and mental disorders (including Alzheimer's disease), anorexia nervosa, bulimia nervosa, attention-deficit disorder with or without hyperactivity disorder bipolar disorder, pain, cyclothymic disorder, depression disorder (including major depressive disorder, refractory depression adolescent depression and minor depression), dysthymic disorder, generalized anxiety disorder (GAD), obesity (i.e., reducing the weight of obese or overweight patients), obsessive compulsive disorders and related spectrum disorders, oppositional defiant disorder, panic disorder, post-traumatic stress disorder, premenstrual dysphoric disorder (i.e., premenstrual syndrome and late luteal phase dysphoric disorder), psychotic disorders (including schizophrenia, schizoaffective and schizophreniform disorders), seasonal affective disorder, sleep disorders (such as narcolepsy and enuresis), social phobia (including social anxiety disorder), selective serotonin reuptake inhibition (SSRI) “poop out” syndrome (i.e., wherein a patient who fails to maintain a satisfactory response to SSRI therapy after an initial period of satisfactory response).

As used herein, “pain,” includes both acute pain and chronic pain, which may be centralized pain, peripheral pain, or combination thereof. The term includes many different types of pains including, but not limited to, neuropathic pain, visceral pain, musculoskeletal pain, bony pain, cancer pain, inflammatory pain, and combinations thereof, such as lower back pain, atypical chest pain, headache such as cluster headache, migraine, herpes neuralgia, phantom limb pain, pelvic pain, myofascial face pain, abdominal pain, neck pain, central pain, dental pain, opioid resistant pain, visceral pain, surgical pain, bone injury pain, pain during labor and delivery, pain resulting from burns, post partum pain, angina pain, neuropathic pain such as peripheral neuropathy and diabetic neuropathy, post-operative pain, and pain which is co-morbid with nervous system disorders described herein.

As used herein, the term “acute pain” refers to centralized or peripheral pain that is intense, localized, sharp, or stinging, and/or dull, aching, diffuse, or burning in nature and that occurs for short periods of time.

As used herein, the term “chronic pain” refers to centralized or peripheral pain that is intense, localized, sharp, or stinging, and/or dull, aching, diffuse, or burning in nature and that occurs for extended periods of time (i.e., persistent and/or regularly reoccurring), including, for the purpose of the present invention, neuropathic pain and cancer pain. Chronic pain includes neuropathic pain, hyperalgesia, and/or allodynia.

As used herein, the term “neuropathic pain” refers to chronic pain caused by damage to or pathological changes in the peripheral or central nervous systems. Examples of pathological changes related to neuropathic pain include prolonged peripheral or central neuronal sensitization, central sensitization related damage to nervous system inhibitory and/or exhibitory functions and abnormal interactions between the parasympathetic and sympathetic nervous systems. A wide range of clinical conditions may be associated with or form the basis for neuropathic pain including, for example, diabetes, post traumatic pain of amputation (nerve damage cause by injury resulting in peripheral and/or central sensitization such as phantom limb pain), lower back pain, cancer, chemical injury, toxins, other major surgeries, peripheral nerve damage due to traumatic injury compression, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, reflex sympathetic dystrophy or post thoracotomy pain, nutritional deficiencies, or viral or bacterial infections such as shingles or human immunodeficiency virus (HIV), and combinations thereof. Also included in the definition of neuropathic pain is a condition secondary to metastatic infiltration, adiposis dolorosa, burns, central pain conditions related to thalamic conditions, and combinations thereof.

As used herein, the term “hyperalgesia” refers to pain where there is an increase in sensitivity to a typically noxious stimulus.

As used herein, the term “allodynia” refers to an increase in sensitivity to a typically non-noxious stimulus.

As used herein, the term “visceral pain” refers to pain associated with or resulting from maladies of the internal organs, such as, for example, ulcerative colitis, irritable bowel syndrome, irritable bladder, Crohn's disease, rheumatologic (arthralgias), tumors, gastritis, pancreatitis, infections of the organs, biliary tract disorders, and combinations thereof.

As used herein, the term “female-specific pain” refers to pain that may be acute and/or chronic pain associated with female conditions. Such groups of pain include those that are encountered solely or predominately by females, including pain associated with menstruation, ovulation, pregnancy or childbirth, miscarriage, ectopic pregnancy, retrograde menstruation, rupture of a follicular or corpus luteum cyst, irritation of the pelvic viscera, uterine fibroids, adenomyosis, endometriosis, infection and inflammation, pelvic organ ischemia, obstruction, intra-abdominal adhesions, anatomic distortion of the pelvic viscera, ovarian abscess, loss of pelvic support, tumors, pelvic congestion or referred pain from non-gynecological causes, and combinations thereof.

In one embodiment, the present invention is directed to methods for treating or preventing vasomotor symptoms in a subject in need thereof, comprising the step of:
administering to said subject an effective amount of at least one compound of formula I, II, or III or pharmaceutically acceptable salt thereof.

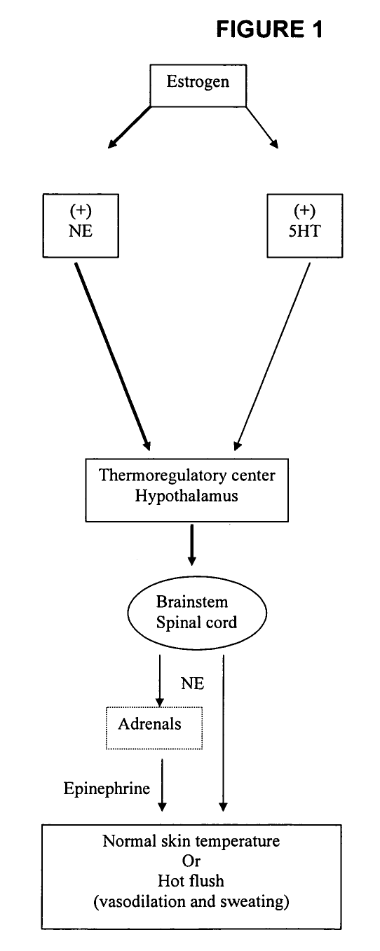

When estrogen levels are low or estrogen is absent, the normal levels between NE and 5-HT is altered and this altered change in neurotransmitter levels may result in changes in the sensitivity of the thermoregulatory center. The altered chemical levels may be translated in the thermoregulatory center as heat sensation and as a response, the hypothalamus may activate the descending autonomic pathways and result in heat dissipation via vasodilation and sweating (hot flush) (FIG. 1). Accordingly, the estrogen deprivation may result in altered norepinephrine activity.

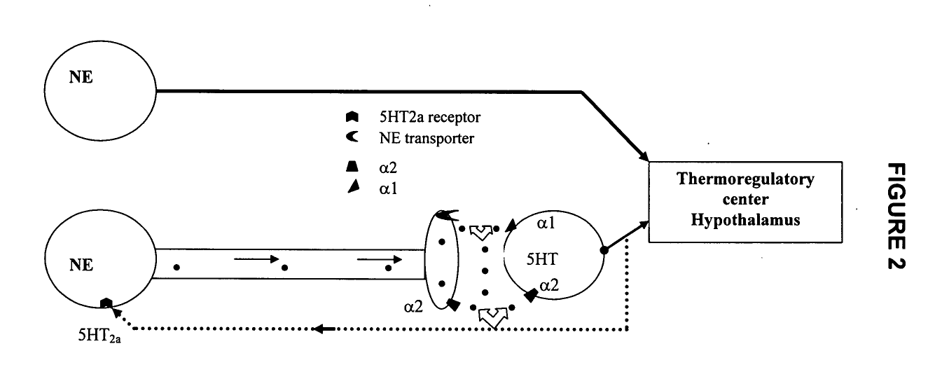

Norepinephrine synthesized in perikarya of the brainstem is released at the nerve terminals in the hypothalamus and brainstem. In the hypothalamus, NE regulates the activity of neurons residing in the thermoregulatory center. In the brainstem, NE innervates serotoninergic neurons (5HT), and acting via adrenergic_{α1} and adrenergic_{α2} postsynaptic receptors, it stimulates the activity of the serotoninergic system. In response, 5-HT neurons also modulate the activity the thermoregulatory center and feedback to NE neurons. Via this feedback connection, 5-HT, acting via 5-HT₂ₐ receptors, inhibit the activity of NE neurons. Norepinephrine in the synaptic cleft is also taken up by NE transporter (NET) located in NE neurons. The transporter recycles NE and makes it available for multiple neurotransmission (FIG. 2).

The present invention provides a treatment for vasomotor symptoms by methods of recovering the reduced activity of norepinephrine. Norepinephrine activity in the hypothalamus or in the brainstem can be elevated by (i) blocking the activity of the NE transporter, (ii) blocking the activity of the presynaptic adrenergic_{α2} receptor with an antagonist, or (iii) blocking the activity of 5-HT on NE neurons with a 5-HT₂ₐ antagonist.

In another embodiment, the present invention is directed to methods for treating or preventing a depression disorder in a subject in need thereof, comprising the step of:
administering to said subject an effective amount of at least one compound of formula I, II, or III or pharmaceutically acceptable salt thereof.

In yet other embodiments, the present invention is directed to methods for treating or preventing sexual dysfunction in a subject in need thereof, comprising the step of:
administering to said subject an effective amount of at least one compound of formula I, II, or III or pharmaceutically acceptable salt thereof.

In another embodiment, the present invention is directed to methods for treating or preventing gastrointestinal or genitourinary disorder, particularly stress incontinence or urge urinary incontinence, in a subject in need thereof, comprising the step of:
administering to said subject an effective amount of a compound of formula I, II, or III or pharmaceutically acceptable salt thereof.

In another embodiment, the present invention is directed to methods for treating or preventing chronic fatigue syndrome in a subject in need thereof, comprising the step of:
administering to said subject an effective amount of a compound of formula I, II, or III or pharmaceutically acceptable salt thereof.

In another embodiment, the present invention is directed to methods for treating or preventing fibromylagia syndrome in a subject in need thereof, comprising the step of:
administering to said subject an effective amount of a compound of formula I, II, or III or pharmaceutically acceptable salt thereof.

In further embodiments, the present invention is directed to methods for treating or preventing pain in a subject in need thereof, comprising the step of:
administering to said subject an effective amount of at least one compound of formula I, II, or III or pharmaceutically acceptable salt thereof.

The pain may be, for example, acute pain (short duration) or chronic pain (regularly reoccurring or persistent). The pain may also be centralized or peripheral.

Examples of pain that can be acute or chronic and that can be treated in accordance with the methods of the present invention include inflammatory pain, musculoskeletal pain, bony pain, lumbosacral pain, neck or upper back pain, visceral pain, somatic pain, neuropathic pain, cancer pain, pain caused by injury or surgery such as burn pain or dental pain, or headaches such as migraines or tension headaches, or combinations of these pains. One skilled in the art will recognize that these pains may overlap one another. For example, a pain caused by inflammation may also be visceral or musculoskeletal in nature.

In a preferred embodiment of the present invention the compounds useful in the present invention are administered in mammals to treat chronic pain such as neuropathic pain associated for example with damage to or pathological changes in the peripheral or central nervous systems; cancer pain; visceral pain associated with for example the abdominal, pelvic, and/or perineal regions or pancreatitis; musculoskeletal pain associated with for example the lower or upper back, spine, fibromylagia, temporomandibular joint, or myofascial pain syndrome; bony pain associated with for example bone or joint degenerating disorders such as osteoarthritis, rheumatoid arthritis, or spinal stenosis; headaches such migraine or tension headaches; or pain associated with infections such as HIV, sickle cell anemia, autoimmune disorders, multiple sclerosis, or inflammation such as osteoarthritis or rheumatoid arthritis.

In a more preferred embodiment, the compounds useful in this invention are used to treat chronic pain that is neuropathic pain, visceral pain, musculoskeletal pain, bony pain, cancer pain or inflammatory pain or combinations thereof, in accordance with the methods described herein. Inflammatory pain can be associated with a variety of medical conditions such as osteoarthritis, rheumatoid arthritis, surgery, or injury. Neuropathic pain may be associated with for example diabetic neuropathy, peripheral neuropathy, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, or nerve damage cause by injury resulting in peripheral and/or central sensitization such as phantom limb pain, reflex sympathetic dystrophy or postthoracotomy pain, cancer, chemical injury, toxins, nutritional deficiencies, or viral or bacterial infections such as shingles or HIV, or combinations thereof. The methods of use for compounds of this invention further include treatments in which the neuropathic pain is a condition secondary to metastatic infiltration, adiposis dolorosa, burns, or central pain conditions related to thalamic conditions.

As mentioned previously, the methods of the present invention may be used to treat pain that is somatic and/or visceral in nature. For example, somatic pain that can be treated in accordance with the methods of the present invention include pains associated with structural or soft tissue injury experienced during surgery, dental procedures, burns, or traumatic body injuries. Examples of visceral pain that can be treated in accordance with the methods of the present invention include those types of pain associated with or resulting from maladies of the internal organs such as ulcerative colitis, irritable bowel syndrome, irritable bladder, Crohn's disease, rheumatologic (arthralgias), tumors, gastritis, pancreatitis, infections of the organs, or biliary tract disorders, or combinations thereof. One skilled in the art will also recognize that the pain treated according to the methods of the present invention may also be related to conditions of hyperalgesia, allodynia, or both. Additionally, the chronic pain may be with or without peripheral or central sensitization.

The compounds useful in this invention may also be used to treat acute and/or chronic pains associated with female conditions, which may also be referred to as female-specific pain. Such groups of pain include those that are encountered solely or predominately by females, including pain associated with menstruation, ovulation, pregnancy or childbirth, miscarriage, ectopic pregnancy, retrograde menstruation, rupture of a follicular or corpus luteum cyst, irritation of the pelvic viscera, uterine fibroids, adenomyosis, endometriosis, infection and inflammation, pelvic organ ischemia, obstruction, intra-abdominal adhesions, anatomic distortion of the pelvic viscera, ovarian abscess, loss of pelvic support, tumors, pelvic congestion or referred pain from non-gynecological causes.

The compounds of formula I, II, or a pharmaceutically acceptable salt thereof, are useful in treating and preventing schizophrenia in a subject in need thereof.

The present invention is further defined in the following Examples, in which all parts and percentages are by weight and degrees are Celsius, unless otherwise stated. It should be understood that these examples, while indicating preferred embodiments of the invention, are given by way of illustration only. From the above discussion and these examples, one skilled in the art can ascertain the essential characteristics of this invention, and without departing from the spirit and scope thereof, can make various changes and modifications of the invention to adapt it to various usages and conditions.

### EXAMPLES

### Example 1

### (1S,2R)-1-[5-(benzyloxy)-1H-indol-1-yl]-3-(methylamino)-1-phenylpropan-2-ol hydrochloride

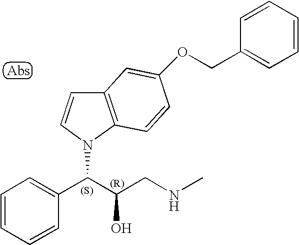

Step 1:

A mixture of diisopropyl D-tartrate (d 1.119, 6.0 mL, 29 mmol), 4 A powdered molecular sieves (28 g, dried overnight at 200° C.) and dry dichloromethane (800 mL) was cooled to −20° C. Titanium (IV) isopropoxide (d 0.97, 5.9 mL, 20 mmol) was added and the mixture was stirred for 15 minutes. Anhydrous tert-butyl hydroperoxide (ca. 5.5 M in decane, 90 mL, ca. 500 mmol), further dried for 15 minutes over 4 A molecular sieve pellets (dried overnight at 200° C.), was added slowly and the mixture was stirred for 45 minutes at −20° C. A solution of cinnamyl alcohol (27 g, 200 mmol) in dry dichloromethane (200 mL) was added during 1 hour at −20° C. After a further 2 hours at −20° C., the reaction mixture was quenched with a cooled (−20° C.) mixture of 30% aqueous sodium hydroxide-saturated aqueous sodium chloride solution (35 mL). Diethyl ether (100 mL) was added and the mixture was vigorously stirred at 0° C. for 1.5 hours. Magnesium sulfate (75 g) was added, the mixture was stirred for 20 minutes, then filtered through silica gel (100 g) and washed with diethyl ether (250 mL). The filtrate was concentrated under vacuum and excess tert-butyl hydroperoxide was azeotroped off with several portions of toluene to provide a cloudy yellow oil. Flash column chromatography (silica 500 g, 25%, 50% ethyl acetate/hexanes) provided a white crystalline solid (27 g). Recrystallization from hot 20% ethyl acetate-hexanes provided [(2R,3R)-3-phenyloxiran-2-yl]methanol (21 g, 70%) as white needles. MS (ES) m/z 133 ([M+H—H₂O]⁺).

Step 2:

A suspension of 5-benzyloxyindole (8.9 g, 40 mmol) in glacial acetic acid (40 mL) was treated with sodium cyanoborohydride (5.0 g, 80 mmol) portionwise at 0° C. After 2 hours at 0° C., the reaction mixture was diluted with water (80 mL) and made alkaline with 40% aqueous sodium hydroxide at 0° C. The aqueous phase was extracted with dichloromethane (3×75 mL) and the combined extracts were washed with saturated brine (100 mL), and dried (sodium sulfate). Filtration through silica gel (50 g) washing with dichloromethane provided 5-(benzyloxy)indoline (7.8 g, 87%) as a clear, yellow oil. MS (ES) m/z 226 ([M+H]⁺).

Step 3:

A mixture of [(2R,3R)-3-phenyloxiran-2-yl]methanol (10.0 g, 66.6 mmol, from step 1) and 5-(benzyloxy)indoline (15.0 g, 66.6 mmol) was heated at 135° C. for 1.5 hours. The mixture was dissolved in dichloromethane (40 mL) and pre-adsorbed on silica gel (40 g). Flash column chromatography (silica 600 g, 30%, 40%, 50%, 80% ethyl acetate/hexanes) provided (2S,3S)-3-[5-(benzyloxy)-2,3-dihydro-1H-indol-1-yl]-3-phenylpropane-1,2-diol (22.0 g, 88%) as an amber oil. MS (ES) m/z 376 ([M+H]⁺).

Step 4:

A solution of (2S,3S)-3-[5-(benzyloxy)-2,3-dihydro-1H-indol-1-yl]-3-phenylpropane-1,2-diol (11.0 g, 29.3 mmol) in dry toluene (150 mL) was treated with a solution of 2,3-dichloro-5,6-dicyano-1,4-benzoquinone (6.65 g, 29.3 mmol) in dry toluene (150 mL) at 0° C. After 1.5 hours, the thick mixture was quenched with 5% aqueous sodium carbonate (370 mL) and stirred vigorously for 5-10 minutes. The mixture was partitioned between ethyl acetate (1.1 L) and 5% aqueous sodium carbonate (1.1 L). The organic phase was separated, washed with 5% aqueous sodium carbonate (4×1.1 L) and saturated brine (1.1 L), dried over magnesium sulfate, filtered and concentrated under reduced pressure to give a crude brown solid (10.4 g). Flash column chromatography (silica 150 g, 40%, 50%, 60%, 80%, 100% ethyl acetate/hexanes) provided (2S,3S)-3-[5-(benzyloxy)-1H-indol-1-yl]-3-phenylpropane-1,2-diol (9.2 g, 84%) as a tan solid. MS (ES) m/z 374 ([M+H]⁺).

Step 5:

A solution of (2S,3S)-3-[5-(benzyloxy)-1H-indol-1-yl]-3-phenylpropane-1,2-diol (7.5 g, 20 mmol) in dry pyridine (55 mL) was treated with p-toluenesulfonyl chloride (3.9 g, 20 mmol) at 23° C. After 21 hours, the reaction mixture was diluted with ethyl acetate (1 L) and the organic phase was washed with 1.0 M aqueous sodium hydroxide (1 L), water (1 L), 1.0 M aqueous hydrochloric acid (1 L) and saturated brine (1 L), dried over sodium sulfate, filtered and concentrated under reduced pressure to give a dark oil (11 g) that was dissolved in dichloromethane and pre-adsorbed on silica gel (15 g). Flash column chromatography (silica 165 g, 20%, 40%, 60% ethyl acetate/hexanes) provided (2S,3S)-toluene-4-sulfonic acid 3-(5-benzyloxy-indol-1-yl)-2-hydroxy-3-phenyl-propyl ester (8.3 g, 78%) as an orange foam. MS (ES) m/z 528 ([M+H]⁺).

Step 6:

(2S,3S)-Toluene-4-sulfonic acid 3-(5-benzyloxy-indol-1-yl)-2-hydroxy-3-phenyl-propyl ester (4.1 g, 7.8 mmol) was treated with a solution of methylamine (2.0 M in methanol, 40 mL, 80 mmol) and the solution was stirred at 23° C. for 24 hours. At this time, the solution was concentrated under reduced pressure and the residue was partitioned between diethyl ether (500 mL) and 1.0 M aqueous sodium hydroxide (500 mL). The organic phase was separated, washed with water (500 mL) and saturated brine (500 mL), dried over sodium sulfate, filtered and concentrated under reduced pressure to provide a tan foam (3.0 g). Flash column chromatography (silica 125 g, 2.5%, 5% ammonia-saturated methanol/dichloromethane) provided (1S,2R)-1-[5-(benzyloxy)-1H-indol-1-yl]-3-(methylamino)-1-phenylpropan-2-ol (2.3 g, 77%) as a pale yellow solid. The solid (0.28 g) was dissolved in warm methanol (2-3 mL) and treated with a solution of hydrogen chloride (4.0 M in 1,4-dioxane, 0.18 mL, 0.72 mmol). The precipitated solid was stirred vigorously with diethyl ether (25 mL) for ca. 1 minute. Vacuum filtration provided (1S,2R)-1-[5-(benzyloxy)-1H-indol-1-yl]-3-(methylamino)-1-phenylpropan-2-ol hydrochloride (0.30 g, 97% recovery) as a white solid. MS (ES) m/z 387 ([M+H]⁺).

### Example 2

### (1S,2R)-1-[4-(benzyloxy)-1H-indol-1-yl]-3-(methylamino)-1-phenylpropan-2-ol hydrochloride

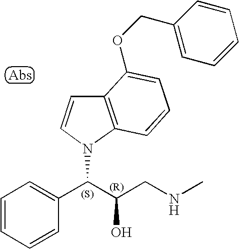

In an analogous manner to Example 1, step 2, 4-(benzyloxy)indoline was prepared from 4-benzyloxyindole. MS (ES) m/z 226 ([M+H]⁺).

In an analogous manner to Example 1, step 3, (2S,3S)-3-[4-(benzyloxy)-2,3-dihydro-1H-indol-1-yl]-3-phenylpropane-1,2-diol was prepared from 4-(benzyloxy)indoline and [(2R,3R)-3-phenyloxiran-2-yl]methanol. MS (ES) m/z 376 ([M+H]⁺).

In an analogous manner to Example 1, step 4, (2S,3S)-3-[4-(benzyloxy)-1H-indol-1-yl]-3-phenylpropane-1,2-diol was prepared from (2S,3S)-3-[4-(benzyloxy)-2,3-dihydro-1H-indol-1-yl]-3-phenylpropane-1,2-diol. MS (ES) m/z 374 ([M+H]⁺).

In an analogous manner to Example 1, step 5, (2S,3S)-toluene-4-sulfonic acid 3-(4-benzyloxy-indol-1-yl)-2-hydroxy-3-phenyl-propyl ester was prepared from ((2S,3S)-3-[4-(benzyloxy)-1H-indol-1-yl]-3-phenylpropane-1,2-diol. MS (ES) m/z 528 ([M+H]⁺).

In an analogous manner to Example 1, step 6, (1S,2R)-1-[4-(benzyloxy)-1H-indol-1-yl]-3-(methylamino)-1-phenylpropan-2-ol hydrochloride was prepared from (2S,3S)-toluene-4-sulfonic acid 3-(4-benzyloxy-indol-1-yl)-2-hydroxy-3-phenyl-propyl ester. MS (ES) m/z 387 ([M+H]⁺).

### Example 3

### (1S,2R)-1-[6-(benzyloxy)-1H-indol-1-yl]-3-(methylamino)-1-phenylpropan-2-ol hydrochloride

WAY-318969-A-1 L31883-135-B

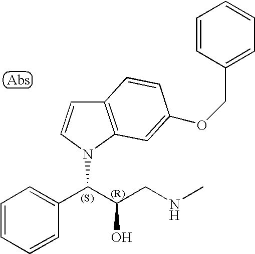

In an analogous manner to Example 1, step 2, 6-(benzyloxy)indoline was prepared from 6-benzyloxyindole. MS (ES) m/z 226 ([M+H]⁺).

In an analogous manner to Example 1, step 3, (2S,3S)-3-[6-(benzyloxy)-2,3-dihydro-1H-indol-1-yl]-3-phenylpropane-1,2-diol was prepared from 6-(benzyloxy)indoline and [(2R,3R)-3-phenyloxiran-2-yl]methanol. MS (ES) m/z 376 ([M+H]⁺).

In an analogous manner to Example 1, step 4, (2S,3S)-3-[6-(benzyloxy)-1H-indol-1-yl]-3-phenylpropane-1,2-diol was prepared from (2S,3S)-3-[6-(benzyloxy)-2,3-dihydro-1H-indol-1-yl]-3-phenylpropane-1,2-diol. MS (ES) m/z 374 ([M+H]⁺).

In an analogous manner to Example 1, step 5, (2S,3S)-toluene-4-sulfonic acid 3-(6-benzyloxy-indol-1-yl)-2-hydroxy-3-phenyl-propyl ester was prepared from ((2S,3S)-3-[6-(benzyloxy)-1H-indol-1-yl]-3-phenylpropane-1,2-diol. MS (ES) m/z 528 ([M+H]⁺).

In an analogous manner to Example 1, step 6, (1S,2R)-1-[6-(benzyloxy)-1H-indol-1-yl]-3-(methylamino)-1-phenylpropan-2-ol hydrochloride was prepared from (2S,3S)-toluene-4-sulfonic acid 3-(6-benzyloxy-indol-1-yl)-2-hydroxy-3-phenyl-propyl ester. MS (ES) m/z 387 ([M+H]⁺).

### Example 4

### (1S,2R)-1-[7-(benzyloxy)-1H-indol-1-yl]-3-(methylamino)-1-phenylpropan-2-ol hydrochloride

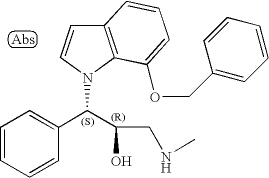

In an analogous manner to Example 1, step 2, 7-(benzyloxy)indoline was prepared from 7-benzyloxyindole. MS (ES) m/z 226 ([M+H]⁺).

In an analogous manner to Example 1, step 3, (2S,3S)-3-[7-(benzyloxy)-2,3-dihydro-1H-indol-1-yl]-3-phenylpropane-1,2-diol was prepared from 7-(benzyloxy)indoline and [(2R,3R)-3-phenyloxiran-2-yl]methanol. MS (ES) m/z 376 ([M+H]⁺).

In an analogous manner to Example 1, step 4, (2S,3S)-3-[7-(benzyloxy)-1H-indol-1-yl]-3-phenylpropane-1,2-diol was prepared from (2S,3S)-3-[7-(benzyloxy)-2,3-dihydro-1H-indol-1-yl]-3-phenylpropane-1,2-diol. MS (ES) m/z 374 ([M+H]⁺).

In an analogous manner to Example 1, step 5, (2S,3S)-toluene-4-sulfonic acid 3-(7-benzyloxy-indol-1-yl)-2-hydroxy-3-phenyl-propyl ester was prepared from ((2S,3S)-3-[7-(benzyloxy)-1H-indol-1-yl]-3-phenylpropane-1,2-diol. MS (ES) m/z 528 ([M+H]⁺).

In an analogous manner to Example 1, step 6, (1S,2R)-1-[7-(benzyloxy)-1H-indol-1-yl]-3-(methylamino)-1-phenylpropan-2-ol hydrochloride was prepared from (2S,3S)-toluene-4-sulfonic acid 3-(7-benzyloxy-indol-1-yl)-2-hydroxy-3-phenyl-propyl ester. MS (ES) m/z 387 ([M+H]⁺).

### Example 5

### (1S,2R)-1-{5-[(2-methoxybenzyl)oxy]-1H-indol-1-yl}-3-(methylamino)-1-phenylpropan-2-ol hydrochloride

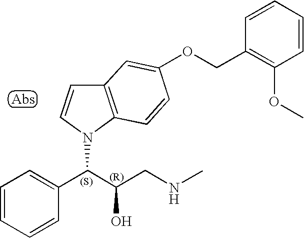

Step 1:

A solution of (1S,2R)-1-[5-(benzyloxy)-1H-indol-1-yl]-3-(methylamino)-1-phenylpropan-2-ol (1.7 g, 4.4 mmol, from Example 1, step 6) in dichloromethane (30 mL) was treated with triethylamine (d 0.726, 1.23 mL, 8.8 mmol) and di-tert-butyl dicarbonate (1.2 g, 5.5 mmol) at 23° C. After 16 hours, the reaction mixture was washed with 1.0 M aqueous potassium hydrogen sulfate (3×15 mL), saturated aqueous sodium bicarbonate (15 mL), 10% (w/v) aqueous citric acid (15 mL) and saturated brine (15 mL), dried over magnesium sulfate, filtered and concentrated under reduced pressure to give an orange foam (2.3 g). Flash column chromatography (silica 40 g, 40% ethyl acetate/hexanes) provided tert-butyl {(2R,3S)-3-[5-(benzyloxy)-1H-indol-1-yl]-2-hydroxy-3-phenylpropyl}methylcarbamate (2.1 g, 100%) as a pale yellow foam. MS (ES) m/z 487 ([M+H]⁺).

Step 2:

A solution of tert-butyl {(2R,3S)-3-[5-(benzyloxy)-1H-indol-1-yl]-2-hydroxy-3-phenylpropyl}methylcarbamate (5.3 g, 11 mmol) in 1:1 v/v ethyl acetate-ethanol (100 mL) was hydrogenated over 10% palladium-on-carbon (1.7 g) at 50 psi. After 16 hours, the catalyst was filtered (Celite) and washed with hot ethanol (3×100 mL). Concentration of the filtrate gave a tan solid (4.3 g) which was triturated overnight with ethyl acetate (30 mL) to provide tert-butyl [(2R,3S)-2-hydroxy-3-(5-hydroxy-1H-indol-1-yl)-3-phenylpropyl]methylcarbamate (3.8 g, 88%) as a white solid. MS (ES) m/z 397 ([M+H]⁺).

Step 3:

A solution of tert-butyl [(2R,3S)-2-hydroxy-3-(5-hydroxy-1H-indol-1-yl)-3-phenylpropyl]methylcarbamate (300 mg, 0.757 mmol) in dry acetonitrile (5 mL) was treated with 2-methoxybenzyl chloride (d 1.125, 105 μL, 0.754 mmol) followed by cesium carbonate (247 mg, 0.758 mmol) and the mixture was heated at 70° C. After 12 hours, the cooled mixture was filtered (Celite), washed with acetonitrile (2×5 mL), and concentrated under reduced pressure. Pre-adsorption on silica (1 g in dichloromethane) and purification via ISCO CombiFlash Companion chromatography (12 g RediSep silica, 30 mL/min, 0-40% ethyl acetate/hexane) provided tert-butyl ((2R,3S)-2-hydroxy-3-{5-[(2-methoxybenzyl)oxy]-1H-indol-1-yl}-3-phenylpropyl)methylcarbamate (181 mg, 46%) as a white foam. MS (ES) m/z 517 ([M+H]⁺).

Step 4:

Tert-butyl ((2R,3S)-2-hydroxy-3-{5-[(2-methoxybenzyl)oxy]-1H-indol-1-yl}-3-phenylpropyl)methylcarbamate (176 mg, 0.341 mmol) was heated at 200° C. with vigorous stirring for 8 minutes. Flash column chromatography (silica 8 g, 1.25%, 2.5%, 5% ammonia-saturated methanol/dichloromethane) provided (1S,2R)-1-{5-[(2-methoxybenzyl)oxy]-1H-indol-1-yl}-3-(methylamino)-1-phenylpropan-2-ol (34 mg, 24%) as a white foam. The foam was dissolved in diethyl ether (3 mL), filtered and methanol (5 drops) was added. The solution was treated with a solution of hydrogen chloride (4.0 M in 1,4-doxane, 0.02 mL, 0.08 mmol) and vigorously stirred for ca. 1 minute. Vacuum filtration provided (1S,2R)-1-{5-[(2-methoxybenzyl)oxy]-1H-indol-1-yl}-3-(methylamino)-1-phenylpropan-2-ol hydrochloride (27 mg, 18%) as an off-white solid. MS (ES) m/z 417 ([M+H]⁺).

### Example 6

### (1S,2R)-1-{5-[(3-methoxybenzyl)oxy]-1H-indol-1-yl}-3-(methylamino)-1-phenylpropan-2-ol hydrochloride

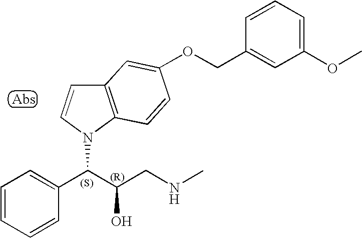

In an analogous manner to Example 5, step 3, tert-butyl ((2R,3S)-2-hydroxy-3-{5-[(3-methoxybenzyl)oxy]-1H-indol-1-yl}-3-phenylpropyl)methylcarbamate was prepared from tert-butyl [(2R,3S)-2-hydroxy-3-(5-hydroxy-1H-indol-1-yl)-3-phenylpropyl]methylcarbamate, substituting 3-methoxybenzyl bromide in place of 2-methoxybenzyl chloride. MS (ES) m/z 517 ([M+H]⁺).

In an analogous manner to Example 5, step 4, (1S,2R)-1-{5-[(3-methoxybenzyl)oxy]-1H-indol-1-yl}-3-(methylamino)-1-phenylpropan-2-ol hydrochloride was prepared from tert-butyl ((2R,3S)-2-hydroxy-3-{5-[(3-methoxybenzyl)oxy]-1H-indol-1-yl}-3-phenylpropyl)methylcarbamate. MS (ES) m/z 417 ([M+H]⁺).

### Example 7

### (1S,2R)-1-{5-[(4-methoxybenzyl)oxy]-1H-indol-1-yl}-3-(methylamino)-1-phenylpropan-2-ol hydrochloride

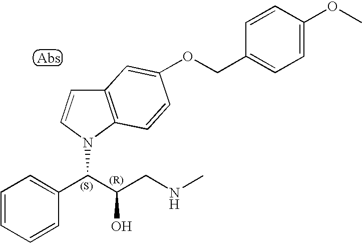

In an analogous manner to Example 5, step 3, tert-butyl ((2R,3S)-2-hydroxy-3-{5-[(4-methoxybenzyl)oxy]-1H-indol-1-yl}-3-phenylpropyl)methylcarbamate was prepared from tert-butyl [(2R,3S)-2-hydroxy-3-(5-hydroxy-1H-indol-1-yl)-3-phenylpropyl]methylcarbamate, substituting 4-methoxybenzyl chloride in place of 2-methoxybenzyl chloride. MS (ES) m/z 517 ([M+H]⁺).

In an analogous manner to Example 5, step 4, (1S,2R)-1-{5-[(4-methoxybenzyl)oxy]-1H-indol-1-yl}-3-(methylamino)-1-phenylpropan-2-ol hydrochloride was prepared from tert-butyl ((2R,3S)-2-hydroxy-3-{5-[(4-methoxybenzyl)oxy]-1H-indol-1-yl}-3-phenylpropyl)methylcarbamate. MS (ES) m/z 417 ([M+H]⁺).

### Example 8

### (1S,2R)-1-{5-[(2-chlorobenzyl)oxy]-1H-indol-1-yl}-3-(methylamino)-1-phenylpropan-2-ol hydrochloride

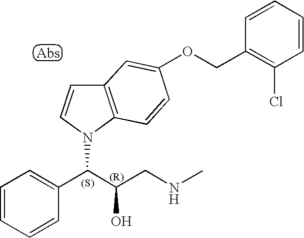

In an analogous manner to Example 5, step 3, tert-butyl ((2R,3S)-3-{5-[(2-chlorobenzyl)oxy]-1H-indol-1-yl}-2-hydroxy-3-phenylpropyl)methylcarbamate was prepared from tert-butyl [(2R,3S)-2-hydroxy-3-(5-hydroxy-1H-indol-1-yl)-3-phenylpropyl]methylcarbamate, substituting 2-chlorobenzyl bromide in place of 2-methoxybenzyl chloride. MS (ES) m/z 521 ([M+H]⁺).

In an analogous manner to Example 5, step 4, (1S,2R)-1-{5-[(2-chlorobenzyl)oxy]-1H-indol-1-yl}-3-(methylamino)-1-phenylpropan-2-ol hydrochloride was prepared from tert-butyl ((2R,3S)-3-{5-[(2-chlorobenzyl)oxy]-1H-indol-1-yl}-2-hydroxy-3-phenylpropyl)methylcarbamate. MS (ES) m/z 421 ([M+H]⁺).

### Example 9

### (1S,2R)-1-{5-[(3-chlorobenzyl)oxy]-1H-indol-1-yl}-3-(methylamino)-1-phenylpropan-2-ol hydrochloride

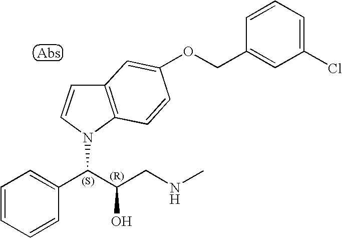

In an analogous manner to Example 5, step 3, tert-butyl ((2R,3S)-3-{5-[(3-chlorobenzyl)oxy]-1H-indol-1-yl}-2-hydroxy-3-phenylpropyl)methylcarbamate was prepared from tert-butyl [(2R,3S)-2-hydroxy-3-(5-hydroxy-1H-indol-1-yl)-3-phenylpropyl]methylcarbamate, substituting 3-chlorobenzyl bromide in place of 2-methoxybenzyl chloride. MS (ES) m/z 521 ([M+H]⁺).

In an analogous manner to Example 5, step 4, (1S,2R)-1-{5-[(3-chlorobenzyl)oxy]-1H-indol-1-yl}-3-(methylamino)-1-phenylpropan-2-ol hydrochloride was prepared from tert-butyl ((2R,3S)-3-{5-[(3-chlorobenzyl)oxy]-1H-indol-1-yl}-2-hydroxy-3-phenylpropyl)methylcarbamate. MS (ES) m/z 421 ([M+H]⁺).

### Example 10

### (1S,2R)-1-{5-[(4-chlorobenzyl)oxy]-1H-indol-1-yl}-3-(methylamino)-1-phenylpropan-2-ol hydrochloride

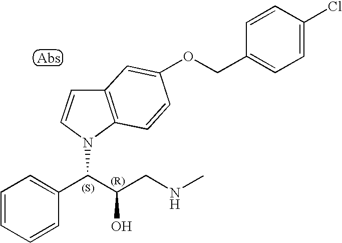

In an analogous manner to Example 5, step 3, tert-butyl ((2R,3S)-3-{5-[(4-chlorobenzyl)oxy]-1H-indol-1-yl}-2-hydroxy-3-phenylpropyl)methylcarbamate was prepared from tert-butyl [(2R,3S)-2-hydroxy-3-(5-hydroxy-1H-indol-1-yl)-3-phenylpropyl]methylcarbamate, substituting 4-chlorobenzyl bromide in place of 2-methoxybenzyl chloride. MS (ES) m/z 521 ([M+H]⁺).

In an analogous manner to Example 5, step 4, (1S,2R)-1-{5-[(4-chlorobenzyl)oxy]-1H-indol-1-yl}-3-(methylamino)-1-phenylpropan-2-ol hydrochloride was prepared from tert-butyl ((2R,3S)-3-{5-[(4-chlorobenzyl)oxy]-1H-indol-1-yl}-2-hydroxy-3-phenylpropyl)methylcarbamate. MS (ES) m/z 421 ([M+H]⁺).

### Example 11

### (1S,2R)-1-{5-[(2-fluorobenzyl)oxy]-1H-indol-1-yl}-3-(methylamino)-1-phenylpropan-2-ol hydrochloride

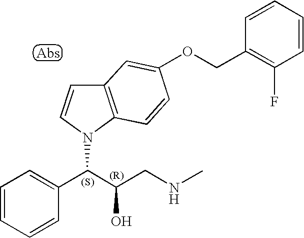

In an analogous manner to Example 5, step 3, tert-butyl ((2R,3S)-3-{5-[(2-fluorobenzyl)oxy]-1H-indol-1-yl}-2-hydroxy-3-phenylpropyl)methylcarbamate was prepared from tert-butyl [(2R,3S)-2-hydroxy-3-(5-hydroxy-1H-indol-1-yl)-3-phenylpropyl]methylcarbamate, substituting 2-fluorobenzyl bromide in place of 2-methoxybenzyl chloride. MS (ES) m/z 505 ([M+H]⁺).

In an analogous manner to Example 5, step 4, (1S,2R)-1-{5-[(2-fluorobenzyl)oxy]-1H-indol-1-yl}-3-(methylamino)-1-phenylpropan-2-ol hydrochloride was prepared from tert-butyl ((2R,3S)-3-{5-[(2-fluorobenzyl)oxy]-1H-indol-1-yl}-2-hydroxy-3-phenylpropyl)methylcarbamate. MS (ES) m/z 405 ([M+H]⁺).

### Example 12

### (1S,2R)-1-{5-[(3-fluorobenzyl)oxy]-1H-indol-1-yl}-3-(methylamino)-1-phenylpropan-2-ol hydrochloride

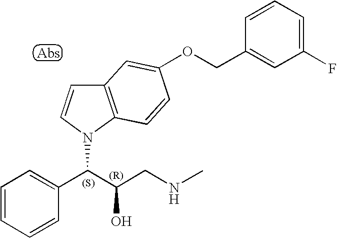

In an analogous manner to Example 5, step 3, tert-butyl ((2R,3S)-3-{5-[(3-fluorobenzyl)oxy]-1H-indol-1-yl}-2-hydroxy-3-phenylpropyl)methylcarbamate was prepared from tert-butyl [(2R,3S)-2-hydroxy-3-(5-hydroxy-1H-indol-1-yl)-3-phenylpropyl]methylcarbamate, substituting 3-fluorobenzyl bromide in place of 2-methoxybenzyl chloride. MS (ES) m/z 505 ([M+H]⁺).

In an analogous manner to Example 5, step 4, (1S,2R)-1-{5-[(3-fluorobenzyl)oxy]-1H-indol-1-yl}-3-(methylamino)-1-phenylpropan-2-ol hydrochloride was prepared from tert-butyl ((2R,3S)-3-{5-[(3-fluorobenzyl)oxy]-1H-indol-1-yl}-2-hydroxy-3-phenylpropyl)methylcarbamate. MS (ES) m/z 405 ([M+H]⁺).

### Example 13

### (1S,2R)-1-{5-[(4-fluorobenzyl)oxy]-1H-indol-1-yl}-3-(methylamino)-1-phenylpropan-2-ol hydrochloride

WAY-318373-A-1 L31883-71-B

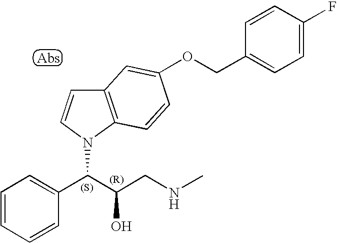

In an analogous manner to Example 5, step 3, tert-butyl ((2R,3S)-3-{5-[(4-fluorobenzyl)oxy]-1H-indol-1-yl}-2-hydroxy-3-phenylpropyl)methylcarbamate was prepared from tert-butyl [(2R,3S)-2-hydroxy-3-(5-hydroxy-1H-indol-1-yl)-3-phenylpropyl]methylcarbamate, substituting 4-fluorobenzyl bromide in place of 2-methoxybenzyl chloride. MS (ES) m/z 505 ([M+H]⁺).

In an analogous manner to Example 5, step 4, (1S,2R)-1-{5-[(4-fluorobenzyl)oxy]-1H-indol-1-yl}-3-(methylamino)-1-phenylpropan-2-ol hydrochloride was prepared from tert-butyl ((2R,3S)-3-{5-[(4-fluorobenzyl)oxy]-1H-indol-1-yl}-2-hydroxy-3-phenylpropyl)methylcarbamate. MS (ES) m/z 405 ([M+H]⁺).

### Example 14

### (1S,2R)-3-(methylamino)-1-{5-[(2-methylbenzyl)oxy]-1H-indol-1-yl}-1-phenylpropan-2-ol hydrochloride

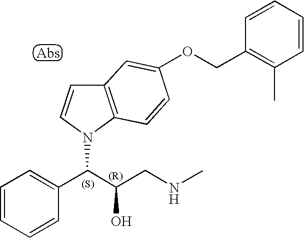

In an analogous manner to Example 5, step 3, tert-butyl ((2R,3S)-2-hydroxy-3-{5-[(2-methylbenzyl)oxy]-1H-indol-1-yl}-3-phenylpropyl)methylcarbamate was prepared from tert-butyl [(2R,3S)-2-hydroxy-3-(5-hydroxy-1H-indol-1-yl)-3-phenylpropyl]methylcarbamate, substituting 2-methylbenzyl bromide in place of 2-methoxybenzyl chloride. MS (ES) m/z 501 ([M+H]⁺).

In an analogous manner to Example 5, step 4, (1S,2R)-3-(methylamino)-1-{5-[(2-methylbenzyl)oxy]-1H-indol-1-yl}-1-phenylpropan-2-ol hydrochloride was prepared from tert-butyl ((2R,3S)-2-hydroxy-3-{5-[(2-methylbenzyl)oxy]-1H-indol-1-yl}-3-phenylpropyl)methylcarbamate. MS (ES) m/z 401 ([M+H]⁺).

### Example 15

### (1S,2R)-3-(methylamino)-1-{5-[(3-methylbenzyl)oxy]-1H-indol-1-yl}-1-phenylpropan-2-ol hydrochloride

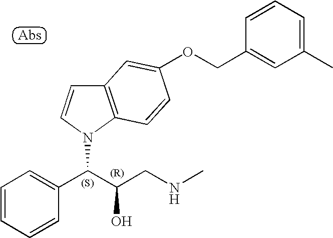

In an analogous manner to Example 5, step 3, tert-butyl ((2R,3S)-2-hydroxy-3-{5-[(3-methylbenzyl)oxy]-1H-indol-1-yl}-3-phenylpropyl)methylcarbamate was prepared from tert-butyl [(2R,3S)-2-hydroxy-3-(5-hydroxy-1H-indol-1-yl)-3-phenylpropyl]methylcarbamate, substituting 3-methylbenzyl bromide in place of 2-methoxybenzyl chloride. MS (ES) m/z 501 ([M+H]⁺).

In an analogous manner to Example 5, step 4, (1S,2R)-3-(methylamino)-1-{5-[(3-methylbenzyl)oxy]-1H-indol-1-yl}-1-phenylpropan-2-ol hydrochloride was prepared from tert-butyl ((2R,3S)-2-hydroxy-3-{5-[(3-methylbenzyl)oxy]-1H-indol-1-yl}-3-phenylpropyl)methylcarbamate. MS (ES) m/z 401 ([M+H]⁺).

### Example 16

### (1S,2R)-3-(methylamino)-1-{5-[(4-methylbenzyl)oxy]-1H-indol-1-yl}-1-phenylpropan-2-ol hydrochloride

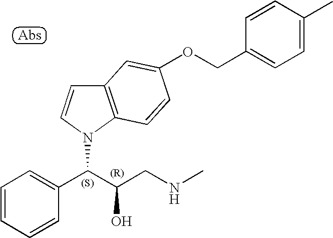

Step 1:

In an analogous manner to Example 5, step 3, tert-butyl ((2R,3S)-2-hydroxy-3-{5-[(4-methylbenzyl)oxy]-1H-indol-1-yl}-3-phenylpropyl)methylcarbamate was prepared from tert-butyl [(2R,3S)-2-hydroxy-3-(5-hydroxy-1H-indol-1-yl)-3-phenylpropyl]methylcarbamate, substituting 4-methylbenzyl bromide in place of 2-methoxybenzyl chloride. MS (ES) m/z 501 ([M+H]⁺).

Step 2:

A solution of tert-butyl ((2R,3S)-2-hydroxy-3-{5-[(4-methylbenzyl)oxy]-1H-indol-1-yl}-3-phenylpropyl)methylcarbamate (288 mg, 0.575 mmol) in diethyl ether (3 mL) was treated with a solution of hydrogen chloride (4.0 M in 1,4-dioxane, 0.17 mL, 0.68 mmol). After 16 hours, additional hydrogen chloride solution (4.0 M in 1,4-dioxane, 0.17 mL, 0.68 mmol) was added. After 5 days, the precipitated solid was vacuum filtered and washed with diethyl ether to provide a light pink solid (216 mg) that was partitioned between dichloromethane (20 mL) and saturated aqueous sodium bicarbonate (20 mL). The organic phase was separated, washed with saturated brine (20 mL), dried over sodium sulfate, filtered and concentrated under reduced pressure to provide a light orange foam (183 mg). Flash column chromatography (silica 13 g, 1%, 2%, 4% ammonia-saturated methanol/dichloromethane) provided (1S,2R)-3-(methylamino)-1-{5-[(4-methylbenzyl)oxy]-1H-indol-1-yl}-1-phenylpropan-2-ol (55 mg, 24%) as a white solid. The solid was dissolved in diethyl ether (3 mL), filtered and treated with a solution of hydrogen chloride (4.0 M in 1,4-dioxane, 0.04 mL, 0.16 mmol) and vigorously stirred for ca. 1 minute. Vacuum filtration provided (1S,2R)-3-(methylamino)-1-{5-[(4-methylbenzyl)oxy]-1H-indol-1-yl}-1-phenylpropan-2-ol hydrochloride (54 mg, 22%) as a light pink solid. MS (ES) m/z 401 ([M+H]⁺).

### Example 17

### (1S,2R)-3-(methylamino)-1-phenyl-1-[5-(1RS)-(1-phenylethoxy)-1H-indol-1-yl]propan-2-ol hydrochloride

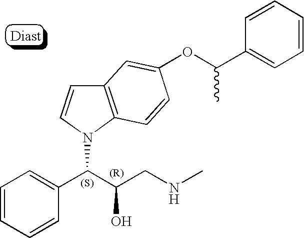

In an analogous manner to Example 5, step 3, tert-butyl {(2R,3S)-2-hydroxy-3-phenyl-3-[5-(1RS)-(1-phenylethoxy)-1H-indol-1-yl]propyl}methylcarbamate was prepared from tert-butyl [(2R,3S)-2-hydroxy-3-(5-hydroxy-1H-indol-1-yl)-3-phenylpropyl]methylcarbamate, substituting (1-bromoethyl)-benzene in place of 2-methoxybenzyl chloride. MS (ES) m/z 501 ([M+H]⁺).

In an analogous manner to Example 5, step 4, (1S,2R)-3-(methylamino)-1-phenyl-1-[5-(1RS)-(1-phenylethoxy)-1H-indol-1-yl]propan-2-ol hydrochloride was prepared from tert-butyl {(2R,3S)-2-hydroxy-3-phenyl-3-[5-(1RS)-(1-phenylethoxy)-1H-indol-1-yl]propyl}methylcarbamate. MS (ES) m/z 401 ([M+H]⁺).

### Example 18

### (1S,2R)-3-(methylamino)-1-phenyl-1-[5-(2-phenylethoxy)-1H-indol-1-yl]propan-2-ol hydrochloride

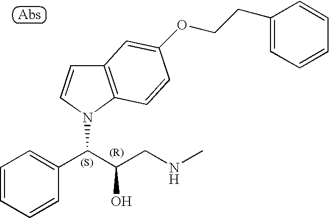

In an analogous manner to Example 5, step 3, tert-butyl {(2R,3S)-2-hydroxy-3-phenyl-3-[5-(2-phenylethoxy)-1H-indol-1-yl]propyl}methylcarbamate was prepared from tert-butyl [(2R,3S)-2-hydroxy-3-(5-hydroxy-1H-indol-1-yl)-3-phenylpropyl]methylcarbamate, substituting (2-bromoethyl)-benzene in place of 2-methoxybenzyl chloride. MS (ES) m/z 501 ([M+H]⁺).

In an analogous manner to Example 5, step 4, (1S,2R)-3-(methylamino)-1-phenyl-1-[5-(2-phenylethoxy)-1H-indol-1-yl]propan-2-ol hydrochloride was prepared from tert-butyl {(2R,3S)-2-hydroxy-3-phenyl-3-[5-(2-phenylethoxy)-1H-indol-1-yl]propyl}methylcarbamate. MS (ES) m/z 401 ([M+H]⁺).

### Example 19

### (1S,2R)-3-(methylamino)-1-[5-(phenoxy)-1H-indol-1-yl]-1-phenylpropan-2-ol hydrochloride

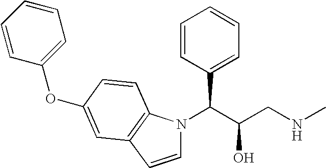

Step 1:

Potassium hydroxide (3.0 g, 53 mmol) was added to molten phenol (15 g, 160 mmol) at 110° C. with stirring. After all the potassium hydroxide had dissolved, the solution was cooled to 23° C. and 5-fluoro-2-nitrotoluene (7.75 g, 50.0 mmol) was added. The mixture was heated at 130° C. for 2 hours. At this time, additional hot potassium phenoxide (5 g phenol, 1 g potassium hydroxide) solution was added. After 3.5 hours (total), the mixture was heated at 150° C. After 5 hours (total), the cooled mixture was poured into 10% aqueous sodium hydroxide (200 mL) and extracted with diethyl ether (2×100 mL). The combined extracts were washed with 10% aqueous sodium hydroxide (2×100 mL) and water (2×100 mL), dried over sodium sulfate, filtered and concentrated under reduced pressure to yield a brown oil (11.6 g) that was taken up in diethyl ether and pre-adsorbed on silica gel (15 g). Purification by flash column chromatography (silica 135 g, 5% ethyl acetate/hexanes) provided 2-methyl-1-nitro-4-phenoxybenzene (11.4 g, 99%) as a clear, light yellow oil. MS (ES) m/z 230 ([M+H]⁺).

Step 2:

A solution of 2-methyl-1-nitro-4-phenoxybenzene (4.6 g, 20 mmol) and N,N-dimethylformamide diethyl acetal (d 0.859, 4.0 mL, 23 mmol) in dry N,N-dimethylformamide (12.5 mL) was heated at 150° C. The light yellow solution turned dark reddish-brown. After 22 hours, the cooled mixture was taken up in diethyl ether (500 mL), washed with water (3×250 mL) and saturated brine (250 mL), dried over sodium sulfate, filtered and concentrated under reduced pressure to provide crude dimethyl-[2-(2-nitro-5-phenoxy-phenyl)-vinyl]-amine (5.5 g, 96%) as a dark red oil.

Step 3:

A solution of dimethyl-[2-(2-nitro-5-phenoxy-phenyl)-vinyl]-amine (5.5 g, 19 mmol) in ethyl acetate (60 mL) was hydrogenated over 10% palladium-on-carbon (0.55 g) at 50 psi. After 2 hours, the catalyst was filtered (Celite) and washed with ethyl acetate (2×30 mL) and the filtrate was concentrated under reduced pressure to yield a brown oil (4.4 g). Purification by flash column chromatography (silica 160 g, 35% dichloromethane/hexanes) provided 5-phenoxy-1H-indole (2.7 g, 68%) as white needles. MS (ES) m/z 210 ([M+H]⁺).

Step 4:

In an analogous manner to Example 1, step 2, 5-phenoxyindoline was prepared from 5-phenoxy-1H-indole. MS (ES) m/z 212 ([M+H]⁺).

Step 5:

In an analogous manner to Example 1, step 3, (2S,3S)-3-[5-(phenoxy)-2,3-dihydro-1H-indol-1-yl]-3-phenylpropane-1,2-diol was prepared from 5-phenoxyindoline. MS (ES) m/z 362 ([M+H]⁺).

Step 6:

In an analogous manner to Example 1, step 4, (2S,3S)-3-[5-(phenoxy)-1H-indol-1-yl]-3-phenylpropane-1,2-diol was prepared from (2S,3S)-3-[5-(phenoxy)-2,3-dihydro-1H-indol-1-yl]-3-phenylpropane-1,2-diol. MS (ES) m/z 360 ([M+H]⁺).

Step 7:

In an analogous manner to Example 1, step 5, (2S,3S)-toluene-4-sulfonic acid 2-hydroxy-3-(5-phenoxy-indol-1-yl)-3-phenyl-propyl ester was prepared from (2S,3S)-3-[5-(phenoxy)-1H-indol-1-yl]-3-phenylpropane-1,2-diol. MS (ES) m/z 514 ([M+H]⁺).

Step 8:

In an analogous manner to Example 1, step 6, (1S,2R)-3-(methylamino)-1-[5-(phenoxy)-1H-indol-1-yl]-1-phenylpropan-2-ol hydrochloride was prepared from (2S,3S)-toluene-4-sulfonic acid 2-hydroxy-3-(5-phenoxy-indol-1-yl)-3-phenyl-propyl ester. MS (ES) m/z 372.9 ([M+H]⁺).

### Example 20

### (1S,2R)-3-(methylamino)-1-[4-(phenoxy)-1H-indol-1-yl]-1-phenylpropan-2-ol hydrochloride

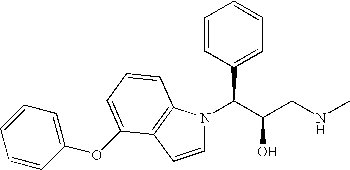

Step 1:

2-Methyl-3-nitrophenol (4.6 g, 30 mmol), phenylboronic acid (7.3 g, 60 mmol), copper (II) acetate (5.5 g, 30 mmol) and 4 A powdered molecular sieves (30 g, dried at 200° C.) were combined in dry dichloromethane (300 mL) at 23° C. Triethylamine (d 0.726, 21 mL, 150 mmol) was added and the mixture was stirred vigorously at 23° C. After 24 hours, additional phenylboronic acid (7.3 g, 60 mmol) was added. After 28 hours. (total), additional copper (II) acetate (2.3 g, 13 mmol) was added. After 48 hours, the mixture was filtered (Celite) and washed with dichloromethane. The filtrate was washed with saturated aqueous EDTA (disodium salt) solution (4×300 mL) and brine (300 mL), dried over sodium sulfate, filtered and concentrated under reduced pressure to yield a tacky brown solid (6.9 g) that was dissolved in dichloromethane and pre-adsorbed on silica gel (15 g). Purification by flash column chromatography (silica 135 g, 1%, 2%, 5%, 10%, 20%, 40% ethyl acetate/hexanes) provided 2-methyl-1-nitro-3-phenoxybenzene (2.9 g, 91% based on recovered 2-methyl-3-nitrophenol) as a clear, light yellow oil. MS (EI) m/z 229 [M+].

Step 2:

In an analogous manner to Example 19, step 2, dimethyl-[2-(2-nitro-6-phenoxy-phenyl)-vinyl]-amine was prepared from 2-methyl-1-nitro-3-phenoxybenzene.

Step 3:

In an analogous manner to Example 19, step 3, 4-phenoxy-1H-indole was prepared from dimethyl-[2-(2-nitro-6-phenoxy-phenyl)-vinyl]-amine. MS (ES) m/z 210 ([M+H]⁺).

Step 4:

In an analogous manner to Example 1, step 2, 4-phenoxyindoline was prepared from 4-phenoxy-1H-indole. MS (ES) m/z 212 ([M+H]⁺).

Step 5:

In an analogous manner to Example 1, step 3, (2S,3S)-3-[4-(phenoxy)-2,3-dihydro-1H-indol-1-yl]-3-phenylpropane-1,2-diol was prepared from 4-phenoxyindoline. MS (ES) m/z 362 ([M+H]⁺).

Step 6:

In an analogous manner to Example 1, step 4, (2S,3S)-3-[4-(phenoxy)-1H-indol-1-yl]-3-phenylpropane-1,2-diol was prepared from (2S,3S)-3-[4-(phenoxy)-2,3-dihydro-1H-indol-1-yl]-3-phenylpropane-1,2-diol. MS (ES) m/z 360 ([M+H]⁺).

Step 7:

In an analogous manner to Example 1, step 5, (2S,3S)-toluene-4-sulfonic acid 2-hydroxy-3-(4-phenoxy-indol-1-yl)-3-phenyl-propyl ester was prepared from (2S,3S)-3-[4-(phenoxy)-1H-indol-1-yl]-3-phenylpropane-1,2-diol. MS (ES) m/z 514 ([M+H]⁺).

Step 8:

In an analogous manner to Example 1, step 6, (1S,2R)-3-(methylamino)-1-[4-(phenoxy)-1H-indol-1-yl]-1-phenylpropan-2-ol hydrochloride was prepared from (2S,3S)-toluene-4-sulfonic acid 2-hydroxy-3-(4-phenoxy-indol-1-yl)-3-phenyl-propyl ester. MS (ES) m/z 372.9 ([M+H]⁺).

### Example 21

### (1S,2R)-3-(methylamino)-1-phenyl-1-(4-phenyl-1H-indol-1-yl)propan-2-ol hydrochloride

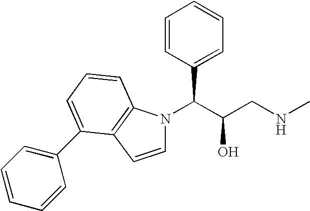

Step 1:

A mixture of 4-bromo-1H-indole (1.57 g, 8.0 mmol), phenylboronic acid (1.17 g, 9.6 mmol) and potassium carbonate (3.32 g, 24 mmol) in 3:1 v/v dioxane:water (40 mL) was purged with a bubbling stream of nitrogen for 15 minutes. Trans-dichlorobis(tri-o-tolylphosphine)palladium(II) (0.314 g, 0.4 mmol) was then added and the reaction mixture stirred at ambient temperature overnight. The mixture was then concentrated under reduced pressure and the residue partitioned between 2.0 N sodium hydroxide solution and ethyl acetate. The layers were separated and the aqueous layer extracted 3 times with ethyl acetate. The combined organic layers were washed once with brine, dried over anhydrous magnesium sulfate, filtered and concentrated under reduced pressure. The crude product was purified by flash chromatography (silica gel 5% ethyl acetate in hexane) to give 1.45 g (94%) of 4-phenyl-1H-indole as cream-colored solid. HRMS: calculated for C₁₄H₁₁N+H⁺, 194.09642; found (ESI, [M+H]⁺), 194.0967).

Step 2:

In an analogous manner to Example 1, Step 2, 4-phenylindoline was prepared from 4-phenyl-1H-indole. HRMS: calculated for C₁₄H₁₃N+H⁺, 196.11207; found (ESI, [M+H]⁺), 196.1129.

Step 3:

In an analogous manner to Example 1, Step 3, (2S,3S)-3-phenyl-3-(4-phenyl-2,3-dihydro-1H-indol-1-yl)propane-1,2-diol was prepared from 4-phenylindoline. HRMS: calculated for C₂₃H₂₃NO₂+H⁺, 346.18016; found (ESI, [M+H]⁺), 346.1807.

Step 4:

In an analogous manner to Example 1, Step 4, (2S,3S)-3-phenyl-3-(4-phenyl-1H-indol-1-yl)propane-1,2-diol was prepared from (2S,3S)-3-phenyl-3-(4-phenyl-2,3-dihydro-1H-indol-1-yl)propane-1,2-diol. HRMS: calculated for C₂₃H₂₁NO₂+H⁺, 344.16451; found (ESI, [M+H]⁺), 344.164.

Step 5:

In an analogous manner to Example 1, Step 5, (2S,3S)-toluene-4-sulfonic acid 3-(4-phenyl-indol-1-yl)-2-hydroxy-3-phenyl-propyl ester was prepared from (2S,3S)-3-phenyl-3-(4-phenyl-1H-indol-1-yl)propane-1,2-diol. MS (ESI) m/z 498.2 ([M+H]⁺).

Step 6:

In an analogous manner to Example 1, Step 6, (1S,2R)-3-(methylamino)-1-phenyl-1-(4-phenyl-1H-indol-1-yl)propan-2-ol hydrochloride was prepared from (2S,3S)-toluene-4-sulfonic acid 3-(4-phenyl-indol-1-yl)-2-hydroxy-3-phenyl-propyl ester. HRMS: calculated for C₂₄H₂₄N₂O+H⁺, 357.19614; found (ESI, [M+H]⁺), 357.1962.

### Example 22

### (1S,2R)-3-(methylamino)-1-phenyl-1-(6-phenyl-1H-indol-1-yl)propan-2-ol hydrochloride

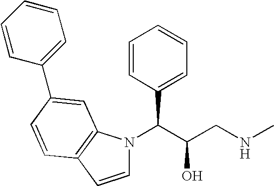

In an analogous manner to Example 21, Step 1, 6-phenyl-1H-indole was prepared from 6-bromo-1H-indole. HRMS: calculated for C₁₄H₁₁N, 193.08915; found (EI, M⁺), 193.0891).

In an analogous manner to Example 1, Step 2, 6-phenylindoline was prepared from 6-phenyl-1H-indole. HRMS: calculated for C₁₄H₁₃N, 195.10480; found (EI, M⁺), 195.1034.

In an analogous manner to Example 1, Step 3, (2S,3S)-3-phenyl-3-(6-phenyl-2,3-dihydro-1H-indol-1-yl)propane-1,2-diol was prepared from 6-phenylindoline. HRMS: calculated for C₂₃H₂₃NO₂+H⁺, 346.18016; found (ESI, [M+H]⁺), 346.1787.

In an analogous manner to Example 1, Step 4, (2S,3S)-3-phenyl-3-(6-phenyl-1H-indol-1-yl)propane-1,2-diol was prepared from (2S,3S)-3-phenyl-3-(6-phenyl-2,3-dihydro-1H-indol-1-yl)propane-1,2-diol. HRMS: calculated for C₂₃H₂₁NO₂+H⁺, 344.16451; found (ESI, [M+H]⁺), 344.1633.

In an analogous manner to Example 1, Step 5, (2S,3S)-toluene-4-sulfonic acid 3-(6-phenyl-indol-1-yl)-2-hydroxy-3-phenyl-propyl ester was prepared from (2S,3S)-3-phenyl-3-(6-phenyl-1H-indol-1-yl)propane-1,2-diol. MS (ESI) m/z 498.2 ([M+H]⁺).

In an analogous manner to Example 1, Step 6, (1S,2R)-3-(methylamino)-1-phenyl-1-(6-phenyl-1H-indol-1-yl)propan-2-ol hydrochloride was prepared from (2S,3S)-toluene-4-sulfonic acid 3-(6-phenyl-indol-1-yl)-2-hydroxy-3-phenyl-propyl ester. HRMS: calculated for C₂₄H₂₄N₂O+H⁺, 357.19614; found (ESI, [M+H]⁺), 357.1958.

### Example 23

### (1S,2R)-3-(methylamino)-1-phenyl-1-(7-phenyl-1H-indol-1-yl)propan-2-ol hydrochloride

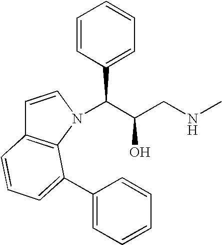

In an analogous manner to Example 21, Step 1, 7-phenyl-1H-indole was prepared from 7-bromo-1H-indole. HRMS: calculated for C₁₄H₁₁N, 193.08915; found (EI, M⁺), 193.0878.

In an analogous manner to Example 1, Step 2, 7-phenylindoline was prepared from 7-phenyl-1H-indole. MS (ESI) m/z 196.2 ([M+H]⁺).

In an analogous manner to Example 1, Step 3, (2S,3S)-3-phenyl-3-(7-phenyl-2,3-dihydro-1H-indol-1-yl)propane-1,2-diol was prepared from 7-phenylindoline. HRMS: calculated for C₂₃H₂₃NO₂+H⁺, 346.18016; found (ESI, [M+H]⁺), 346.1816.

In an analogous manner to Example 1, Step 4, (2S,3S)-3-phenyl-3-(7-phenyl-1H-indol-1-yl)propane-1,2-diol was prepared from (2S,3S)-3-phenyl-3-(7-phenyl-2,3-dihydro-1H-indol-1-yl)propane-1,2-diol. HRMS: calculated for C₂₃H₂₁NO₂+H⁺, 344.16451; found (ESI, [M+H]⁺), 344.1626.

In an analogous manner to Example 1, Step 5, (2S,3S)-toluene-4-sulfonic acid 3-(7-phenyl-indol-1-yl)-2-hydroxy-3-phenyl-propyl ester was prepared from (2S,3S)-3-phenyl-3-(7-phenyl-1H-indol-1-yl)propane-1,2-diol. MS (ESI) m/z 498.2 ([M+H]⁺).

In an analogous manner to Example 1, Step 6, (1S,2R)-3-(methylamino)-1-phenyl-1-(7-phenyl-1H-indol-1-yl)propan-2-ol hydrochloride was prepared from (2S,3S)-toluene-4-sulfonic acid 3-(7-phenyl-indol-1-yl)-2-hydroxy-3-phenyl-propyl ester. HRMS: calculated for C₂₄H₂₄N₂O+H⁺, 357.19614; found (ESI, [M+H]⁺), 357.1971.

### Example 24

### (1S,2R)-1-[5-(benzyloxy)-1H-indol-1-yl]-1-(3-fluorophenyl)-3-(methylamino)propan-2-ol hydrochloride

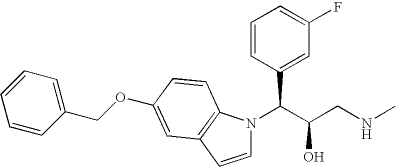

Step 1:

An oven-dried, three-neck, 2-L round bottomed flask fitted with two oven-dried addition funnels and a rubber septum was charged with diisopropyl D-tartrate (11.55 g, 49.3 mmol, 0.30 equiv.), 4 A powdered, activated molecular sieves (40 g) and anhydrous dichloromethane (800 mL) under nitrogen. After being cooled to −25° C., to the reaction mixture was added titanium isopropoxide (9.6 mL, 33 mmol, 0.20 equiv.) slowly via a hypodermic syringe. After stirring for 10 minutes, anhydrous t-butyl hydroperoxide (5.5 M in decane, 75.0 mL, 413 mmol, 2.5 equiv.) was added at a moderate rate via an addition funnel. The resulting mixture was stirred at −25° C. for 30 minutes. trans-3-Fluorocinnamyl alcohol (25.0 g, 164 mmol) in anhydrous dichloromethane (50 mL) was added dropwise via an addition funnel while maintaining the temperature at −25° C. After the addition, the reaction mixture was stirred at −25° C. for 1 hour and at −20° C. for additional 3 hours. After the reaction was complete, cooled aqueous sodium hydroxide solution (30%, 20 mL) saturated with sodium chloride was added slowly at −20° C. After diethyl ether (150 mL) was added, the cold bath was removed and the mixture was warmed to ˜5° C. and stirred for 1 hour. Anhydrous magnesium sulfate (50 g) was added and the mixture was stirred for 20 minutes, then filtered through a pad of silica gel, and washed with diethyl ether (300 mL). The filtrate was concentrated and toluene was used to azeotropically remove excess t-butyl hydroperoxide. The residual oil was purified on silica gel (0-30% ethyl acetate/hexane) to give 24.80 g (90%) of [(2R,3R)-3-(3-fluorophenyl)oxiran-2-yl]methanol as a viscous colorless oil. Percent ee: >96.5%. MS (ESI) m/z 169.1 ([M+H]⁺).

Step 2:

A mixture of sodium hydride (60% in mineral oil, 0.40 g, 10 mmol) and tert-butanol (5 mL) was stirred for 15 minutes under nitrogen at room temperature. 5-Benzyloxyindole (2.23 g, 10 mmol) in methylene chloride (2 mL) was then added and the mixture was stirred for an additional 30 minutes at room temperature. A pre-mixed solution of titanium isopropoxide (3.55 mL, 12 mmol) and [(2R,3R)-3-(3-fluorophenyl)oxiran-2-yl]methanol (68 g, 10 mmol) in methylene chloride (2 mL) was added, and the reaction mixture was stirred at room temperature for 15 hours until no epoxide remained as determined by tic. The mixture was filtered through a Celite pad, and the filtrate was then treated with a 2N aqueous solution of hydrochloric acid (50 mL) with stirring over 30 minutes. The organic layer was separated and the aqueous layer was extracted with methylene chloride several times. The combined extracts were washed with water, dried over anhydrous sodium sulfate, filtered, and concentrated under reduced pressure. The crude product was purified via Biotage Horizon (FlasH 40 M, silica, gradient from 10% ethyl acetate/hexane to 65% ethyl acetate/hexane) to yield (2S,3S)-3-(5-benzyloxy-1H-indol-1-yl)-3-(3-fluorophenyl)propane-1,2-diol as an oil. MS (ESI) m/z 392 ([M+H]⁺).

In an analogous manner to Example 1, step 5 (2S,3S)-toluene-4-sulfonic acid 3-(5-benzyloxy-indol-1-yl)-2-hydroxy-3-(3-fluorophenyl)-propyl ester was prepared from (2S,3S)-3-(5-benzyloxy-1H-indol-1-yl)-3-(3-fluorophenyl)-propane-1,2-diol as an oil. MS (ESI) m/z 546 ([M+H]⁺).

In an analogous manner to Example 1, step 6, (1S,2R)-1-(5-benzyloxy-1H-indol-1-yl)-1-(3-fluorophenyl)-3-(methylamino)propan-2-ol hydrochloride was prepared from (2S,3S)-toluene-4-sulfonic acid 3-(5-benzyloxy-indol-1-yl)-2-hydroxy-3-(3-fluorophenyl)-propyl ester and methylamine solution (2.0 M in methanol) as an off-white solid. MS (ES) m/z 405.2; HRMS: calculated for C₂₅H₂₅FN₂O₂+H⁺, 405.19728; found (ESI, [M+H]⁺), 405.1989.

### Example 25

### (1S,2R)-1-[5-(benzyloxy)-2,3-dihydro-1H-indol-1-yl]-1-(3-fluorophenyl)-3-(methylamino)propan-2-ol hydrochloride

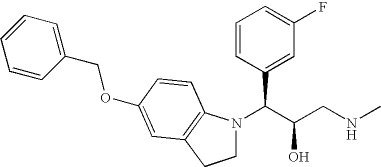

Step 1:

To a mixture of trans-3-fluorocinnamic acid (50 g, 300 mmol) and iodomethane (300 mL) in acetone (1 L) was added portionwise cesium carbonate (147 g, 450 mmol, 1.5 equiv.), and the mixture was heated at 65° C. for 1.5 hours in a sealed reaction vessel. Upon cooling to room temperature, the reaction mixture was diluted with ethyl acetate (1 L), filtered through a pad of silica gel, and concentrated under reduced pressure to give 47.33 g (87%) of trans-3-fluorocinnamic acid methyl ester as a colorless oil. MS (ES) m/z 180.0 (M⁺).

Step 2:

To a solution of trans-3-fluorocinnamic acid methyl ester (69.61 g, 386 mmol) in dry dichloromethane (1 L) at −78° C. under nitrogen was added dropwise diisobutylaluminum hydride (neat, 172 mL, 965 mmol, 2.5 equiv.) via an addition funnel. After the addition was complete, the reaction mixture was allowed to warm to −30° C. and stirred for an additional 1 hour, then quenched with methanol (150 mL). Upon warming to room temperature, the reaction mixture was treated with saturated aqueous of sodium/potassium tartrate solution (300 mL) and stirred for 30 minutes. The organic layer was washed sequentially with 1 N aqueous hydrochloric acid solution, saturated aqueous sodium bicarbonate solution and brine, dried over sodium sulfate, filtered and concentrated ender reduced pressure. The crude oil was purified by silica gel chromatography (0-50% ethyl acetate:hexane) to give 53.07 g (90%) of trans-3-fluorocinnamyl alcohol as a colorless oil. MS (ES) m/z 152.1 (M⁺).

Step 3:

An oven-dried, 3-neck, 2-L round bottom flask fitted with two oven-dried addition funnels and a rubber septum was charged with diisopropyl D-tartrate (11.55 g, 49.3 mmol, 0.30 equiv.), 4 Å powdered, activated molecular sieves (40 g) and dry dichloromethane (800 mL) under nitrogen. After being cooled to −25° C., to the reaction mixture was added titanium isopropoxide (9.6 mL, 33 mmol, 0.20 equiv.) slowly via a hypodermic syringe. After stirring for 10 minutes, anhydrous t-butyl hydroperoxide solution (5.5 M in decane, 75.0 mL, 413 mmol, 2.5 equiv.) was added at a moderate rate via an addition funnel. The resulting mixture was stirred at −25° C. for 30 min. trans-3-Fluorocinnamyl alcohol (25.0 g, 164 mmol) in dry dichloromethane (50 mL) was then added dropwise via an addition funnel while maintaining the temperature at −25° C. After the addition, the reaction mixture was stirred at −25° C. for 1 hour and at −20° C. for another 3 hours. After the reaction was complete, cooled aqueous sodium hydroxide solution (30%, 20 mL) saturated with sodium chloride was added slowly at −20° C. After diethyl ether (150 mL) was added, the cold bath was removed and the mixture was allowed to warm to ˜5° C. and stirred for 1 hour. Magnesium sulfate (anhydrous, 50 g) was added and the mixture was stirred for 20 minutes, then filtered through a pad of silica gel, and washed with diethyl ether (300 mL). The filtrate was concentrated and toluene was used to azeotropically remove excess t-butyl hydroperoxide. The residual oil was purified on silica gel (0-30% ethyl acetate:hexane) to give 24.80 g (90%) of [(2R,3R)-3-(3-fluorophenyl)oxiran-2-yl]methanol as a viscous, colorless oil. Percent ee: >96.5%. MS (ESI) m/z 169.1 ([M+H]⁺).

Step 4:

In an analogous manner to Example 1, step 3, (2S,3S)-3-[5-(benzyloxy)-2,3-dihydro-1H-indol-1-yl]-3-(3-fluorophenyl)propane-1,2-diol was prepared from 5-(benzyloxy)indoline (Example 1, step 2) and [(2R,3R)-3-(3-fluorophenyl)oxiran-2-yl]methanol as an amber colored oil. MS (ESI) m/z 394.2 ([M+H]⁺); HRMS: calculated for C₂₄H₂₄FNO₃+H⁺, 394.1813; found (ESI, [M+H]⁺), 394.1808.

Step 5:

To a solution of (2S,3S)-3-[5-(benzyloxy)-2,3-dihydro-1H-indol-1-yl]-3-(3-fluorophenyl)propane-1,2-diol (348 mg, 0.884 mmol) in dichloromethane (3 mL) under nitrogen was added triethylamine (0.62 mL, 4.43 mmol, 5 equiv.). The mixture was cooled to 0° C., and para-toluenesulfonyl chloride (219 mg, 1.15 mmol) was added portionwise. The reaction mixture was stirred at 0° C. for 6 hours and methylamine solution (33% in absolute ethanol, 5 mL) was added and the reaction mixture was sealed, and stirred overnight while warming to room temperature. All volatiles were removed under reduced pressure. The oily residue was dissolved in dichloromethane (20 mL), washed with aqueous potassium carbonate (5 mL), dried over sodium sulfate, filtered and concentrated under reduced pressure. Purification by Biotage chromatography (FlasH12i, silica, 0-15% MeOH/dichloromethane/0.5% triethylamine) gave 282 mg (78%) (1S,2R)-1-[5-(benzyloxy)-2,3-dihydro-1H-indol-1-yl]-1-(3-fluorophenyl)-3-(methylamino)propan-2-ol, which was dissolved dichloromethane (5 mL) and treated with hydrogen chloride solution (1.0 M in diethyl ether, 0.80 mL, 0.80 mmol). To the resulting solution was added hexane until white powder formed, which was collected, washed with hexane, and dried in vacuo to yield (1S,2R)-1-[5-(benzyloxy)-2,3-dihydro-1H-indol-1-yl]-1-(3-fluorophenyl)-3-(methylamino)propan-2-ol hydrochloride as a white powder. MS (ES) m/z 407.0 ([M+H]⁺); HRMS: calculated for C₂₅H₂₇FN₂O₂+H⁺, 407.2129; found (ESI, [M+H]⁺), 407.2131.

### Example 26

### (1S,2R)-1-[5-(benzyloxy)-2,3-dihydro-1H-indol-1-yl]-3-(methylamino)-1-phenylpropan-2-ol hydrochloride

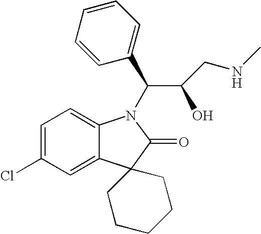

In an analogous manner to Example 25, step 5, (1S,2R)-1-[5-(benzyloxy)-2,3-dihydro-1H-indol-1-yl]-3-(methylamino)-1-phenylpropan-2-ol hydrochloride was prepared from (2S,3S)-3-[5-(benzyloxy)-2,3-dihydro-1H-indol-1-yl]-3-phenylpropane-1,2-diol (Example 1, step 3) as a white powder. MS (ES) m/z 389.2 ([M+H]⁺); HRMS: calculated for C₂₅H₂₈N₂O₂+H⁺, 389.2224; found (ESI, [M+H]⁺), 389.2220.

### Example 27

### 5′-chloro-1′-[(1S,2R)-2-hydroxy-3-(methylamino)-1-phenylpropyl]spiro[cyclohexane-1,3′-indol]-2′(1′H)-one hydrochloride

Step 1:

5-Chlorooxindole (1 g, 6.0 mmol) and lithium chloride (0.63 g, 14.8 mmol) were suspended in tetrahydrofuran (50 mL) and the mixture cooled to 0° C. n-Butylithium (6.2 mL, 12.6 mmol) was added slowly and the mixture was stirred for 20 minutes, then dibromopentane (0.82 mL, 6.0 mmol) was added. The mixture was warmed to 25° C. and stirred for 16 hours. The reaction was quenched with saturated aqueous ammonium chloride and diluted with diethyl ether. The organics were washed with water and saturated brine, dried over magnesium sulfate, filtered and concentrated under reduced pressure. Purification by flash chromatography (0-20% hexane/ethyl acetate) afforded 700 mg (50%) of 5′-chlorospiro[cyclohexane-1,3′-indol]-2′(1′H)-one as a white solid. MS (ESI) m/z 236 ([M+H]⁺).

Step 2:

5′-chlorospiro[cyclohexane-1,3′-indol]-2′(1′H)-one (700 mg, 3.0 mmol) was dissolved in N,N-dimethylformamide (10 mL) and sodium hydride (244 mg, 6.1 mmol, 60% wt suspension in mineral oil) was added in portions over 15 minutes and the mixture stirred for an additional 30 minutes. In a separate flask, [(2R,3R)-3-phenyloxiran-2-yl]methanol (0.8 g, 5.3 mmol, from Example 1 Step 1) was dissolved in N,N-dimethylformamide (10 mL) and titanium isopropoxide (1.6 mL, 5.3 mmol) was added and the mixture stirred 30 minutes. The titanium isopropoxide/epoxide solution was then added to the solution of oxindole sodium salt dropwise and the mixture stirred at room temperature for 16 hours. The mixture was then carefully quenched with 2.0 N aqueous hydrochloric acid and diluted with 200 mL of 2.0 N aqueous hydrochloric acid. The mixture was extracted with ethyl acetate and then the organic layers were combined, washed with water, and saturated brine, dried over anhydrous magnesium sulfate, filtered and concentrated under reduced pressure. The crude product was purified via Isco chromatography (Redisep, silica, gradient 20% to 100% ethyl acetate in hexane) to afford 0.5 g (43%) of 5′-chloro-1′-[(1S,2S)-2,3-dihydroxy-1-phenylpropyl]spiro[cyclohexane-1,3′-indol]-2′(1′H)-one as a foaming solid. MS (ES) m/z 385.9 ([M+H]⁺).

Step 3:

5′-chloro-1′-[(1S,2S)-2,3-dihydroxy-1-phenylpropyl]spiro[cyclohexane-1,3′-indol]-2′(1′H)-one (0.5 g, 1.3 mmol) was dissolved in pyridine (4 mL), p-toluenesufonyl chloride (310 mg, 1.6 mmol) added and the mixture stirred for 4 hours. The reaction mixture was then diluted with diethyl ether and washed with water, 2.0 N aqueous hydrochloric acid, saturated copper sulfate, 2.0 N hydrochloric acid, and saturated brine. The organic layer was separated, dried over anhydrous magnesium sulfate, filtered and concentrated under reduced pressure. The crude product was immediately dissolved in a solution of methylamine (8.0 M in ethanol, 10 mL, 80 mmol) and stirred for 16 hours. The mixture was then concentrated under reduced pressure and purified via flash chromatography (0% to 10% methanol in dichloromethane) to give 5′-chloro-1′-[(1S,2R)-2-hydroxy-3-(methylamino)-1-phenylpropyl]spiro[cyclohexane-1,3′-indol]-2′(1′H)-one as a colorless oil. The freebase was dissolved in diethyl ether (10 mL) and treated with a solution of hydrogen chloride (2.0 M in diethyl ether, 1.1 equivalent). The white precipitate was collected and dried under vacuum to give 180 mg (32% over three steps) of 5′-chloro-1′-[(1S,2R)-2-hydroxy-3-(methylamino)-1-phenylpropyl]spiro[cyclohexane-1,3′-indol]-2′(1′H)-one hydrochloride. HRMS: calculated for C₂₃H₂₇ClN₂O₂+H⁺, 399.18338; found (ESI, [M+H]⁺), 399.1822.

### Example 28

### 6′-Chloro-1′-[(1S,2R)-2-hydroxy-3-(methylamino)-1-phenylpropyl]spiro[cyclohexane-1,3′-indol]-2′(1′H)-one hydrochloride

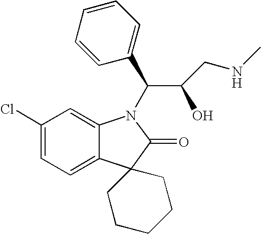

In an analogous manner to Example 27, Step 1, 6′-chlorospiro[cyclohexane-1,3′-indol]-2′(1′H)-one was prepared from 6-chlorooxindole. MS (ES) m/z 236.0 ([M+H]⁺).

In an analogous manner to Example 27, Step 2, 6′-chloro-1′-[(1S,2S)-2,3-dihydroxy-1-phenylpropyl]spiro[cyclohexane-1,3′-indol]-2′(1′H)-one was prepared from 6′-chlorospiro[cyclohexane-1,3′-indol]-2′(1′H)-one. MS (ES) m/z 385.8 ([M+H]⁺).

In an analogous manner to Example 27, Step 3, 6′-chloro-1′-[(1S,2R)-2-hydroxy-3-(methylamino)-1-phenylpropyl]spiro[cyclohexane-1,3′-indol]-2′(1′H)-one hydrochloride was prepared from 6′-chlorospiro[cyclohexane-1,3′-indol]-2′(1′H)-one. HRMS: calculated for C₂₃H₂₇ClN₂O₂+H⁺, 399.18338; found (ESI, [M+H]⁺), 399.182.

### Example 29

### 6′-fluoro-1′-[(1S,2R)-2-hydroxy-3-(methylamino)-1-phenylpropyl]spiro[cyclohexane-1,3′-indol]-2′(1′H)-one hydrochloride

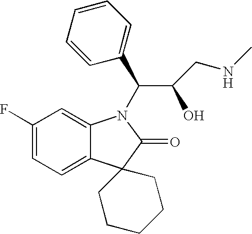

Step 1:

In an analogous manner to Example 27, Step 1, 6′-fluorospiro[cyclohexane-1,3′-indol]-2′(1′H)-one was prepared from 6-fluorooxindole. MS (ES) m/z 219.9 ([M+H]⁺).

Step 2:

In an analogous manner to Example 27, Step 2, 1′-[(1S,2S)-2,3-dihydroxy-1-phenylpropyl]-6′-fluorospiro[cyclohexane-1,3′-indol]-2′(1′H)-one was prepared from 6′-fluorospiro[cyclohexane-1,3′-indol]-2′(1′H)-one.

Step 3:

In an analogous manner to Example 27, Step 3, 6′-fluoro-1′-[(1S,2R)-2-hydroxy-3-(methylamino)-1-phenylpropyl]spiro[cyclohexane-1,3′-indol]-2′(1′H)-one hydrochloride was prepared from 1′-[(1S,2S)-2,3-dihydroxy-1-phenylpropyl]-6′-fluorospiro[cyclohexane-1,3′-indol]-2′(1′H)-one. HRMS: calculated for C₂₃H₂₇FN₂O₂+H⁺, 383.21293; found (ESI, [M+H]⁺), 383.2139.

### Example 30

### 5′-fluoro-1′-[(1S,2R)-2-hydroxy-3-(methylamino)-1-phenylpropyl]spiro[cyclohexane-1,3′-indol]-2′(1′H)-one hydrochloride

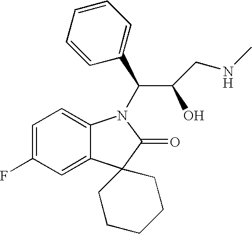

Step 1:

In an analogous manner to Example 27, Step 1, 5′-fluorospiro[cyclohexane-1,3′-indol]-2′(1′H)-one was prepared form 5-fluorooxiindole. MS (ES) m/z 219.9 ([M+H]⁺).

Step 2:

In an analogous manner to Example 27, Step 2, 1′-[(1S,2S)-2,3-dihydroxy-1-phenylpropyl]-5′-fluorospiro[cyclohexane-1,3′-indol]-2′(1′H)-one was prepared from 5′-fluorospiro[cyclohexane-1,3′-indol]-2′(1′H)-one. MS (ES) m/z 369.8 ([M+H]⁺).

Step 3:

In an analogous manner to Example 27, Step 3, 5′-fluoro-1′-[(1S,2R)-2-hydroxy-3-(methylamino)-1-phenylpropyl]spiro[cyclohexane-1,3′-indol]-2′(1′H)-one hydrochloride was prepared from 1′-[(1S,2S)-2,3-dihydroxy-1-phenylpropyl]-5′-fluorospiro[cyclohexane-1,3′-indol]-2′(1′H)-one. HRMS: calculated for C₂₃H₂₇FN₂O₂+H⁺, 383.21293; found (ESI, [M+H]⁺), 383.2125.

### Example 31

### 7′-Chloro-1′-[(1S,2R)-2-hydroxy-3-(methylamino)-1-phenylpropyl]spiro[cyclohexane-1,3′-indol]-2′(1′H)-one hydrochloride

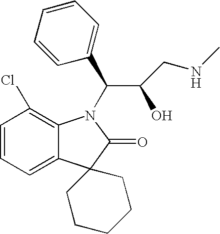

In an analogous manner to Example 27, Step 1, 7′-chlorospiro[cyclohexane-1,3′-indol]-2′(1′H)-one was synthesized from 7-chlorooxindole. MS (ES) m/z 236.0 ([M+H]⁺).

In an analogous manner to Example 27, Step 2, 7′-chloro-1′-[(1S,2S)-2,3-dihydroxy-1-phenylpropyl]spiro[cyclohexane-1,3′-indol]-2′(1′H)-one was prepared from 7′-chlorospiro[cyclohexane-1,3′-indol]-2′(1′H)-one. MS (ES) m/z 385.8 ([M+H]⁺).

In an analogous manner to Example 27, Step 3, 7′-chloro-1′-[(1S,2R)-2-hydroxy-3-(methylamino)-1-phenylpropyl]spiro[cyclohexane-1,3′-indol]-2′(1′H)-one hydrochloride was prepared from 7′-chlorospiro[cyclohexane-1,3′-indol]-2′(1′H)-one. HRMS: calculated for C₂₃H₂₇ClN₂O₂+H⁺, 399.18338; found (ESI, [M+H]⁺), 399.1837.

### Example 32

### 6′-fluoro-1′-[(1S,2R)-1-(3-fluorophenyl)-2-hydroxy-3-(methylamino)propyl]spiro[cyclohexane-1,3′-indol]-2′(1′H)-one hydrochloride

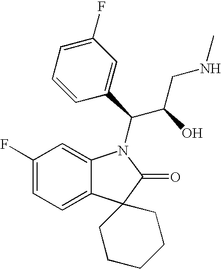

In an analogous manner to Example 27, Step 2, 1′-[(1S,2S)-2,3-dihydroxy-1-(3-fluorophenyl)-propyl]-6′-fluorospiro[cyclohexane-1,3′-indol]-2′(1′H)-one was prepared from 6′-fluorospiro[cyclohexane-1,3′-indol]-2′(1′H)-one (from Example 29 Step 1) and [(2R,3R)-3-(3-fluorophenyl)oxiran-2-yl]methanol.

In an analogous manner to Example 27, Step 3, 6′-fluoro-1′-[(1S,2R)-1-(3-fluorophenyl)-2-hydroxy-3-(methylamino)propyl]spiro[cyclohexane-1,3′-indol]-2′(1′H)-one hydrochloride was prepared from 1′-[(1S,2S)-2,3-dihydroxy-1-(3-fluorophenyl)propyl]-6′-fluorospiro[cyclohexane-1,3′-indol]-2′(1′H)-one. HRMS: calculated for C₂₃H₂₆F₂N₂O₂+H⁺, 401.20351; found (ESI, [M+H]⁺), 401.2005.

### Example 33

### (1S,2R)-3-(methylamino)-1-phenyl-1-spiro[cyclohexane-1,3′-indol]-1′(2′H)-ylpropan-2-ol hydrochloride

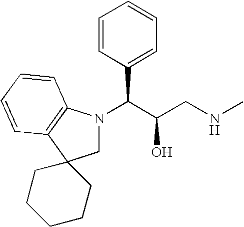

In an analogous manner to Example 1, step 3, (2S,3S)-3-phenyl-3-spiro[cyclohexane-1,3′-indol]-1′(2′H)-ylpropane-1,2-diol was prepared from spiro[cyclohexane-1,3′-indoline]¹ and [(2R,3R)-3-phenyloxiran-2-yl]methanol (from Example 1, step 1) as a white solid. MS (ESI) m/z 338.2 ([M+H]⁺); HRMS: calculated for C₂₂H₂₇NO₂+H⁺, 338.2115; found (ESI, [M+H]⁺), 338.2115.
¹ Kucerovy, A.; Hathaway, J. S.; Mattner, P. G.; Repic, O. Synth. Commun. 1992, 22, 729-733.

In an analogous manner to Example 25, step 5, (1S,2R)-3-(methylamino)-1-phenyl-1-spiro[cyclohexane-1,3′-indol]-1′(2′H)-ylpropan-2-ol hydrochloride was prepared from (2S,3S)-3-phenyl-3-spiro[cyclohexane-1,3′-indol]-1′(2′H)-ylpropane-1,2-diol as a white powder. MS (ES) m/z 351.2 ([M+H]⁺); HRMS: calculated for C₂₃H₃₀N₂O+H⁺, 351.2431; found (ESI, [M+H]⁺), 351.2421.

### Example 34

### (1S,2R)-1-(3-fluorophenyl)-3-(methylamino)-1-{3-[2-(trifluoromethoxy)phenyl]-1H-indol-1-yl}propan-2-ol hydrochloride

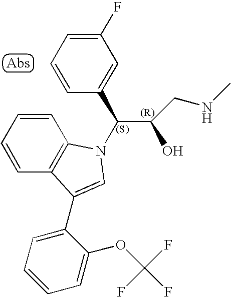

Step 1:

A mixture of indoline (1.42 g, 11.89 mmol) and [(2R,3R)-3-(3-fluorophenyl)oxiran-2-yl]methanol (2.0 g, 11.89 mmol, from Example 25, Step 3) was heated at 125° C. for 5 hours in a sealed reaction vial. Upon cooling, the crude product was dissolved in ethyl acetate, absorbed on Fluorocil, and purified by Biotage chromatography (FlasH40i, silica, 0-55% EtOAc/hexane) to give 2.55 g (75%) of (2S,3S)-3-(2,3-dihydro-1H-indol-1-yl)-3-(3-fluorophenyl)propane-1,2-diol as a colorless oil. MS (ESI) m/z 288.1 ([M+H]⁺).

Step 2:

A mixture of (2S,3S)-3-(2,3-dihydro-1H-indol-1-yl)-3-(3-fluorophenyl)propane-1,2-diol (2.00 g, 6.96 mmol) and activated manganese dioxide (20.0 g, 230 mmol) in dichloromethane (30 mL) was stirred at 20° C. for 3 hours. The mixture was diluted with ethyl acetate (15 mL), filtered through a pad of silica gel, and concentrated under reduced pressure. The crude product was purified by Biotage chromatography (FlasH40i, silica, 0-70% EtOAc/hexane) to give 1.40 g (71%) of (2S,3S)-3-(3-fluorophenyl)-3-(1H-indol-1-yl)propane-1,2-diol as a colorless oil. MS (ESI) m/z 286.0 ([M+H]⁺). HRMS: calculated for C₁₇H₁₆FNO₂+H⁺, 286.1238; found (ESI, [M+H]⁺), 286.1239.

Step 3:

To a solution of (2S,3S)-3-(3-fluorophenyl)-3-(1H-indol-1-yl)propane-1,2-diol (1.34 g, 4.56 mmol) in N,N-dimethylformamide (20 mL) was added pulverized solid potassium hydroxide (0.76 g, 13.68 mmol). The mixture was stirred for 15 minutes under nitrogen at room temperature, whereupon iodine (1.21 g, 4.72 mmol) was added in one portion. The mixture was stirred for 30 minutes at room temperature and then poured into 5% aqueous sodium thiosulfate solution (100 mL). The solution was extracted 3 times with ethyl acetate and the combined extracts were washed 3 times with water. The organic layer was dried over anhydrous sodium sulfate, filtered, and concentrated under reduced pressure. The crude product was purified via Biotage chromatography (FlasH40i, silica, 40% ethyl acetate/hexane) to yield 0.91 g (48%) of (2S,3S)-3-(3-fluorophenyl)-3-(3-iodo-1H-indol-1-yl)propane-1,2-diol as a dark brown oil. MS (ES) m/z 411.9 ([M+H]⁺).

Step 4:

A mixture of (2S,3S)-3-(3-fluorophenyl)-3-(3-iodo-1H-indol-1-yl)propane-1,2-diol (0.51 g, 1.24 mmol), 2-(trifluoromethoxy)phenylboronic acid (0.38 g, 1.85 mmol), and potassium phosphate (0.78 g, 3.72 mmol) in N,N-dimethylformamide (10 mL) was degassed with nitrogen for 5 minutes then a catalytic amount (0.02 g) of [1,4-bis-(diphenylphosphine)butane]palladium (II) dichloride was added. The solution was heated to 90° C. for 3 hours then cooled and poured into water (100 mL). The aqueous mixture was extracted 3 times with ethyl acetate and the combined extracts were then washed 2 times with water. The ethyl acetate phase was dried by filtration through a plug of silica gel then concentrated under reduced pressure. The residue was purified by Biotage chromatography (FlasH40i, silica, 40% ethyl acetate/hexane) to yield 0.17 g of (2S,3S)-3-(3-fluorophenyl)-3-{3-[2-(trifluoromethoxy)phenyl]-1H-indol-1-yl}propane-1,2-diol as an oil, which was used in the next step without further purification.

Step 5:

In an analogous manner to Example 27, step 3, (1S,2R)-1-(3-fluorophenyl)-3-(methylamino)-1-{3-[2-(trifluoromethoxy)phenyl]-1H-indol-1-yl}propan-2-ol hydrochloride was prepared from (2S,3S)-3-(3-fluorophenyl)-3-{3-[2-(trifluoromethoxy)phenyl]-1H-indol-1-yl}propane-1,2-diol. MS (ES) m/z 459.1 ([M+H]⁺); HRMS: calculated for C₂₅H₂₂F₄N₂O₂+H⁺, 459.16902; found (ESI, [M+H]⁺), 459.1706.

### Example 35

### (1S,2R)-1-(3-fluorophenyl)-3-(methylamino)-1-{3-[2-(isopropoxy)phenyl]-1H-indol-1-yl}propan-2-ol hydrochloride

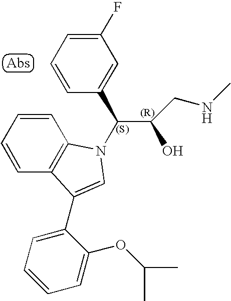

In an analogous manner to Example 34, step 4, (2S,3S)-3-(3-fluorophenyl)-3-{3-[2-(isopropoxy)phenyl]-1H-indol-1-yl}propane-1,2-diol was prepared from (2S,3S)-3-(3-fluorophenyl)-3-(3-iodo-1H-indol-1-yl)propane-1,2-diol (from Example 34, step 3) and 2-(isopropoxyphenyl)boronic acid.

In an analogous manner to Example 27, step 3, (1S,2R)-1-(3-fluorophenyl)-3-(methylamino)-1-{3-[2-(isopropoxy)phenyl]-1H-indol-1-yl}propan-2-ol hydrochloride was prepared from (2S,3S)-3-(3-fluorophenyl)-3-{3-[2-(isopropoxy)phenyl]-1H-indol-1-yl}propane-1,2-diol. MS (ES) m/z 433.2 ([M+H]⁺); HRMS: calculated for C₂₇H₂₉FN₂O₂+H⁺, 433.22858; found (ESI, [M+H]⁺), 433.2221.

### Example 36

### (1S,2R)-1-(3-fluorophenyl)-1-[3-(4-fluorophenyl)-1H-indol-1-yl]-3-(methylamino)propan-2-ol hydrochloride

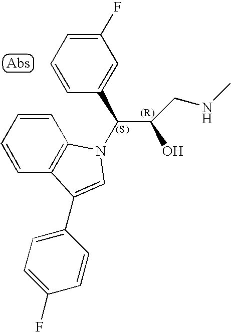

In an analogous manner to Example 34, step 4, (2S,3S)-3-(3-fluorophenyl)-3-{3-[4-fluorophenyl]-1H-indol-1-yl}propane-1,2-diol was prepared from (2S,3S)-3-(3-fluorophenyl)-3-(3-iodo-1H-indol-1-yl)propane-1,2-diol (from Example 34, step 3) and 4-(fluorophenyl)boronic acid.

In an analogous manner to Example 27, step 3, (1S,2R)-1-(3-fluorophenyl)-1-[3-(4-fluorophenyl)-1H-indol-1-yl]-3-(methylamino)propan-2-ol hydrochloride was prepared from (2S,3S)-3-(3-fluorophenyl)-3-{3-[4-fluorophenyl]-1H-indol-1-yl}propane-1,2-diol. MS (ES) m/z 393.2 ([M+H]⁺); HRMS: calculated for C₂₄H₂₂F₂N₂O+H⁺, 393.17729; found (ESI, [M+H]⁺), 393.1767.

### Example 37

### (1S,2R)-1-(3-fluorophenyl)-3-(methylamino)-1-[3-(2-phenoxyphenyl)-1H-indol-1-yl]propan-2-ol hydrochloride

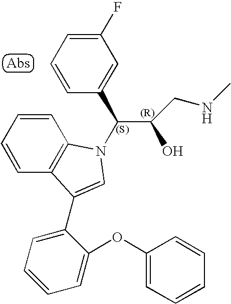

In an analogous manner to Example 34, step 4, (2S,3S)-3-(3-fluorophenyl)-3-{3-[2-phenoxyphenyl]-1H-indol-1-yl}propane-1,2-diol was prepared from (2S,3S)-3-(3-fluorophenyl)-3-(3-iodo-1H-indol-1-yl)propane-1,2-diol (from Example 34, step 3) and 2-(phenoxyphenyl)boronic acid.

In an analogous manner to Example 27, step 3, (1S,2R)-1-(3-fluorophenyl)-1-[3-(2-phenoxyphenyl)-1H-indol-1-yl]-3-(methylamino)propan-2-ol hydrochloride was prepared from (2S,3S)-3-(3-fluorophenyl)-3-{3-[2-phenoxyphenyl]-1H-indol-1-yl}propane-1,2-diol. MS (ES) m/z 467.2 ([M+H]⁺); HRMS: calculated for C₃₀H₂₇FN₂O₂+H⁺, 467.21293; found (ESI, [M+H]⁺), 467.2131.

### Example 38

### (1S,2R)-1-[3-(2,4-difluorophenyl)-1H-indol-1-yl]-1-(3-fluorophenyl)-3-(methylamino)propan-2-ol hydrochloride

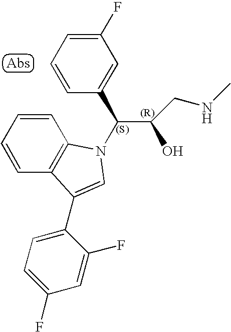

In an analogous manner to Example 34, step 4, (2S,3S)-3-(3-(2,4-difluorophenyl)-1H-indol-1-yl)-3-(3-fluorophenyl)propane-1,2-diol was prepared from (2S,3S)-3-(3-fluorophenyl)-3-(3-iodo-1H-indol-1-yl)propane-1,2-diol (from Example 34, step 3) and 2,4-(difluorophenyl)boronic acid.

In an analogous manner to Example 27, step 3, (1S,2R)-1-[3-(2,4-difluorophenyl)-1H-indol-1-yl]-1-(3-fluorophenyl)-3-(methylamino)propan-2-ol hydrochloride was prepared from (2S,3S)-3-(3-(2,4-difluorophenyl)-1H-indol-1-yl)-3-(3-fluorophenyl)propane-1,2-diol. MS (ES) m/z 411.2 ([M+H]⁺); HRMS: calculated for C₂₄H₂₁F₃N₂O+H⁺, 411.16787; found (ESI, [M+H]⁺), 411.167

### Example 39

### (1S,2R)-1-[3-(2,5-difluorophenyl)-1H-indol-1-yl]-1-(3-fluorophenyl)-3-(methylamino)propan-2-ol hydrochloride

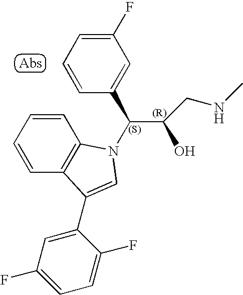

In an analogous manner to Example 34, step 4, (2S,3S)-3-(3-(2,5-difluorophenyl)-1H-indol-1-yl)-3-(3-fluorophenyl)propane-1,2-diol was prepared from (2S,3S)-3-(3-fluorophenyl)-3-(3-iodo-1H-indol-1-yl)propane-1,2-diol (from Example 34, step 3) and 2,5-(difluorophenyl)boronic acid.

In an analogous manner to Example 27 step 3, (1S,2R)-1-[3-(2,5-difluorophenyl)-1H-indol-1-yl]-1-(3-fluorophenyl)-3-(methylamino)propan-2-ol hydrochloride was prepared from (2S,3S)-3-(3-(2,5-difluorophenyl)-1H-indol-1-yl)-3-(3-fluorophenyl)propane-1,2-diol. MS (ES) m/z 411.2 ([M+H]⁺); HRMS: calculated for C₂₄H₂₁F₃N₂O+H⁺, 411.16787; found (ESI, [M+H]⁺), 411.1663.

### Example 40

### (1S,2R)-1-[3-(2,3-dimethoxyphenyl)-1H-indol-1-yl]-1-(3-fluorophenyl)-3-(methylamino)propan-2-ol hydrochloride

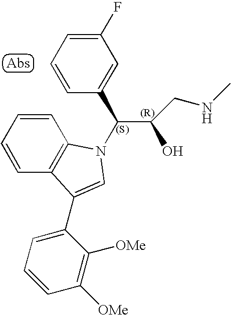

In an analogous manner to Example 34, step 4, (2S,3S)-3-(3-(2,3-dimethoxyphenyl)-1H-indol-1-yl)-3-(3-fluorophenyl)propane-1,2-diol was prepared from (2S,3S)-3-(3-fluorophenyl)-3-(3-iodo-1H-indol-1-yl)propane-1,2-diol (from Example 34, step 3) and 2,3-(dimethoxyphenyl)boronic acid.

In an analogous manner to Example 27, step 3, (1S,2R)-1-[3-(2,3-dimethoxyphenyl)-1H-indol-1-yl]-1-(3-fluorophenyl)-3-(methylamino)propan-2-ol hydrochloride was prepared from (2S,3S)-3-(3-(2,3-dimethoxyphenyl)-1H-indol-1-yl)-3-(3-fluorophenyl)propane-1,2-diol. MS (ES) m/z 435.1 ([M+H]⁺); HRMS: calculated for C₂₆H₂₇FN₂O₃+H⁺, 435.20785; found (ESI, [M+H]⁺), 435.2067.

### Example 41

### (1S,2R)-1-[3-(2,4-dichlorophenyl)-1H-indol-1-yl]-1-(3-fluorophenyl)-3-(methylamino)propan-2-ol hydrochloride

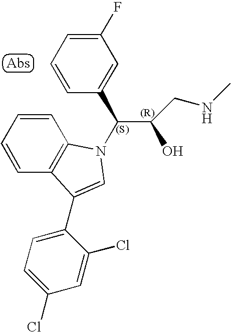

In an analogous manner to Example 34, step 4, (2S,3S)-3-(3-(2,4-dichlorophenyl)-1H-indol-1-yl)-3-(3-fluorophenyl)propane-1,2-diol was prepared from (2S,3S)-3-(3-fluorophenyl)-3-(3-iodo-1H-indol-1-yl)propane-1,2-diol (from Example 34, step 3) and 2,4-(dichlorophenyl)boronic acid MS (ES) m/z 429.6 ([M+H]⁺).

In an analogous manner to Example 27, step 3, (1S,2R)-1-[3-(2,4-dichlorophenyl)-1H-indol-1-yl]-1-(3-fluorophenyl)-3-(methylamino)propan-2-ol hydrochloride was prepared from (2S,3S)-3-(3-(2,4-dichlorophenyl)-1H-indol-1-yl)-3-(3-fluorophenyl)propane-1,2-diol. MS (ES) m/z 442.7 ([M+H]⁺); HRMS: calculated for C₂₄H₂₁Cl₂FN₂O+H⁺, 443.10877; found (ESI, [M+H]⁺), 443.1086.

### Example 42

### (1S,2R)-1-[3-(2-ethoxyphenyl)-1H-indol-1-yl]-1-(3-fluorophenyl)-3-(methylamino)propan-2-ol hydrochloride

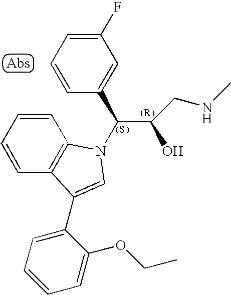

In an analogous manner to Example 34, step 4, (2S,3S)-3-(3-fluorophenyl)-3-{3-[2-(ethoxy)phenyl]-1H-indol-1-yl}propane-1,2-diol was prepared from (2S,3S)-3-(3-fluorophenyl)-3-(3-iodo-1H-indol-1-yl)propane-1,2-diol (from Example 34, step 3) and 2-(Ethoxyphenyl)boronic acid.

In an analogous manner to Example 27, step 3, (1S,2R)-1-[3-(2-ethoxyphenyl)-1H-indol-1-yl]-1-(3-fluorophenyl)-3-(methylamino)propan-2-ol hydrochloride was prepared from (2S,3S)-3-(3-fluorophenyl)-3-{3-[2-(ethoxy)phenyl]-1H-indol-1-yl}propane-1,2-diol. MS (ES) m/z 419.0 ([M+H]⁺); HRMS: calculated for C₂₆H₂₇FN₂O₂+H⁺, 419.21293; found (ESI, [M+H]⁺), 419.2132.

### Example 43

### (1S,2R)-1-(7-chloro-5-methoxy-1H-pyrrolo[2,3-c]pyridin-1-yl)-1-(3-fluorophenyl)-3-(methylamino)propan-2-ol hydrochloride

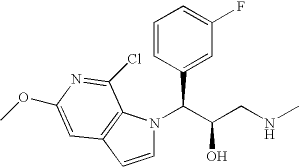

Step 1:

2-Chloro-6-methoxy-3-nitropyridine (5 g, 0.027 mol) was dissolved in anhydrous tetrahydrofuran (200 mL) under nitrogen and the solution was cooled to −78° C. Excess of vinylmagnesium bromide (1.0 M in tetrahydrofuran, 100 mL, 100 mmol) was added and the reaction mixture was stirred at −20° C. for 8 hours and then the reaction mixture was quenched with 20% aqueous ammonium chloride (150 mL). The aqueous layer was extracted with ethyl acetate and the combined extracts were dried over anhydrous magnesium sulfate, filtered, and concentrated under reduced pressure. The crude product was purified via Biotage Horizon (FlasH 40 M, silica, gradient from 20% ethyl acetate/hexane to 60% ethyl acetate/hexane) to yield 7-chloro-5-methoxy-1H-pyrrolo[2,3-c]pyridine² as a yellow solid. MS (ESI) m/z 183 ([M+H]⁺).
² Zhang, Z.; et al., J. Org. Chem. 2002, 76, 2345-2347.

Step 2:

In an analogous manner to Example 24, step 2 (2S,3S)-3-(7-chloro-5-methoxy-1H-pyrrolo[2,3-c]pyridin-1-yl)-3-(3-fluorophenyl)propane-1,2-diol was prepared from 7-chloro-5-methoxy-1H-pyrrolo[2,3-c]pyridine and [(2R,3R)-3(3-fluorophenyl)oxiran-2-yl]methanol (Example 24, step 1) as an oil. MS (ESI) m/z 351 ([M+H]⁺).

In an analogous manner to Example 1, step 5 (2S,3S)-toluene-4-sulfonic acid 3-(7-chloro-5-methoxy-1H-pyrrolo[2,3-c]pyridin-1-yl)-2-hydroxy-3-(3-fluorophenyl)-propyl ester was prepared from (2S,3S)-3-(7-chloro-5-methoxy-1H-pyrrolo[2,3-c]pyridin-1-yl)-3-(3-fluorophenyl)-propane-1,2-diol as a yellow fluffy solid. MS (ESI) m/z 505 ([M+H]⁺).

In an analogous manner to Example 1, step 6 (1S,2R)-1-(7-chloro-5-methoxy-1H-pyrrolo[2,3-c]pyridin-1-yl)-1-(3-fluorophenyl)-3-(methylamino)propan-2-ol hydrochloride was prepared from (2S,3S)-toluene-4-sulfonic acid 3-(7-chloro-5-methoxy-1H-pyrrolo[2,3-c]pyridin-1-yl)-2-hydroxy-3-(3-fluorophenyl)-propyl ester and methylamine solution (2.0 M in methanol) as a white solid. HRMS: calculated for C₁₈H₁₉ClFN₃O₂+H⁺, 364.12226; found (ESI, [M+H]⁺), 364.1218.

### Example 44

### (1S,2R)-1-(7-chloro-5-methyl-1H-pyrrolo[2,3-c]pyridin-1-yl)-3-(methylamino)-1-phenylpropan-2-ol hydrochloride

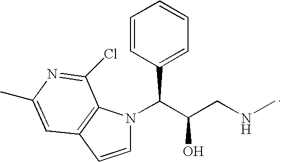

Step 1:

In an analogous manner to Example 43, step 1, 7-chloro-5-methyl-1H-pyrrolo[2,3-c]pyridine was prepared from 2-chloro-3-nitro-6-picoline and vinylmagnesium bromide as a yellow solid. MS (ESI) m/z 167 ([M+H]⁺).

In an analogous manner to Example 24, step 2, (2S,3S)-3-(7-chloro-5-methyl-1H-pyrrolo[2,3-c]pyridin-1-yl)-3-phenyl-propane-1,2-diol was prepared from 7-chloro-5-methyl-1H-pyrrolo[2,3-c]pyridine and [(2R,3R)-3-phenyloxiran-2-yl]methanol (from Example 1, step 1) as an oil. MS (ESI) m/z 317 ([M+H]⁺).

In an analogous manner to Example 1, step 5, (2S,3S)-toluene-4-sulfonic acid 3-(7-chloro-5-methyl-1H-pyrrolo[2,3-c]pyridin-1-yl)-2-hydroxy-3-phenyl-propyl ester was prepared from (2S,3S)-3-(7-chloro-5-methyl-1H-pyrrolo[2,3-c]pyridin-1-yl)-3-phenyl-propane-1,2-diol as an oil. MS (ESI) m/z 471 ([M+H]⁺).

In an analogous manner to Example 1, step 6, (1S,2R)-1-(7-chloro-5-methyl-1H-pyrrolo[2,3-c]pyridin-1-yl)-3-(methylamino)-1-phenylpropan-2-ol hydrochloride was prepared from (2S,3S)-toluene-4-sulfonic acid 3-(7-chloro-5-methyl-1H-pyrrolo[2,3-c]pyridin-1-yl)-2-hydroxy-3-phenyl-propyl ester and methylamine solution (2.0 M in methanol) as an off-white solid. HRMS: calculated for C₁₈H₂₀ClN₃O+H⁺, 330.13677; found (ESI, [M+H]⁺), 330.1355.

### Example 45

### (1S,2R)-1-(5-methoxy-1H-pyrrolo[2,3-c]pyridin-1-yl)-3-(methylamino)-1-phenylpropan-2-ol hydrochloride

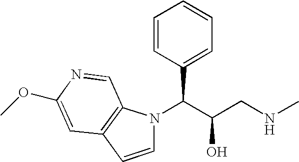

Step 1:

In an analogous manner to Example 24, step 2, (2S,3S)-3-(7-chloro-5-methoxy-1H-pyrrolo[2,3-c]pyridin-1-yl)-3-phenyl-propane-1,2-diol was prepared from 7-chloro-5-methoxy-1H-pyrrolo[2,3-c]pyridine (from Example 43, step 1) and [(2R,3R)-3-phenyloxiran-2-yl]methanol (from Example 1, step 1) as an oil. MS (ESI) m/z 333 ([M+H]⁺).

Step 2:

In an analogous manner to Example 1, step 5, (2S,3S)-toluene-4-sulfonic acid 3-(7-chloro-5-methoxy-1H-pyrrolo[2,3-c]pyridin-1-yl)-2-hydroxy-3-phenyl-propyl ester was prepared from (2S,3S)-3-(7-chloro-5-methoxy-1H-pyrrolo[2,3-c]pyridin-1-yl)-3-phenyl-propane-1,2-diol as an oil. MS (ESI) m/z 487 ([M+H]⁺).

Step 3:

In an analogous manner to Example 1, step 6, (1S,2R)-1-(7-chloro-5-methoxy-1H-pyrrolo[2,3-c]pyridin-1-yl)-3-(methylamino)-1-phenylpropan-2-ol hydrochloride was prepared from (2S,3S)-toluene-4-sulfonic acid 3-(7-chloro-5-methoxy-1H-pyrrolo[2,3-c]pyridin-1-yl)-2-hydroxy-3-phenyl-propyl ester and methylamine solution (2.0 M in methanol) as a white solid. HRMS: calculated for C₁₈H₂₀ClN₃O₂+H⁺, 346.13168; found (ESI, [M+H]⁺), 346.1229.

Step 4:

(1S,2R)-1-(7-chloro-5-methoxy-1H-pyrrolo[2,3-c]pyridin-1-yl)-3-(methylamino)-1-phenylpropan-2-ol (0.13 g, 0.38 mmol) was dissolved in ethanol (20 mL) and treated with 10% palladium on carbon. The reaction mixture was placed under 50 psi of hydrogen on a Parr shaker for 15 hours. The reaction mixture was then filtered through a Celite pad and the filtrate was concentrated under reduced pressure. The crude product was purified via Biotage Horizon (Flash 25 S, silica, gradient from 30% to 100% of 0.9% ammonium hydroxide in 10% methanol-methylene chloride/methylene chloride) to give a white solid as the free base of the expected product. The free base was dissolved in a minimum amount of ethanol and treated with hydrogen chloride solution (1.0 M in diethyl ether) until the solution was pH=3 followed by diethyl ether. The product was then crystallized by adding a minimum amount of ethyl acetate to afford (1S,2R)-1-(5-methoxy-1H-pyrrolo[2,3-c]pyridin-1-yl)-3-(methylamino)-1-phenylpropan-2-ol hydrochloride as a white solid. MS (ES) m/z 311.8 ([M+H]⁺).

### Example 46

### (1S,2R)-1-(3-fluorophenyl)-1-(5-methoxy-1H-pyrrolo[2,3-c]pyridin-1-yl)-3-(methylamino)propan-2-ol hydrochloride

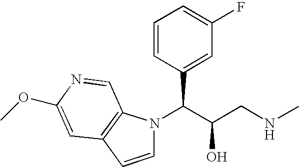

In an analogous manner to Example 45, step 4, (1S,2R)-1-(5-methoxy-1H-pyrrolo[2,3-c]pyridin-1-yl)-1-(3-fluorophenyl)-3-(methylamino)propan-2-ol hydrochloride was prepared from (1S,2R)-1-(7-chloro-5-methoxy-1H-pyrrolo[2,3-c]pyridin-1-yl)-1-(3-fluorophenyl)-3-(methylamino)propan-2-ol (Example 43) as an off-white solid. HRMS: calculated for C₁₈H₂₀FN₃O₂+H⁺, 330.16123; found (ESI, [M+H]⁺), 330.1596.

### Example 47

### (1S,2R)-3-(methylamino)-1-(5-methyl-1H-pyrrolo[2.3-c]pyridin-1-yl)-1-phenylpropan-2-ol hydrochloride

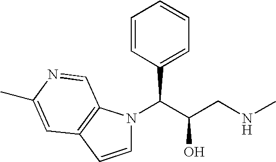

In an analogous manner to Example 45, step 4, (1S,2R)-1-(5-methyl-1H-pyrrolo[2,3-c]pyridin-1-yl)-3-(methylamino)-1-phenylpropan-2-ol hydrochloride was prepared from (1S,2R)-1-(7-chloro-5-methyl-1H-pyrrolo[2,3-c]pyridin-1-yl)-3-(methylamino)-1-phenylpropan-2-ol (Example 44) as an off-white solid. MS (ESI) m/z 296 ([M+H]⁺).

### Example 48

### (1S,2R)-1-(3-fluorophenyl)-3-(methylamino)-1-(5-methyl-1H-pyrrolo[2,3-c]pyridin-1-yl)propan-2-ol hydrochloride

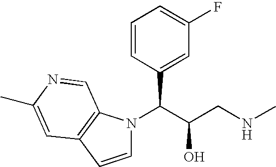

In an analogous manner to Example 24, step 2, (2S,3S)-3-(7-chloro-5-methyl-1H-pyrrolo[2,3-c]pyridin-1-yl)-3-(3-fluorophenyl)-propane-1,2-diol was prepared from 7-chloro-5-methyl-1H-pyrrolo[2,3-c]pyridine (Example 44, step 1) and [(2R,3R)-3-(3-fluorophenyl)oxiran-2-yl]methanol (Example 24, step 1) as an oil. MS (ESI) m/z 335 ([M+H]⁺).

In an analogous manner to Example 1, step 5, (2S,3S)-toluene-4-sulfonic acid 3-(7-chloro-5-methyl-1H-pyrrolo[2,3-c]pyridin-1-yl)-2-hydroxy-3-(3-fluorophenyl)-propyl ester was prepared from (2S,3S)-3-(7-chloro-5-methyl-1H-pyrrolo[2,3-c]pyridin-1-yl)-3-(3-fluorophenyl)-propane-1,2-diol as an oil. MS (ESI) m/z 489 ([M+H]⁺).

In an analogous manner to Example 1, step 6, (1S,2R)-1-(7-chloro-5-methyl-1H-pyrrolo[2,3-c]pyridin-1-yl)-1-(3-fluorophenyl)-3-(methylamino)propan-2-ol was prepared from (2S,3S)-toluene-4-sulfonic acid 3-(7-chloro-5-methyl-1H-pyrrolo[2,3-c]pyridin-1-yl)-2-hydroxy-3-(3-fluorophenyl)-propyl ester and methylamine solution (2.0 M in methanol) as an oil. HRMS: calculated for C₁₈H₁₉ClFN₃O+H⁺, 348.12734; found (ESI, [M+H]⁺), 348.1262.

In an analogous manner to Example 45, step 4, (1S,2R)-1-(3-fluorophenyl)-3-(methylamino)-1-(5-methyl-1H-pyrrolo[2,3-c]pyridin-1-yl)propan-2-ol hydrochloride was prepared from (1S,2R)-1-(7-chloro-5-methyl-1H-pyrrolo[2,3-c]pyridin-1-yl)-1-(3-fluorophenyl)-3-(methylamino)propan-2-ol as an off-white solid. HRMS: calculated for C₁₈H₂₀FN₃O+H⁺, 314.16632; found (ESI, [M+H]⁺), 314.1599.

### Example 49

### (1S,2R)-3-(methylamino)-1-(7-methyl-1H-pyrrolo[2,3-c]pyridin-1-yl)-1-phenylpropan-2-ol hydrochloride

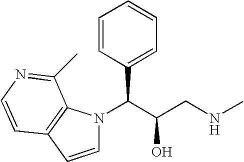

In an analogous manner to Example 43, step 1, 5-chloro-7-methyl-1H-pyrrolo[2,3-c]pyridine was prepared from 2-chloro-5-nitro-6-picoline and yinylmagnesium bromide as an oily solid. MS (ESI) m/z 167 ([M+H]⁺).

In an analogous manner to Example 24, step 2, (2S,3S)-3-(5-chloro-7methyl-1H-pyrrolo[2,3-c]pyridin-1-yl)-3-phenyl-propane-1,2-diol was prepared from 5-chloro-7-methyl-1H-pyrrolo[2,3-c]pyridine and [(2R,3R)-3-phenyloxiran-2-yl]methanol (from Example 1, step 1) as an off-white solid. MS (ESI) m/z 317 ([M+H]⁺).

In an analogous manner to Example 1, step 5, (2S,3S)-toluene-4-sulfonic acid 3-(5-chloro-7-methyl-1H-pyrrolo[2,3-c]pyridin-1-yl)-2-hydroxy-3-phenyl-propyl ester was prepared from (2S,3S)-3-(5-chloro-7-methyl-1H-pyrrolo[2,3-c]pyridin-1-yl)-3-phenyl-propane-1,2-diol as an oil. MS (ESI) m/z 471 ([M+H]⁺).

In an analogous manner to Example 1, step 6, (1S,2R)-1-(5-chloro-7-methyl-1H-pyrrolo[2,3-c]pyridin-1-yl)-3-(methylamino)-1-phenylpropan-2-ol was prepared from (2S,3S)-toluene-4-sulfonic acid 3-(7-methyl-5-chloro-1H-pyrrolo[2,3-c]pyridin-1-yl)-2-hydroxy-3-phenyl-propyl ester and methylamine solution (2.0 M in methanol) as an oil.

HRMS: calculated for C₁₈H₂₀ClN₃O+H⁺, 330.13677; found (ESI, [M+H]⁺), 330.1354.

In an analogous manner to Example 45, step 4, (1S,2R)-1-(7-methyl-1H-pyrrolo[2,3-c]pyridin-1-yl)-3-(methylamino)-1-phenylpropan-2-ol hydrochloride was prepared from (1 S,2R)-1-(5-chloro-7-methyl-1H-pyrrolo[2,3-c]pyridin-1-yl)-3-(methylamino)-1-phenylpropan-2-ol as a white solid. HRMS: calculated for C₁₈H₂₁N₃O+H⁺, 296.17574; found (ESI, [M+H]⁺), 296.1758.

### Example 50

### (1S,2R)-1-(3-fluorophenyl)-3-(methylamino)-1-(7-methyl-1H-pyrrolo[2,3-c]pyridin-1-yl)propan-2-ol hydrochloride

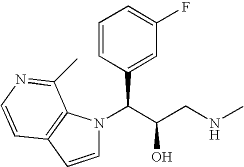

In an analogous manner to Example 24, step 2, (2S,3S)-3-(5-chloro-7-methyl-1H-pyrrolo[2,3-c]pyridin-1-yl)-3-(3-fluorophenyl)-propane-1,2-diol was prepared from 5-chloro-7-methyl-1H-pyrrolo[2,3-c]pyridine (from Example 49, step 1) and [(2R,3R)-3-(3-fluorophenyl)oxiran-2-yl]methanol (from Example 24, step 1) as an off-white solid. MS (ESI) m/z 335 ([M+H]⁺).

In an analogous manner to Example 1, step 5, (2S,3S)-toluene-4-sulfonic acid 3-(5-chloro-7-methyl-1H-pyrrolo[2,3-c]pyridin-1-yl)-2-hydroxy-3-(3-fluorophenyl)-propyl ester was prepared from (2S,3S)-3-(5-chloro-7-methyl-1H-pyrrolo[2,3-c]pyridin-1-yl)-3-(3-fluorophenyl)-propane-1,2-diol as an oil. MS (ESI) m/z 489 ([M+H]⁺).

In an analogous manner to Example 1, step 6, (1S,2R)-1-(5-chloro-7-methyl-1H-pyrrolo[2,3-c]pyridin-1-yl)-1-(3-fluorophenyl)-3-(methylamino)propan-2-ol was prepared from (2S,3S)-toluene-4-sulfonic acid 3-(5-chloro-7-methyl 1H-pyrrolo[2,3-c]pyridin-1-yl)-2-hydroxy-3-(3-fluorophenyl)-propyl ester and methylamine solution (2.0 M in methanol) as an oil. HRMS: calculated for C₁₈H₁₉ClFN₃O+H⁺, 348.12734; found (ESI, [M+H]⁺), 348.1287.

In an analogous manner to Example 45, step 4, (1S,2R)-1-(3-fluorophenyl)-3-(methylamino)-1-(7-methyl-1H-pyrrolo[2,3-c]pyridin-1-yl)propan-2-ol hydrochloride was prepared from (1S,2R)-1-(7-methyl-5-chloro-1H-pyrrolo[2,3-c]pyridin-1-yl)-1-(3-fluorophenyl)-3-(methylamino)propan-2-ol as a white solid. HRMS: calculated for C₁₈H₂₀FN₃O+H⁺, 314.16632; found (ESI, [M+H]⁺), 314.1628.

### Example 51

### (1S,2R)-1-(3,3-diethyl-2,3-dihydro-1H-indol-1-yl)-1-(3-fluorophenyl)-3-(methylamino)propan-2-ol hydrochloride

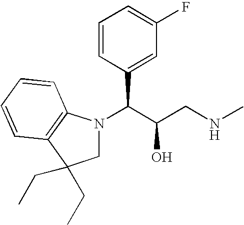

In an analogous manner to Example 1, step 3, (2S,3S)-3-(3,3-diethyl-2,3-dihydro-1H-indol-1-yl)-3-(3-fluorophenyl)propane-1,2-diol was prepared from 3,3-diethylindoline¹ and [(2R,3R)-3-(3-fluorophenyl)oxiran-2-yl]methanol (Example 25, step 3) as an amber colored oil. MS (ESI) m/z 344.2 ([M+H]⁺); HRMS: calculated for C₂₁H₂₆FNO₂+H⁺, 344.2026; found (ESI, [M+H]⁺), 344.2048.
¹ Kucerovy, A.; Hathaway, J. S.; Mattner, P. G.; Repic, O. Synth Commun. 1992, 22, 729-733.

In an analogous manner to Example 25, step 5, (1S,2R)-1-(3,3-diethyl-2,3-dihydro-1H-indol-1-yl)-1-(3-fluorophenyl)-3-(methylamino)propan-2-ol hydrochloride was prepared from (2S,3S)-3-(3,3-diethyl-2,3-dihydro-1H-indol-1-yl)-3-(3-fluorophenyl)propane-1,2-diol as a white powder. MS (ES) m/z 357.3 ([M+H]⁺); HRMS: calculated for C₂₂H₂₉FN₂O+H⁺, 357.2337; found (ESI, [M+H]⁺), 357.2340.

### Example 52

### (1S,2R)-1-(6-fluoro-3,3-dimethyl-2,3-dihydro-1H-indol-1yl)-1-(3-fluorophenyl)-3-(methylamino)propan-2-ol hydrochloride

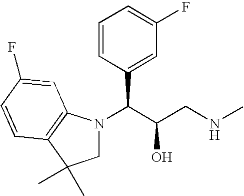

Step 1:

In an analogous manner to Example 27, step 1, 6-fluoro-3,3-dimethyloxindole was prepared from 6-fluorooxindole and iodomethane (2 equiv.) as a yellowish solid. MS (EI) m/z 179.1 ([M]⁺); HRMS: calculated for C₁₀H₁₀FNO, 179.0746; found (EI, [M]⁺), 179.0742.

Step 2:

A mixture of 6-fluoro-3,3-dimethyloxindole (1.00 g, 5.58 mmol) in toluene (10 mL) under nitrogen was heated at 80° C. Vitride (65 wt % in toluene, 2.7 mL, 8.9 mmol) was added dropwise via an addition funnel. The resulting solution was stirred at 80° C. for an additional 1.5 hours, then cooled in an ice bath. Aqueous sodium hydroxide solution (1N, 15 mL) was added slowly to quench the reaction. Water (15 mL) was added and the reaction mixture was extracted with ethyl acetate (20 mL). The organic layer was washed with brine, dried over sodium sulfate, filtered through a pad of silica gel, and concentrated under reduced pressure to yield 728 mg (79%) of 6-fluoro-3,3-dimethylindoline as an amber colored oil. MS (ES) m/z 166.2 ([M+H]⁺).

Step 3:

In an analogous manner to Example 1, step 3, (2S,3S)-3-(6-fluoro-3,3-dimethyl-2,3-dihydro-1H-indol-1-yl)-3-(3-fluorophenyl)propane-1,2-diol was prepared from 6-fluoro-3,3-dimethylindoline and [(2R,3R)-3-(3-fluorophenyl)oxiran-2-yl]methanol (from Example 25, step 3) as a brown solid. MS (ESI) m/z 334.2 ([M+H]⁺); HRMS: calculated for C₁₉H₂₁F₂NO₂+H⁺, 334.1613; found (ESI, [M+H]⁺), 334.1597.

Step 4:

In an analogous manner to Example 25, step 5, (1S,2R)-1-(6-fluoro-3,3-dimethyl-2,3-dihydro-1H-indol-1-yl)-1-(3-fluorophenyl)-3-(methylamino)propan-2-ol hydrochloride was prepared from (2S,3S)-3-(6-fluoro-3,3-dimethyl-2,3-dihydro-1H-indol-1-yl)-3-(3-fluorophenyl)propane-1,2-diol as a white powder. HRMS: calculated for C₂₀H₂₄F₂N₂O+H⁺, 347.1929; found (ESI, [M+H]⁺), 347.1914.

### Example 53

### (1S,2R)-1-(4-benzyl-3,4-dihydroquinoxalin-1(2H)-yl)-1-(3-fluorophenyl)-3-(methylamino)propan-2-ol hydrochloride

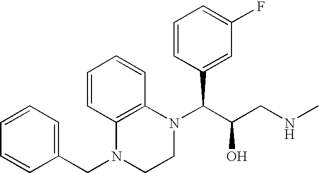

In an analogous manner to Example 1, step 3, (2S,3S)-3-(4-benzyl-3,4-dihydroquinoxalin-1(2H)-yl)-3-(3-fluorophenyl)propane-1,2-diol was prepared from 1-benzyl-1,2,3,4-tetrahydroquinoxaline³ and [(2R,3R)-3-(3-fluorophenyl)oxiran-2-yl]methanol (from Example 25, step 3) as a viscous, brown oil. MS (ESI) m/z 393.2 ([M+H]⁺); HRMS: calculated for C₂₄H₂₅FN₂O₂+H⁺, 393.1973; found (ESI, [M+H]⁺), 393.1967.
³ Smith, R. F.; Rebel, W. J.; Beach, T. N.; J. Org. Chem. 1959, 24, 205-207.

³ Smith, R. F.; Rebel W. J.; Beach, T. N. J. Org. Chem. 1959, 24, 205-207.

In an analogous manner to Example 25, step 5, (1S,2R)-1-(4-benzyl-3,4-dihydroquinoxalin-1(2H)-yl)-1-(3-fluorophenyl)-3-(methylamino)propan-2-ol hydrochloride was prepared from (2S,3S)-3-(4-benzyl-3,4-dihydroquinoxalin-1(2H)-yl)-3-(3-fluorophenyl)propane-1,2-diol as a white powder. MS (ES) m/z 406.2 ([M+H]⁺).

### Example 54

### (1S,2R)-1-(5-fluoro-3,3-dimethyl-2,3-dihydro-1H-indol-1-yl)-1-(3-fluorophenyl)-3-(methylamino)propan-2-ol hydrochloride

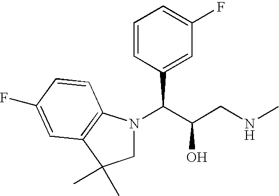

In an analogous manner to Example 27, step 1,5-fluoro-3,3-dimethyloxindole was prepared from 5-fluorooxindole and iodomethane (2 equiv.) as a white crystals. HRMS: calculated for C₁₀H₁₀FNO+H⁺, 180.0825; found (ESI, [M+H]⁺), 180.0832.

In an analogous manner to Example 52, step 2,5-fluoro-3,3-dimethylindoline was prepared from 5-fluoro-3,3-dimethyloxindole as an amber colored oil. MS (ES) m/z 166.2 ([M+H]⁺); HRMS: calculated for C₁₀H₁₂FN+H⁺, 166.1027; found (ESI, [M+H]⁺), 166.1024.

In an analogous manner to Example 1, step 3, (2S,3S)-3-(5-fluoro-3,3-dimethyl-2,3-dihydro-1H-indol-1-yl)-3-(3-fluorophenyl)propane-1,2-diol was prepared from 5-fluoro-3,3-dimethylindoline and [(2R,3R)-3-(3-fluorophenyl)oxiran-2-yl]methanol (from Example 25, step 3) as a viscous, colorless oil. MS (ESI) m/z 334.2 ([M+H]⁺); HRMS: calculated for C₁₉H₂₁F₂NO₂+H⁺, 334.1613; found (ESI, [M+H]⁺), 334.1606.

In an analogous manner to Example 25, step 5, (1S,2R)-1-(5-fluoro-3,3-dimethyl-2,3-dihydro-1H-indol-1-yl)-1-(3-fluorophenyl)-3-(methylamino)propan-2-ol hydrochloride was prepared from (2S,3S)-3-(5-fluoro-3,3-dimethyl-2,3-dihydro-1H-indol-1-yl)-3-(3-fluorophenyl)propane-1,2-diol as a white powder. MS (ESI) m/z 347.3 ([M+H]⁺); HRMS: calculated for C₂₀H₂₄F₂N₂O+H⁺, 347.1929; found (ESI, [M+H]⁺), 347.1940.

### Example 55

### (1S,2R)-1-(3-fluorophenyl)-3-(methylamino)-1-[(3S)-3-methyl-2,3-dihydro-1H-indol-1-yl]propan-2-ol hydrochloride

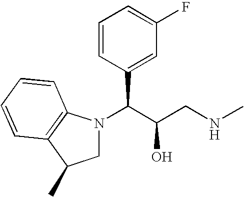

Step 1:

In an analogous manner to Example 1, step 3, (2S,3S)-3-(3-fluorophenyl)-3-(3-methyl-2,3-dihydro-1H-indol-1-yl)propane-1,2-diol was prepared from 3-methylindoline⁴ and [(2R,3R)-3-(3-fluorophenyl)oxiran-2-yl]methanol (from Example 25, step 3) as a viscous, yellowish liquid. MS (ES) m/z 301.8 ([M+H]⁺); HRMS: calculated for C₁₈H₂₀FNO₂+H⁺, 302.1551; found (ESI, [M+H]⁺), 302.1539.
⁴ Gribble, G. W.; Hoffman, J. H. Synthesis 1977, 12, 859-860.

Step 2:

In an analogous manner to Example 25, step 5, (1S,2R)-1-(3-fluorophenyl)-3-(methylamino)-1-(3-methyl-2,3-dihydro-1H-indol-1-yl)propan-2-ol was prepared from (2S,3S)-3-(3-fluorophenyl)-3-(3-methyl-2,3-dihydro-1H-indol-1-yl)propane-1,2-diol as a viscous, colorless liquid.

Step 3:

The diastereomeric mixture of (1S,2R)-1-(3-fluorophenyl)-3-(methylamino)-1-(3-methyl-2,3-dihydro-1H-indol-1-yl)propan-2-ol was dissolved in methanol. The resulting solution was injected onto the Supercritical Fluid Chromatography instrument. The baseline resolved diastereomers, using the conditions described below, were collected.
SFC Instrument: Berger MultiGram Prep SFC (Berger Instruments, Inc. Newark, DE 19702.
Column: Chiralpak AD-H; 250 mm L×20 mm ID
Column temperature: 35° C.
SFC Modifier: 10% MeOH, 90% CO₂, with 0.2% diethylamine
Flow rate: 50 mL/minute
Outlet Pressure: 100 bar
Detector: UV at 254 nm

Step 4:

(1S,2R)-1-(3-fluorophenyl)-3-(methylamino)-1-[(3S)-3-methyl-2,3-dihydro-1H-indol-1-yl]propan-2-ol, isolated as peak 1, was subjected to hydrochloride salt formation in an analogous manner to Example 25, step 5 to give (1S,2R)-1-(3-fluorophenyl)-3-(methylamino)-1-[(3S)-3-methyl-2,3-dihydro-1H-indol-1-yl]propan-2-ol hydrochloride as a white powder. The stereochemistry at the C3 of the indoline ring is arbitrarily assigned. MS (ES) m/z 315.2 ([M+H]⁺); HRMS: calculated for C₁₉H₂₃FN₂O+H⁺, 315.1873; found (ESI, [M+H]⁺), 315.1885.

### Example 56

### (1S,2R)-1-(3-fluorophenyl)-3-(methylamino)-1-[(3R)-3-methyl-2,3-dihydro-1H-indol-1-yl]propan-2-ol hydrochloride

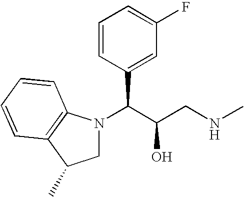

In an analogous manner to Example 55, step 4, (1S,2R)-1-(3-fluorophenyl)-3-(methylamino)-1-[(3R)-3-methyl-2,3-dihydro-1H-indol-1-yl]propan-2-ol hydrochloride was prepared as a white powder from (1S,2R)-1-(3-fluorophenyl)-3-(methylamino)-1-[(3R)-3-methyl-2,3-dihydro-1H-indol-1-yl]propan-2-ol, which was isolated as peak 2 of the diastereomeric separation (Example 55, step 3). The stereochemistry at the C3 of the indoline ring is arbitrarily assigned. MS (ES) m/z 314.9 ([M+H]⁺); HRMS: calculated for C₁₉H₂₃FN₂O+H⁺, 315.1873; found (ESI, [M+H]⁺), 315.1880.

### Example 57

### (1S,2R)-1-(3-fluorophenyl)-1-(3-isopropyl-2,3-dihydro-1H-indol-1-yl)-3-(methylamino)propan-2-ol hydrochloride

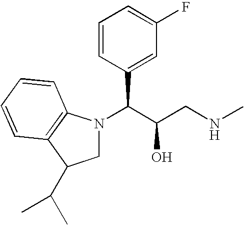

Step 1:

In an analogous manner to Example 1, step 2, 3-isopropylindoline was prepared from 3-isopropylindole⁵ as a colorless oil. MS (ESI) m/z 162.2 ([M+H]⁺).
⁵ Odle, R.; Blevins, B.; Ratcliff, M.; Hegedus, L. S. J. Org. Chem. 1980, 45, 2709-2710.

Step 2:

In an analogous manner to Example 1, step 3, (2S,3S)-3-(3-fluorophenyl)-3-(3-isopropyl-2,3-dihydro-1H-indol-1-yl)propane-1,2-diol was prepared from 3-isopropylindoline and [(2R,3R)-3-(3-fluorophenyl)oxiran-2-yl]methanol (Example 25, step 3) as a colorless oil. MS (ESI) m/z 330.3 ([M+H]⁺); HRMS: calculated for C₂₀H₂₄FNO₂+H⁺, 330.1864; found (ESI, [M+H]⁺), 330.1855.

Step 3:

In an analogous manner to Example 25, step 5, (1S,2R)-1-(3-fluorophenyl)-1-(3-isopropyl-2,3-dihydro-1H-indol-1-yl)-3-(methylamino)propan-2-ol hydrochloride was prepared from (2S,3S)-3-(3-fluorophenyl)-3-(3-isopropyl-2,3-dihydro-1H-indol-1-yl)propane-1,2-diol as a white powder. MS (ESI) m/z 343.0 ([M+H]⁺); HRMS: calculated for C₂₁H₂₇FN₂O+H⁺, 343.2180; found (ESI, [M+H]⁺), 343.2191.

### Example 58

### (1S,2R)-1-(3-ethyl-2,3-dihydro-1H-indol-1-yl)-1-(3-fluorophenyl)-3-(methylamino)propan-2-ol hydrochloride

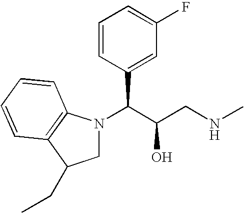

Step 1:

In an analogous manner to Example 1, step 2, 3-ethylindoline was prepared from 3-ethylindole⁵ as a colorless oil. MS (EI) m/z 147.0 ([M]^{+.}); HRMS: calculated for C₁₀H₁₃N, 147.1048; found (EI, [M]^{+.}), 147.1043.
⁵ Odle, R.; Blevins, B.; Ratcliff, M.; Hegedus, L. S. J. Org. Chem. 1980, 45, 2709-2710.

Step 2:

In an analogous manner to Example 1, step 3, (2S,3S)-3-(3-ethyl-2,3-dihydro-1H-indol-1-yl)-3-(3-fluorophenyl)propane-1,2-diol was prepared from 3-ethylindoline and [(2R,3R)-3-(3-fluorophenyl)oxiran-2-yl]methanol (from Example 25, step 3) as a colorless oil. MS (ESI) m/z 316.2 ([M+H]⁺); HRMS: calculated for C₁₉H₂₂FNO₂+H⁺, 316.1707; found (ESI, [M+H]⁺), 316.1699.

Step 3:

In an analogous manner to Example 25, step 5, (1S,2R)-1-(3-ethyl-2,3-dihydro-1H-indol-1-yl)-1-(3-fluorophenyl)-3-(methylamino)propan-2-ol hydrochloride was prepared from (2S,3S)-3-(3-ethyl-2,3-dihydro-1H-indol-1-yl)-3-(3-fluorophenyl)propane-1,2-diol as a white powder. MS (ESI) m/z 329.0 ([M+H]⁺); HRMS: calculated for C₂₀H₂₅FN₂O+H⁺, 329.2024; found (ESI, [M+H]⁺), 329.2023.

### Example 59

### (1S,2R)-1-(3-ethyl-2,3-dihydro-1H-indol-1-yl)-3-(methylamino)-1-phenylpropan-2-ol hydrochloride

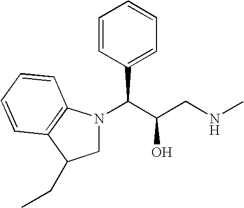

In an analogous manner to Example 1, step 3, (2S,3S)-3-(3-ethyl-2,3-dihydro-1H-indol-1-yl)-3-phenylpropane-1,2-diol was prepared from 3-ethylindoline (from Example 58, step 1) and [(2R,3R)-3-phenyloxiran-2-yl]methanol (Example 1, step 1) as a white solid. MS (ESI) m/z 297.8 ([M+H]⁺); HRMS: calculated for C₁₉H₂₃NO₂+H⁺, 298.1802; found (ESI, [M+H]⁺), 298.1816.

In an analogous manner to Example 25, step 5, (1S,2R)-1-(3-ethyl-2,3-dihydro-1H-indol-1-yl)-3-(methylamino)-1-phenylpropan-2-ol hydrochloride was prepared from (2S,3S)-3-(3-ethyl-2,3-dihydro-1H-indol-1-yl)-3-phenylpropane-1,2-diol as a tan powder. MS (ESI) m/z 311.0 ([M+H]⁺).

### Example 60

### (1S,2R)-1-(3-isopropyl-2,3-dihydro-1H-indol-1-yl)-3-(methylamino)-1-phenylpropan-2-ol hydrochloride

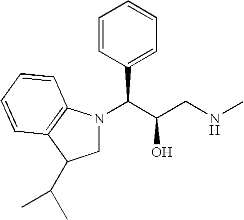

In an analogous manner to Example 1, step 3, (2S,3S)-3-(3-isopropyl-2,3-dihydro-1H-indol-1-yl)-3-phenylpropane-1,2-diol was prepared from 3-isopropylindoline (from Example 57, step 1) and [(2R,3R)-3-phenyloxiran-2-yl]methanol (from Example 1, step 1) as a colorless oil. MS (ESI) m/z 312.0 ([M+H]⁺); HRMS: calculated for C₂₀H₂₅NO₂+H⁺, 312.1964; found (ESI, [M+H]⁺), 312.1981.

In an analogous manner to Example 25, step 5, (1S,2R)-1-(3-isopropyl-2,3-dihydro-1H-indol-1-yl)-3-(methylamino)-1-phenylpropan-2-ol hydrochloride was prepared from (2S,3S)-3-(3-isopropyl-2,3-dihydro-1H-indol-1-yl)-3-phenylpropane-1,2-diol as a white powder. MS (ESI) m/z 325.0 ([M+H]⁺).

### Example 61

### (1S,2R)-3-amino-1-(3,5-difluorophenyl)-1-(3,3-dimethyl-2,3-dihydro-1H-indol-1-yl)propan-2-ol hydrochloride

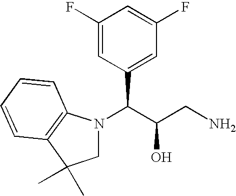

Step 1:

In an analogous manner to Example 25, step 1, trans-3,5-difluorocinnamic acid methyl ester was prepared from trans-3,5-difluorocinnamic acid as a white solid. Yield: 5.387 g (99%). MS (ESI) m/z 198.0 (M⁺); HRMS: calculated for C₁₀H₈F₂O₂, 198.0492; found (ESI, [M]⁺), 198.0489.

Step 2:

In an analogous manner to Example 25, step 2, trans-3,5-difluorocinnamyl alcohol was prepared from trans-3,5-difluorocinnamic acid methyl ester as a colorless oil. Yield: 8.64 g (95%).

Step 3:

In an analogous manner to Example 25, step 3, [(2R,3R)-3-(3,5-difluorophenyl)oxiran-2-yl]methanol was prepared from trans-3,5-difluorocinnamyl alcohol as a colorless liquid. Yield: 4.566 g (70%). Percent ee: 97.9%. MS (ESI) m/z 186.0 (M⁺); HRMS: calculated for C₉H₈F₂O₂, 186.0492; found (ESI, [M]⁺), 186.0501.

Step 4:

In an analogous manner to Example 1, step 3, (2S,3S)-3-(3,5-difluorophenyl)-3-(3,3-dimethyl-2,3-dihydro-1H-indol-1-yl)propane-1,2-diol was prepared from 3,3-dimethylindoline⁶ and [(2R,3R)-3-(3,5-difluorophenyl)oxiran-2-yl]methanol as a brown gum. MS (ESI) m/z 334.0 ([M+H]⁺); HRMS: calculated for C₁₉H₂₁F₂NO₂+H⁺, 334.1619; found (ESI, [M+H]⁺), 334.1619.
⁶ Ramsay, T. W.; Slater, G. R.; Smith, P. Synth. Commun. 1995, 25, 4029-4033.

Step 5:

In an analogous manner to Example 25, step 5, (1S,2R)-3-amino-1-(3,5-difluorophenyl)-1-(3,3-dimethyl-2,3-dihydro-1H-indol-1-yl)propan-2-ol hydrochloride was prepared from (2S,3S)-3-(3,5-difluorophenyl)-3-(3,3-dimethyl-2,3-dihydro-1H-indol-1-yl)propane-1,2-diol as a white powder substituting ammonia solution (7.0 M in methanol) with heating at 50° C., in place of methylamine solution (33% in absolute ethanol). MS (ESI) m/z 333.0 ([M+H]⁺); HRMS: calculated for C₁₉H₂₂F₂N₂O+H⁺, 333.1773; found (ESI, [M+H]⁺), 333.1764.

### Example 62

### 1-[(1S,2R)-1-(3,5-difluorophenyl)-2-hydroxy-3-(methylamino)propyl]-7-fluoro-3,3-dimethyl-1,3-dihydro-2H-indol-2-one hydrochloride

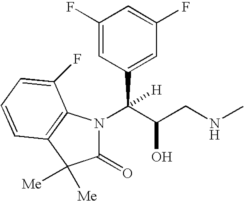

Step 1:

To a solution of 2,6-difluoronitrobenzene (5.0 g, 31.44 mmol) in dry N,N-dimethylformamide (50 mL) was added potassium carbonate (4.41 g, 32 mmol) and dimethylmalonate (3.6 mL, 31.44 mmol). The reaction mixture was heated to 65° C. and stirred for 24 hours. After cooling to room temperature, the mixture was neutralized with a dilute aqueous solution of hydrochloric acid and extracted with diethyl ether. The ethereal layer was dried over anhydrous magnesium sulfate, filtered and concentrated under reduced pressure. Crystallization from 5% ethyl acetate/hexane gave 4.6 g (54%) of dimethyl (3-fluoro-2-nitrophenyl)malonate. MS (ESI) m/z 272 [M+H]⁺).

Step 2:

Dimethyl (3-fluoro-2-nitrophenyl)malonate (12 g, 44 mmol) in a 6 N aqueous solution of hydrochloric acid (200 mL) was heated at reflux for 4 hours. The mixture was cooled, diluted with water (250 mL) and extracted with diethyl ether. The ethereal layer was dried over anhydrous magnesium sulfate, filtered and concentrated under reduced pressure. Crystallization from 5% ethyl acetate/hexane gave 7.6 g (54%) of (6-fluoro-2-nitro-phenyl)-acetic acid. MS (ESI) m/z 200 ([M+H]⁺).

Step 3:

A mixture of (6-fluoro-2-nitro-phenyl)-acetic acid (9.6 g, 48 mmol) and 10% palladium on carbon (1.3 g) in acetic acid (100 mL) was hydrogenated at 50 psi for 24 hours. The catalyst was removed by filtration through Celite and the solvent was evaporated. The residue was then dissolved in ethanol (100 mL) and pyridinium para-toluenesulfonate (50 mg) was added and the mixture heated at reflux for 1 hour. The mixture was cooled, poured into water, extracted with ethyl acetate and dried over anhydrous magnesium sulfate, filtered and concentrated under reduced pressure. The resulting solid was triturated with 5% ethyl acetate/hexane to give 6.0 g (83%) of 7-fluoro-1,3-dihydro-indol-2-one. MS (ESI) m/z 152, [M+H]⁺).

Step 4:

7-Fluoro-1,3-dihydro-indol-2-one (7.3 g, 48 mmol) and lithium chloride (6.67 g, 158 mmol) were dissolved in tetrahydrofuran (200 mL). The solution was cooled to −78° C. and n-butyllithium (40 mL, 100 mmol) was added slowly over a 15 minute period. After 20 minutes at −78° C., methyl iodide (6 mL, 96 mmol) was added and the mixture allowed to warm to room temperature. After 24 hours, the mixture was poured into water and extracted with ethyl acetate. The organic layer was dried over anhydrous magnesium sulfate, filtered and concentrated under reduced pressure. Purification of the crude product via Biotage chromatography (Flash40i, silica, 10% then 20% ethyl acetate/hexane) gave 4.1 g (48%) of 7-fluoro-3,3-dimethyl-1,3-dihydro-2H-indol-2-one. MS (ESI) m/z 180 ([M+H]⁺).

Step 5:

7-fluoro-3,3-dimethyl-1,3-dihydro-2H-indol-2-one (2.12 g, 12 mmol) was dissolved in N,N-dimethylformamide (12 mL) and sodium hydride (0.92 g, 24 mmol, 60% wt suspension in mineral oil) was added in portions over 15 minutes and the mixture was stirred an additional 30 minutes. In a separate flask, [(2R,3R)-3-(3,5-difluorophenyl)oxiran-2-yl]methanol (4.76 g, 25.6 mmol, from Example 61, Step 3) was dissolved in N,N-dimethylformamide (12 mL) and titanium isopropoxide (7.0 mL, 25.6 mmol) was added and the mixture was stirred 30 minutes. The titanium isopropoxide/epoxide solution was then added to the solution of oxindole sodium salt dropwise and the mixture was stirred at room temperature for 24 hours. The mixture was then carefully quenched with 2 N aqueous hydrochloric acid and diluted with 200 mL of 2 N aqueous hydrochloric acid (use of hydrochloric acid is essential to prevent precipitation of titanium salts and subsequent emulsification). The mixture was extracted with ethyl acetate and then the organic layers were combined, washed with water, and saturated brine, dried over anhydrous magnesium sulfate, filtered, and concentrated under reduced pressure. The crude product was purified via Isco chromatography (Redisep, silica, gradient 20% to 100% ethyl acetate in hexane) to afford 4.0 g (91%) of 7-fluoro-1-[(1S,2S)-1-(3,5-difluorophenyl)-2,3-dihydroxypropyl]-3,3-dimethyl-1,3-dihydro-2H-indol-2-one as a sticky oil.

Step 6:

7-fluoro-1-[(1S,2S)-1-(3,5-difluorophenyl)-2,3-dihydroxypropyl]-3,3-dimethyl-1,3-dihydro-2H-indol-2-one (2.3 g, 6.3 mmol) was dissolved in pyridine (15 mL) and p-toluenesufonyl chloride (1.3 g, 6.9 mmol) was added and the mixture stirred for 4 hours. The reaction mixture then was diluted with diethyl ether and washed with water, 2 N aqueous hydrochloric acid, saturated copper sulfate, 2 N aqueous hydrochloric acid, and saturated brine. The organic layer was separated, dried over anhydrous magnesium sulfate, filtered, and concentrated under reduced pressure. The crude product was immediately dissolved in methylamine solution (8.0 M in ethanol, 30 mL) and stirred for 16 hours. The mixture was concentrated under reduced pressure and purified via chromatography (silica, 5% methanol saturated with ammonia in chloroform) to give 1-[(1S,2R)-1-(3,5-difluorophenyl)-2-hydroxy-3-(methylamino)-propyl]-7-fluoro-3,3-dimethyl-1,3-dihydro-2H-indol-2-one as a colorless oil (0.14 g). The freebase was dissolved in ether (10 mL) and treated with hydrogen chloride solution (1.0 M in diethyl ether, 0.36 mL, 1.0 equivalent). The white precipitate was collected and dried under vacuum then dissolved in 10 mL of water and lyophilized to give 110 mg (4% over three steps) of 1-[(1S,2R)-1-(3,5-difluorophenyl)-2-hydroxy-3-(methylamino)propyl]-7-fluoro-3,3-dimethyl-1,3-dihydro-2H-indol-2-one hydrochloride. HRMS: calculated for C₂₀H₂₁F₃N₂O₂+H⁺, 379.16279; found (ESI, [M+H]⁺), 379.1642.

### Example 63

### 5,7-difluoro-1-[(1S,2R)-1-(3-fluorophenyl)-2-hydroxy-3-(methylamino)propyl]-3,3-dimethyl-1,3-dihydro-2H-indol-2-one hydrochloride

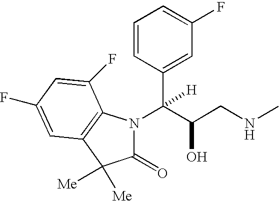

Step 1:

5,7-Difluorooxindole (prepared in a manner analogous to Example 62, steps 1-4 using 2,4,6-trifluoronitrobenzene instead of 2,6-difluoronitrobenzene) (0.64 g, 3.2 mmol) was dissolved in N,N-dimethylformamide (3 mL) and sodium hydride (0.24 g, 6.4 mmol, 60% wt suspension in mineral oil) was added in portions over 15 minutes and the mixture was stirred an additional 30 minutes. In a separate flask, [(2R,3R)-3-(3-fluorophenyl)oxiran-2-yl]methanol (1.08 g, 6.4 mmol, from Example 25 Step 3) was dissolved in N,N-dimethylformamide (3 mL) and titanium isopropoxide (1.89 mL, 6.4 mmol) was added and the mixture was stirred 30 minutes. The titanium isopropoxide/epoxide solution was then added to the solution of oxindole sodium salt dropwise and the mixture was stirred at room temperature for 24 hours. The mixture was then carefully quenched with 2 N aqueous hydrochloric acid and diluted with 200 mL of 2 N aqueous hydrochloric acid (use of hydrochloric acid is essential to prevent precipitation of titanium salts and subsequent emulsification). The mixture was extracted with ethyl acetate, the organic layers combined, washed with water, and saturated brine, dried over anhydrous magnesium sulfate, filtered, and concentrated under reduced pressure. The crude product was purified via Isco chromatography (Redisep, silica, gradient 20% to 100% ethyl acetate in hexane) to afford 1.02 g (87%) of 5,7-difluoro-1-[(1S,2S)-1-(3-fluorophenyl)-2,3-dihydroxypropyl]-3,3-dimethyl-1,3-dihydro-2H-indol-2-one as a sticky oil. MS (ES) m/z 365.12 (M⁺).

Step 2:

5,7-difluoro-1-[(1S,2S)-1-(3-fluorophenyl)-2,3-dihydroxypropyl]-3,3-dimethyl-1,3-dihydro-2H-indol-2-one (1.01 g, 2.76 mmol) was dissolved in pyridine (5 mL) and p-toluenesulfonyl chloride (570 mg, 3.0 mmol) was added and the mixture stirred for 4 hours. The reaction mixture was then diluted with diethyl ether and washed with water, 2 N aqueous hydrochloric acid, saturated copper sulfate, 2 N aqueous hydrochloric acid, and saturated brine. The organic layer was separated, dried over anhydrous magnesium sulfate, filtered, and concentrated under reduced pressure. The crude product was immediately dissolved in methylamine solution (8.0 M in ethanol, 30 mL) and stirred for 16 hours. The mixture was concentrated under reduced pressure and purified via chromatography (silica, 5% methanol saturated with ammonia in chloroform) to give 5,7-difluoro-1-[(1S,2R)-1-(3-fluorophenyl)-2-hydroxy-3-(methylamino)propyl]-3,3-dimethyl-1,3-dihydro-2H-indol-2-one as a colorless oil (0.29 g). The freebase was dissolved in ether (10 mL) and treated with hydrogen chloride solution (1.0 M in diethyl ether, 0.74 mL, 1.1 equivalent). The white precipitate was collected and dried under vacuum then dissolved in 10 mL of water and lyophilized to give 305 mg (26% over three steps) of 5,7-difluoro-1-[(1S,2R)-1-(3-fluorophenyl)-2-hydroxy-3-(methylamino)-propyl]-3,3-dimethyl-1,3-dihydro-2H-indol-2-one hydrochloride. MS (ES) m/z 379 (M+H⁺).

### Example 64

### 1-[(1S,2R)-1-(3,5-difluorophenyl)-2-hydroxy-3-(methylamino)propyl]-3,3-dimethyl-1,3-dihydro-2H-indol-2-one hydrochloride

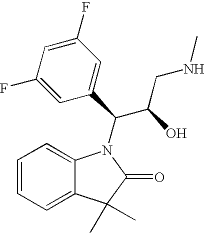

Step 1:

Oxindole (20.0 g; 0.15 mol) and lithium chloride (21.0 g; 0.49 mol) were suspended in tetrahydrofuran (400 mL) and the mixture cooled to −78° C. n-Butyllithium (120.0 mL; 0.30 mol, 2.5M in hexanes) was added slowly and the mixture was stirred for 20 minutes, then iodomethane (18.7 mL; 0.30 mol) was added. The mixture was warmed to 25° C., stirred overnight and then quenched with saturated aqueous ammonium chloride and diluted with diethyl ether. The organic layer was washed with water and brine, dried over magnesium sulfate, filtered and concentrated under reduced pressure. Purification by flash chromatography (0-20% ethyl acetate-hexane) afforded 10.0 g (41%) of 3,3-dimethyl-1,3-dihydro-indol-2-one as a yellow solid. MS (ESI) m/z 162 ([M+H]⁺).

Step 2:

In an analogous manner to Example 27, step 2, 1-[(1S,2S)-1-(3,5-difluorophenyl)-2,3-dihydroxypropyl]-3,3-dimethyl-1,3-dihydro-2H-indol-2-one was prepared from 3,3-dimethyl-1,3-dihydro-indol-2-one and [(2R,3R)-3-(3,5-difluorophenyl)oxiran-2-yl]methanol (from Example 61, Step 3). MS (ESI) m/z 348 ([M+H]⁺).

Step 3:

In an analogous manner to Example 27, step 3, 1-[(1S,2R)-1-(3,5-difluorophenyl)-2-hydroxy-3-(methylamino)propyl]-3,3-dimethyl-1,3-dihydro-2H-indol-2-one was prepared from 1-[(1S,2S)-1-(3,5-difluorophenyl)-2,3-dihydroxypropyl]-3,3-dimethyl-1,3-dihydro-2H-indol-2-one as a green oil. The free base was purified by reverse phase HPLC (Phenomenex Gemini, 19×150 mm, 60% methanol-40% water w/0.05% ammonium hydroxide). The pure free base was concentrated under reduced pressure and dissolved in diethyl ether. A solution of hydrogen chloride (1.0 M in diethyl ether, 1.2 equivalents) was added and the resulting white precipitate collected and dried under vacuum to give 36 mg (5% yield over two steps) of 1-[(1S,2R)-1-(3,5-difluorophenyl)-2-hydroxy-3-(methylamino)propyl]-3,3-dimethyl-1,3-dihydro-2H-indol-2-one hydrochloride. MS (ESI) m/z 361 ([M+H]⁺). HRMS: calculated for C₂₀H₂₂F₂N₂O₂+H⁺, 361.17221; found (ESI, [M+H]⁺), 361.1721.

### Example 65

### 1-[(1S,2R)-2-hydroxy-3-(methylamino)-1-phenylpropyl]-1H-indol-5-ol hydrochloride

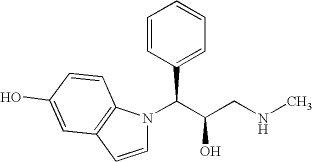

(1S,2R)-1-(5-Benzyloxy-1H-indol-1-yl)-3-(methylamino)-1-phenylpropan-2-ol (Example 1, 0.12 g, 0.3 mmol) was dissolved in methanol (20 mL) and treated with 10% palladium on carbon. The reaction mixture was placed under 52 psi of hydrogen on a Parr shaker for 15 hours. The reaction mixture was filtered through a Celite pad and the filtrate was concentrated under reduced pressure. The crude product was purified via Biotage Horizon (Flash 25 S, silica, gradient from 10% to 100% of 0.9% ammonium hydroxide in 10% methanol-methylene chloride/methylene chloride) to give a white solid as the free base of the expected product. The free base was dissolved in a minimum amount of ethanol and treated with hydrogen chloride solution (1.0 M in diethyl ether) until the solution was pH=3 followed by diethyl ether. The product was then crystallized by adding a minimum amount of ethyl acetate to afford 1-[(1S,2R)-2-hydroxy-3-(methylamino)-1-phenylpropyl]-1H-indol-5-ol hydrochloride as a white solid. HRMS: calculated for C₁₈H₂₀N₂O₂+H⁺, 297.15975; found (ESI, [M+H]⁺), 297.1599.

### Example 66

### 1-[(1S,2R)-1-(3-fluorophenyl)-2-hydroxy-3-(methylamino)propyl]-1H-indol-5-ol hydrochloride

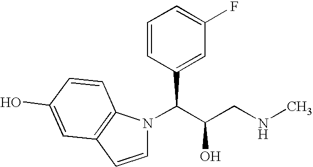

In an analogous manner to Example 65, 1-[(1S,2R)-1-(3-fluorophenyl)-2-hydroxy-3-(methylamino)propyl]-1H-indol-5-ol hydrochloride was prepared from (1S,2R)-1-(5-benzyloxy-1H-indol-1-yl)-3-(methylamino)-1-(3-fluorophenyl)propan-2-ol (Example 24) as a white solid. HRMS: calculated for C₁₈H₁₉FN₂O₂+H⁺, 315.15033; found (ESI, [M+H]⁺), 315.1516.

### Example 67

### 5′-(benzyloxy)-1′-[(1S,2R)-2-hydroxy-3-(methylamino)-1-phenylpropyl]spiro[cyclohexane-1,3′-indol]-2′(1′H)-one hydrochloride

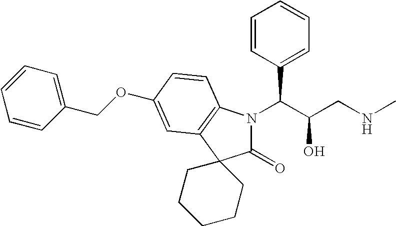

Step 1:

To a solution of spiro[cyclohexane-1,3′-[3H]indol]-2′(1′H)-one⁷ (5 g, 24.8 mmol) in trifluoroacetic acid (19 mL) and chloroform (240 mL) was added [bis(trifluoroacetoxy)iodo]benzene (12.8 g, 29.8 mmol) at room temperature and the reaction mixture stirred for 12 hours. The solution was then poured into saturated sodium bicarbonate (50 mL) and extracted with ethyl acetate (50 mL). The organic layer was separated, dried over anhydrous sodium sulfate, filtered and concentrated under reduced pressure. The residue was purified by flash column chromatography (10% to 80% ethyl acetate gradient in hexane) to give 5′-hydroxyspiro[cyclohexane-1,3′-indol]-2′(1′H)-one. MS (ES) m/z 218 ([M+H]⁺).
⁷ Fensome, A.; Miller, L. L.; Ullrich, J. W.; Bender, R. H. W.; Zhang, P.; Wrobel, J. E.; Zhi, L.; Jones, T. K.; Marschke, K. B.; Tegley, C. M. PCT Int. Appl. 2000, 127 pp. WO2000066556.

Step 2:

To a mixture of 5′-hydroxyspiro[cyclohexane-1,3′-indol]-2′(1′H)-one (0.62 g, 2.9 mmol) and potassium carbonate (1.2 g, 8.6 mmol) in N,N-dimethylformamide (10 mL) was added benzyl chloride (1 mL, 8.6 mmol) at room temperature under nitrogen. The resultant reaction mixture was stirred for 12 hours at room temperature, poured into a solution of saturated sodium chloride (50 mL), and extracted with ethyl acetate (50 mL). The organic layer was separated, dried over anhydrous magnesium sulfate, filtered and concentrated under reduced pressure. The crude product was purified by flash column chromatography (10% to 80% ethyl acetate gradient in hexane) to give 5′-benzyloxyspiro[cyclohexane-1,3′-indol]-2′(1′H)-one as a clear oil. MS (ES) m/z 308 ([M+H]⁺).

Step 3:

To a vigorously stirred mixture of 5′-benzyloxyspiro[cyclohexane-1,3′-indol]-2′(1′H)-one (0.37 g, 1.2 mmol) and 60% sodium hydride (0.053 g, 1.3 mmol) in N,N-dimethylformamide (4 mL) was added a solution of [(2R,3R)-3-phenyloxiran-2-yl]methanol (0.24 g, 1.69 mmol, from Example 1, Step 1) and titanium isopropoxide (0.48 mL, 1.6 mmol) in N,N-dimethylformamide (8 mL) which was prepared separately and aged for 15 minutes. The reaction mixture was stirred for 12 hours under nitrogen at room temperature, poured into an 3 N aqueous hydrochloric acid solution (100 mL), and extracted with ethyl acetate (2×50 mL). The combined organic layers were concentrated under reduced pressure and the residue purified via flash column chromatography (20% to 80% ethyl acetate gradient in hexane) to give 5′-benzyloxy-1′-[(1S,2S)-2,3-dihydroxy-1-phenylpropyl]spiro[cyclohexane-1,3′indol]2′(1′H)-one as a clear oil. MS (ES) m/z 458 ([M+H]⁺).

Step 4:

A solution of 5′-benzyloxy-1′-[(1S,2S)-2,3-dihydroxy-1-phenylpropyl]spiro[cyclohexane-1,3′indol]2′(1′H)-one (0.38 g, 0.83 mmol) in dry pyridine (3 mL) was treated with p-toluenesulfonyl chloride (0.24 g, 1.3 mmol). After 12 hours, the reaction mixture was diluted with ethyl acetate (25 mL) and the organic phase was washed with 1 N aqueous hydrochloric acid solution (25 mL) followed by a solution of saturated aqueous sodium bicarbonate (25 mL). The organic layer was separated, dried over sodium sulfate, filtered and concentrated under reduced pressure to give a clear oil that was dissolved in methanol (10 mL) and treated with an excess of methyl amine (33% by weight in absolute ethanol, 5 mL). The reaction solution was stirred in a sealed tube at room temperature for 12 hours, poured into saturated aqueous sodium bicarbonate solution (25 mL), extracted with ethyl acetate (25 mL), dried with anhydrous sodium sulfate, filtered and concentrated under reduced pressure. The residue was purified by flash column chromatography (0 to 80% methanol gradient in chloromethane) to give 5′-benzyloxy-1′-[(1S,2R)-2-hydroxy-3-(methylamino)-1-phenylpropyl]spiro[cyclohexane-1,3′indol]2′(1′H)-one as a clear oil. The oil was dissolved in ethanol (2-3 mL) and treated with hydrogen chloride solution (1.0 M in diethyl ether, 1.1 equivalents). The ethanol was removed to give 5′-benzyloxy-1′-[(1S,2R)-2-hydroxy-3-(methylamino)-1-phenylpropyl]spiro[cyclohexane-1,3′indol]2′(1′H)-one hydrochloride as an amorphous solid. MS (ES) m/z 471 ([M+H]⁺).

### Example 68

### 5-benzyloxy-1-[(1S,2R)-2-hydroxy-3-(methylamino)-1-phenylpropyl]-3,3-dimethyl-1,3-dihydro-2H-indol-2-one hydrochloride

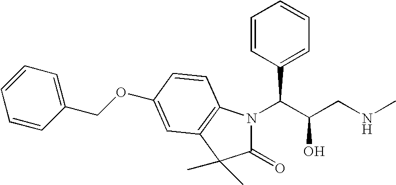

In an analogous manner to Example 1, step 1, 5-hydroxy-3,3-dimethyl-1,3-dihydro-2H-indol-2-one was prepared from 3,3-dimethyl-1,3-dihydro-2H-indol-2-one. MS (ES) m/z 178 ([M+H]⁺).

In an analogous manner to Example 1, step 2, 5-benzyloxy-3,3-dimethyl-1,3-dihydro-2H-indol-2-one was prepared from 5-hydroxy-3,3-dimethyl-1,3-dihydro-2H-indol-2-one. MS (ES) m/z 268 ([M+H]⁺).

In an analogous manner to Example 1, step 3, 1-[(1S,2S)-2,3-dihydroxy-1-phenylpropyl]-5-benzyloxy-3,3-dimethyl-1,3-dihydro-2H-indol-2-one was prepared from 5-benzyloxy-3,3-dimethyl-1,3-dihydro-2H-indol-2-one and [(2R,3R)-3-phenyloxiran-2-yl]methanol. MS (ES) m/z 418 ([M+H]⁺).

In an analogous manner to Example 1, step 4, 5-benzyloxy-1-[(1S,2R)-2-hydroxy-3-(methylamino)-1-phenylpropyl]-3,3-dimethyl-1,3-dihydro-2H-indol-2-one hydrochloride was prepared from 1-[(1S,2S)-2,3-dihydroxy-1-phenylpropyl]-5-benzyloxy-3,3-dimethyl-1,3-dihydro-2H-indol-2-one. MS (ES) m/z 431 ([M+H]⁺).

### Example 69

### 1-[(1S,2R)-1-(3-chlorophenyl)-2-hydroxy-3-(methylamino)propyl]-7-fluoro-3,3-dimethyl-1,3-dihydro-2H-indol-2-one hydrochloride

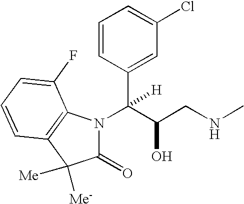

Step 1:

To a solution of 2,6-difluoronitrobenzene (5.0 g, 31.44 mmol) in dry N,N-dimethylformamide (50 mL) was added potassium carbonate (4.41 g, 32 mmol) and dimethylmalonate (3.6 mL, 31.44 mmol). The reaction mixture was heated to 65° C. and stirred for 24 hours. After cooling to room temperature, the mixture was neutralized with a dilute aqueous solution of hydrochloric acid and extracted with diethyl ether. The ethereal layer was dried over anhydrous magnesium sulfate, and concentrated under reduced pressure. Crystallization from 5% ethyl acetate/hexane gave 4.6 g (54%) of 2-(6-fluoro-2-nitro-phenyl)-malonic acid dimethyl ester. MS (ESI) m/z 272 [M+H]⁺).

Step 2:

2-(6-Fluoro-2-nitro-phenyl)-malonic acid dimethyl ester (12 g, 44 mmol) in a 6N aqueous solution of hydrochloric acid (200 mL) was heated at reflux for 4 hours. The mixture was cooled, diluted with 250 mL of water and extracted with diethyl ether. The ethereal layer was dried over anhydrous magnesium sulfate, and concentrated under reduced pressure. Crystallization from 5% ethyl acetate/hexane gave 7.6 g (54%) of (6-fluoro-2-nitro-phenyl)-acetic acid. MS (ESI) m/z 200 ([M+H]⁺).

Step 3:

A mixture of (6-fluoro-2-nitro-phenyl)-acetic acid (9.6 g, 48 mmol) and 10% palladium on carbon (1.3 g) in acetic acid (100 mL) was hydrogenated at 50 psi for 24 hours. The catalyst was removed by filtration through Celite and the solvent was evaporated. The residue was then dissolved in ethanol (100 mL) and pyridinium para-toluenesulfonate (50 mg) was added and the mixture heated at reflux for 1 hour. The mixture was cooled, poured into water, extracted with ethyl acetate and the organic extract dried over anhydrous magnesium sulfate, filtered and concentrated under reduced pressure. The solid was triturated with 5% ethyl acetate/hexane to give 6.0 g (83%) 7-fluoro-1,3-dihydro-indol-2-one. MS (ESI) m/z 152, [M+H]⁺).

Step 4:

7-Fluoro-1,3-dihydro-indol-2-one (7.3 g, 48 mmol) and lithium chloride (6.67 g, 158 mmol) were dissolved in tetrahydrofuran (200 mL). The solution was cooled to −78° C. and n-butyl lithium (40 mL, 100 mmol) was added slowly over a 15 minute period. After 20 minutes at −78° C., methyl iodide (6 mL, 96 mmol) was added and the mixture allowed to warm to room temperature. After 24 hours, the mixture was poured into water and extracted with ethyl acetate. The organic layer was dried over anhydrous magnesium sulfate, filtered and concentrated under reduced pressure. The crude product was purified via Biotage chromatography (Flash40i, silica, 10% then 20% ethyl acetate/hexane) to give 4.1 g (48%) 7-fluoro-3,3-dimethyl-1,3-dihydro-2H-indol-2-one. MS (ESI) m/z 180, [M+H]⁺).

Step 5:

7-fluoro-3,3-dimethyl-1,3-dihydro-2H-indol-2-one (0.09 g, 0.50 mmol) was dissolved in N,N-dimethylformamide (1.0 mL) and sodium hydride (0.029 g, 0.75 mmol, 60% wt suspension in mineral oil) was added and the mixture was stirred an additional 30 minutes. In a separate flask, [(2R,3R)-3-(3-chlorophenyl)oxiran-2-yl]methanol (0.184 g, 1.0 mmol—prepared in a method analogous to Example 1, Step 1) was dissolved in N,N-dimethylformamide (1 mL) and titanium isopropoxide (0.15 mL, 0.50 mmol) was added and the mixture was stirred for 30 minutes. The titanium isopropoxide/epoxide solution was then added to the solution of oxindole sodium salt dropwise and the mixture was stirred at room temperature for 24 hours. The mixture was then carefully quenched with 2 N aqueous hydrochloric acid and diluted with 50 mL of 2 N aqueous hydrochloric acid (the use of aqueous hydrochloric acid is essential to prevent precipitation of titanium salts and subsequent emulsification). The mixture was extracted with ethyl acetate the organic layers combined, washed with water, and saturated brine, dried over anhydrous magnesium sulfate, filtered, and concentrated under reduced pressure. The crude product was purified via Isco chromatography (Redisep, silica, gradient 20% to 100% ethyl acetate in hexane) to afford 0.155 g (85%) of 7-fluoro-1-[(1S,2S)-1-(3-chlorophenyl)-2,3-dihydroxypropyl]-3,3-dimethyl-1,3-dihydro-2H-indol-2-one as a sticky oil.

Step 6:

7-fluoro-1-[(1S,2S)-1-(3-chlorophenyl)-2,3-dihydroxypropyl]-3,3-dimethyl-1,3-dihydro-2H-indol-2-one (145 mg, 0.4 mmol) was dissolved in pyridine (2 mL) and toluenesulfonyl chloride (76 mg, 0.4 mmol) was added. The reaction mixture was stirred for 4 hours then the mixture was diluted with diethyl ether and washed with water, 2 N aqueous hydrochloric acid, saturated aqueous copper sulfate, 2 N aqueous hydrochloric acid, and saturated brine. The organic layer was separated, dried over anhydrous magnesium sulfate, filtered, and concentrated under reduced pressure. The crude product was immediately dissolved in a solution of methylamine (8 M in ethanol, 10 mL) and stirred for 16 hours. The mixture was concentrated under reduced pressure and purified via chromatography (silica, 5% methanol saturated with ammonia in chloroform) to give 44 mg of 1-[(1S,2R)-1-(3-chlorophenyl)-2-hydroxy-3-(methylamino)propyl]-7-fluoro-3,3-dimethyl-1,3-dihydro-2H-indol-2-one as a colorless oil. The freebase was dissolved in ether (5 mL) and treated with a solution of hydrogen chloride (1.0 M in diethyl ether, 0.12 mL, 1.0 equivalent). The white precipitate was collected and dried under vacuum to give 38 mg (24% over three steps) of 1-[(1S,2R)-1-(3-chlorophenyl)-2-hydroxy-3-(methylamino)propyl]-7-fluoro-3,3-dimethyl-1,3-dihydro-2H-indol-2-one hydrochloride.

HPLC purity 100% at 210-370 nm, 7.8 min.; Xterra RP18, 3.5 u, 150×4.6 mm column, 1.2 mL/min, 85115-5/95 (Ammon. Form. Buff. Ph=3.5/MeCN+MeOH) for 10 minutes, hold 4 minutes.

MS (ES) m/z 377.1; ([M+H]⁺).

### Example 70

### (1S,2R)-1-(3-chloro-5-fluorophenyl)-1-(1H-indol-1-yl)-3-(methylamino)propan-2-ol hydrochloride

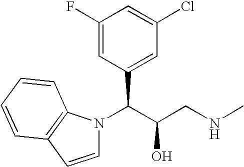

Step 1:

To a suspension of NaH (60% in mineral oil, 3.0 g, 75.7 mmol) in dry tetrahydrofuran (460 mL) was added triethyl phosphonoacetate (16.97 g, 75.7 mmol) at room temperature. After stirring for 1 hour, 3-chloro-5-fluorobenzaldehyde (10.0 g, 63.07 mmol) in tetrahydrofuran (20 mL) was added dropwise. The reaction was stirred for 12 hours, quenched with water (30 mL) and concentrated. The crude residue was then taken up in ethyl acetate, washed with water, and brine, dried over sodium sulfate, filtered and concentrated under reduced pressure to give 6 g (96%) of ethyl (2E)-3-(3-chloro-5-fluorophenyl)acrylate as a white solid. HRMS: calculated for C₁₁H₁₀ClFO₂, 228.0353; found (EI, [M⁺], 228.0340.

Step 2:

To a solution of ethyl (2E)-3-(3-chloro-5-fluorophenyl)acrylate (13.76 g, 228.65 mmol) in dry dichloromethane (200 mL) at −78° C. under nitrogen was added dropwise diisobutylaluminum hydride (neat, 21.7 mL, 120 mmol, 2 equiv.) via an addition funnel. The reaction mixture was stirred for an additional 30 minutes, then slowly quenched with methanol (75 mL). Upon warming to room temperature, the mixture was treated with saturated aqueous solution of sodium/potassium tartrate (75 mL) and stirred for 30 minutes. Ethyl acetate was added and the organic layer was washed sequentially with 1 N aqueous hydrochloric acid, saturated aqueous sodium bicarbonate, and brine, dried over sodium sulfate, filtered and concentrated under reduced pressure. The crude oil was purified on silica gel (0-50% ethyl acetate:hexane) to give 7.30 g (65%) of (2E)-3-(3-chloro-5-fluorophenyl)prop-2-en-1-ol as a colorless oil. MS (ESI) m/z 168.9 ([M+H−H₂O]⁺).

Step 3:

In an analogous manner to Example 25, Step 3, [(2R,3R)-3-(3-chloro-5-fluorophenyl)oxiran-2-yl]methanol was prepared from (2E)-3-(3-chloro-5-fluorophenyl)prop-2-en-1-ol . MS (ESI) m/z 244 ([M+CH₃CN+H]⁺).

Step 4:

In an analogous manner to Example 1, Step 3, (2S,3S)-3-(3-chloro-5-fluorophenyl)-3-(2,3-dihydro-1H-indol-1-yl)propane-1,2-diol was prepared from indoline and [(2R,3R)-3-(3-chloro-5-fluorophenyl)oxiran-2-yl]methanol. MS (ES) m/z 322.0 ([M+H]⁺).

Step 5:

In an analogous manner to Example 1, Step 4, (2S,3S)-3-(3-chloro-5-fluorophenyl)-3-(1H-indol-1-yl)propane-1,2-diol was prepared from (2S,3S)-3-(3-chloro-5-fluorophenyl)-3-(2,3-dihydro-1H-indol-1-yl)propane-1,2-diol. MS (ES) m/z 320.0 ([M+H]⁺).

Step 6:

In an analogous manner to Example 69, Step 6, (1S,2R)-1-(3-chloro-5-fluorophenyl)-1-(1H-indol-1-yl)-3-(methylamino)propan-2-ol hydrochloride, was prepared from (2S,3S)-3-(3-chloro-5-fluorophenyl)-3-(1H-indol-1-yl)propane-1,2-diol and methylamine. MS (ES) m/z 333 ([M+H]⁺).

### Example 71

### 3-chloro-N-{1-[(1S,2R)-2-hydroxy-3-(methylamino)-1-phenylpropyl]-1H-indol-5-yl}-4-methylbenzamide hydrochloride

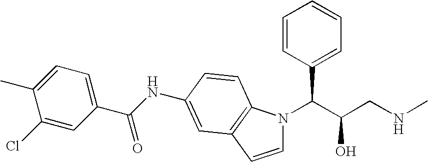

Step 1:

A mixture of 5-aminoindole (1.32 g, 10 mmol), 1-hydroxybenzotriazole (1.49 g, 11 mmol), and 1-(3-dimethylaminopropyl)-3-ethyl-carbodiimide hydrochloride (2.11 g, 11 mmol) was dissolved in N,N-dimethylformamide (30 mL). To this was added 3-chloro-4-methylbenzoic acid (1.71 g, 10 mmol) and the reaction mixture was stirred for 2 hours until the reaction was complete. The mixture was then partitioned between water and dichloromethane solution. The organic layer was separated and the aqueous layer was extracted with dichloromethane several times. The combined extracts were washed with water and brine, dried over anhydrous sodium sulfate, filtered, and concentrated under reduced pressure. The crude product was purified via Biotage Horizon (FlasH 40 M, silica, gradient from 0% ethyl acetate/hexane to 70% ethyl acetate/hexane) to give 3-chloro-N-(1H-indol-5-yl)-4-methylbenzamide as a light tan solid. MS (ESI) m/z 284.9 ([M+H]⁺).

In an analogous manner to Example 1, Step 2, 3-chloro-N-(indolin-5-yl)-4-methylbenzamide was prepared from 3-chloro-N-(1H-indol-5-yl)-4-methyl benzamide as a light tan solid. MS (ESI) m/z 286.9 ([M+H]⁺).

In an analogous manner to Example 1, Step 3, 3-chloro-N-{1-[(1S,2S)-2,3-dihydroxy-1-phenylpropyl]indolin-5-yl}-4-methylbenzamide was prepared from 3-chloro-N-(indolin-5-yl)-4-methylbenzamide as a white fluffy solid. MS (ESI) m/z 437 ([M+H]⁺).

In an analogous manner to Example 1, Step 4, 3-chloro-N-{1-[(1S,2S)-2,3-dihydroxy-1-phenylpropyl]-1H-indol-5-yl}-4-methylbenzamide was prepared from 3-chloro-N-{1-[(1S,2S)-2,3-dihydroxy-1-phenylpropyl]indolin-5-yl}-4-methyl benzamide as an oil. MS (ESI) m/z 435.1 ([M+H]⁺).

In an analogous manner to Example 1, Step 5, (2S,3S)-3-[5-(3-chloro-4-methylbenzamido)-1H-indol-1-yl]-2-hydroxy-3-phenylpropyl 4-methylbenzene sulfonate was prepared from 3-chloro-N-{1-[(1S,2S)-2,3-dihydroxy-1-phenylpropyl]-1H-indol-5-yl}-4-methylbenzamide as an oil. MS (ESI) m/z 589 ([M+H]⁺).

In an analogous manner to Example 1, Step 6, 3-chloro-N-{1-[(1S,2R)-2-hydroxy-3-(methylamino)-1-phenylpropyl]-1H-indol-5-yl}-4-methylbenzamide hydrochloride was prepared from (2S,3S)-3-[5-(3-chloro-4-methylbenzamido)-1H-indol-1-yl]-2-hydroxy-3-phenylpropyl 4-methylbenzenesulfonate and methylamine (2N solution in methanol) as a tan solid. MS (ESI) m/z 448 ([M+H]⁺); HPLC purity 100% at 210-370 nm, 8.9 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calculated for C₂₆H₂₆ClN₃O₂+H⁺, 448.17863; found (ESI, [M+H]⁺), 448.1692.

### Example 72

### 3-Chloro-N-{1-[(1S,2R)-2-hydroxy-3-(methylamino)-1-phenylpropyl]-2,3-dihydro-1H-indol-5-yl}benzamide hydrochloride

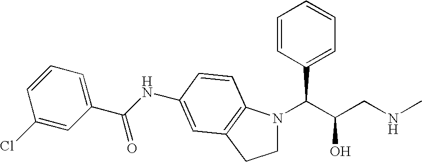

Step 1:

In an analogous manner to Example 71, Step 1, 3-chloro-N-(1H-indol-5-yl)benzamide was prepared from 5-aminoindole and 3-chlorobenzoic acid as a dark tan solid. MS (ESI) m/z 270.9 ([M+H]⁺).

Step 2:

In an analogous manner to Example 1, Step 2, 3-chloro-N-(indolin-5-yl) benzamide was prepared from 3-chloro-N-(1H-indol-5-yl)benzamide as a light tan solid. MS (ESI) m/z 272.9 ([M+H]⁺).

Step 3:

In an analogous manner to Example 1, Step 3, 3-chloro-N-{1-[(1S,2S)-2,3-dihydroxy-1-phenylpropyl]indolin-5-yl}benzamide was prepared from 3-chloro-N-(indolin-5-yl)benzamide as a pale yellow solid. MS (ESI) m/z 423 ([M+H]⁺).

Step 4:

In an analogous manner to Example 1, Step 5, (2S,3S)-3-[5-(3-chlorobenzamido)indolin-1-yl]-2-hydroxy-3-phenylpropyl 4-methylbenzene sulfonate was prepared from 3-chloro-N-{1-[(1S,2S)-2,3-dihydroxy-1-phenylpropyl]indolin-5-yl}benzamide as an oil. MS (ESI) m/z 578 ([M+H]⁺).

Step 5:

In an analogous manner to Example 1, Step 6, 3-chloro-N-{1-[(1S,2R)-2-hydroxy-3-(methylamino)-1-phenylpropyl]-2,3-dihydro-1H-indol-5-yl}benzamide hydrochloride was prepared from (2S,3S)-3-[5-(3-chlorobenzamido)indolin-1-yl]-2-hydroxy-3-phenylpropyl 4-methylbenzenesulfonate and methylamine (2N solution in methanol) as a pale yellow solid. MS (ES) m/z 436.1 ([M+H]⁺); HPLC purity 100% at 210-370 nm, 8.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calculated for C₂₅H₂₆ClN₃O₂+H⁺, 436.17863; found (ESI, [M+H]⁺), 434.1618.

### Example 73

### 3-Chloro-N-{1-[(1S,2R)-2-hydroxy-3-(methylamino)-1-phenylpropyl]-1H-indol-5-yl}benzamide hydrochloride

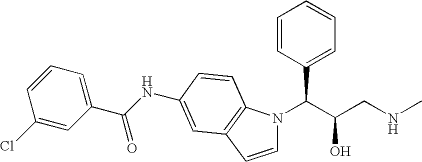

In an analogous manner to Example 1, Step 4, 3-chloro-N-{1-[(1S,2S)-2,3-dihydroxy-1-phenylpropyl]-1H-indol-5-yl}benzamide was prepared from 3-chloro-N-{1-[(1S,2S)-2,3-dihydroxy-1-phenylpropyl]indolin-5-yl}benzamide (from Example 72, Step 3) as an oil. MS (ES) m/z 421.1 ([M+H]⁺).

In an analogous manner to Example 1, Step 5, (2S,3S)-3-[5-(3-chlorobenz-amido)-1H-indol-1-yl]-2-hydroxy-3-phenylpropyl 4-methylbenzenesulfonate was prepared from 3-chloro-N-{1-[(1S,2S)-2,3-dihydroxy-1-phenylpropyl]-1H-indol-5-yl}benzamide as an oil. MS (ESI) m/z 576 ([M+H]⁺).

In an analogous manner to Example 1, Step 6, 3-chloro-N-{1-[(1S,2R)-2-hydroxy-3-(methylamino)-1-phenylpropyl]-1H-indol-5-yl}benzamide hydrochloride was prepared from (2S,3S)-3-[5-(3-chlorobenzamido)-1H-indol-1-yl]-2-hydroxy-3-phenylpropyl 4-methylbenzenesulfonate and methylamine (2N solution in methanol) as a white solid. MS (ES) m/z 434.1 ([M+H]⁺); HPLC purity 100% at 210-370 nm, 8.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calculated for C₂₅H₂₄ClN₃O₂+H⁺, 434.16298; found (ESI, [M+H]⁺), 434.1617.

### Example 74

### N-{1-[(1S,2R)-2-Hydroxy-3-(methylamino)-1-phenylpropyl]-2,3-dihydro-1H-indol-5-yl}benzamide hydrochloride

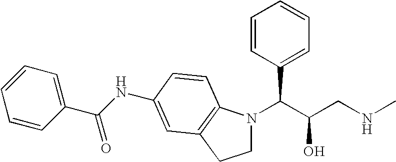

Step 1:

In an analogous manner to Example 71, Step 1, N-(1H-Indol-5-yl)benzamide was prepared from 5-aminoindole and benzoic acid as a light tan solid. MS (ESI) m/z 237 ([M+H]⁺).

Step 2:

In an analogous manner to Example 1, Step 2, N-(indolin-5-yl)benzamide was prepared from N-(1H-indol-5-yl)benzamide as a light tan solid. MS (ESI) m/z 239.0 ([M+H]⁺).

Step 3:

In an analogous manner to Example 1, Step 3, N-{1-[(1S,2S)-2,3-dihydroxy-1-phenylpropyl]indolin-5-yl}benzamide was prepared from N-(indolin-5-yl)benzamide as a pale yellow solid. MS (ESI) m/z 389.1([M+H]⁺).

Step 4:

In an analogous manner to Example 1, Step 5, (2S,3S)-3-(5-benzamidoindolin-1-yl)-2-hydroxy-3-phenylpropyl 4-methylbenzenesulfonate was prepared from N-{1-[(1S,2S)-2,3-dihydroxy-1-phenylpropyl]indolin-5-yl}benzamide as an oil. MS (ESI) m/z 543 ([M+H]⁺).

Step 5:

In an analogous manner to Example 1, Step 6, N-{1-[(1S,2R)-2-hydroxy-3-(methylamino)-1-phenylpropyl]-2,3-dihydro-1H-indol-5-yl}benzamide hydrochloride was prepared from (2S,3S)-3-(5-benzamidoindolin-1-yl)-2-hydroxy-3-phenylpropyl 4-methylbenzenesulfonate and methylamine (2N solution in methanol) as a light tan solid. MS (ES) m/z 402.1 ([M+H]⁺); HPLC purity 96.8% at 210-370 nm, 7.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calculated for C₂₅H₂₇N₃O₂+H⁺, 402.21760; found (ESI, [M+H]⁺), 402.212.

### Example 75

### N-{1-[(1S,2R)-2-Hydroxy-3-(methylamino)-1-phenylpropyl]-1H-indol-5-yl}benzamide hydrochloride

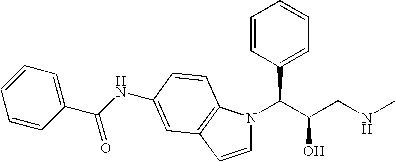

In an analogous manner to Example 1, Step 4, N-{1-[(1S,2S)-2,3-dihydroxy-1-phenylpropyl]-1H-indol-5-yl}benzamide was prepared from N-{1-[(1S,2S)-2,3-dihydroxy-1-phenylpropyl]indolin-5-yl}benzamide (from Example 74, step 3) as an oil. MS (ES) m/z 387.1 ([M+H]⁺).

In an analogous manner to Example 1 Step 5, (2S,3S)-3-(5-benzamido-1H-indol-1-yl)-2-hydroxy-3-phenylpropyl 4-methylbenzenesulfonate was prepared from N-{1-[(1S,2S)-2,3-dihydroxy-1-phenylpropyl]-1H-indol-5-yl}benzamide as an oil. MS (ESI) m/z 541 ([M+H]⁺).

In an analogous manner to Example 1, Step 6, N-{1-[(1S,2R)-2-hydroxy-3-(methylamino)-1-phenylpropyl]-1H-indol-5-yl}benzamide hydrochloride was prepared from (2S,3S)-3-(5-(benzamido-1H-indol-1-yl)-2-hydroxy-3-phenylpropyl 4-methylbenzenesulfonate and methylamine (2N solution in methanol) as an off-white solid. MS (ES) m/z 400.1 ([M+H]⁺); HPLC purity 100% at 210-370 nm, 7.4 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calculated for C₂₅H₂₅N₃O₂+H⁺, 400.20195; found (ESI, [M+H]⁺), 400.2034.

### Example 76

### N-{1-[(1S,2R)-2-Hydroxy-3-(methylamino)-1-phenylpropyl]-2,3-dihydro-1H-indol-5-yl}cyclohexanecarboxamide hydrochloride

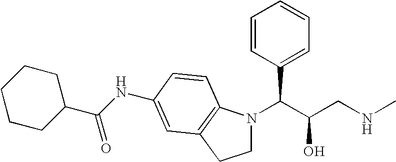

Step 1:

In an analogous manner to Example 71, Step 1, N-(1H-indol-5-yl)cyclohexanecarboxamide was prepared from 5-aminoindole and cyclohexanecarboxylic acid as an oil. MS (ESI) m/z 243.0 ([M+H]⁺).

Step 2:

In an analogous manner to Example 1, Step 2, N-(indolin-5-yl)cyclohexanecarboxamide was prepared from N-(1H-indol-5-yl)cyclohexanecarboxamide as an oil. MS (ESI) m/z 245 ([M+H]⁺).

Step 3:

In an analogous manner to Example 1, Step 3, N-{1-[(1S,2S)-2,3-dihydroxy-1-phenylpropyl]indolin-5-yl}cyclohexanecarboxamide was prepared from N-(indolin-5-yl)cyclohexanecarboxamide as a white solid. MS (ESI) m/z 395.1 ([M+H]⁺).

Step 4:

In an analogous manner to Example 1, Step 5, (2S,3S)-3-[5-(cyclohexanecarboxamido)indolin-1-yl]-2-hydroxy-3-phenylpropyl 4-methylbenzenesulfonate was prepared from N-{1-[(1S,2S)-2,3-dihydroxy-1-phenylpropyl]indolin-5-yl}cyclohexanecarboxamide as an oil. MS (ESI) m/z 547 ([M+H]⁺).

Step 5:

In an analogous manner to Example 1, Step 6, N-{1-[(1S,2R)-2-hydroxy-3-(methylamino)-1-phenylpropyl]-2,3-dihydro-1H-indol-5-yl}cyclohexane carboxamide hydrochloride was prepared from (2S,3S)-3-[5-{cyclohexanecarboxamido]indolin-1-yl}-2-hydroxy-3-phenylpropyl 4-methylbenzenesulfonate and methylamine (2N solution in methanol) as an off-white solid. MS (ES) m/z 408.2 ([M+H]⁺); HPLC purity 100% at 210-370 nm, 7.9 min.; Xterra RP18, 3.5 u, 150×4.6 mm column, 1.2 mL/min, 85/15-5195 (Ammon. Form. Buff. Ph=3.5/CAN+MeOH) for 10 min, hold 4 min.

### Example 77

### N-{1-[(1S,2R)-2-Hydroxy-3-(methylamino)-1-phenylpropyl]-1H-indol-5-yl}cyclohexanecarboxamide hydrochloride

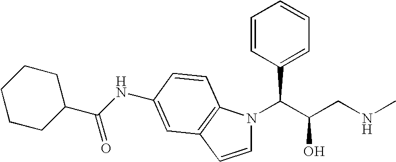

In an analogous manner to Example 1, Step 4, N-{1-[(1S,2S)-2,3-dihydroxy-1-phenylpropyl]-1H-indol-5-yl}cyclohexanecarboxamide was prepared from N-{1-[(1S,2S)-2,3-dihydroxy-1-phenylpropyl]indolin-5-yl}cyclohexanecarboxamide (from Example 76, Step 3) as an oil. MS (ES) m/z 393.1 ([M+H]⁺).

In an analogous manner to Example 1, Step 5, (2S,3S)-3-[5-(cyclohexanecarboxamido)-1H-indol-1-yl]-2-hydroxy-3-phenylpropyl 4-methylbenzene sulfonate was prepared from N-{1-[(1S,2S)-2,3-dihydroxy-1-phenylpropyl]-1H-indol-5-yl}cyclohexanecarboxamide as an oil. MS (ESI) m/z 547 ([M+H]⁺).

In an analogous manner to Example 1, Step 6, N-{1-[(1S,2R)-2-hydroxy-3-(methylamino)-1-phenylpropyl]-1H-indol-5-yl}cyclohexanecarboxamide hydrochloride was prepared from (2S,3S)-3-[5-(cyclohexanecarboxamido)-1H-indol-1-yl]-2-hydroxy-3-phenylpropyl-4-methylbenzenesulfonate and methylamine (2N solution in methanol) as an off-white solid. MS (ES) m/z 406.1 ([M+H]⁺); HPLC purity 100% at 210-370 nm, 8.0 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calculated for C₂₅H₃₁N₃O₂+H⁺, 406.24890; found (ESI, [M+H]⁺), 406.2492.

### Example 78

### N-(3-Chlorophenyl)-1-[(1S,2R)-2-hydroxy-3-(methylamino)-1-phenylpropyl]indoline-5-carboxamide hydrochloride

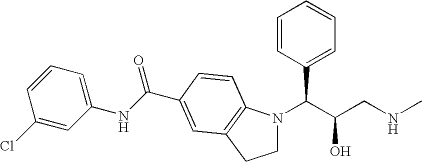

Step 1:

In an analogous manner to Example 71, Step 1, N-(3-chlorophenyl)-1H-indole-5-carboxamide was prepared from 1H-indole-5-carboxylic acid and 3-chloroaniline as a oily tan solid. MS (ESI) m/z 270.9 ([M+H]⁺).

Step 2:

In an analogous manner to Example 1, Step 2, N-(3-chlorophenyl)indoline-5-carboxamide was prepared from N-(3-chlorophenyl)-1H-indole-5-carboxamide as a light tan solid. MS (ESI) m/z 272.9 ([M+H]⁺).

Step 3:

In an analogous manner to Example 1, Step 3, N-(3-chlorophenyl)-1-[(1S,2S)-2,3-dihydroxy-1-phenylpropyl]indoline-5-carboxamide was prepared from N-(3-chlorophenyl)indoline-5-carboxamide as a white solid. MS (ESI) m/z 423 ([M+H]⁺).

Step 4:

In an analogous manner to Example 1, Step 5, (2S,3S)-3-[5-(3-chlorophenyl carbamoyl)indolin-1-yl]-2-hydroxy-3-phenylpropyl 4-methylbenzene sulfonate was prepared from N-(3-chlorophenyl)-1-[(1S,2S)-2,3-dihydroxy-1-phenylpropyl]indoline-5-carboxamide as an oil. MS (ESI) m/z 577 ([M+H]⁺).

Step 5:

In an analogous manner to Example 1, Step 6, N-(3-chlorophenyl)-1-[(1S,2R)-2-hydroxy-3-(methylamino)-1-phenylpropyl]indoline-5-carboxamide hydrochloride was prepared from (2S,3S)-3-[5-(3-chlorophenylcarbamoyl)indolin-1-yl]-2-hydroxy-3-phenylpropyl 4-methylbenzene sulfonate and methylamine (2N solution in methanol) as a pale yellow solid. MS (ES) m/z 436.1 ([M+H]⁺); HPLC purity 100% at 210-370 nm, 8.6 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 minutes. HRMS: calculated for C₂₅H₂₆ClN₃O₂+H⁺, 436.17863; found (ESI, [M+H]⁺), 436.1802.

### Example 79

### N-(3-Chlorophenyl)-1-[(1S,2R)-2-hydroxy-3-(methylamino)-1-phenylpropyl]-1H-indole-5-carboxamide hydrochloride

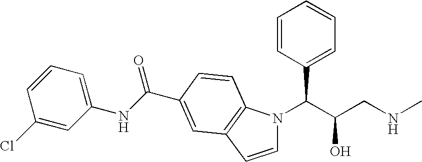

In an analogous manner to Example 1, Step 4, N-(3-chlorophenyl)-1-[(1S,2S)-2,3-dihydroxy-1-phenylpropyl]-1H-indole-5-carboxamide was prepared from N-(3-chlorophenyl)-1-[(1S,2S)-2,3-dihydroxy-1-phenylpropyl]indoline-5-carboxamide (from Example 78, Step 3) as an oil. MS (ESI) m/z 421.1.

In an analogous manner to Example 1, Step 5, (2S,3S)-3-[5-(3-chlorophenyl carbamoyl)-1H-indol-1-yl]-2-hydroxy-3-phenylpropyl 4-methylbenzenesulfonate was prepared from N-(3-chlorophenyl)-1-[(1S,2S)-2,3-dihydroxy-1-phenylpropyl]-1H-indole-5-carboxamide as an oil. MS (ESI) m/z 575 ([M+H]⁺).

In an analogous manner to Example 1, Step 6, N-(3-chlorophenyl)-1-[(1S,2R)-2-hydroxy-3-(methylamino)-1-phenylpropyl]-1H-indole-5-carboxamide hydrochloride was prepared from (2S,3S)-3-[5-(3-chlorophenylcarbamoyl)-1H-indol-1-yl]-2-hydroxy-3-phenylpropyl 4-methylbenzenesulfonate and methylamine (2N solution in methanol) as a white solid. MS (ES) m/z 434.1 ([M+H]⁺); HPLC purity 100% at 210-370 nm, 8.7 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calculated for C₂₅H₂₄ClN₃O₂+H⁺, 434.16298; found (ESI, [M+H]⁺), 434.1634.

### Example 80

### (1S,2R)-3-(methylamino)-1-(6-phenoxy-1H-indol-1-yl)-1-phenylpropan-2-ol hydrochloride

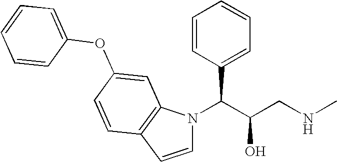

In an analogous manner to Example 20, Step 1, 2-methyl-1-nitro-5-phenoxybenzene was prepared from 4-methyl-3-nitrophenol. ¹H NMR (400 MHz, (CD₃)₂SO) δ 2.48 (s, 3H), 7.10 (d, 2H), 7.23 (t, 1H), 7.31 (dd, 1H), 7.45 (t, 2H), 7.52 (d, 1H), and 7.55 (m, 1H).

In an analogous manner to Example 19, Step 2, dimethyl-[2-(2-nitro-4-phenoxy-phenyl)-vinyl]-amine was prepared from 2-methyl-1-nitro-5-phenoxybenzene. ¹H NMR (400 MHz, (CD₃)₂SO) δ 2.88 (s, 6H), 5.66 (d, 1H), 7.05 (d, 2H), 7.15-7.20 (m, 2H), 7.32 (d, 1H), 7.39-7.43 (m, 3H), 7.71 (d, 1H).

In an analogous manner to Example 19, Step 3, 6-phenoxy-1H-indole was prepared from dimethyl-[2-(2-nitro-4-phenoxy-phenyl)-vinyl]-amine. MS (ES) m/z 210 ([M+H]⁺).

In an analogous manner to Example 1, Step 2, 6-phenoxyindoline was prepared from 6-phenoxy-1H-indole. MS (ES) m/z 212 ([M+H]⁺).

In an analogous manner to Example 1, Step 3, (2S,3S)-3-(6-phenoxy-2,3-dihydro-1H-indol-1-yl)-3-phenylpropane-1,2-diol was prepared from 6-phenoxyindoline. MS (ES) m/z 362 ([M+H]⁺).

In an analogous manner to Example 1, Step 4, (2S,3S)-3-(6-phenoxy-1H-indol-1-yl)-3-phenylpropane-1,2-diol was prepared from (2S,3S)-3-(6-phenoxy-2,3-dihydro-1H-indol-1-yl)-3-phenylpropane-1,2-diol. MS (ES) m/z 360 ([M+H]⁺).

In an analogous manner to Example 25, Step 5, (1S,2R)-3-(methylamino)-1-(6-phenoxy-1H-indol-1-yl)-1-phenylpropan-2-ol hydrochloride was prepared from (2S,3S)-3-(6-phenoxy-1H-indol-1-yl)-3-phenylpropane-1,2-diol. MS (ES) m/z 373 ([M+H]⁺); HRMS: calculated for C₂₄H₂₄N₂O₂+H⁺, 373.19105; found (ESI, [M+H]⁺), 373.1916.

### Example 81

### (1S,2R)-3-(methylamino)-1-(7-phenoxy-1H-indol-1-yl)-1-phenylpropan-2-ol hydrochloride

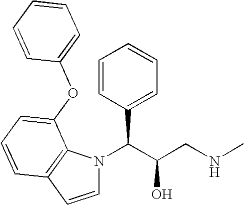

In an analogous manner to Example 20, Step 1, 2-methyl-1-nitro-6-phenoxybenzene was prepared from 3-methyl-2-nitrophenol. ¹H NMR (400 MHz, (CD₃)₂SO) δ 2.34 (s, 3H), 6.93 (d, 1H), 7.07 (d, 2H), 7.22 (t, 2H), and 7.41-7.49 (m, 3H).

In an analogous manner to Example 19, Step 2, dimethyl-[2-(2-nitro-3-phenoxy-phenyl)-vinyl]-amine was prepared from 2-methyl-1-nitro-6-phenoxybenzene. ¹H NMR (400 MHz, (CD₃)₂SO) δ 2.83 (s, 6H), 4.66 (d, 1H), 6.48 (d, 1H), 7.04 (d, 2H), 7.19 (t, 1H), 7.25 (t, 1H), and 7.32-7.43 (m, 4H).

In an analogous manner to Example 19, Step 3, 7-phenoxy-1H-indole was prepared from dimethyl-[2-(2-nitro-3-phenoxy-phenyl)-vinyl]-amine. MS (ES) m/z 210 ([M+H]⁺).

In an analogous manner to Example 1, Step 2, 7-phenoxyindoline was prepared from 7-phenoxy-1H-indole. MS (ES) m/z 212 ([M+H]⁺).

In an analogous manner to Example 1, Step 3, (2S,3S)-3-(7-phenoxy-2,3-dihydro-1H-indol-1-yl)-3-phenylpropane-1,2-diol was prepared from 7-phenoxyindoline. MS (ES) m/z 362 ([M+H]⁺).

In an analogous manner to Example 1, Step 4, (2S,3S)-3-(7-phenoxy-1H-indol-1-yl)-3-phenylpropane-1,2-diol was prepared from (2S,3S)-3-(7-phenoxy-2,3-dihydro-1H-indol-1-yl)-3-phenylpropane-1,2-diol. MS (ES) m/z 360 ([M+H]⁺).

In an analogous manner to Example 25, Step 5, (1S,2R)-3-(methylamino)-1-(7-phenoxy-1H-indol-1-yl)-1-phenylpropan-2-ol hydrochloride was prepared from (2S,3S)-3-(7-phenoxy-1H-indol-1-yl)-3-phenylpropane-1,2-diol. MS (ES) m/z 373 ([M+H]⁺); HRMS: calculated for C₂₄H₂₄N₂O₂+H⁺, 373.19105; found (ESI, [M+H]⁺), 373.1912.

### Example 82

### (1S,2R)-3-amino-1-[5-(benzyloxy)-1H-indol-1H-yl]-1-phenylpropan-2-ol hydrochloride

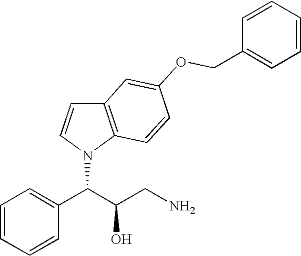

Step 1:

In an analogous manner to Example 1, Step 4, (2S,3S)-3-[5-(benzyloxy)-1H-indol-1-yl]-3-phenylpropane-1,2-diol was prepared from (2S,3S)-3-[5-(benzyloxy)-2,3-dihydro-1H-indol-1-yl]-3-phenylpropane-1,2-diol. MS (ES) m/z 374 [(M+H)⁺].

Step 2:

In an analogous manner to Example 1, Step 5, (2S,3S)-3-(5-(benzyloxy)-1H-indol-1-yl)-2-hydroxy-3-phenylpropyl 4-methylbenzenesulfonate was prepared from (2S,3S)-3-[5-(benzyloxy)-1H-indol-1-yl]-3-phenylpropane-1,2-diol. MS (ES) m/z 528 [(M+H)⁺].

Step 3:

In an analogous manner to Example 1, Step 6, (1S,2R)-3-amino-1-[5-(benzyloxy)-1H-indol-1-yl]-1-phenylpropan-2-ol hydrochloride was prepared from (2S,3S)-3-(5-(benzyloxy)-1H-indol-1-yl)-2-hydroxy-3-phenylpropyl-4-methylbenzenesulfonate, substituting ammonia in methanol solution in place of methylamine in methanol solution. MS (ES) m/z 373 [(M+H)⁺].

### Example 83

### (1S,2R)-1-[5-(benzyloxy)-1H-indol-1-yl]-3-(ethylamino)-1-phenylpropan-2-ol hydrochloride

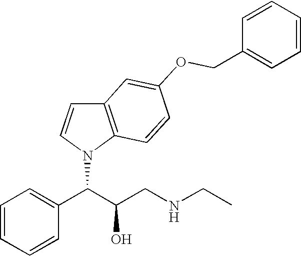

In an analogous manner to Example 1, Step 6, (1S,2R)-1-[5-(benzyloxy)-1H-indol-1-yl]-3-(ethylamino)-1-phenylpropan-2-ol hydrochloride was prepared from (2S,3S)-3-(5-(benzyloxy)-1H-indol-1-yl)-2-hydroxy-3-phenylpropyl 4-methylbenzenesulfonate (from Example 82, Step 2), substituting ethylamine in place of methylamine. MS (ES) m/z 401 [(M+H)⁺].

### Example 84

### (1S,2R)-1-[5-(benzyloxy)-1H-indol-1-yl]-1-phenyl-3-(propylamino)propan-2-ol hydrochloride

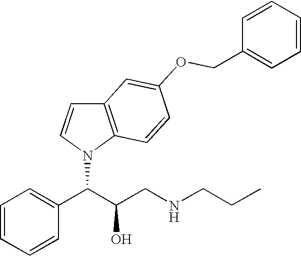

In an analogous manner to Example 1, Step 6, (1S,2R)-1-[5-(benzyloxy)-1H-indol-1-yl]-1-phenyl-3-(propylamino)propan-2-ol hydrochloride was prepared from (2S,3S)-3-(5-(benzyloxy)-1H-indol-1-yl)-2-hydroxy-3-phenylpropyl 4-methylbenzenesulfonate (from Example 82, Step 2), substituting propylamine in place of methylamine. MS (ES) m/z 415 [(M+H)⁺].

### Example 85

### (1S,2R)-1-[5-(benzyloxy)-1H-indol-1-yl]-3-(isopropylamino)-1-phenylpropan-2-ol hydrochloride

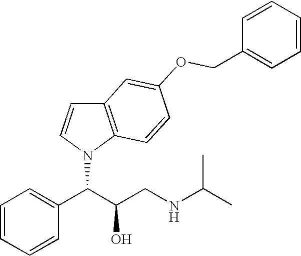

In an analogous manner to Example 1, Step 6, (1S,2R)-1-[5-(benzyloxy)-1H-indol-1-yl]-3-(isopropylamino)-1-phenylpropan-2-ol hydrochloride was prepared from (2S,3S)-3-(5-(benzyloxy)-1H-indol-1-yl)-2-hydroxy-3-phenylpropyl 4-methylbenzenesulfonate (from Example 82, Step 2), substituting isopropylamine in place of methylamine. MS (ES) m/z 415 [(M+H)⁺].

### Example 86

### (1S,2R)-1-[5-(benzyloxy)-1H-indol-1-yl]-3-(dimethylamino)-1-phenylpropan-2-ol hydrochloride

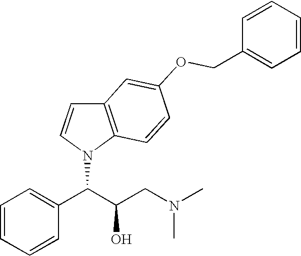

In an analogous manner to Example 1, Step 6, (1S,2R)-1-[5-(benzyloxy)-1H-indol-1-yl]-3-(dimethylamino)-1-phenylpropan-2-ol hydrochloride was prepared from (2S,3S)-3-(5-(benzyloxy)-1H-indol-1-yl)-2-hydroxy-3-phenylpropyl 4-methylbenzenesulfonate (from Example 82, Step 2), substituting N,N-dimethylamine in place of methylamine. MS (ES) m/z 401 [(M+H)⁺].

### Example 87

### (1S,2R)-1-[5-(benzyloxy)-1H-indol-1-yl]-3-[ethyl(methyl)amino]-1-phenylpropan-2-ol hydrochloride

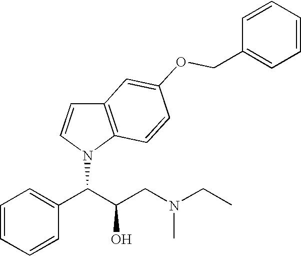

In an analogous manner to Example 1, step 6, (1S,2R)-1-[5-(benzyloxy)-1H-indol-1-yl]-3-[ethyl(methyl)amino]-1-phenylpropan-2-ol hydrochloride was prepared from (2S,3S)-3-(5-(benzyloxy)-1H-indol-1-yl)-2-hydroxy-3-phenylpropyl 4-methylbenzenesulfonate (from Example 82, Step 2), substituting N-ethylmethylamine in place of methylamine. MS (ES) m/z 415 [(M+H)⁺].

### Example 88

### (1S,2R)-1-[5-(benzyloxy)-1H-indol-1-yl]-3-(diethylamino)-1-phenylpropan-2-ol hydrochloride

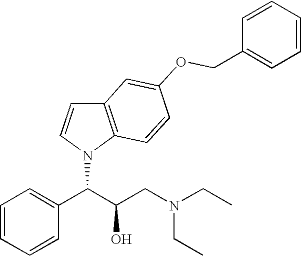

In an analogous manner to Example 1, Step 6, (1S,2R)-1-[5-(benzyloxy)-1H-indol-1-yl]-3-(diethylamino)-1-phenylpropan-2-ol hydrochloride was prepared from (2S,3S)-3-(5-(benzyloxy)-1H-indol-1-yl)-2-hydroxy-3-phenylpropyl 4-methylbenzenesulfonate (from Example 82, Step 2), substituting diethylamine in place of methylamine. MS (ES) m/z 429 [(M+H)⁺].

### Example 89

### (1S,2R)-1-[5-(benzyloxy)-1H-indol-1-yl]-1-phenyl-3-pyrrolidin-1-ylpropan-2-ol hydrochloride

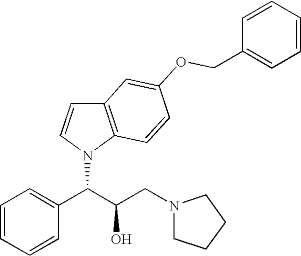

In an analogous manner to Example 1, Step 6, (1S,2R)-1-[5-(benzyloxy)-1H-indol-1-yl]-1-phenyl-3-pyrrolidin-1-ylpropan-2-ol hydrochloride was prepared from (2S,3S)-3-(5-(benzyloxy)-1H-indol-1-yl)-2-hydroxy-3-phenylpropyl 4-methylbenzene sulfonate (from Example 82, Step 2), substituting pyrrolidine in place of methylamine. MS (ES) m/z 427 [(M+H)⁺].

### Example 90

### (1S,2R)-1-[5-(benzyloxy)-1H-indol-1-yl]-1-phenyl-3-piperidin-1-ylpropan-2-ol hydrochloride

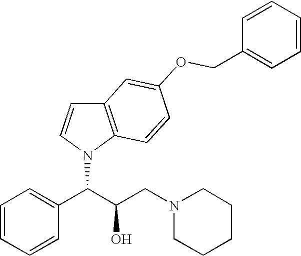

In an analogous manner to Example 1, Step 6, (1S,2R)-1-[5-(benzyloxy)-1H-indol-1-yl]-1-phenyl-3-piperidin-1-ylpropan-2-ol hydrochloride was prepared from (2S,3S)-3-(5-(benzyloxy)-1H-indol-1-yl)-2-hydroxy-3-phenylpropyl 4-methylbenzene sulfonate (from Example 82, Step 2), substituting piperidine in place of methylamine. MS (ES) m/z 441 [(M+H)⁺].

### Example 91

### (1S,2R)-1-[5-(benzyloxy)-1H-indol-1-yl]-3-(4-methylpiperazin-1-yl)-1-phenylpropan-2-ol hydrochloride

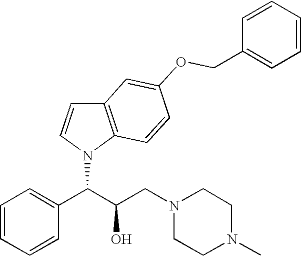

In an analogous manner to Example 1, Step 6, (1S,2R)-1-[5-(benzyloxy)-1H-indol-1-yl]-3-(4-methylpiperazin-1-yl)-1-phenylpropan-2-ol hydrochloride was prepared from (2S,3S)-3-(5-(benzyloxy)-1H-indol-1-yl)-2-hydroxy-3-phenylpropyl 4-methylbenzenesulfonate (from Example 82, Step 2), substituting 1-methylpiperazine in place of methylamine. MS (ES) m/z 456 [(M+H)⁺].

### Example 92

### (1S,2R)-3-(methylamino)-1-phenyl-1-[5-(pyridin-2-ylmethoxy)-1H-indol-1-yl]propan-2-ol hydrochloride

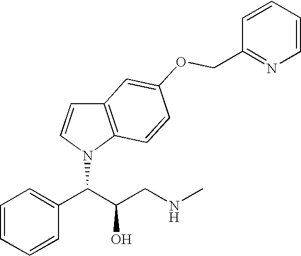

In an analogous manner to Example 5, Step 3, tert-butyl {(2R,3S)-2-hydroxy-3-phenyl-3-[5-(pyridin-2-ylmethoxy)-1H-indol-1-yl]propyl}methylcarbamate was prepared from tert-butyl [(2R,3S)-2-hydroxy-3-(5-hydroxy-1H-indol-1-yl)-3-phenylpropyl]methylcarbamate (from Example 5, step 2), substituting 2-(bromomethyl)pyridine hydrobromide in place of 2-methoxybenzyl chloride. MS (ES) m/z 488 [(M+H)⁺].

In an analogous manner to Example 5, step 4, (1S,2R)-3-(methylamino)-1-phenyl-1-[5-(pyridin-2-ylmethoxy)-1H-indol-1-yl]propan-2-ol hydrochloride was prepared from tert-butyl {(2R,3S)-2-hydroxy-3-phenyl-3-[5-(pyridin-2-ylmethoxy)-1H-indol-1-yl]propyl}methylcarbamate. MS (ES) m/z 388 [(M+H)⁺].

### Example 93

### (1S,2R)-3-(methylamino)-1-phenyl-1-[5-(phenylethynyl)-1H-indol-1-yl]propan-2-ol hydrochloride

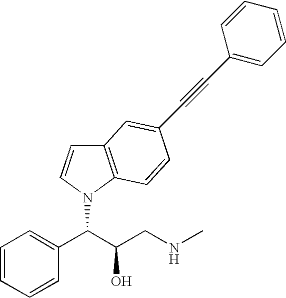

In an analogous manner to Example 1, Step 2, 5-bromoindoline was prepared from 5-bromoindole. MS (ES) m/z 198 [(M+H)⁺].

In an analogous manner to Example 1, Step 3, (2S,3S)-3-(5-bromo-2,3-dihydro-1H-indol-1-yl)-3-phenylpropane-1,2-diol was prepared from [(2R,3R)-3-phenyloxiran-2-yl]methanol (from Example 1, step 1), substituting 5-bromoindoline in place of 5-(benzyloxy)indoline. MS (ES) m/z 348 [(M+H)⁺].

In an analogous manner to Example 1, Step 4, (2S,3S)-3-(5-bromo-1H-indol-1-yl)-3-phenylpropane-1,2-diol was prepared from (2S,3S)-3-(5-bromo-2,3-dihydro-1H-indol-1-yl)-3-phenylpropane-1,2-diol. MS (ESI) m/z 346 [(M+H)⁺].

A mixture of (2S,3S)-3-(5-bromo-1H-indol-1-yl)-3-phenylpropane-1,2-diol (500 mg, 1.44 mmol), phenylacetylene (d 0.930, 0.32 mL, 2.9 mmol), copper (I) iodide (27 mg, 0.14 mmol), potassium carbonate (398 mg, 2.9 mmol) and [1,1′-bis(diphenylphosphino)ferrocene]dichloropalladium (II) (57 mg, 0.007 mmol) in N,N-dimethylformamide (10 mL) was purged with nitrogen for 30 minutes and then heated at 100° C. After 15 hours, the cooled mixture was filtered through Celite and washed with ethyl acetate (30 mL). The filtrate was diluted with ethyl acetate (120 mL), washed with water (5×100 mL) and saturated brine (100 mL), dried over anhydrous sodium sulfate, filtered and concentrated under reduced pressure. The resulting dark oil was dissolved in dichloromethane and pre-adsorbed on silica gel (2.5 g). ISCO CombiFlash Companion chromatography (40 g RediSep silica, 40 mL/min, 30-50% ethyl acetate/hexane) provided (2S,3S)-3-phenyl-3-[5-(phenylethynyl)-1H-indol-1-yl]propane-1,2-diol (452 mg, 85%) as a tan solid. MS (ES) m/z 368 [(M+H)⁺].

In an analogous manner to Example 1, Step 5, (2S,3S)-2-hydroxy-3-phenyl-3-[5-(phenylethynyl)-1H-indol-1-yl]propyl 4-methylbenzenesulfonate was prepared from (2S,3S)-3-phenyl-3-[5-(phenylethynyl)-1H-indol-1-yl]propane-1,2-diol. MS (ES) m/z 522 [(M+H)⁺].

In an analogous manner to Example 1, step 6, (1S,2R)-3-(methylamino)-1-phenyl-1-[5-(phenylethynyl)-1H-indol-1-yl]propan-2-ol hydrochloride was prepared from (2S,3S)-2-hydroxy-3-phenyl-3-[5-(phenylethynyl)-1H-indol-1-yl]propyl 4-methylbenzenesulfonate. MS (ES) m/z 381 [(M+H)⁺].

### Example 94

### (1S,2R)-3-(methylamino)-1-phenyl-1-[5-(2-phenylethyl)-1H-indol-1-yl]propan-2-ol hydrochloride

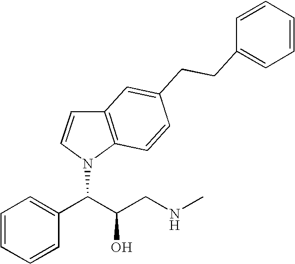

A solution of (2S,3S)-3-phenyl-3-[5-(phenylethynyl)-1H-indol-1-yl]propane-1,2-diol, Example 93, Step 4, (1.2 g, 3.3 mmol) in ethyl acetate (40 mL) was hydrogenated over 10% palladium-on-carbon (0.24 g) at 50 psi. After 24 hours, the reaction mixture was filtered through Celite and washed with ethyl acetate. The filtrate was concentrated under reduced pressure and the residue was dissolved in warm ethyl acetate (<5 mL) and pre-adsorbed on silica gel (3 g). ISCO CombiFlash Companion chromatography (80 g RediSep silica, 60 mL/min, 30-100% ethyl acetate/hexane) provided (2S,3S)-3-phenyl-3-[5-(2-phenylethyl)-1H-indol-1-yl]propane-1,2-diol (0.96 g, 80%) as a light yellow solid. MS (ES) m/z 372 [(M+H)⁺].

In an analogous manner to Example 1, Step 5, (2S,3S)-2-hydroxy-3-phenyl-3-[5-(2-phenylethyl)-1H-indol-1-yl]propyl 4-methylbenzenesulfonate was prepared from (2S,3S)-3-phenyl-3-[5-(2-phenylethyl)-1H-indol-1-yl]propane 1,2-diol. MS (ES) m/z 526 [(M+H)⁺].

In an analogous manner to Example 1, Step 6, (1S,2R)-3-(methylamino)-1-phenyl-1-[5-(2-phenylethyl)-1H-indol-1-yl]propan-2-ol hydrochloride was prepared from (2S,3S)-2-hydroxy-3-phenyl-3-[5-(2-phenylethyl)-1H-indol-1-yl]propyl 4-methylbenzenesulfonate. MS (ES) m/z 385 [(M+H)⁺].

### Example 95

### 1′-[(1S,2R)-3-amino-2-hydroxy-1-phenylpropyl]-6′-fluorospiro[cyclohexane-1,3′-indol]-2′(1′H)-one hydrochloride

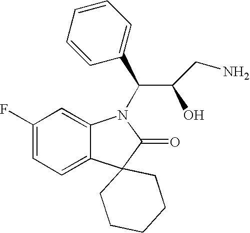

In an analogous manner to Example 27, Step 3, 1′-[(1S,2R)-3-amino-2-hydroxy-1-phenylpropyl]-6′-fluorospiro[cyclohexane-1,3′-indol]-2′(1′H)-one hydrochloride was prepared from 1′-[(1S,2S)-2,3-dihydroxy-1-phenylpropyl]-6′-fluorospiro[cyclohexane-1,3′-indol]-2′(1′H)-one (from Example 29, Step 2), substituting ammonium hydroxide in place of methylamine in ethanol solution. MS (ES) m/z 369.1 ([M+H]⁺); HRMS: calculated for C₂₂H₂₅FN₂O₂+H⁺, 369.19728; found (ESI, [M+H]⁺), 369.1977.

### Example 96

### 1′-[(1S,2R)-3-(ethylamino)-2-hydroxy-1-phenylpropyl]-6′-fluorospiro[cyclohexane-1,3′-indol]-2′(1′H)-one hydrochloride

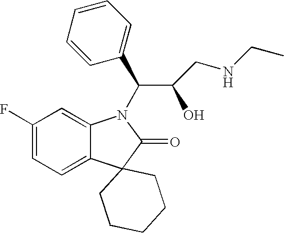

In an analogous manner to Example 27, Step 3, 1′-[(1S,2R)-3-(ethylamino)-2-hydroxy-1-phenylpropyl]-6′-fluorospiro[cyclohexane-1,3′-indol]-2′(1′H)-one hydrochloride was prepared from 1′-[(1S,2S)-2,3-dihydroxy-1-phenylpropyl]-6′-fluorospiro[cyclohexane-1,3′-indol]-2′(1′H)-one (from Example 29, Step 2), substituting ethylamine in place of methylamine in ethanol solution. MS (ES) m/z 397.2 ([M+H]⁺); HRMS: calculated for C₂₄H₂₉FN₂O₂+H⁺, 397.22858; found (ESI, [M+H]⁺), 397.2275.

### Example 97

### 6′-fluoro-1′-[(1S,2R)-2-hydroxy-3-(isopropylamino)-1-phenylpropyl]spiro[cyclohexane-1,3′-indol]-2′(1′H)-one hydrochloride

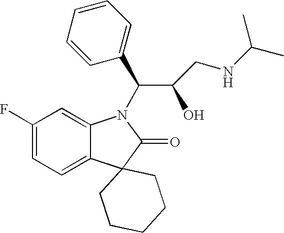

In an analogous manner to Example 27, Step 3, 6′-fluoro-1′-[(1S,2R)-2-hydroxy-3-(isopropylamino)-1-phenylpropyl]spiro[cyclohexane-1,3′-indol]-2′(1′H)-one hydrochloride was prepared from 1′-[(1S,2S)-2,3-dihydroxy-1-phenylpropyl]-6′-fluorospiro[cyclohexane-1,3′-indol]-2′(1′H)-one (from Example 29, Step 2), substituting isopropyl amine in place of methylamine in ethanol solution. MS (ES) m/z 411.2 ([M+H]⁺); HRMS: calculated for C₂₅H₃₁FN₂O₂+H⁺, 411.24423; found (ESI, [M+H]⁺), 411.2413.

### Example 98

### 6′-fluoro-1′-[(1S,2R)-2-hydroxy-1-phenyl-3-(propylamino)propyl]spiro[cyclohexane-1,3′-indol]-2′(1′H)-one hydrochloride

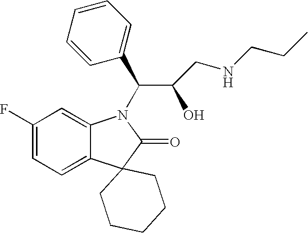

In an analogous manner to Example 27, Step 3, 6′-fluoro-1′-[(1S,2R)-2-hydroxy-1-phenyl-3-(propylamino)propyl]spiro[cyclohexane-1,3′-indol]-2′(1′H)-one hydrochloride was prepared from 1′-[(1S,2S)-2,3-dihydroxy-1-phenylpropyl]-6′-fluorospiro[cyclohexane-1,3′-indol]-2′(1′H)-one (from Example 29, Step 2), substituting propylamine in place of methylamine in ethanol solution. MS (ES) m/z 411.2 ([M+H]⁺); HRMS: calculated for C₂₅H₃₁FN₂O₂+H⁺, 411.24423; found (ESI, [M+H]⁺), 411.2413.

### Example 99

### 1′-[(1S,2R)-3-amino-2-hydroxy-1-phenylpropyl]-5′-fluorospiro[cyclohexane-1,3′-indol]-2′(1′H)-one hydrochloride

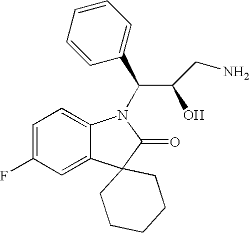

In an analogous manner to Example 27, Step 3, 1′-[(1S,2R)-3-amino-2-hydroxy-1-phenylpropyl]-5′-fluorospiro[cyclohexane-1,3′-indol]-2′(1′H)-one hydrochloride was prepared from 1′-[(1S,2S)-2,3-dihydroxy-1-phenylpropyl]-5′-fluorospiro[cyclohexane-1,3′-indol]-2′(1′H)-one (from Example 30, Step 2), substituting ammonium hydroxide in place of methylamine in ethanol solution. MS (ES) m/z 369.1 ([M+H]⁺); HRMS: calculated for C₂₂H₂₅FN₂O₂+H⁺, 369.19728; found (ESI, [M+H]⁺), 369.1982.

### Example 100

### 1′-[(1S,2R)-3-(ethylamino)-2-hydroxy-1-phenylpropyl]-5′-fluorospiro[cyclohexane-1,3′-indol]-2′(1′H)-one hydrochloride

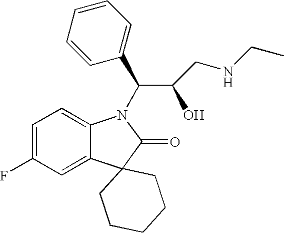

In an analogous manner to Example 27, Step 3, 1′-[(1S,2R)-3-(ethylamino)-2-hydroxy-1-phenylpropyl]-5′-fluorospiro[cyclohexane-1,3′-indol]-2′(1′H)-one hydrochloride was prepared from 1′-[(1S,2S)-2,3-dihydroxy-1-phenylpropyl]-5′-fluorospiro[cyclohexane-1,3′-indol]-2′(1′H)-one (from Example 30, Step 2), substituting ethylamine in place of methylamine in ethanol solution. MS (ES) m/z 397.2 ([M+H]⁺); HRMS: calculated for C₂₄H₂₉FN₂O₂+H⁺, 397.22858; found (ESI, [M+H]⁺), 397.229.

### Example 101

### 5′-fluoro-1′-[(1S,2R)-2-hydroxy-3-(isopropylamino)-1-phenylpropyl]spiro[cyclohexane-1,3′-indol]-2′(1′H)-one hydrochloride

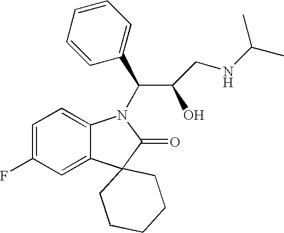

In an analogous manner to Example 27, Step 3, 5′-fluoro-1′-[(1S,2R)-2-hydroxy-3-(isopropylamino)-1-phenylpropyl]spiro[cyclohexane-1,3′-indol]-2′(1′H)-one hydrochloride was prepared from 1′-[(1S,2S)-2,3-dihydroxy-1-phenylpropyl]-5′-fluorospiro[cyclohexane-1,3′-indol]-2′(1′H)-one (from Example 30, Step 2), substituting isopropylamine in place of methylamine in ethanol solution. MS (ES) m/z 411.2 ([M+H]⁺); HRMS: calculated for C₂₅H₃₁FN₂O₂+H⁺, 411.24423; found (ESI, [M+H]⁺), 411.2433.

### Example 102

### 5′-fluoro-1′-[(1S,2R)-2-hydroxy-1-phenyl-3-(propylamino)propyl]spiro[cyclohexane-1,3′-indol]-2′(1′H)-one hydrochloride

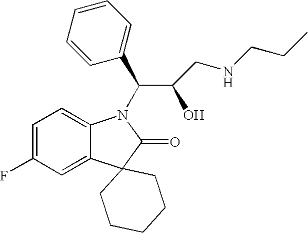

In an analogous manner to Example 27, Step 3, 5′-fluoro-1′-[(1S,2R)-2-hydroxy-1-phenyl-3-(propylamino)propyl]spiro[cyclohexane-1,3′-indol]-2′(1′H)-one hydrochloride was prepared from 1′-[(1S,2S)-2,3-dihydroxy-1-phenylpropyl]-5′-fluorospiro[cyclohexane-1,3′-indol]-2′(1′H)-one (from Example 30, Step 2), substituting propylamine in place of methylamine in ethanol solution. MS (ES) m/z 411.2; HRMS: calculated for C₂₅H₃₁FN₂O₂+H⁺, 411.24423; found (ESI, [M+H]⁺), 411.2438.

### Example 103

### 1′-[(1S,2R)-3-(dimethylamino)-2-hydroxy-1-phenylpropyl]-5′-fluorospiro[cyclohexane-1,3′-indol]-2′(1′H)-one hydrochloride

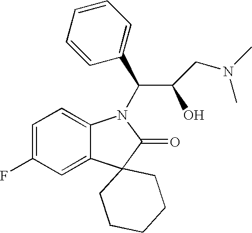

In an analogous manner to Example 27, Step 3, 1′-[(1S,2R)-3-(dimethylamino)-2-hydroxy-1-phenylpropyl]-5′-fluorospiro[cyclohexane-1,3′-indol]-2′(1′H)-one hydrochloride was prepared from 1′-[(1S,2S)-2,3-dihydroxy-1-phenylpropyl]-5′-fluorospiro[cyclohexane-1,3′-indol]-2′(1′H)-one (from Example 30, Step 2), substituting dimethylamine in place of methylamine in ethanol solution. MS (ES) m/z 397.2 ([M+H]⁺); HRMS: calculated for C₂₄H₂₉FN₂O₂+H⁺, 397.22858; found (ESI, [M+H]⁺), 397.2283.

### Example 104

### 5′-fluoro-1′-[(1S,2R)-2-hydroxy-3-morpholin-4-yl-1-phenylpropyl]spiro[cyclohexane-1,3′-indol]-2′(1′H)-one hydrochloride

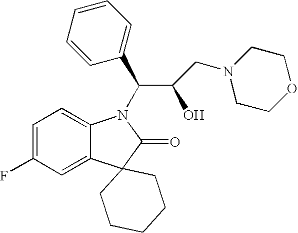

In an analogous manner to Example 27, Step 3, 5′-fluoro-1′-[(1S,2R)-2-hydroxy-3-morpholin-4-yl-1-phenylpropyl]spiro[cyclohexane-1,3′-indol]-2′(1′H)-one hydrochloride was prepared from 1′-[(1S,2S)-2,3-dihydroxy-1-phenylpropyl]-5′-fluorospiro[cyclohexane-1,3′-indol]-2′(1′H)-one (from Example 30, Step 2), substituting morpholine in place of methylamine in ethanol solution. MS (ES) m/z 439.1 ([M+H]⁺); HRMS: calculated for C₂₆H₃₁FN₂O₃+H⁺, 439.23915; found (ESI, [M+H]⁺), 439.2392.

### Example 105

### 1′-[(1S,2R)-2-hydroxy-3-(methylamino)-1-phenylpropyl]-5′-methoxyspiro[cyclohexane-1,3′-indol]-2′(1′H)-one hydrochloride

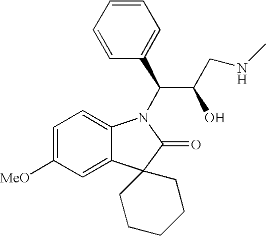

In an analogous manner to Example 27, Step 1, 5′-methoxyspiro[cyclohexane-1,3′-indol]-2′(1′H)-one was prepared from 5-methoxyoxindole.

In an analogous manner to Example 27, Step 2, 1′-[(1S,2S)-2,3-dihydroxy-1-phenylpropyl]-5′-methoxyspiro[cyclohexane-1,3′-indol]-2′(1′H)-one was prepared from 5′-methoxyspiro[cyclohexane-1,3′-indol]-2′(1′H)-one.

In an analogous manner to Example 27, Step 3, 1′-[(1S,2R)-2-hydroxy-3-(methylamino)-1-phenylpropyl]-5′-methoxyspiro[cyclohexane-1,3′-indol]-2′(1′H)-one hydrochloride was prepared from 1′-[(1S,2S)-2,3-dihydroxy-1-phenylpropyl]-5′-methoxyspiro[cyclohexane-1,3′-indol]-2′(1′H)-one. MS (ES) m/z 395.2 ([M+H]⁺); HRMS: calculated for C₂₄H₃₀N₂O₃+H⁺, 395.23292; found (ESI, [M+H]⁺), 395.2313.

### Example 106

### 1′-[(1S,2R)-2-hydroxy-3-(methylamino)-1-phenylpropyl]-6′-methoxyspiro[cyclohexane-1,3′-indol]-2′(1′H)-one hydrochloride

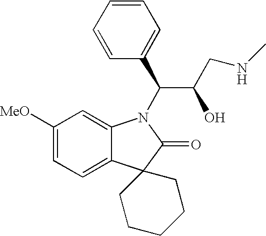

In an analogous manner to Example 27, Step 1, 6′-methoxyspiro[cyclohexane-1,3′-indol]-2′(1′H)-one was prepared from 6-methoxyoxindole.

In an analogous manner to Example 27, Step 2, 1′-[(1S,2S)-2,3-dihydroxy-1-phenylpropyl]-6′-methoxyspiro[cyclohexane-1,3′-indol]-2′(1′H)-one was prepared from 6′-methoxyspiro[cyclohexane-1,3′-indol]-2′(1′H)-one.

In an analogous manner to Example 27, Step 3, 1′-[(1S,2R)-2-hydroxy-3-(methylamino)-1-phenylpropyl]-6′-methoxyspiro[cyclohexane-1,3′-indol]-2′(1′H)-one hydrochloride was prepared from 1′-[(1S,2S)-2,3-dihydroxy-1-phenylpropyl]-6′-methoxyspiro[cyclohexane-1,3′-indol]-2′(1′H)-one. MS (ES) m/z 395.1 ([M+H]⁺); HRMS: calculated for C₂₄H₃₀N₂O₃+H⁺, 395.23292; found (ESI, [M+H]⁺), 395.2317.

### Example 107

### 1′-[(1S,2R)-2-hydroxy-3-(methylamino)-1-phenylpropyl]-2′-oxo-1′,2′-dihydrospiro[cyclohexane-1,3′-indole]-5′-carbonitrile hydrochloride

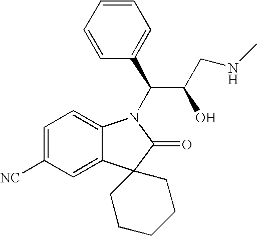

In an analogous manner to Example 27, Step 1, 2′-oxo-1′,2′-dihydrospiro[cyclohexane-1,3′-indole]-5′-carbonitrile was prepared from 5-cyano-oxindole. MS (ES) m/z 225.0 ([M−H]⁻).

In an analogous manner to Example 27, Step 2, 1′-[(1S,2S)-2,3-dihydroxy-1-phenylpropyl]-2′-oxo-1′,2′-dihydrospiro[cyclohexane-1,3′-indole]-5′-carbonitrile was prepared from 2′-oxo-1′,2′-dihydrospiro[cyclohexane-1,3′-indole]-5′-carbonitrile. MS (ES) m/z 377.1 ([M+H]⁺).

In an analogous manner to Example 27, Step 3, 1′-[(1S,2R)-2-hydroxy-3-(methylamino)-1-phenylpropyl]-2′-oxo-1′,2′-dihydrospiro[cyclohexane-1,3′-indole]-5′-carbonitrile hydrochloride was prepared from 1′-[(1S,2S)-2,3-dihydroxy-1-phenylpropyl]-2′-oxo-1′,2′-dihydrospiro[cyclohexane-1,3′-indole]-5′-carbonitrile. MS (ES) m/z 390.1 ([M+H]⁺); HRMS: calculated for C₂₄H₂₇N₃O₂+H⁺, 390.21760; found (ESI, [M+H]⁺), 390.2184.

### Example 108

### 1′-[(1S,2R)-2-hydroxy-3-(methylamino)-1-phenylpropyl]-2′-oxo-1′,2′-dihydrospiro[cyclohexane-1,3′-indole]-6′-carbonitrile hydrochloride

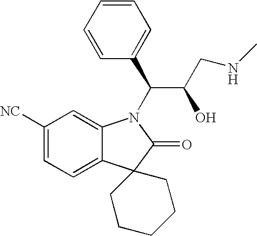

In an analogous manner to Example 27, Step 1, 2′-oxo-1′,2′-dihydrospiro[cyclohexane-1,3′-indole]-6′-carbonitrile was prepared from 6-cyano-oxindole. MS (ES) m/z 225.0 ([M−H]⁻).

In an analogous manner to Example 27, Step 2, 1′-[(1S,2S)-2,3-dihydroxy-1-phenylpropyl]-2′-oxo-1′,2′-dihydrospiro[cyclohexane-1,3′-indole]-6′-carbonitrile was prepared from 2′-oxo-1′,2′-dihydrospiro[cyclohexane-1,3′-indole]-6′-carbonitrile. MS (ES) m/z 377.1 ([M+H]⁺).

In an analogous manner to Example 27, Step 3, 1′-[(1S,2R)-2-hydroxy-3-(methylamino)-1-phenylpropyl]-2′-oxo-1′,2′-dihydrospiro[cyclohexane-1,3′-indole]-6′-carbonitrile hydrochloride was prepared from 1′-[(1S,2S)-2,3-dihydroxy-1-phenylpropyl]-2′-oxo-1′,2′-dihydrospiro[cyclohexane-1,3′-indole]-6′-carbonitrile. MS (ES) m/z 390.2 ([M+H]⁺); HRMS: calculated for C₂₄H₂₇N₃O₂+H⁺, 390.21760; found (ESI, [M+H]⁺), 390.2186.

### Example 109

### 4′,5′-difluoro-1′-[(1S,2R)-2-hydroxy-3-(methylamino)-1-phenylpropyl]spiro[cyclohexane-1,3′-indol]-2′(1′H)-one hydrochloride

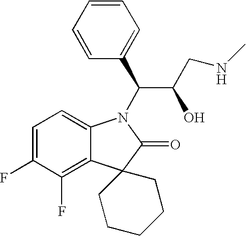

In an analogous manner to Example 27, Step 1, 4′,5′-difluorospiro[cyclohexane-1,3′-indol]-2′(1′H)-one was prepared from 4,5-difluoro-oxindole. MS (ES) m/z 238.1 ([M+H]⁺).

In an analogous manner to Example 27, Step 2, 1′-[(1S,2S)-2,3-dihydroxy-1-phenylpropyl]-4′,5′-difluorospiro[cyclohexane-1,3′-indol]-2′(1′H)-one was prepared from 4′,5′-difluorospiro[cyclohexane-1,3′-indol]-2′(1′H)-one. MS (ES) m/z 388.1 ([M+H]⁺).

In an analogous manner to Example 27, Step 3, 4′,5′-difluoro-1′-[(1S,2R)-2-hydroxy-3-(methylamino)-1-phenylpropyl]spiro[cyclohexane-1,3′-indol]-2′(1′H)-one hydrochloride was prepared from 1′-[(1S,2S)-2,3-dihydroxy-1-phenylpropyl]-4′,5′-difluorospiro[cyclohexane-1,3′-indol]-2′(1′H)-one. MS (ES) m/z 401.2 ([M+H]⁺); HRMS: calculated for C₂₃H₂₆F₂N₂O₂+H⁺, 401.20351; found (ESI, [M+H]⁺), 401.204.

### Example 110

### 7′-fluoro-1′-[(1S,2R)-1-(3-fluorophenyl)-2-hydroxy-3-(methylamino)propyl]spiro[cyclohexane-1,3′-indol]-2′(1′H)-one hydrochloride

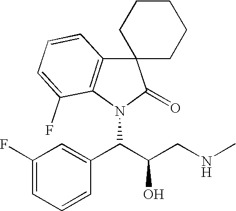

In an analogous manner to Example 62, Step 4, 7′-fluorospiro[cyclohexane-1,3′-indol]-2′(1′H)-one was prepared from 7-fluoro-1,3-dihydro-2H-indol-2-one (from Example 62, Step 3), substituting 1,5-dibromopentane in place of methyl iodide. MS (ES) m/z 220 [(M+H)⁺].

In an analogous manner to Example 62, Step 5, 7′-fluoro-1′-((1S,2S)-1-(3-fluorophenyl)-2,3-dihydroxypropyl)spiro[cyclohexane-1,3′-indolin]-2′-one was prepared from 7′-fluorospiro[cyclohexane-1,3′-indol]-2′(1′H)-one, substituting [(2R,3R)-3-(3-fluorophenyl)oxiran-2-yl]methanol (from Example 24, Step 1) in place of [(2R,3R)-3-(3,5-difluorophenyl)oxiran-2-yl]methanol. MS (ES) m/z 388 [(M+H)⁺].

In an analogous manner to Example 62, Step 6, 7′-fluoro-1′-[(1S,2R)-1-(3-fluorophenyl)-2-hydroxy-3-(methylamino)propyl]spiro[cyclohexane-1,3′-indol]-2′(1′H)-one hydrochloride was prepared from 7′-fluoro-1′-((1S,2S)-1-(3-fluorophenyl)-2,3-dihydroxypropyl)spiro[cyclohexane-1,3′-indolin]-2′-one. MS (ES) m/z 401 [(M+H)⁺].

### Example 111

### 1′-[(1S,2R)-1-(3-chlorophenyl)-2-hydroxy-3-(methylamino)propyl]-6′-fluorospiro[cyclohexane-1,3′-indol]-2′(1′H)-one hydrochloride

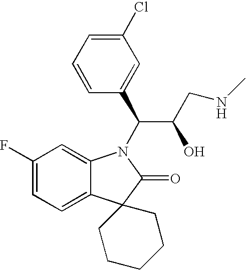

In an analogous manner to Example 27, Step 2, 1′-[(1S,2S)-1-(3-chlorophenyl)-2,3-dihydroxypropyl]-6′-fluorospiro[cyclohexane-1,3′-indol]-2′(1′-one was prepared from 6′-fluorospiro[cyclohexane-1,3′-indol]-2′(1′H)-one (from Example 29, Step 1) and [(2R,3R)-3-(3-chlorophenyl)oxiran-2-yl]methanol (from Example 69, Step 5). MS (ES) m/z 403.9 ([M+H]⁺).

In an analogous manner to Example 27, Step 3, 1′-[(1S,2R)-1-(3-chlorophenyl)-2-hydroxy-3-(methylamino)propyl]-6′-fluorospiro[cyclohexane-1,3′-indol]-2′(1′H)-one hydrochloride was prepared from 1′-[(1S,2S)-1-(3-chlorophenyl)-2,3-dihydroxypropyl]-6′-fluorospiro[cyclohexane-1,3′-indol]-2′(1′H)-one. MS (ES) m/z 417.1 ([M+H]⁺); HRMS: calculated for C₂₃H₂₆ClFN₂O₂+H⁺, 417.17396; found (ESI, [M+H]⁺), 417.1739.

### Example 112

### 1-[(1S,2R)-1-(3-chloro-5-fluorophenyl)-2-hydroxy-3-(methylamino)propyl]-7-fluoro-3,3-dimethyl-1,3-dihydro-2H-indol-2-one hydrochloride

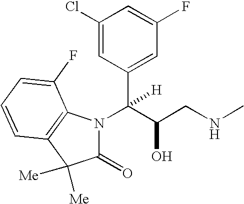

7-Fluoro-3,3-dimethyl-1,3-dihydro-2H-indol-2-one (0.52 9, 3.0 mmol, from Example 69, Step 4) was dissolved in N,N-dimethylformamide (3 mL) and sodium hydride (0.17 g, 4.4 mmol, 60% wt suspension in mineral oil) was added in portions over 15 minutes and the mixture was stirred an additional 30 minutes. In a separate flask, [(2R,3R)-3-(3-fluoro-5-chlorophenyl)oxiran-2-yl]methanol (1.2 g, 5.9 mmol, from Example 70, Step 3) was dissolved in N,N-dimethylformamide (3 mL) and titanium isopropoxide (1.76 mL, 5.9 mmol) was added and the mixture was stirred 30 minutes. The titanium isopropoxide/epoxide solution was then added to the solution of oxindole sodium salt dropwise and the mixture was stirred at room temperature for 24 hours. The mixture was then carefully quenched with 2 N aqueous hydrochloric acid and diluted with 200 mL of 2 N aqueous hydrochloric acid (use of hydrochloric acid is essential to prevent precipitation of titanium salts and subsequent emulsification). The mixture was extracted with ethyl acetate and then the organic layers were combined, washed with water, and saturated brine, dried over anhydrous magnesium sulfate, filtered, and concentrated under reduced pressure. The crude product was purified via Isco chromatography (Redisep, silica, gradient 20% to 100% ethyl acetate in hexane) to afford 1.01 g of 1-[(1S,2S)-1-(3-chloro-5-fluorophenyl)-2,3-dihydroxypropyl]-7-fluoro-3,3-dimethyl-1,3-dihydro-2H-indol-2-one as a sticky oil of 82% purity. MS (ES) m/z 382.0 ([M+H]⁺).

1-[(1S,2S)-1-(3-chloro-5-fluorophenyl)-2,3-dihydroxypropyl]-7-fluoro-3,3-dimethyl-1,3-dihydro-2H-indol-2-one (1.0 g, 2.6 mmol) was dissolved in pyridine (3 mL) and p-toluenesulfonyl chloride (0.55 g, 2.9 mmol) was added and the mixture stirred for 4 hours. The reaction mixture then was diluted with diethyl ether and washed with water, 2 N aqueous hydrochloric acid, saturated copper sulfate, 2 N aqueous hydrochloric acid, and saturated brine. The organic layer was separated, dried over anhydrous magnesium sulfate, filtered, and concentrated under reduced pressure. The crude product was immediately dissolved in methylamine solution (8.0 M in ethanol, 20 mL) and stirred for 16 hours. The mixture was concentrated under reduced pressure and purified via chromatography (silica, 5% methanol saturated with ammonia in chloroform) to give 1-[(1S,2R)-1-(3-chloro-5-fluorophenyl)-2-hydroxy-3-(methylamino)propyl]-7-fluoro-3,3-dimethyl-1,3-dihydro-2H-indol-2-one (0.098 g) as a colorless oil. The freebase was dissolved in methanol (10 mL) and treated with hydrogen chloride solution (1.0 M in diethyl ether, 1.0 equivalent). The mixture was concentrated under vacuum then dissolved in 10 mL of water and lyophilized to give 87 mg of 1-[(1S,2R)-1-(3-chloro-5-fluorophenyl)-2-hydroxy-3-(methylamino)propyl]-7-fluoro-3,3-dimethyl-1,3-dihydro-2H-indol-2-one hydrochloride. MS (ES) m/z 395.0 ([M+H]⁺). HPLC purity 100.0% at 210-370 nm, 8.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min.

### Example 113

### (1S,2R)-1-(3-chloro-5-fluorophenyl)-1-(2,3-dihydro-1H-indol-1-yl)-3-(methylamino)propan-2-ol hydrochloride

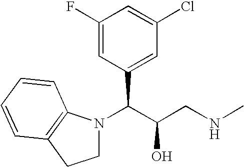

In an analogous manner to Example 25, Step 5, (1S,2R)-1-(3-chloro-5-fluorophenyl)-1-(2,3-dihydro-1H-indol-1-yl)-3-(methylamino)propan-2-ol hydrochloride was prepared from (2S,3S)-3-(3-chloro-5-fluorophenyl)-3-(2,3-dihydro-1H-indol-1-yl)propane-1,2-diol (from Example 70, Step 4) as a white powder. HRMS: calculated for C₁₈H₂₀ClFN₂O+H⁺, 335.1321; found (ESI, [M+H]⁺), 335.1318.

### Example 114

### (1S,2R)-1-(3-chloro-5-fluorophenyl)-1-(7-fluoro-3,3-dimethyl-2,3-dihydro-1H-indol-1-yl)-3-(methylamino)propan-2-ol hydrochloride

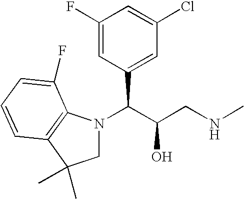

In an analogous manner to Example 52, Step 2, 7-fluoro-3,3-dimethylindoline was prepared from 7-fluoro-3,3-dimethyl-1,3-dihydro-2H-indol-2-one (from Example 69, Step 4) as a white powder. MS (ES) m/z 166.1 ([M+H]⁺); HRMS: calculated for C₁₀H₁₂FN+H⁺, 166.1032; found (ESI, [M+H]⁺), 166.1040.

In an analogous manner to Example 1, Step 3, (2S,3S)-3-(3-chloro-5-fluorophenyl)-3-(7-fluoro-3,3-dimethyl-2,3-dihydro-1H-indol-1-yl)propane-1,2-diol was prepared from 7-fluoro-3,3-dimethylindoline and [(2R,3R)-3-(3-chloro-5-fluorophenyl)oxiran-2-yl]methanol (from Example 70, Step 3) as an amber gum. MS (ESI) m/z 368.1 ([M+H]⁺); HRMS: calculated for C₁₉H₂₀ClF₂NO₂+H⁺, 368.1223; found (ESI, [M+H]⁺), 368.1234.

In an analogous manner to Example 25, Step 5, (1S,2R)-1-(3-chloro-5-fluorophenyl)-1-(7-fluoro-3,3-dimethyl-2,3-dihydro-1H-indol-1-yl)-3-(methylamino)propan-2-ol hydrochloride was prepared from (2S,3S)-3-(3-chloro-5-fluorophenyl)-3-(7-fluoro-3,3-dimethyl-2,3-dihydro-1H-indol-1-yl)propane-1,2-diol as an ivory solid. MS (ES) m/z 381.1 ([M+H]⁺); HRMS: calculated for C₂₀H₂₃ClF₂N₂O+H⁺, 381.1540; found (ESI, [M+H]⁺), 381.1533.

### Example 115

### (1S,2R)-1-(3-chloro-5-fluorophenyl)-1-(3,3-dimethyl-2,3-dihydro-1H-indol-1-yl)-3-(methylamino)propan-2-ol hydrochloride

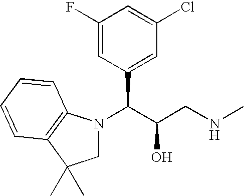

In an analogous manner to Example 1, Step 3, (2S,3S)-3-(3-chloro-5-fluorophenyl)-3-(3,3-dimethyl-2,3-dihydro-1H-indol-1-yl)propane-1,2-diol was prepared from 3,3-dimethylindoline and [(2R,3R)-3-(3-chloro-5-fluorophenyl)oxiran-2-yl]methanol (from Example 70, step 3) as a light brown gum. MS (ESI) m/z 350.0 ([M+H]⁺); HRMS: calculated for C₁₉H₂₁ClFNO₂+H⁺, 350.1318; found (ESI, [M+H]⁺), 350.1293.

In an analogous manner to Example 25, Step 5, (1S,2R)-1-(3-chloro-5-fluorophenyl)-1-(3,3-dimethyl-2,3-dihydro-1H-indol-1-yl)-3-(methylamino)propan-2-ol hydrochloride was prepared from (2S,3S)-3-(3-chloro-5-fluorophenyl)-3-(3,3-dimethyl-2,3-dihydro-1H-indol-1-yl)propane-1,2-diol as a white powder. MS (ES) m/z 363.1 ([M+H]⁺); HRMS: calculated for C₂₀H₂₄ClFN₂O+H⁺, 363.1634; found (ESI, [M+H]⁺), 363.1622.

### Example 116

### 7′-fluoro-1′-[(1S,2R)-1-(3-fluorophenyl)-2-hydroxy-3-(methylamino)propyl]spiro[cyclobutane-1,3′-indol]-2′(1′H)-one hydrochloride

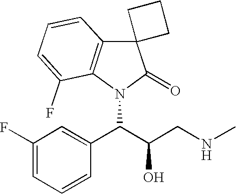

In an analogous manner to Example 62, Step 4, 7′-fluorospiro[cyclobutane-1,3′-indol]-2′(1′H)-one was prepared from 7-fluoro-1,3-dihydro-2H-indol-2-one (from Example 62, Step 3), substituting 1,3-dibromopropane in place of methyl iodide. MS (ES) m/z 192 [(M+H)⁺].

In an analogous manner to Example 62, Step 5, 7′-fluoro-1′-((1S,2S)-1-(3-fluorophenyl)-2,3-dihydroxypropyl)spiro[cyclobutane-1,3′-indolin]-2′-one was prepared from 7′-fluorospiro[cyclobutane-1,3′-indol]-2′(1′H)-one, substituting [(2R,3R)-3-(3-fluorophenyl)oxiran-2-yl]methanol (from Example 24, Step 1) in place of [(2R,3R)-3-(3,5-difluorophenyl)oxiran-2-yl]methanol. MS (ES) m/z 360 [(M+H)⁺].

In an analogous manner to Example 62, step 6, 7′-fluoro-1′-[(1S,2R)-1-(3-fluorophenyl)-2-hydroxy-3-(methylamino)propyl]spiro[cyclobutane-1,3′-indol]-2′(1′H)-one hydrochloride was prepared from 7′-fluoro-1′-((1S,2S)-1-(3-fluorophenyl)-2,3-dihydroxypropyl)spiro[cyclobutane-1,3′-indolin]-2′-one. MS (ES) m/z 373 [(M+H)⁺].

### Example 117

### 7′-fluoro-1′-[(1S,2R)-1-(3-fluorophenyl)-2-hydroxy-3-(methylamino)propyl]spiro[cyclopentane-1,3′-indol]-2′(1′H)-one hydrochloride

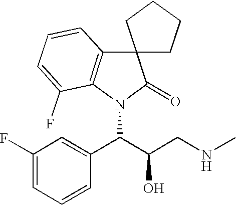

In an analogous manner to Example 62, Step 4, 7′-fluorospiro[cyclopentane-1,3′-indol]-2′(1′H)-one was prepared from 7-fluoro-1,3-dihydro-2H-indol-2-one (from Example 62, Step 3), substituting 1,4-dibromobutane in place of methyl iodide. MS (ES) m/z 206 [(M+H)⁺].

In an analogous manner to Example 62, Step 5, 7′-fluoro-1′-((1S,2S)-1-(3-fluorophenyl)-2,3-dihydroxypropyl)spiro[cyclopentane-1,3′-indolin]-2′-one was prepared from 7′-fluorospiro[cyclopentane-1,3′-indol]-2′(1′H)-one, substituting [(2R,3R)-3-(3-fluorophenyl)oxiran-2-yl]methanol (from Example 24, Step 1) in place of [(2R,3R)-3-(3,5-difluorophenyl)oxiran-2-yl]methanol. MS (ES) m/z 374 [(M+H)⁺].

In an analogous manner to Example 62, Step 6, 7′-fluoro-1′-[(1S,2R)-1-(3-fluorophenyl)-2-hydroxy-3-(methylamino)propyl]spiro[cyclopentane-1,3′-indol]-2′(1′H)-one hydrochloride was prepared from 7′-fluoro-1′-((1S,2S)-1-(3-fluorophenyl)-2,3-dihydroxypropyl)spiro[cyclopentane-1,3′-indolin]-2′-one. MS (ES) m/z 387 [(M+H)⁺].

### Example 118

### 6-fluoro-1-[(1S,2R)-1-(3-fluorophenyl)-2-hydroxy-3-(methylamino)propyl]-3,3-dimethyl-1,3-dihydro-2H-indol-2-one hydrochloride

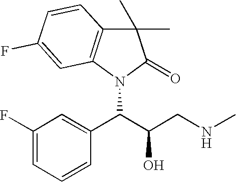

To a hexanes-washed (2×) suspension of sodium hydride (60% in oil, 14 g, 350 mmol) in dimethyl sulfoxide (300 mL) was added dimethyl malonate (46 g, 350 mmol) dropwise at 23° C. The reaction mixture was heated at 100° C. for 45 minutes, then cooled to 23° C. and 2,5-difluoronitrobenzene (25 g, 160 mmol) was added. The mixture was stirred at 23° C. for 30 minutes, then heated at 100° C. for 1 hour. The cooled mixture was poured into a mixture of saturated aqueous ammonium chloride (1.2 L), ethyl acetate (250 mL) and hexanes (250 mL). The organic phase was separated and washed with saturated aqueous ammonium chloride (500 mL), water (3×500 mL) and saturated brine (500 mL), and dried over anhydrous magnesium sulfate. Concentration under reduced pressure gave an oily yellow solid (47 g) that was recrystallized from boiling 20% ethyl acetate-hexanes (ca. 300 mL) to provide dimethyl (4-fluoro-2-nitrophenyl)malonate (35 g, 81%) as shiny white prisms. MS (ES) m/z 270 [(M−H)⁻].

Dimethyl (4-fluoro-2-nitrophenyl)malonate (5.0 g, 18 mmol), lithium chloride (1.6 g, 38 mmol) and water (0.33 g, 18 mmol) were combined in dimethyl sulfoxide (100 mL) and heated at 100° C. After 21 hours, the cooled solution was poured into a stirred mixture of saturated brine (200 mL) and ethyl acetate (200 mL). The phases were separated and the aqueous phase was extracted with ethyl acetate (200 mL). The combined organic extracts were washed with saturated brine (2×200 mL), dried over anhydrous sodium sulfate, filtered and concentrated under reduced pressure to give a dark oil (4.0 g) that was dissolved in dichloromethane and pre-adsorbed on silica gel (10 g). Flash column chromatography (silica 190 g, 5%, 10%, 20% ethyl acetate/hexanes) provided methyl (4-fluoro-2-nitrophenyl)acetate (2.1 g, 54%) as a yellow oil. MS (ES) m/z 212 [(M−H)⁻].

Methyl (4-fluoro-2-nitrophenyl)acetate (7.1 g, 33 mmol) and iron powder (7.4 g, 130 mmol) were combined in glacial acetic acid (65 mL) and heated at 100° C. After 2 hours, the cooled mixture was concentrated under reduced pressure. The residue was dissolved in hot ethyl acetate (100 mL), filtered through Celite and washed with hot ethyl acetate (100 mL). The filtrate was washed with 1 N aqueous hydrochloric acid (3×100 mL) and saturated brine (100 mL), dried over anhydrous sodium sulfate, filtered and concentrated under reduced pressure to give a brown solid. Trituration with 5% ethyl acetate-hexanes (100 mL) provided 6-fluoro-1,3-dihydro-2H-indol-2-one (4.8 g, 96%) as a tan solid. MS (ES) m/z 150 [(M−H)⁻].

In an analogous manner to Example 62, Step 4, 6-fluoro-3,3-dimethyl-1,3-dihydro-2H-indol-2-one was prepared from 6-fluoro-1,3-dihydro-2H-indol-2-one. MS (ES) m/z 180 [(M+H)⁺].

In an analogous manner to Example 62, Step 5, 6-fluoro-1-((1S,2S)-1-(3-fluorophenyl)-2,3-dihydroxypropyl)-3,3-dimethylindolin-2-one was prepared from 6-fluoro-3,3-dimethyl-1,3-dihydro-2H-indol-2-one, substituting [(2R,3R)-3-(3-fluorophenyl)oxiran-2-yl]methanol (from Example 24, step 1) in place of [(2R,3R)-3-(3,5-difluorophenyl)oxiran-2-yl]methanol. MS (ES) m/z 348 [(M+H)⁺].

A solution of 6-fluoro-1-((1S,2S)-1-(3-fluorophenyl)-2,3-dihydroxy propyl)-3,3-dimethylindolin-2-one (0.32 g, 0.92 mmol) in tetrahydrofuran (4.5 mL) was treated with triphenylphosphine (0.30 g, 1.1 mmol) at 23° C. When a solution had formed, N-chlorosuccinimide (0.15 g, 1.1 mmol) was added. After a further 1 hour, the reaction solution was concentrated under vacuum to a small volume and pre-adsorbed on silica gel (1 g). ISCO CombiFlash Companion chromatography (12 g RediSep silica, 30 mL/min, 0-30% ethyl acetate/hexane) provided 1-((1S,2S)-3-chloro-1-(3-fluorophenyl)-2-hydroxypropyl)-6-fluoro-3,3-dimethylindolin-2-one (0.12 g, 35%) as a clear, almost colorless oil. MS (ES) m/z 366 [(M+H)⁺].

In an analogous manner to Example 1, Step 6, 6-fluoro-1-[(1S,2R)-1-(3-fluorophenyl)-2-hydroxy-3-(methylamino)propyl]-3,3-dimethyl-1,3-dihydro-2H-indol-2-one hydrochloride was prepared from 1-((1S,2S)-3-chloro-1-(3-fluorophenyl)-2-hydroxypropyl)-6-fluoro-3,3-dimethylindolin-2-one. MS (ES) m/z 361 [(M+H)⁺].

### Example 119

### (1S,2R)-1-(7-Fluoro-2,3-dihydro-1H-indol-1-yl)-3-(methylamino)-1-phenylpropan-2-ol hydrochloride

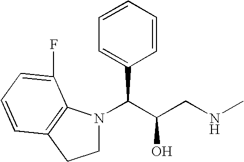

In an analogous manner to Example 1, Step 2, 7-fluoroindoline was prepared from 7-fluoroindole as a clear liquid. MS (ESI) m/z 138 ([M+H]⁺).

In an analogous manner to Example 1, Step 3, (2S,3S)-3-(7-fluoroindolin-1-yl)-3-phenylpropane-1,2-diol was prepared from 7-fluoroindoline as a white solid. MS (ESI) m/z 288.1 ([M+H]⁺).

A mixture of (2S,3S)-3-(7-fluoroindolin-1-yl)-3-phenylpropane-1,2-diol (1.09 g, 3.8 mmol) and triphenylphosphine (1.49 g, 5.7 mmol) was dissolved in tetrahydrofuran (30 mL). To this was added N-chlorosuccinimide (0.76 g, 5.7 mmol) and the reaction mixture was further stirred at room temperature for 30 minutes. The mixture was then concentrated under reduced pressure and the residue was purified via Biotage Horizon (FlasH 40 M, silica, gradient from 0% ethyl acetate/hexane to 40% ethyl acetate/hexane) to give (1S,2S)-3-chloro-1-(7-fluoroindolin-1-yl)-1-phenylpropan-2-ol as a clear oil. MS (ESI) m/z 306 ([M+H]⁺).

(1S,2S)-3-chloro-1-(7-fluoroindolin-1-yl)-1-phenylpropan-2-ol (0.49 g, 1.6 mmol) was treated with a solution of methylamine in ethanol (2.0 M, 8 ml, 16 mmol) and the solution was stirred in a sealed vessel at room temperature for 15 hours. After dilution with a saturated aqueous solution of sodium bicarbonate, the mixture was extracted with a solution of dichloromethane/isopropanol (3/1). The extract was washed with water and brine, dried over anhydrous sodium sulfate, filtered, and concentrated under reduced pressure. The crude product was crystallized from dichloromethane by adding minimum amount of ethyl acetate and diethyl ether to afford the title compound (1S,2R)-1-(7-fluoro-2,3-dihydro-1H-indol-1-yl)-3-(methylamino)-1-phenylpropan-2-ol hydrochloride as a white solid. MS (ES) m/z 300.9 ([M+H]⁺); HPLC purity 92.9% at 210-370 nm, 7.3 min.; Xterra RP18, 3.5 u, 150×4.6 mm column, 1.2 mL/min, 85/15-5/95 (Ammon. Form. Buff. Ph=3.5/ACN+MeOH) for 10 min, hold 4 min. HRMS: calculated for C₁₈H₂₁FN₂O+H⁺, 301.17107; found (ESI, [M+H]⁺), 301.1695.

### Example 120

### 4-fluoro-3-[(1S,2R)-1-(3-fluorophenyl)-2-hydroxy-3-(methylamino)propyl]-1-phenyl-1,3-dihydro-2H-benzimidazol-2-one hydrochloride

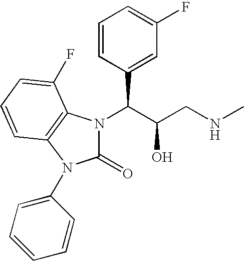

Step 1:

To a solution of 2,6-difluoronitrobenzene (2.0 g, 6.28 mmol) and aniline (d 1.022, 1.15 mL, 12.6 mmol) in dry N,N-dimethylformamide (10 mL) was added potassium tert-butoxide (1.40 g, 12.5 mmol) in portions. After 16 hours at room temperature, the reaction mixture was poured into saturated aqueous ammonium chloride solution and extracted with dichloromethane (2×50 mL). The combined organic layers were washed with water (1×50 mL), dried over anhydrous magnesium sulfate, filtered and concentrated under reduced pressure to afford crude 3-fluoro-2-nitro-N-phenylaniline (1.15 g, 78%), which was used in the next step without further purification.

Step 2:

A mixture of 3-fluoro-2-nitro-N-phenylaniline (1.15 g, 4.9 mmol) and palladium on charcoal (10%, ca. 200 mg) in methanol (30 mL) was hydrogenated (50 psi H₂) in a Parr shaker apparatus. After 2 hours, the catalyst was removed by filtration through a pad of celite, and the celite washed with fresh methanol (20 mL). The combined methanol layers were concentrated under reduced pressure and the residue purified by column chromatography (silica, 1:0 to 9:1 hexanes:ethyl acetate) to afford 3-fluoro-N1-phenylbenzene-1,2-diamine (0.47 g, 47%). MS (ES) m/z 203.2 ([M+H]⁺).

Step 3:

To a stirred solution of 3-fluoro-N1-phenylbenzene-1,2-diamine (0.247 g, 1.22 mmol) in dry tetrahydrofuran (10 mL) was added carbonyl diimidazole (0.21 g, 1.30 mmol) under nitrogen. After 30 minutes, 4-dimethylaminopyridine (catalytic amount) was added and the reaction stirred over night. After 16 hours a further portion of carbonyl diimidazole was added (0.21 g, 1.3 mmol) and stirring continued. After 48 hours, the reaction mixture was diluted with ethyl acetate (ca. 50 mL) and extracted with sodium hydroxide solution (2N, 2×25 mL). The combined basic extracts were washed with ethyl acetate and then acidified (hydrochloric acid, pH 1). The product was collected by filtration and was then washed with water, hexanes and air dried to afford 4-fluoro-1-phenyl-1H-benzo[d]imidazol-2(3H)-one (0.117 g, 42%) as a white solid. MS (ES) m/z 228.9 ([M+H]⁺).

Step 4:

Sodium hydride (60% in oil, 33 mg, 0.89 mmol) was added to 4-fluoro-1-phenyl-1H-benzo[d]imidazol-2(3H)-one (0.102 g, 0.447 mmol) in dry N,N-dimethylformamide (3 mL) under nitrogen, and the mixture stirred for 20 minutes. In a separate flask, [(2R,3R)-3-(3-fluorophenyl)oxiran-2-yl]methanol (from Example 24, Step 1, 0.15 g, 0.89 mmol) in dry dimethylformamide (3 mL) was treated with titanium tetra iso-propoxide (0.26 mL, 0.89 mmol). After 20 minutes this mixture was added to that prepared first. After 16 hours the reaction mixture was quenched by the addition of 2 N aqueous hydrochloric acid solution, extracted with ethyl acetate, dried over anhydrous sodium sulfate, filtered and concentrated under reduced pressure. The residue was then purified by column chromatography (silica, 1:0 to 0:1 hexanes:ethyl acetate) to afford 4-fluoro-3-((1S,2S)-1-(3-fluorophenyl)-2,3-dihydroxypropyl)-1-phenyl-1H-benzo[d]imidazol-2(3H)-one (0.146 g, 82%), which was used without further evaluation.

Step 5:

To a solution of 4-fluoro-3-((1S,2S)-1-(3-fluorophenyl)-2,3-dihydroxypropyl)-1-phenyl-1H-benzo[d]imidazol-2(3H)-one (0.146 g, 0.37 mmol) in dry pyridine (3 mL) was added p-toluenesulfonyl chloride (0.076 g, 0.39 mmol). After 3 hours, a further portion of p-toluenesulfonyl chloride (0.050 g, 0.27 mmol) was added and the reaction stirred over night. After 16 hours the mixture was diluted with ethyl acetate and washed with saturated aqueous copper II sulfate solution (×2) and water, dried over anhydrous sodium sulfate, filtered and concentrated under reduced pressure. The residue was then dissolved in methylamine solution (8M in ethanol, 10 mL) and stirred over night. After 16 hours, the mixture was evaporated under reduced pressure and the residue dissolved in ethyl acetate, washed with 2 N aqueous sodium hydroxide solution (10 mL), and water, dried over anhydrous sodium sulfate, filtered and concentrated under reduced pressure. The crude product was purified by column chromatography (silica, 100:0 to 95:5 dichloromethane: methanol saturated with ammonia) to afford 4-fluoro-3-[(1S,2R)-1-(3-fluorophenyl)-2-hydroxy-3-(methylamino)propyl]-1-phenyl-1,3-dihydro-2H-benzimidazol-2-one (0.027 g, 16%). The solid was then dissolved in ethanol and treated with 2 N hydrochloric acid solution (0.1 mL) concentrated under reduced pressure and triturated with diethyl ether to afford 4-fluoro-3-[(1S,2R)-1-(3-fluorophenyl)-2-hydroxy-3-(methylamino)propyl]-1-phenyl-1,3-dihydro-2H-benzimidazol-2-one hydrochloride (6 mg) as a white solid. HRMS: calculated for C₂₃H₂₁F₂N₃O₂+H⁺, 410.16746; found (ESI, [M+H]⁺), 410.1662.

### Example 121

### 4-fluoro-1-(3-fluorophenyl)-3-[(1S,2R)-1-(3-fluorophenyl)-2-hydroxy-3-(methylamino)propyl]-1,3-dihydro-2H-benzimidazol-2-one hydrochloride

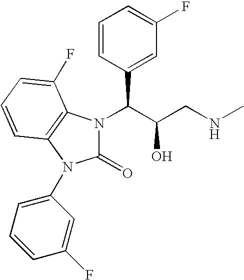

2,6-difluoronitrobenzene (5 g, 31.4 mmol), potassium tert-butoxide (3.5 g, 31.3 mmol), and 3-fluoroaniline (3.47 g, 31.3 mmol) in anhydrous dimethylsulfoxide (20 mL) was stirred at room temperature. Upon completion, the reaction was partitioned between saturated ammonium chloride solution (50 mL) and ethyl acetate (50 mL). The organic phase was separated, dried over anhydrous magnesium sulfate, filtered and concentrated under reduced pressure. The product was purified on silica gel to give (3-fluoro-2-nitro-phenyl)-(3-fluoro-phenyl)-amine that was directly in the next step.

A solution of (3-Fluoro-2-nitro-phenyl)-(3-fluoro-phenyl)-amine (3.27 g, 13 mmol) in methanol (50 mL) was hydrogenated over 10% palladium-on-carbon (ca. 200 mg) at 50 psi. Upon complete reduction, the reaction was filtered through a pad of celite and concentrated onto silica gel. The product was purified on silica gel to give 3-fluoro-N1-(3-fluorophenyl)benzene-1,2-diamine (1.26 g, 44%). MS (ES) m/z 221 ([M+H]⁺); HRMS: calculated for C₁₂H₁₀F₂N₂+H⁺, 221.08848; found (ESI, [M+H]⁺), 221.0858.

3-Fluoro-N1-(3-fluorophenyl)benzene-1,2-diamine (1.15 g, 5.22 mmol) and carbonyl diimidazole (1.46 g, 9 mmol) in dioxane (20 mL) was stirred at room temperature for 16 hours. Upon completion, the reaction was partitioned between 1 N hydrochloric acid (100 mL) and ethyl acetate (100 mL). The organics were dried over anhydrous sodium sulfate, filtered and concentrated under reduced pressure to give 4-fluoro-1-(3-fluorophenyl)-1,3-dihydro-2H-benzimidazol-2-one (0.75 g, 59%). MS (ES) m/z 247.0 ([M+H]⁺).

In an analogous manner to Example 120, Step 4, 4-fluoro-1-(3-fluorophenyl)-3-[(1S,2S)-1-(3-fluorophenyl)-2,3-dihydroxypropyl]-1,3-dihydro-2H-benzimidazol-2-one was prepared from 4-fluoro-1-(3-fluorophenyl)-1,3-dihydro-2H-benzimidazol-2-one and [(2R,3R)-3-(3-fluorophenyl)oxiran-2-yl]methanol. MS (ES) m/z 415.0 ([M+H]⁺); HRMS: calculated for C₂₂H₁₇F₃N₂O₃+H⁺, 415.12640; found (ESI, [M+H]⁺), 415.1263.

In an analogous manner to Example 25, Step 5, 4-fluoro-1-(3-fluorophenyl)-3-[(1S,2R)-1-(3-fluorophenyl)-2-hydroxy-3-(methylamino)propyl]-1,3-dihydro-2H-benzimidazol-2-one hydrochloride was prepared from 4-fluoro-1-(3-fluorophenyl)-3-[(1S,2S)-1-(3-fluorophenyl)-2,3-dihydroxypropyl]-1,3-dihydro-2H-benzimidazol-2-one. HRMS: calculated for C₂₃H₂₀F₃N₃O₂+H⁺, 428.15804; found (ESI, [M+H]⁺), 428.1581.

### Example 122

### 1-[(1S,2R)-3-amino-1-(3,5-difluorophenyl)-2-hydroxypropyl]-7-fluoro-3,3-dimethyl-1,3-dihydro-2H-indol-2-one

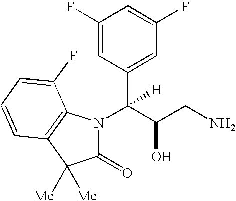

To a solution of 7-fluoro-1-[(1S,2S)-1-(3,5-difluorophenyl)-2,3-dihydroxypropyl]-3,3-dimethyl-1,3-dihydro-2H-indol-2-one (2.21g, 6.05 mmol, from Example 62, Step 5) in tetrahydrofuran (30 mL) was added triphenylphosphine (1.98 g, 7.56 mmol). The mixture was stirred at ambient temperature until all the triphenylphosphine was dissolved. To this solution was then added N-chlorosuccinimide (1.01 g, 7.56 mmol) and the resultant mixture was allowed to stir at ambient temperature for 50 minutes. The mixture was concentrated under reduced pressure and residue purified using silica gel column (eluting with a gradient of 0% to 40% ethyl acetate in hexane) to afford the chloride intermediate (1.85 g, 80%).

To a solution of the above chloride (0.35 g, 0.9 mmol) in dry N,N-dimethylformamide (5 mL) was added sodium iodide (0.15 g, 1 mmol) and sodium azide (0.16 g, 2.3 mmol). The mixture was heated at 70° C. for 18 hours, then poured into a saturated solution of ammonium chloride (80 mL). The aqueous mixture was extracted with ethyl acetate (3×20 mL), the combined organic extracts dried over anhydrous magnesium sulfate, filtered and concentrated under reduced pressure. The residue obtained was then taken up in methanol (20 mL) and 5% palladium on carbon added. The mixture was subject to hydrogenation (40 psi H₂) for 2 hours and then filtered through a pad of celite to remove palladium on carbon. The filtrate was concentrated and purified on a silica gel column (9% of methanol in methylene chloride) to give 1-[(1S,2R)-3-amino-1-(3,5-difluorophenyl)-2-hydroxypropyl]-7-fluoro-3,3-dimethyl-1,3-dihydro-2H-indol-2-one as an oil. The freebase was dissolved in ether (10 mL) and treated with hydrogen chloride solution (1.0 M in diethyl ether, 1.0 equivalent). The white precipitate was collected and dried under vacuum then dissolved in 10 mL of water and lyophilized to 1-[(1S,2R)-3-amino-1-(3,5-difluorophenyl)-2-hydroxypropyl]-7-fluoro-3,3-dimethyl-1,3-dihydro-2H-indol-2-one hydrochloride. MS (ES) m/z 364.9 ([M+H]⁺).

Cell Lines, Culture Reagents, and Assays

MDCK-Net6 cells, stably transfected with human hNET (Pacholczyk, T., R. D. Blakely, and S. G. Amara, Nature, 1991, 350(6316): p. 350-4) were cultured in growth medium containing high glucose DMEM (Gibco, Cat. No. 11995), 10% FBS (dialyzed, heat-inactivated, US Bio-Technologies, Lot FBD1129HI) and 500 □g/ml G418 (Gibco, Cat. No. 10131). Cells were plated at 300,000/T75 flask and cells were split twice weekly. The JAR cell line (human placental choriocarcinoma) was purchased from ATCC (Cat. No. HTB-144). The cells were cultured in growth medium containing RPMI 1640 (Gibco, Cat. No. 72400), 10% FBS (Irvine, Cat. No. 3000), 1% sodium pyruvate (Gibco, Cat. No. 1136) and 0.25% glucose. Cells were plated at 250,000 cells/T75 flask and split twice weekly. For all assays, cells were plated in Wallac 96-well sterile plates (PerkinElmer, Cat. No. 3983498).

Norepinephrine (NE) Uptake Assay

On day 1, cells were plated at 3,000 cells/well in growth medium and maintained in a cell incubator (37° C., 5% CO₂). On day 2, growth medium was replaced with 200 μl of assay buffer (25 mM HEPES; 120 mM NaCl; 5 mM KCl; 2.5 mM CaCl₂; 1.2 mM MgSO₄; 2 mg/ml glucose (pH 7.4, 37° C.)) containing 0.2 mg/ml ascorbic acid and 10 μM pargyline. Plates containing cells with 200 μl of assay buffer were equilibrated for 10 minutes at 37° C. prior to addition of compounds. A stock solution of desipramine was prepared in DMSO (10 mM) and delivered to triplicate wells containing cells for a final test concentration of 1 μM. Data from these wells were used to define non-specific NE uptake (minimum NE uptake). Test compounds were prepared in DMSO (10 mM) and diluted in assay buffer according to test range (1 to 10,000 nM). Twenty-five microliters of assay buffer (maximum NE uptake) or test compound were added directly to triplicate wells containing cells in 200 μl of assay buffer. The cells in assay buffer with test compounds were incubated for 20 minutes at 37° C. To initiate the NE uptake, [³H]NE diluted in assay buffer (120 nM final assay concentration) was delivered in 25 μl aliquots to each well and the plates were incubated for 5 minutes (37° C.). The reaction was terminated by decanting the supernatant from the plate. The plates containing cells were washed twice with 200 μl assay buffer (37° C.) to remove free radioligand. The plates were then inverted, left to dry for 2 minutes, then reinverted and air-dried for an additional 10 minutes. The cells were lysed in 25 μl of 0.25 N NaOH solution (4° C.), placed on a shake table and vigorously shaken for 5 minutes. After cell lysis, 75 μl of scintillation cocktail was added to each well and the plates were sealed with film tape. The plates were returned to the shake table and vigorously shaken for a minimum of 10 minutes to ensure adequate partitioning of organic and aqueous solutions. The plates were counted in a Wallac Microbeta counter (PerkinElmer) to collect the raw cpm data.

Serotonin (5-HT) Uptake Assay

The methods for 5-HT functional reuptake using the JAR cell line were modified using a previous literature report (Prasad, et al., Placenta, 1996. 17(4): 201-7). On day 1, cells were plated at 15,000 cells/well in 96-well plates containing growth medium (RPMI 1640 with 10% FBS) and maintained in a cell incubator (37° C., 5% CO₂). On day 2, cells were stimulated with staurosporine (40 nM) to increase the expression of the 5-HT transporter [17]. On day 3, cells were removed from the cell incubator two hours prior to assay and maintained at room temperature to equilibrate the growth medium to ambient oxygen concentration. Subsequently, the growth medium was replaced with 200 μl of assay buffer (25 mM HEPES; 120 mM NaCl; 5 mM KCl; 2.5 mM CaCl₂; 1.2 mM MgSO₄; 2 mg/ml glucose (pH 7.4, 37° C.)) containing 0.2 mg/ml ascorbic acid and 10 μM pargyline. A stock solution of paroxetine (AHR-4389-1) was prepared in DMSO (10 mM) and delivered to triplicate wells containing cells for a final test concentration of 1 μM. Data from these wells were used to define non-specific 5-HT uptake (minimum 5-HT uptake). Test compounds were prepared in DMSO (10 mM) and diluted in assay buffer according to test range (1 to 1,000 nM). Twenty-five microliters of assay buffer (maximum 5-HT uptake) or test compound were added directly to triplicate wells containing cells in 200 μl of assay buffer. The cells were incubated with the compound for 10 minutes (37° C.). To initiate the reaction, [³H]hydroxytryptamine creatinine sulfate diluted in assay buffer was delivered in 25 μl aliquots to each well for a final test concentration of 15 nM. The cells were incubated with the reaction mixture for 5 minutes at 37° C. The 5-HT uptake reaction was terminated by decanting the assay buffer. The cells were washed twice with 200 μl assay buffer (37° C.) to remove free radioligand. The plates were inverted and left to dry for 2 minutes, then reinverted and air-dried for an additional 10 minutes. Subsequently, the cells were lysed in 25 μl of 0.25 N NaOH (4° C.) then placed on a shaker table and shaken vigorously for 5 minutes. After cell lysis, 75 μl of scintillation cocktail was added to the wells, the plates were sealed with film tape and replaced on the shake table for a minimum of 10 minutes. The plates were counted in a Wallac Microbeta counter (PerkinElmer) to collect the raw cpm data.

Evaluation of Results

For each experiment, a data stream of cpm values collected from the Wallac Microbeta counter was downloaded to a Microsoft Excel statistical application program. Calculations of EC₅₀ values were made using the transformed-both-sides logistic dose response program written by Wyeth Biometrics Department. The statistical program uses mean cpm values from wells representing maximum binding or uptake (assay buffer) and mean cpm values from wells representing minimum binding or uptake ((1 μM desipramine (hNET) or 1 μM paroxetine (hSERT)). Estimation of the EC₅₀ value was completed on a log scale and the line was fit between the maximum and minimum binding or uptake values. All graphic data representation was generated by normalizing each data point to a mean percent based on the maximum and minimum binding or uptake values. The EC₅₀ values reported from multiple experiments were calculated by pooling the raw data from each experiment and analyzing the pooled data as one experiment.

5-HT_{2A} FLIPR Assay

Cell Conditions:

CHO cells transfected with cDNA expressing the human 5-HT_{2A} receptor are cultured in Dulbecco's modified Eagle's medium (Gibco #11995-065) supplemented with 10% fetal bovine serum, non-essential amino acids and selection markers. Cells are washed with PBS without Ca²⁺ and 3 mL Trypsin is added to dissociate cells. After 3 minute incubation, 7 mL Trypsin Neutralizing Solution is added. Cells are then aspirated from flask and mixed in a 50 mL conical tube. 10 μL sample is used to count cells on a hemacytometer. Cells are then plated at 40,000 cells per well into sterile black 96 well plates with clear bottoms (VWR #29443-152) for 24 hours.

Drug Plate Preparation:

Two 96-well drug plates are prepared for each cell plate. Plate 1 will contain compounds to be tested and plate 2 will contain the agonist DOI (3 nM) to activate a calcium response. Specific details of compound preparation are listed below. All compounds are made in 1×HBSS (Gibco #14175-095) supplemented with 20 mM HEPES (Gibco #15630-080). Outside wells are not used due to an edge effect seen in these cells.

The reference compounds DOI and 5-HT are used as standard 5HT agonists. MDL and Mianserin are used as standard 5HT_{2A} selective receptor antagonists.

Preparation of Plate 1: Test Compound Plate

For screening test compounds at 1 μM, a 1 mM stock is diluted to 19 μM (FLIPR will make final dilution) and added to 4 wells in the test plate at 50 μL per well. Standards for plate one are Vehicle, 1 μM DOI, and 3 nM MDL.

For IC₅₀ value determination, concentrations are generated by serial dilution of a 1 mM stock solution. On the day of the assay, test compound solutions of appropriate concentrations are diluted in assay buffer as described for single concentration testing. This procedure is followed to ensure that the solvent concentration is consistent across dilutions. The typical concentration testing range of compounds is 10⁻¹⁰—10⁻⁵ M in half log or full log increments.

Preparation of Plate 2: Agonist (DOI) Plate.

A 10 μM DOI stock is diluted to 60 nM and added to the respective wells. The pipeting station of the FLIPR will make an additional 20-fold dilution for a final concentration of 3 nM. Standards for this plate include Vehicle and 3 nM DOI.

Calcium Dye Preparation:

Contents of dye vial (Molecular Devices #R8090) are dissolved in 100 mL of 1×HBSS supplemented with 20 mM HEPES. Aliquots can be frozen at −20° C. for up to one week for future use. On the day of assay, dye is thawed and diluted to half concentration. Probenecid (Sigma #P-8761), a calcium anion exchange inhibitor, is made fresh from powder on the day of the experiment and added to the Calcium Buffer at a 2.5 mM final concentration prior to addition to the cells.

FLIPR Machine Loading:

Cells are allowed to adhere for 24 hours in 96-well plates. At time of assay, the cultured media is removed from the cells and replaced with 180 μL per well of Calcium 3 Assay Buffer and incubated for 1 hour at 37° C. with 5% CO₂.

Cell, compound and DOI plates are loaded into the FLIPR machine. The baseline fluorescence level is read once every second for 1 minute. Compound (10 μL) is transferred from the compound plate to the cells and the fluorescence level recorded every 6 seconds for 2 minutes to determine any agonist activity. Baseline fluorescence is recorded again every second for 10 seconds. For antagonist determination, 10 μL of 3 nM DOI is transferred from the DOI plate to the cells and the fluorescence level recorded every 6 seconds for 5 minutes. The pipetting unit of the FLIPR machine completes all transfers.

Analysis of Results:

Single Concentration

Agonist stimulation is expressed as a percentage of the response observed with 1 uM DOI.

Antagonist inhibition of 3 nM DOI stimulation is expressed as a percentage of the response observed with 3 nM DOI alone.

Concentration Curve

A 4-parameter logistic function is used to generate the EC₅₀ values. The data are log transformed prior to analysis.

The results of the standard experimental test procedures described in the preceding paragraphs are shown in Table 1:

TABLE 1

| | hNET | 5-HT_{2A} | |
|---|---|---|---|
| Example | EC₅₀ (nM) | IC₅₀ (nM) | Name |
| 1 | 18 | 234 | (1S,2R)-1-[5-(benzyloxy)-1H-indol-1-yl]-3- |
| | | | (methylamino)-1-phenylpropan-2-ol |
| | | | hydrochloride |
| 2 | 310 | 29%* | (1S,2R)-1-[4-(benzyloxy)-1H-indol-1-yl]-3- |
| | | | (methylamino)-1-phenylpropan-2-ol |
| | | | hydrochloride |
| 3 | 2%* | 39%* | (1S,2R)-1-[6-(benzyloxy)-1H-indol-1-yl]-3- |
| | | | (methylamino)-1-phenylpropan-2-ol |
| | | | hydrochloride |
| 4 | 20%* | 1468 | (1S,2R)-1-[7-(benzyloxy)-1H-indol-1-yl]-3- |
| | | | (methylamino)-1-phenylpropan-2-ol |
| | | | hydrochloride |
| 5 | 196 | 678 | (1S,2R)-1-{5-[(2-methoxybenzyl)oxy]-1H- |
| | | | indol-1-yl}-3-(methylamino)-1-phenylpropan- |
| | | | 2-ol hydrochloride |
| 6 | 17 | 888 | (1S,2R)-1-{5-[(3-methoxybenzyl)oxy]-1H- |
| | | | indol-1-yl}-3-(methylamino)-1-phenylpropan- |
| | | | 2-ol hydrochloride |
| 7 | 32 | 281 | (1S,2R)-1-{5-[(4-methoxybenzyl)oxy]-1H- |
| | | | indol-1-yl}-3-(methylamino)-1-phenylpropan- |
| | | | 2-ol hydrochloride |
| 8 | 147 | 658 | (1S,2R)-1-{5-[(2-chlorobenzyl)oxy]-1H-indol- |
| | | | 1-yl}-3-(methylamino)-1-phenylpropan-2-ol |
| | | | hydrochloride |
| 9 | 89 | 39%* | (1S,2R)-1-{5-[(3-chlorobenzyl)oxy]-1H-indol- |
| | | | 1-yl}-3-(methylamino)-1-phenylpropan-2-ol |
| | | | hydrochloride |
| 10 | 67 | 712 | (1S,2R)-1-{5-[(4-chlorobenzyl)oxy]-1H-indol- |
| | | | 1-yl}-3-(methylamino)-1-phenylpropan-2-ol |
| | | | hydrochloride |
| 11 | 52 | 1258 | (1S,2R)-1-{5-[(2-fluorobenzyl)oxy]-1H-indol- |
| | | | 1-yl}-3-(methylamino)-1-phenylpropan-2-ol |
| | | | hydrochloride |
| 12 | 36 | 979 | (1S,2R)-1-{5-[(3-fluorobenzyl)oxy]-1H-indol- |
| | | | 1-yl}-3-(methylamino)-1-phenylpropan-2-ol |
| | | | hydrochloride |
| 13 | 31 | 468 | (1S,2R)-1-{5-[(4-fluorobenzyl)oxy]-1H-indol- |
| | | | 1-yl}-3-(methylamino)-1-phenylpropan-2-ol |
| | | | hydrochloride |
| 14 | 109 | 442 | (1S,2R)-3-(methylamino)-1-{5-[(2- |
| | | | methylbenzyl)oxy]-1H-indol-1-yl}-1- |
| | | | phenylpropan-2-ol hydrochloride |
| 15 | 88 | 809 | (1S,2R)-3-(methylamino)-1-{5-[(3- |
| | | | methylbenzyl)oxy]-1H-indol-1-yl}-1- |
| | | | phenylpropan-2-ol hydrochloride |
| 16 | 37 | 4121 | (1S,2R)-3-(methylamino)-1-{5-[(4- |
| | | | methylbenzyl)oxy]-1H-indol-1-yl}-1- |
| | | | phenylpropan-2-ol hydrochloride |
| 17 | 873 | 2645 | (1S,2R)-3-(methylamino)-1-phenyl-1-[5-(1- |
| | | | phenylethoxy)-1H-indol-1-yl]propan-2-ol |
| | | | hydrochloride |
| 18 | 219 | 1197 | (1S,2R)-3-(methylamino)-1-phenyl-1-[5-(2- |
| | | | phenylethoxy)-1H-indol-1-yl]propan-2-ol |
| | | | hydrochloride |
| 19 | 27%* | 39%* | (1S,2R)-3-(methylamino)-1-(5-phenoxy-1H- |
| | | | indol-1-yl)-1-phenylpropan-2-ol hydrochloride |
| 20 | 227 | 30%* | (1S,2R)-3-(methylamino)-1-(4-phenoxy-1H- |
| | | | indol-1-yl)-1-phenylpropan-2-ol hydrochloride |
| 21 | 56%* | 27%* | (1S,2R)-3-(methylamino)-1-phenyl-1-(4- |
| | | | phenyl-1H-indol-1-yl)propan-2-ol |
| | | | hydrochloride |
| 22 | 35 | 16%* | (1S,2R)-3-(methylamino)-1-phenyl-1-(6- |
| | | | phenyl-1H-indol-1-yl)propan-2-ol |
| | | | hydrochloride |
| 23 | 48%* | 2596 | (1S,2R)-3-(methylamino)-1-phenyl-1-(7- |
| | | | phenyl-1H-indol-1-yl)propan-2-ol |
| | | | hydrochloride |
| 24 | 36 | 20%* | (1S,2R)-1-[5-(benzyloxy)-1H-indol-1-yl]-1-(3- |
| | | | fluorophenyl)-3-(methylamino)propan-2-ol |
| | | | hydrochloride |
| 25 | 18 | 23%* | (1S,2R)-1-[5-(benzyloxy)-2,3-dihydro-1H- |
| | | | indol-1-yl]-1-(3-fluorophenyl)-3- |
| | | | (methylamino)propan-2-ol hydrochloride |
| 26 | 45 | 23%* | (1S,2R)-1-[5-(benzyloxy)-2,3-dihydro-1H- |
| | | | indol-1-yl]-3-(methylamino)-1-phenylpropan- |
| | | | 2-ol hydrochloride |
| 27 | 89 | 423 | 5′-chloro-1′-[(1S,2R)-2-hydroxy-3- |
| | | | (methylamino)-1- |
| | | | phenylpropyl]spiro[cyclohexane-1,3′-indol]- |
| | | | 2′(1′H)-one hydrochloride |
| 28 | 448 | 213 | 6′-chloro-1′-[(1S,2R)-2-hydroxy-3- |
| | | | (methylamino)-1- |
| | | | phenylpropyl]spiro[cyclohexane-1,3′-indol]- |
| | | | 2′(1′H)-one hydrochloride |
| 29 | 43 | 106 | 6′-fluoro-1′-[(1S,2R)-2-hydroxy-3- |
| | | | (methylamino)-1- |
| | | | phenylpropyl]spiro[cyclohexane-1,3′-indol]- |
| | | | 2′(1′H)-one hydrochloride |
| 30 | 41 | 283 | 5′-fluoro-1′-[(1S,2R)-2-hydroxy-3- |
| | | | (methylamino)-1- |
| | | | phenylpropyl]spiro[cyclohexane-1,3′-indol]- |
| | | | 2′(1′H)-one hydrochloride |
| 31 | 32 | 203 | 7′-chloro-1′-[(1S,2R)-2-hydroxy-3- |
| | | | (methylamino)-1- |
| | | | phenylpropyl]spiro[cyclohexane-1,3′-indol]- |
| | | | 2′(1′H)-one hydrochloride |
| 32 | 43 | 95%* | 6′-fluoro-1′-[(1S,2R)-1-(3-fluorophenyl)-2- |
| | | | hydroxy-3- |
| | | | (methylamino)propyl]spiro[cyclohexane-1,3′- |
| | | | indol]-2′(1′H)-one hydrochloride |
| 33 | 29 | 13%* | (1S,2R)-3-(methylamino)-1-phenyl-1- |
| | | | spiro[cyclohexane-1,3′-indol]-1′(2′H)- |
| | | | ylpropan-2-ol hydrochloride |
| 34 | 327 | ND | (1S,2R)-1-(3-fluorophenyl)-3-(methylamino)- |
| | | | 1-{3-[2-(trifluoromethoxy)phenyl]-1H-indol-1- |
| | | | yl}propan-2-ol hydrochloride |
| 35 | 169 | ND | (1S,2R)-1-(3-fluorophenyl)-1-[3-(2- |
| | | | isopropoxyphenyl)-1H-indol-1-yl]-3- |
| | | | (methylamino)propan-2-ol hydrochloride |
| 36 | 711 | ND | (1S,2R)-1-(3-fluorophenyl)-1-[3-(4- |
| | | | fluorophenyl)-1H-indol-1-yl]-3- |
| | | | (methylamino)propan-2-ol hydrochloride |
| 37 | 26%* | ND | (1S,2R)-1-(3-fluorophenyl)-3-(methylamino)- |
| | | | 1-[3-(2-phenoxyphenyl)-1H-indol-1- |
| | | | yl]propan-2-ol hydrochloride |
| 38 | 135 | ND | (1S,2R)-1-[3-(2,4-difluorophenyl)-1H-indol-1- |
| | | | yl]-1-(3-fluorophenyl)-3- |
| | | | (methylamino)propan-2-ol hydrochloride |
| 39 | 70 | ND | (1S,2R)-1-[3-(2,5-difluorophenyl)-1H-indol-1- |
| | | | yl]-1-(3-fluorophenyl)-3- |
| | | | (methylamino)propan-2-ol hydrochloride |
| 40 | 41 | ND | (1S,2R)-1-[3-(2,3-dimethoxyphenyl)-1H- |
| | | | indol-1-yl]-1-(3-fluorophenyl)-3- |
| | | | (methylamino)propan-2-ol hydrochloride |
| 41 | 193 | ND | (1S,2R)-1-[3-(2,4-dichlorophenyl)-1H-indol-1- |
| | | | yl]-1-(3-fluorophenyl)-3- |
| | | | (methylamino)propan-2-ol hydrochloride |
| 42 | 71 | ND | (1S,2R)-1-[3-(2-ethoxyphenyl)-1H-indol-1-yl]- |
| | | | 1-(3-fluorophenyl)-3-(methylamino)propan-2- |
| | | | ol hydrochloride |
| 43 | 299 | ND | (1S,2R)-1-(7-chloro-5-methoxy-1H- |
| | | | pyrrolo[2,3-c]pyridin-1-yl)-1-(3-fluorophenyl)- |
| | | | 3-(methylamino)propan-2-ol hydrochloride |
| 44 | 416 | ND | (1S,2R)-1-(7-chloro-5-methyl-1H-pyrrolo[2,3- |
| | | | c]pyridin-1-yl)-3-(methylamino)-1- |
| | | | phenylpropan-2-ol hydrochloride |
| 45 | 43%* | ND | (1S,2R)-1-(5-methoxy-1H-pyrrolo[2,3- |
| | | | c]pyridin-1-yl)-3-(methylamino)-1- |
| | | | phenylpropan-2-ol hydrochloride |
| 46 | 46%* | ND | (1S,2R)-1-(3-fluorophenyl)-1-(5-methoxy-1H- |
| | | | pyrrolo[2,3-c]pyridin-1-yl)-3- |
| | | | (methylamino)propan-2-ol hydrochloride |
| 47 | 35%* | ND | (1S,2R)-3-(methylamino)-1-(5-methyl-1H- |
| | | | pyrrolo[2,3-c]pyridin-1-yl)-1-phenylpropan-2- |
| | | | ol hydrochloride |
| 48 | 51%* | ND | (1S,2R)-1-(3-fluorophenyl)-3-(methylamino)- |
| | | | 1-(5-methyl-1H-pyrrolo[2,3-c]pyridin-1- |
| | | | yl)propan-2-ol hydrochloride |
| 49 | 51%* | ND | (1S,2R)-3-(methylamino)-1-(7-methyl-1H- |
| | | | pyrrolo[2,3-c]pyridin-1-yl)-1-phenylpropan-2- |
| | | | ol hydrochloride |
| 50 | 521 | ND | (1S,2R)-1-(3-fluorophenyl)-3-(methylamino)- |
| | | | 1-(7-methyl-1H-pyrrolo[2,3-c]pyridin-1- |
| | | | yl)propan-2-ol hydrochloride |
| 51 | 150 | ND | (1S,2R)-1-(3,3-diethyl-2,3-dihydro-1H-indol- |
| | | | 1-yl)-1-(3-fluorophenyl)-3- |
| | | | (methylamino)propan-2-ol hydrochloride |
| 52 | 19 | ND | (1S,2R)-1-(6-fluoro-3,3-dimethyl-2,3-dihydro- |
| | | | 1H-indol-1-yl)-1-(3-fluorophenyl)-3- |
| | | | (methylamino)propan-2-ol hydrochloride |
| 53 | 49%* | ND | (1S,2R)-1-(4-benzyl-3,4-dihydroquinoxalin- |
| | | | 1(2H)-yl)-1-(3-fluorophenyl)-3- |
| | | | (methylamino)propan-2-ol hydrochloride |
| 54 | 28 | ND | (1S,2R)-1-(5-fluoro-3,3-dimethyl-2,3-dihydro- |
| | | | 1H-indol-1-yl)-1-(3-fluorophenyl)-3- |
| | | | (methylamino)propan-2-ol hydrochloride |
| 55 | 13 | ND | (1S,2R)-1-(3-fluorophenyl)-3-(methylamino)- |
| | | | 1-[(3S)-3-methyl-2,3-dihydro-1H-indol-1- |
| | | | yl]propan-2-ol hydrochloride |
| 56 | 4 | ND | (1S,2R)-1-(3-fluorophenyl)-3-(methylamino)- |
| | | | 1-[(3R)-3-methyl-2,3-dihydro-1H-indol-1- |
| | | | yl]propan-2-ol hydrochloride |
| 57 | 41 | ND | (1S,2R)-1-(3-fluorophenyl)-1-(3-isopropyl- |
| | | | 2,3-dihydro-1H-indol-1-yl)-3- |
| | | | (methylamino)propan-2-ol hydrochloride |
| 58 | 13 | ND | (1S,2R)-1-(3-ethyl-2,3-dihydro-1H-indol-1- |
| | | | yl)-1-(3-fluorophenyl)-3- |
| | | | (methylamino)propan-2-ol hydrochloride |
| 59 | 16 | ND | (1S,2R)-1-(3-ethyl-2,3-dihydro-1H-indol-1- |
| | | | yl)-3-(methylamino)-1-phenylpropan-2-ol |
| | | | hydrochloride |
| 60 | 67 | ND | (1S,2R)-1-(3-isopropyl-2,3-dihydro-1H-indol- |
| | | | 1-yl)-3-(methylamino)-1-phenylpropan-2-ol |
| | | | hydrochloride |
| 61 | 216 | ND | (1S,2R)-3-amino-1-(3,5-difluorophenyl)-1- |
| | | | (3,3-dimethyl-2,3-dihydro-1H-indol-1- |
| | | | yl)propan-2-ol hydrochloride |
| 62 | 4 | ND | 1-[(1S,2R)-1-(3,5-difluorophenyl)-2-hydroxy- |
| | | | 3-(methylamino)propyl]-7-fluoro-3,3- |
| | | | dimethyl-1,3-dihydro-2H-indol-2-one |
| | | | hydrochloride |
| 63 | 51 | ND | 5,7-difluoro-1-[(1S,2R)-1-(3-fluorophenyl)-2- |
| | | | hydroxy-3-(methylamino)propyl]-3,3- |
| | | | dimethyl-1,3-dihydro-2H-indol-2-one |
| | | | hydrochloride |
| 64 | 20 | ND | 1-[(1S,2R)-1-(3,5-difluorophenyl)-2-hydroxy- |
| | | | 3-(methylamino)propyl]-3,3-dimethyl-1,3- |
| | | | dihydro-2H-indol-2-one hydrochloride |
| 65 | 55 | ND | 1-[(1S,2R)-2-hydroxy-3-(methylamino)-1- |
| | | | phenylpropyl]-1H-indol-5-ol hydrochloride |
| 66 | 3 | ND | 1-[(1S,2R)-1-(3-fluorophenyl)-2-hydroxy-3- |
| | | | (methylamino)propyl]-1H-indol-5-ol |
| | | | hydrochloride |
| 67 | 839 | ND | 5′-(benzyloxy)-1′-[(1S,2R)-2-hydroxy-3- |
| | | | (methylamino)-1- |
| | | | phenylpropyl]spiro[cyclohexane-1,3′-indol]- |
| | | | 2′(1′H)-one |
| 68 | 240 | ND | 5-(benzyloxy)-1-[(1S,2R)-2-hydroxy-3- |
| | | | (methylamino)-1-phenylpropyl]-3,3-dimethyl- |
| | | | 1,3-dihydro-2H-indol-2-one hydrochloride |
| 69 | 92 | ND | 1-[(1S,2R)-1-(3-chlorophenyl)-2-hydroxy-3- |
| | | | (methylamino)propyl]-7-fluoro-3,3-dimethyl- |
| | | | 1,3-dihydro-2H-indol-2-one hydrochloride |
| 70 | 62 | ND | (1S,2R)-1-(3-chloro-5-fluorophenyl)-1-(1H- |
| | | | indol-1-yl)-3-(methylamino)propan-2-ol |
| | | | hydrochloride |
| 71 | 92 | 44%* | 3-chloro-N-{1-[(1S,2R)-2-hydroxy-3- |
| | | | (methylamino)-1-phenylpropyl]-1H-indol-5- |
| | | | yl}-4-methylbenzamide hydrochloride |
| 71 | 92 | 44%* | 3-chloro-N-{1-[(1S,2R)-2-hydroxy-3- |
| | | | (methylamino)-1-phenylpropyl]-1H-indol-5- |
| | | | yl}-4-methylbenzamide hydrochloride |
| 72 | 95 | 60%* | 3-chloro-N-{1-[(1S,2R)-2-hydroxy-3- |
| | | | (methylamino)-1-phenylpropyl]-2,3-dihydro- |
| | | | 1H-indol-5-yl}benzamide hydrochloride |
| 73 | 91 | 625 | 3-chloro-N-{1-[(1S,2R)-2-hydroxy-3- |
| | | | (methylamino)-1-phenylpropyl]-1H-indol-5- |
| | | | yl}benzamide hydrochloride |
| 74 | 365 | 24%* | N-{1-[(1S,2R)-2-hydroxy-3-(methylamino)-1- |
| | | | phenylpropyl]-2,3-dihydro-1H-indol-5- |
| | | | yl}benzamide hydrochloride |
| 75 | 299 | 34%* | N-{1-[(1S,2R)-2-hydroxy-3-(methylamino)-1- |
| | | | phenylpropyl]-1H-indol-5-yl}benzamide |
| | | | hydrochloride |
| 76 | 54%* | 45%* | N-{1-[(1S,2R)-2-hydroxy-3-(methylamino)-1- |
| | | | phenylpropyl]-2,3-dihydro-1H-indol-5- |
| | | | yl}cyclohexanecarboxamide hydrochloride |
| 77 | 60%* | 51%* | N-{1-[(1S,2R)-2-hydroxy-3-(methylamino)-1- |
| | | | phenylpropyl]-1H-indol-5- |
| | | | yl}cyclohexanecarboxamide hydrochloride |
| 78 | 362 | 691 | N-(3-chlorophenyl)-1-[(1S,2R)-2-hydroxy-3- |
| | | | (methylamino)-1-phenylpropyl]indoline-5- |
| | | | carboxamide hydrochloride |
| 79 | 328 | 84 | N-(3-chlorophenyl)-1-[(1S,2R)-2-hydroxy-3- |
| | | | (methylamino)-1-phenylpropyl]-1H-indole-5- |
| | | | carboxamide hydrochloride |
| 80 | 12%* | 32%* | (1S,2R)-3-(methylamino)-1-(6-phenoxy-1H- |
| | | | indol-1-yl)-1-phenylpropan-2-ol hydrochloride |
| 81 | 1021 | 372 | (1S,2R)-3-(methylamino)-1-(7-phenoxy-1H- |
| | | | indol-1-yl)-1-phenylpropan-2-ol hydrochloride |
| 82 | 279 | 796 | (1S,2R)-3-amino-1-[5-(benzyloxy)-1H-indol- |
| | | | 1-yl]-1-phenylpropan-2-ol hydrochloride |
| 83 | 43%* | 380 | (1S,2R)-1-[5-(benzyloxy)-1H-indol-1-yl]-3- |
| | | | (ethylamino)-1-phenylpropan-2-ol |
| | | | hydrochloride |
| 84 | 11%* | 50%* | (1S,2R)-1-[5-(benzyloxy)-1H-indol-1-yl]-1- |
| | | | phenyl-3-(propylamino)propan-2-ol |
| | | | hydrochloride |
| 85 | 4%* | 32%* | (1S,2R)-1-[5-(benzyloxy)-1H-indol-1-yl]-3- |
| | | | (isopropylamino)-1-phenylpropan-2-ol |
| | | | hydrochloride |
| 86 | 22%* | 263 | (1S,2R)-1-[5-(benzyloxy)-1H-indol-1-yl]-3- |
| | | | (dimethylamino)-1-phenylpropan-2-ol |
| | | | hydrochloride |
| 87 | 16%* | 1120 | (1S,2R)-1-[5-(benzyloxy)-1H-indol-1-yl]-3- |
| | | | [ethyl(methyl)amino]-1-phenylpropan-2-ol |
| | | | hydrochloride |
| 88 | 21%* | 33%* | (1S,2R)-1-[5-(benzyloxy)-1H-indol-1-yl]-3- |
| | | | (diethylamino)-1-phenylpropan-2-ol |
| | | | hydrochloride |
| 89 | 14%* | 32%* | (1S,2R)-1-[5-(benzyloxy)-1H-indol-1-yl]-1- |
| | | | phenyl-3-pyrrolidin-1-ylpropan-2-ol |
| | | | hydrochloride |
| 90 | 12%* | 25%* | (1S,2R)-1-[5-(benzyloxy)-1H-indol-1-yl]-1- |
| | | | phenyl-3-piperidin-1-ylpropan-2-ol |
| | | | hydrochloride |
| 91 | 22%* | 29%* | (1S,2R)-1-[5-(benzyloxy)-1H-indol-1-yl]-3-(4- |
| | | | methylpiperazin-1-yl)-1-phenylpropan-2-ol |
| | | | hydrochloride |
| 92 | 9 | ND | (1S,2R)-3-(methylamino)-1-phenyl-1-[5- |
| | | | (pyridin-2-ylmethoxy)-1H-indol-1-yl]propan-2- |
| | | | ol hydrochloride |
| 93 | 287 | 36%* | (1S,2R)-3-(methylamino)-1-phenyl-1-[5- |
| | | | (phenylethynyl)-1H-indol-1-yl]propan-2-ol |
| | | | hydrochloride |
| 94 | 86 | 636 | (1S,2R)-3-(methylamino)-1-phenyl-1-[5-(2- |
| | | | phenylethyl)-1H-indol-1-yl]propan-2-ol |
| | | | hydrochloride |
| 95 | 7%* | 44%* | 1′-[(1S,2R)-3-amino-2-hydroxy-1- |
| | | | phenylpropyl]-6′-fluorospiro[cyclohexane- |
| | | | 1,3′-indol]-2′(1′H)-one hydrochloride |
| 96 | 48%* | 462 | 1′-[(1S,2R)-3-(ethylamino)-2-hydroxy-1- |
| | | | phenylpropyl]-6′-fluorospiro[cyclohexane- |
| | | | 1,3′-indol]-2′(1′H)-one hydrochloride |
| 97 | 0%* | 44%* | 6′-fluoro-1′-[(1S,2R)-2-hydroxy-3- |
| | | | (isopropylamino)-1- |
| | | | phenylpropyl]spiro[cyclohexane-1,3′-indol]- |
| | | | 2′(1′H)-one hydrochloride |
| 98 | 0%* | 672 | 6′-fluoro-1′-[(1S,2R)-2-hydroxy-1-phenyl-3- |
| | | | (propylamino)propyl]spiro[cyclohexane-1,3′- |
| | | | indol]-2′(1′H)-one hydrochloride |
| 99 | 11%* | 7%* | 1′-[(1S,2R)-3-amino-2-hydroxy-1- |
| | | | phenylpropyl]-5′-fluorospiro[cyclohexane- |
| | | | 1,3′-indol]-2′(1′H)-one hydrochloride |
| 100 | 31%* | 378 | 1′-[(1S,2R)-3-(ethylamino)-2-hydroxy-1- |
| | | | phenylpropyl]-5′-fluorospiro[cyclohexane- |
| | | | 1,3′-indol]-2′(1′H)-one hydrochloride |
| 101 | 0%* | 22%* | 5′-fluoro-1′-[(1S,2R)-2-hydroxy-3- |
| | | | (isopropylamino)-1- |
| | | | phenylpropyl]spiro[cyclohexane-1,3′-indol]- |
| | | | 2′(1′H)-one hydrochloride |
| 102 | 0%* | 895 | 5′-fluoro-1′-[(1S,2R)-2-hydroxy-1-phenyl-3- |
| | | | (propylamino)propyl]spiro[cyclohexane-1,3′- |
| | | | indol]-2′(1′H)-one hydrochloride |
| 103 | 19%* | 77 | 1′-[(1S,2R)-3-(dimethylamino)-2-hydroxy-1- |
| | | | phenylpropyl]-5′-fluorospiro[cyclohexane- |
| | | | 1,3′-indol]-2′(1′H)-one hydrochloride |
| 104 | 13%* | 22%* | 5′-fluoro-1′-[(1S,2R)-2-hydroxy-3-morpholin- |
| | | | 4-yl-1-phenylpropyl]spiro[cyclohexane-1,3′- |
| | | | indol]-2′(1′H)-one hydrochloride |
| 105 | 81 | 37%* | 1′-[(1S,2R)-2-hydroxy-3-(methylamino)-1- |
| | | | phenylpropyl]-5′-methoxyspiro[cyclohexane- |
| | | | 1,3′-indol]-2′(1′H)-one hydrochloride |
| 106 | 908 | 615 | 1′-[(1S,2R)-2-hydroxy-3-(methylamino)-1- |
| | | | phenylpropyl]-6′-methoxyspiro[cyclohexane- |
| | | | 1,3′-indol]-2′(1′H)-one hydrochloride |
| 107 | 349 | 168 | 1′-[(1S,2R)-2-hydroxy-3-(methylamino)-1- |
| | | | phenylpropyl]-2′-oxo-1′,2′- |
| | | | dihydrospiro[cyclohexane-1,3′-indole]-5′- |
| | | | carbonitrile hydrochloride |
| 108 | 1262 | 11%* | 1′-[(1S,2R)-2-hydroxy-3-(methylamino)-1- |
| | | | phenylpropyl]-2′-oxo-1′,2′- |
| | | | dihydrospiro[cyclohexane-1,3′-indole]-6′- |
| | | | carbonitrile hydrochloride |
| 109 | 107 | 1479 | 4′,5′-difluoro-1′-[(1S,2R)-2-hydroxy-3- |
| | | | (methylamino)-1- |
| | | | phenylpropyl]spiro[cyclohexane-1,3′-indol]- |
| | | | 2′(1′H)-one hydrochloride |
| 110 | 6 | ND | 7′-fluoro-1′-[(1S,2R)-1-(3-fluorophenyl)-2- |
| | | | hydroxy-3- |
| | | | (methylamino)propyl]spiro[cyclohexane-1,3′- |
| | | | indol]-2′(1′H)-one hydrochloride |
| 111 | 186 | ND | 1′-[(1S,2R)-1-(3-chlorophenyl)-2-hydroxy-3- |
| | | | (methylamino)propyl]-6′- |
| | | | fluorospiro[cyclohexane-1,3′-indol]-2′(1′H)- |
| | | | one hydrochloride |
| 112 | 2 | ND | 1-[(1S,2R)-1-(3-chloro-5-fluorophenyl)-2- |
| | | | hydroxy-3-(methylamino)propyl]-7-fluoro-3,3- |
| | | | dimethyl-1,3-dihydro-2H-indol-2-one |
| | | | hydrochloride |
| 113 | 33 | ND | (1S,2R)-1-(3-chloro-5-fluorophenyl)-1-(2,3- |
| | | | dihydro-1H-indol-1-yl)-3- |
| | | | (methylamino)propan-2-ol hydrochloride |
| 114 | 166 | ND | (1S,2R)-1-(3-chloro-5-fluorophenyl)-1-(7- |
| | | | fluoro-3,3-dimethyl-2,3-dihydro-1H-indol-1- |
| | | | yl)-3-(methylamino)propan-2-ol |
| | | | hydrochloride |
| 115 | 133 | ND | (1S,2R)-1-(3-chloro-5-fluorophenyl)-1-(3,3- |
| | | | dimethyl-2,3-dihydro-1H-indol-1-yl)-3- |
| | | | (methylamino)propan-2-ol hydrochloride |
| 116 | 10 | ND | 7′-fluoro-1′-[(1S,2R)-1-(3-fluorophenyl)-2- |
| | | | hydroxy-3- |
| | | | (methylamino)propyl]spiro[cyclobutane-1,3′- |
| | | | indol]-2′(1′H)-one hydrochloride |
| 117 | 2 | ND | 7′-fluoro-1′-[(1S,2R)-1-(3-fluorophenyl)-2- |
| | | | hydroxy-3- |
| | | | (methylamino)propyl]spiro[cyclopentane-1,3′- |
| | | | indol]-2′(1′H)-one hydrochloride |
| 118 | 40 | ND | 6-fluoro-1-[(1S,2R)-1-(3-fluorophenyl)-2- |
| | | | hydroxy-3-(methylamino)propyl]-3,3- |
| | | | dimethyl-1,3-dihydro-2H-indol-2-one |
| | | | hydrochloride |
| 119 | 39 | ND | (1S,2R)-1-(7-fluoro-2,3-dihydro-1H-indol-1- |
| | | | yl)-3-(methylamino)-1-phenylpropan-2-ol |
| | | | hydrochloride |
| 120 | 92 | ND | 4-fluoro-3-[(1S,2R)-1-(3-fluorophenyl)-2- |
| | | | hydroxy-3-(methylamino)propyl]-1-phenyl- |
| | | | 1,3-dihydro-2H-benzimidazol-2-one |
| | | | hydrochloride |
| 121 | 189 | ND | 4-fluoro-1-(3-fluorophenyl)-3-[(1S,2R)-1-(3- |
| | | | fluorophenyl)-2-hydroxy-3- |
| | | | (methylamino)propyl]-1,3-dihydro-2H- |
| | | | benzimidazol-2-one hydrochloride |
| 122 | 207 | ND | 1-[(1S,2R)-3-amino-1-(3,5-difluorophenyl)-2- |
| | | | hydroxypropyl]-7-fluoro-3,3-dimethyl-1,3- |
| | | | dihydro-2H-indol-2-one hydrochloride |

*Percentage inhibition at 1 μM
ND = Not determined

When ranges are used herein for physical properties, such as molecular weight, or chemical properties, such as chemical formulae, all combinations and subcombinations of ranges specific embodiments therein are intended to be included.

The disclosures of each patent, patent application and publication cited or described in this document are hereby incorporated herein by reference, in its entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

What is claimed is:

1. A compound of formula 1: 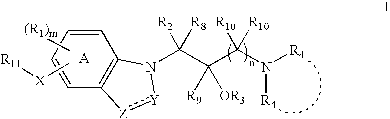
or a pharmaceutically acceptable salt thereof;
wherein:
the dotted line between Y and Z represents an optional second bond;
the dotted line between the two R₄ groups represents an optional heterocyclic ring of 4 to 6 ring atoms that may be formed between the two R₄ groups, together with the nitrogen through which they are attached;
X is —(C(R₁₂)₂)ₒ—, —O(C(R₁₂)₂)ₒ—, —(C(R₁₂)₂)ₒO—, —S(O)ₚ(C(R₁₂)₂)ₒ—, —(C(R₁₂)₂)ₒS(O)ₚ—, —N(R₁₃)C(O)(C(R₁₂)₂)ₒ—, —(C(R₁₂)₂)ₒC(O)N(R₁₃)—, —C(O)N(R₁₃)(C(R₁₂)₂)ₒ—, —(C(R₁₂)₂)ₒN(R₁₃)C(O)—, —(C(R₁₂)₂)ₒN(R₁₃)S(O)₂—, —S(O)₂N(R₁₃)(C(R₁₂)₂)ₒ—, —N(R₁₃)S(O)₂(C(R₁₂)₂)ₒ—, —(C(R₁₂)₂)ₒS(O)₂N(R₁₃)—, —NR₇(C(R₁₂)₂)ₒ—, —(C(R₁₂)₂)ₒNR₇—, or —C≡C—;
Y is N, C(R₆)₂, CR₆, or C═O;
Z is O, S(O)ₚ, N, NR₇, CR₅, or C(R₅)₂;
R₁ is, independently at each occurrence, alkyl, alkoxy, halo, CF₃, OCF₃, hydroxy, alkanoyloxy, nitro, cyano, alkenyl, alkynyl, alkylsulfoxide, alkylsulfone, alkylsulfonamide, or alkylamido; or two adjacent R₁ also represent methylenedioxy;
R₂ is aryl substituted with 0-3 R₁₄ or heteroaryl substituted with 0-3 R₁₄;
R₃ is H or C₁-C₄ alkyl;
R₄ is, independently at each occurrence, H, C₁-C₄ alkyl, C₃-C₆ cycloalkyl, arylalkyl, heteroarylmethyl, cycloheptylmethyl, cyclohexylmethyl, cyclopentylmethyl, or cyclobutylmethyl, or
both R₄ groups, together with the nitrogen through which they are attached, form a heterocyclic ring of 4 to 6 ring atoms, where one carbon may be optionally replaced with N, O, S, or SO₂, and where any carbon ring atom or additional N atom may be optionally substituted with C₁-C₄ alkyl, F, or CF₃;
R₅ is, independently at each occurrence, H, C₁-C₄ alkyl, aryl substituted with 0-3 R₁₄, heteroaryl substituted with 0-3 R₁₄, or cyano; or when two R₅ are present, they may form a carbocyclic ring of 3-5 carbons;
R₆ is, independently at each occurrence, H, C₁-C₄ alkyl, or cyano;
R₇ is H, C₁-C₆ alkyl, C₃-C₆ cycloalkyl, aryl substituted with 0-3 R₁₄; or heteroaryl substituted with 0-3 R₁₄;
R₈ is H, or C₁-C₄ alkyl;
R₉ is H, or C₁-C₄ alkyl;
R₁₀ is, independently at each occurrence, H, or C₁-C₄ alkyl; or R₁₀ and R₄ together with the nitrogen to which R₄ is attached form a nitrogen-containing ring containing 3-6 carbon atoms;
R₁₁ is aryl substituted with 0-3 R₁ or heteroaryl substituted with 0-3 R₁;
R₁₂ is, independently at each occurrence, H, C₁-C₄ alkyl;
R₁₃ is H or C₁-C₄ alkyl;
R₁₄ is, independently at each occurrence, alkyl, alkoxy, halo, CF₃, OCF₃, arylalkyloxy substituted with 0-3 R₁, aryloxy substituted with 0-3 R₁, aryl substituted with 0-3 R₁, heteroaryl substituted with 0-3 R₁, hydroxy, alkanoyloxy, nitro, cyano, alkenyl, alkynyl, alkylsulfoxide, phenylsulfoxide substituted with 0-3 R₁, alkylsulfone, phenylsulfone substituted with 0-3 R₁, alkylsulfonamide, phenylsulfonamide substituted with 0-3 R₁, heteroaryloxy substituted with 0-3 R₁, heteroarylmethyloxy substituted with 0-3 R₁, alkylamido, or arylamido substituted with 0-3 R₁; or two adjacent R₁ also represent methylenedioxy;
m is an integer from 0 to 3;
n is an integer from 1 to 2;
o is an integer from 0 to 3; and
p is an integer from 0 to 2;
wherein 1-3 carbon atoms in ring A may optionally be replaced with N.

2. A compound according to claim 1, wherein:
the dotted line between Y and Z represents a second bond.

3. A compound according to claim 1, wherein:
X is —(C(R₁₂)₂)ₒ, —O(C(R₁₂)₂)ₒ—, —C≡C—.

4. A compound according to claim 1, wherein:
Y is C(R₆)₂, CR₆, or C═O.

5. A compound according to claim 1, wherein:
Z is CR₅ or C(R₅)₂.

6. A compound according to claim 1, wherein:
R₁ is, independently at each occurrence, alkyl, alkoxy, halo, CF₃, OCF₃, hydroxy, alkanoyloxy, nitro, or cyano.

7. A compound according to claim 1, wherein:
R₂ is aryl substituted with 0-2 R₁₄.

8. A compound according to claim 1, wherein:
R₂ is phenyl, fluorophenyl, or difluorophenyl.

9. A compound according to claim 1, wherein:
R₃ is H.

10. A compound according to claim 1, wherein:
R₄ is H or methyl.

11. A compound according to claim 1, wherein:
R₅ is, independently at each occurrence, H, C₁-C₄ alkyl, aryl substituted with 0-3 R₁₄.

12. A compound according to claim 1, wherein:
R₅ is, independently at each occurrence, H, methyl, ethyl, n-propyl, isopropyl, aryl substituted with alkoxy, aryl substituted with aryloxy or phenyl substituted with 1-2 halo.

13. A compound according to claim 1, wherein:
R₆ is, independently at each occurrence, H, methyl, ethyl, n-propyl, or isopropyl.

14. A compound according to claim 1, wherein:
R₇ is H, C₁-C₆ alkyl, or aryl substituted with 0-3 R₁₄.

15. A compound according to claim 1, wherein:
R₈ is H.

16. A compound according to claim 1, wherein:
R₉ is H.

17. A compound according to claim 1, wherein:
R₁₀ is H.

18. A compound according to claim 1, wherein
R₁₁ is aryl substituted with 0-3 R₁.

19. A compound according to claim 1, wherein
R₁₁ is phenyl, or aryl substituted with 1-2 halo or alkoxy.

20. A compound according to claim 1, wherein
R₁₁ is aryl substituted with 0-2 R₁.

21. A compound according to claim 1, wherein
n is 1.

22. A compound of formula II: 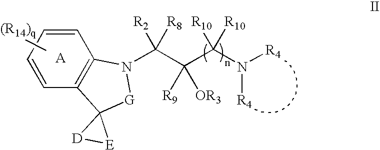
or a pharmaceutically acceptable salt thereof;
wherein:
D and E, together with the carbon atom through which they are attached, form a carbocyclic ring of 6 to 8 atoms or a heterocyclic ring of 5 to 8 atoms containing 1 to 2 heteroatoms selected from O, S(O)ₚ, and NR₇, where any carbon ring atom may be optionally substituted with C₁-C₄ alkyl, F, or CF₃;
the dotted line between the two R₄ groups represents an optional heterocyclic ring of 4 to 6 ring atoms that may be formed between the two R₄ groups, together with the nitrogen through which they are attached;
G is NR₇, C(R₆)₂, or C═O;
R₁ is, independently at each occurrence, alkyl, alkoxy, halo, CF₃, OCF₃, hydroxy, alkanoyloxy, nitro, cyano, alkenyl, alkynyl, alkylsulfoxide, alkylsulfone, alkylsulfonamide, or alkylamido; or two adjacent R₁ also represent methylenedioxy;
R₂ is aryl substituted with 0-3 R₁₄ or heteroaryl substituted with 0-3 R₁₄;
R₃ is H or C₁-C₄ alkyl;
R₄ is, independently at each occurrence, H, C₁-C₄ alkyl, C₃-C₆ cycloalkyl, arylalkyl, heteroarylmethyl, cycloheptylmethyl, cyclohexylmethyl, cyclopentylmethyl, or cyclobutylmethyl, or
both R₄ groups, together with the nitrogen through which they are attached, form a heterocyclic ring of 4 to 6 ring atoms, where one carbon may be optionally replaced with N, O, S, or SO₂, and where any carbon ring atom or additional N atom may be optionally substituted with C₁-C₄ alkyl, F, or CF₃;
R₆ is, independently at each occurrence, H, C₁-C₄ alkyl, or cyano;
R₇ is H, C₁-C₆ alkyl, C₃-C₆ cycloalkyl, aryl substituted with 0-3 R₁₄; or heteroaryl substituted with 0-3 R₁₄.
R₈ is H, or C₁-C₄ alkyl;
R₉ is H, or C₁-C₄ alkyl;
R₁₀ is, independently at each occurrence, H, or C₁-C₄ alkyl; or R₁₀ and R₄ together with the nitrogen to which R₄ is attached form a nitrogen-containing ring containing 3-6 carbon atoms;
R₁₄ is, independently at each occurrence, alkyl, alkoxy, halo, CF₃, OCF₃, arylalkyloxy substituted with 0-3 R₁, aryloxy substituted with 0-3 R₁, aryl substituted with 0-3 R₁, heteroaryl substituted with 0-3 R₁, hydroxy, alkanoyloxy, nitro, cyano, alkenyl, alkynyl, alkylsulfoxide, phenylsulfoxide substituted with 0-3 R₁, alkylsulfone, phenylsulfone substituted with 0-3 R₁, alkylsulfonamide, phenylsulfonamide substituted with 0-3 R₁, heteroaryloxy substituted with 0-3 R₁, heteroarylmethyloxy substituted with 0-3 R₁, alkylamido, or arylamido substituted with 0-3 R₁; or two adjacent R₁ also represent methylenedioxy;
n is an integer from 1 to 2;
p is an integer from 0 to 2; and
q is an integer from 0 to 4;
wherein 1-3 carbon atoms in ring A may optionally be replaced with N.

23. A compound according to claim 22, wherein:
R₁ is, independently at each occurrence, alkyl, alkoxy, halo, CF₃, OCF₃, hydroxy, alkanoyloxy, nitro, or cyano.

24. A compound according to claim 22, wherein:
R₂ is aryl substituted with 0-2 R₁₄.

25. A compound according to claim 22, wherein:
R₂ is phenyl, fluorophenyl, or difluorophenyl.

26. A compound according to claim 22, wherein:
R₃ is H.

27. A compound according to claim 22, wherein:
R₄ is H or methyl.

28. A compound according to claim 22, wherein:
R₆ is, independently at each occurrence, H, methyl, ethyl, n-propyl, or isopropyl.

29. A compound according to claim 22, wherein:
R₇ is H, C₁-C₆ alkyl, or aryl substituted with 0-3 R₁₄.

30. A compound according to claim 22, wherein:
R₈ is H.

31. A compound according to claim 22, wherein:
R₉ is H.

32. A compound according to claim 22, wherein:
R₁₀ is H.

33. A compound according to claim 22, wherein
R₁₄ is, independently at each occurrence, alkyl, alkoxy, halo, CF₃, OCF₃, hydroxy, alkanoyloxy, nitro, or cyano.

34. A compound according to claim 22, wherein
n is 1.

35. A compound according to claim 22, wherein
p is 0 or 1.

36. A compound of formula III: 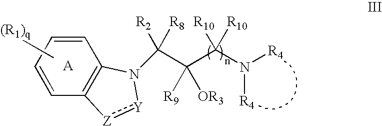
or a pharmaceutically acceptable salt thereof;
wherein:
the dotted line between Y and Z represents an optional second bond;
the dotted line between the two R₄ groups represents an optional heterocyclic ring of 4 to 6 ring atoms that may be formed between the two R₄ groups, together with the nitrogen through which they are attached;
Y is N, C(R₆)₂, CR₆, or C═O;
Z is O, S(O)ₚ, N, NR₇, CR₅, or C(R₅)₂;
R₁ is, independently at each occurrence, alkyl, alkoxy, halo, CF₃, OCF₃, hydroxy, alkanoyloxy, nitro, cyano, alkenyl, alkynyl, alkylsulfoxide, alkylsulfone, alkylsulfonamide, or alkylamido; or two adjacent R₁ also represent methylenedioxy;
R₂ is aryl substituted with 0-3 R₁₄ or heteroaryl substituted with 0-3 R₁₄;
R₃ is H or C₁-C₄ alkyl;
R₄ is, independently at each occurrence, H, C₁-C₄ alkyl, C₃-C₆ cycloalkyl, arylalkyl, heteroarylmethyl, cycloheptylmethyl, cyclohexylmethyl, cyclopentylmethyl, or cyclobutylmethyl, or
both R₄ groups, together with the nitrogen through which they are attached, form a heterocyclic ring of 4 to 6 ring atoms, where one carbon may be optionally replaced with N, O, S, or SO₂, and where any carbon ring atom or additional N atom may be optionally substituted with C₁-C₄ alkyl, F, or CF₃;
R₅ is, independently at each occurrence, H, C₁-C₄ alkyl, aryl substituted with 0-3 R₁₄, heteroaryl substituted with 0-3 R₁₄, or cyano; or when two R₅ are present, they may form a carbocyclic ring of 3-5 carbons;
R₆ is, independently at each occurrence, H, C₁-C₄ alkyl, or cyano;
R₇ is H, C₁-C₆ alkyl, C₃-C₆ cycloalkyl, aryl substituted with 0-3 R₁₄, or heteroaryl substituted with 0-3 R₁₄;
R₈ is H, or C₁-C₄ alkyl;
R₉ is H, or C₁-C₄ alkyl;
R₁₀ is, independently at each occurrence, H, or C₁-C₄ alkyl; or R₁₀ and R₄ together with the nitrogen to which R₄ is attached form a nitrogen-containing ring containing 3-6 carbon atoms;
R₁₄ is, independently at each occurrence, alkyl, alkoxy, halo, CF₃, OCF₃, arylalkyloxy substituted with 0-3 R₁, aryloxy substituted with 0-3 R₁, aryl substituted with 0-3 R₁, heteroaryl substituted with 0-3 R₁, hydroxy, alkanoyloxy, nitro, cyano, alkenyl, alkynyl, alkylsulfoxide, phenylsulfoxide substituted with 0-3 R₁, alkylsulfone, phenylsulfone substituted with 0-3 R₁, alkylsulfonamide, phenylsulfonamide substituted with 0-3 R₁, heteroaryloxy substituted with 0-3 R₁, heteroarylmethyloxy substituted with 0-3 R₁, alkylamido, or arylamido substituted with 0-3 R₁; or two adjacent R₁ also represent methylenedioxy;
n is an integer from 1 to 2; and
q is an integer from 0 to 4;
wherein 1-3 carbon atoms in ring A may optionally be replaced with N.

37. A compound according to claim 36, wherein:
the dotted line between Y and Z represents a second bond.

38. A compound according to claim 36, wherein:
Y is C(R₆)₂, CR₆, or C═O.

39. A compound according to claim 36, wherein:
Z is CR₅ or C(R₅)₂.

40. A compound according to claim 36, wherein:
R₁ is, independently at each occurrence, alkyl, alkoxy, halo, CF₃, OCF₃, hydroxy, alkanoyloxy, nitro, or cyano.

41. A compound according to claim 36, wherein:
R₂ is aryl substituted with 0-2 R₁₄.

42. A compound according to claim 36, wherein:
R₂ is phenyl, fluorophenyl, or difluorophenyl.

43. A compound according to claim 36, wherein:
R₃ is H.

44. A compound according to claim 36, wherein:
R₄ is H or methyl.

45. A compound according to claim 36, wherein:
R₅ is, independently at each occurrence, H, C₁-C₄ alkyl, aryl substituted with 0-3 R₁₄.

46. A compound according to claim 36, wherein:
R₅ is, independently at each occurrence, H, methyl, ethyl, n-propyl, isopropyl, aryl substituted with alkoxy, aryl substituted with aryloxy or phenyl substituted with 1-2 halo.

47. A compound according to claim 36, wherein:
R₆ is, independently at each occurrence, H, methyl, ethyl, n-propyl, or isopropyl.

48. A compound according to claim 36, wherein:
R₇ is H, C₁-C₆ alkyl, or aryl substituted with 0-3 R₁₄.

49. A compound according to claim 36, wherein:
R₈ is H.

50. A compound according to claim 36, wherein:
R₉ is H.

51. A compound according to claim 36, wherein:
R₁₀ is H.

52. A compound according to claim 36, wherein:
n is 1.

53. A compound according to claim 36, wherein:
q is an integer from 0 to 2.

54. A compound selected from the group consisting of:
1-[5-(benzyloxy)-1H-indol-1-yl]-3-(methylamino)-1-phenylpropan-2-ol;
1-[4-(benzyloxy)-1H-indol-1-yl]-3-(methylamino)-1-phenylpropan-2-ol;
1-[6-(benzyloxy)-1H-indol-1-yl]-3-(methylamino)-1-phenylpropan-2-ol;
1-[7-(benzyloxy)-1H-indol-1-yl]-3-(methylamino)-1-phenylpropan-2-ol;
1-{5-[(2-methoxybenzyl)oxy]-1H-indol-1-yl}-3-(methylamino)-1-phenylpropan-2-ol;
1-{5-[(3-methoxybenzyl)oxy]-1H-indol-1-yl}-3-(methylamino)-1-phenylpropan-2-ol;
1-{5-[(4-methoxybenzyl)oxy]-1H-indol-1-yl}-3-(methylamino)-1-phenylpropan-2-ol;
1-{5-[(2-chlorobenzyl)oxy]-1H-indol-1-yl}-3-(methylamino)-1-phenylpropan-2-ol;
1-{5-[(3-chlorobenzyl)oxy]-1H-indol-1-yl}-3-(methylamino)-1-phenylpropan-2-ol;
1-{5-[(4-chlorobenzyl)oxy]-1H-indol-1-yl}-3-(methylamino)-1-phenylpropan-2-ol;
1-{5-[(2-fluorobenzyl)oxy]-1H-indol-1-yl}-3-(methylamino)-1-phenylpropan-2-ol;
1-{5-[(3-fluorobenzyl)oxy]-1H-indol-1-yl}-3-(methylamino)-1-phenylpropan-2-ol;
1-{5-[(4-fluorobenzyl)oxy]-1H-indol-1-yl}-3-(methylamino)-1-phenylpropan-2-ol;
3-(methylamino)-1-{5-[(2-methylbenzyl)oxy]-1H-indol-1-yl}-1-phenylpropan-2-ol;
3-(methylamino)-1-{5-[(3-methylbenzyl)oxy]-1H-indol-1-yl}-1-phenylpropan-2-ol;
3-(methylamino)-1-{5-[(4-methylbenzyl)oxy]-1H-indol-1-yl}-1-phenylpropan-2-ol;
3-(methylamino)-1-phenyl-1-[5-(1-phenylethoxy)-1H-indol-1-yl]propan-2-ol;
3-(methylamino)-1-phenyl-1-[5-(2-phenylethoxy)-1H-indol-1-yl]propan-2-ol;
3-(methylamino)-1-(5-phenoxy-1H-indol-1-yl)-1-phenylpropan-2-ol;
3-(methylamino)-1-(4-phenoxy-1H-indol-1-yl)-1-phenylpropan-2-ol;
3-(methylamino)-1-phenyl-1-(4-phenyl-1H-indol-1-yl)propan-2-ol;
3-(methylamino)-1-phenyl-1-(6-phenyl-1H-indol-1-yl)propan-2-ol;
3-(methylamino)-1-phenyl-1-(7-phenyl-1H-indol-1-yl)propan-2-ol;
1-[5-(benzyloxy)-1H-indol-1-yl]-1-(3-fluorophenyl)-3-(methylamino)propan-2-ol;
1-[5-(benzyloxy)-2,3-dihydro-1H-indol-1-yl]-1-(3-fluorophenyl)-3-(methylamino)propan-2-ol;
1-[5-(benzyloxy)-2,3-dihydro-1H-indol-1-yl]-3-(methylamino)-1-phenylpropan-2-ol;
5′-chloro-1′-[2-hydroxy-3-(methylamino)-1-phenylpropyl]spiro[cyclohexane-1,3′-indol]-2′(1′H)-one;
6′-chloro-1′-[(2-hydroxy-3-(methylamino)-1-phenylpropyl]spiro[cyclohexane-1,3′-indol]-2′(1′H)-one;
6′-fluoro-1′-[2-hydroxy-3-(methylamino)-1-phenylpropyl]spiro[cyclohexane-1,3′-indol]-2′(1′H)-one;
5′-fluoro-1′-[2-hydroxy-3-(methylamino)-1-phenylpropyl]spiro[cyclohexane-1,3′-indol]-2′(1′H)-one;
7′-chloro-1′-[2-hydroxy-3-(methylamino)-1-phenylpropyl]spiro[cyclohexane-1,3′-indol]-2′(1′H)-one;
6′-fluoro-1′-[1-(3-fluorophenyl)-2-hydroxy-3-(methylamino)propyl]spiro[cyclohexane-1,3′-indol]-2′(1′H)-one;
3-(methylamino)-1-phenyl-1-spiro[cyclohexane-1,3′-indol]-1′(2′H)-ylpropan-2-ol;
1-(3-fluorophenyl)-3-(methylamino)-1-{3-[2-(trifluoromethoxy)phenyl]-1H-indol-1-yl}propan-2-ol;
1-(3-fluorophenyl)-1-[3-(2-isopropoxyphenyl)-1H-indol-1-yl]-3-(methylamino)propan-2-ol;
1-(3-fluorophenyl)-1-[3-(4-fluorophenyl)-1H-indol-1-yl]-3-(methylamino)propan-2-ol;
1-(3-fluorophenyl)-3-(methylamino)-1-[3-(2-phenoxyphenyl)-1H-indol-1-yl]propan-2-ol;
1-[3-(2,4-difluorophenyl)-1H-indol-1-yl]-1-(3-fluorophenyl)-3-(methylamino)propan-2-ol;
1-[3-(2,5-difluorophenyl)-1H-indol-1-yl]-1-(3-fluorophenyl)-3-(methylamino)propan-2-ol;
1-[3-(2,3-dimethoxyphenyl)-1H-indol-1-yl]-1-(3-fluorophenyl)-3-(methylamino)propan-2-ol;
1-[3-(2,4-dichlorophenyl)-1H-indol-1-yl]-1-(3-fluorophenyl)-3-(methylamino)propan-2-ol;
1-[3-(2-ethoxyphenyl)-1H-indol-1-yl]-1-(3-fluorophenyl)-3-(methylamino)propan-2-ol;
1-(7-chloro-5-methoxy-1H-pyrrolo[2,3-c]pyridin-1-yl)-1-(3-fluorophenyl)-3-(methylamino)propan-2-ol;
1-(7-chloro-5-methyl-1H-pyrrolo[2,3-c]pyridin-1-yl)-3-(methylamino)-1-phenylpropan-2-ol;
1-(5-methoxy-1H-pyrrolo[2,3-c]pyridin-1-yl)-3-(methylamino)-1-phenylpropan-2-ol;
1-(3-fluorophenyl)-1-(5-methoxy-1H-pyrrolo[2,3-c]pyridin-1-yl)-3-(methylamino)propan-2-ol;
3-(methylamino)-1-(5-methyl-1H-pyrrolo[2,3-c]pyridin-1-yl)-1-phenylpropan-2-ol;
1-(3-fluorophenyl)-3-(methylamino)-1-(5-methyl-1H-pyrrolo[2,3-c]pyridin-1-yl)propan-2-ol;
3-(methylamino)-1-(7-methyl-1H-pyrrolo[2,3-c]pyridin-1-yl)-1-phenylpropan-2-ol;
1-(3-fluorophenyl)-3-(methylamino)-1-(7-methyl-1H-pyrrolo[2,3-c]pyridin-1-yl)propan-2-ol;
1-(3,3-diethyl-2,3-dihydro-1H-indol-1-yl)-1-(3-fluorophenyl)-3-(methylamino)propan-2-ol;
1-(6-fluoro-3,3-dimethyl-2,3-dihydro-1H-indol-1-yl)-1-(3-fluorophenyl)-3-(methylamino)propan-2-ol;
1-(4-benzyl-3,4-dihydroquinoxalin-1(2H)-yl)-1-(3-fluorophenyl)-3-(methylamino)propan-2-ol;
1-(5-fluoro-3,3-dimethyl-2,3-dihydro-1H-indol-1-yl)-1-(3-fluorophenyl)-3-(methylamino)propan-2-ol;
1-(3-fluorophenyl)-3-(methylamino)-1-[(3S)-3-methyl-2,3-dihydro-1H-indol-1-yl]propan-2-ol;
1-(3-fluorophenyl)-3-(methylamino)-1-[(3R)-3-methyl-2,3-dihydro-1H-indol-1-yl]propan-2-ol;
1-(3-fluorophenyl)-1-(3-isopropyl-2,3-dihydro-1H-indol-1-yl)-3-(methylamino)propan-2-ol;
1-(3-ethyl-2,3-dihydro-1H-indol-1-yl)-1-(3-fluorophenyl)-3-(methylamino)propan-2-ol;
1-(3-ethyl-2,3-dihydro-1H-indol-1-yl)-3-(methylamino)-1-phenylpropan-2-ol;
1-(3-isopropyl-2,3-dihydro-1H-indol-1-yl)-3-(methylamino)-1-phenylpropan-2-ol;
3-amino-1-(3,5-difluorophenyl)-1-(3,3-dimethyl-2,3-dihydro-1H-indol-1-yl)propan-2-ol;
1-[1-(3,5-difluorophenyl)-2-hydroxy-3-(methylamino)propyl]-7-fluoro-3,3-dimethyl-1,3-dihydro-2H-indol-2-one;
5,7-difluoro-1-[1-(3-fluorophenyl)-2-hydroxy-3-(methylamino)propyl]-3,3-dimethyl-1,3-dihydro-2H-indol-2-one;
1-[1-(3,5-difluorophenyl)-2-hydroxy-3-(methylamino)propyl]-3,3-dimethyl-1,3-dihydro-2H-indol-2-one;
1-[2-hydroxy-3-(methylamino)-1-phenylpropyl]-1H-indol-5-ol;
1-[1-(3-fluorophenyl)-2-hydroxy-3-(methylamino)propyl]-1H-indol-5-ol:
5′-(benzyloxy)-1′-[2-hydroxy-3-(methylamino)-1-phenylpropyl]spiro[cyclohexane-1,3′-indol]-2′(1′H)-one;
5-(benzyloxy)-1-[2-hydroxy-3-(methylamino)-1-phenylpropyl]-3,3-dimethyl-1,3-dihydro-2H-indol-2-one; and
pharmaceutically acceptable salts thereof.

55. A compound selected from the group consisting of:
1-[1-(3-chlorophenyl)-2-hydroxy-3-(methylamino)propyl]-7-fluoro-3,3-dimethyl-1,3-dihydro-2H-indol-2-one;
1-(3-chloro-5-fluorophenyl)-1-(1H-indol-1-yl)-3-(methylamino)propan-2-ol;
3-chloro-N-{1-[2-hydroxy-3-(methylamino)-1-phenylpropyl]-1H-indol-5-yl}-4-methylbenzamide;
3-chloro-N-{1-[2-hydroxy-3-(methylamino)-1-phenylpropyl]-2,3-dihydro-1H-indol-5-yl}benzamide;
3-chloro-N-{1-[2-hydroxy-3-(methylamino)-1-phenylpropyl]-1H-indol-5-yl}benzamide;
N-{1-[2-hydroxy-3-(methylamino)-1-phenylpropyl]-2,3-dihydro-1H-indol-5-yl}benzamide;
N-{1-[2-hydroxy-3-(methylamino)-1-phenylpropyl]-1H-indol-5-yl}benzamide;
N-{1-[2-hydroxy-3-(methylamino)-1-phenylpropyl]-2,3-dihydro-1H-indol-5-yl}cyclohexanecarboxamide;
N-{1-[2-hydroxy-3-(methylamino)-1-phenylpropyl]-1H-indol-5-yl}cyclohexanecarboxamide;
N-(3-chlorophenyl)-1-[2-hydroxy-3-(methylamino)-1-phenylpropyl]indoline-5-carboxamide;
N-(3-chlorophenyl)-1-[2-hydroxy-3-(methylamino)-1-phenylpropyl]-1H-indole-5-carboxamide;
3-(methylamino)-1-(6-phenoxy-1H-indol-1-yl)-1-phenylpropan-2-ol;
3-(methylamino)-1-(7-phenoxy-1H-indol-1-yl)-1-phenylpropan-2-ol;
3-amino-1-[5-(benzyloxy)-1H-indol-1-yl]-1-phenylpropan-2-ol;
1-[5-(benzyloxy)-1H-indol-1-yl]-3-(ethylamino)-1-phenylpropan-2-ol;
1-[5-(benzyloxy)-1H-indol-1-yl]-1-phenyl-3-(propylamino)propan-2-ol;
1-[5-(benzyloxy)-1H-indol-1-yl]-3-(isopropylamino)-1-phenylpropan-2-ol;
1-[5-(benzyloxy)-1H-indol-1-yl]-3-(dimethylamino)-1-phenylpropan-2-ol;
1-[5-(benzyloxy)-1H-indol-1-yl]-3-[ethyl(methyl)amino]-1-phenylpropan-2-ol;
1-[5-(benzyloxy)-1H-indol-1-yl]-3-(diethylamino)-1-phenylpropan-2-ol;
1-[5-(benzyloxy)-1H-indol-1-yl]-1-phenyl-3-pyrrolidin-1-ylpropan-2-ol;
1-[5-(benzyloxy)-1H-indol-1-yl]-1-phenyl-3-piperidin-1-ylpropan-2-ol;
1-[5-(benzyloxy)-1H-indol-1-yl]-3-(4-methylpiperazin-1-yl)-1-phenylpropan-2-ol hydrochloride
3-(methylamino)-1-phenyl-1-[5-(pyridin-2-ylmethoxy)-1H-indol-1-yl]propan-2-ol;
3-(methylamino)-1-phenyl-1-[5-(phenylethynyl)-1H-indol-1-yl]propan-2-ol;
3-(methylamino)-1-phenyl-1-[5-(2-phenylethyl)-1H-indol-1-yl]propan-2-ol;
1′-[3-amino-2-hydroxy-1-phenylpropyl]-6′-fluorospiro[cyclohexane-1,3′-indol]-2′(1′H)-one;
1′-[3-(ethylamino)-2-hydroxy-1-phenylpropyl]-6′-fluorospiro[cyclohexane-1,3′-indol]-2′(1′H)-one;
6′-fluoro-1′-[2-hydroxy-3-(isopropylamino)-1-phenylpropyl]spiro[cyclohexane-1,3′-indol]-2′(1′H)-one;
6′-fluoro-1′-[2-hydroxy-1-phenyl-3-(propylamino)propyl]spiro[cyclohexane-1,3′-indol]-2′(1′H)-one;
1′-[3-amino-2-hydroxy-1-phenylpropyl]-5′-fluorospiro[cyclohexane-1,3′-indol]-2′(1′H)-one;
1′-[3-(ethylamino)-2-hydroxy-1-phenylpropyl]-5′-fluorospiro[cyclohexane-1,3′-indol]-2′(1′H)-one;
5′-fluoro-1′-[2-hydroxy-3-(isopropylamino)-1-phenylpropyl]spiro[cyclohexane-1,3′-indol]-2′(1′H)-one;
5′-fluoro-1′-[2-hydroxy-1-phenyl-3-(propylamino)propyl]spiro[cyclohexane-1,3′-indol]-2′(1′H)-one;
1′-[3-(dimethylamino)-2-hydroxy-1-phenylpropyl]-5′-fluorospiro[cyclohexane-1,3′-indol]-2′(1′H)-one;
5′-fluoro-1′-[2-hydroxy-3-morpholin-4-yl-1-phenylpropyl]spiro[cyclohexane-1,3′-indol]-2′(1′H)-one;
1′-[2-hydroxy-3-(methylamino)-1-phenylpropyl]-5′-methoxyspiro[cyclohexane-1,3′-indol]-2′(1′H)-one;
1′-[2-hydroxy-3-(methylamino)-1-phenylpropyl]-6′-methoxyspiro[cyclohexane-1,3′-indol]-2′(1′H)-one;
1′-[2-hydroxy-3-(methylamino)-1-phenylpropyl]-2′-oxo-1′,2′-dihydrospiro[cyclohexane-1,3′-indole]-5′-carbonitrile;
1′-[2-hydroxy-3-(methylamino)-1-phenylpropyl]-2′-oxo-1′,2′-dihydrospiro[cyclohexane-1,3′-indole]-6′-carbonitrile;
4′,5′-difluoro-1′-[2-hydroxy-3-(methylamino)-1-phenylpropyl]spiro[cyclohexane-1,3′-indol]-2′(1′H)-one;
7′-fluoro-1′-[1-(3-fluorophenyl)-2-hydroxy-3-(methylamino)propyl]spiro[cyclohexane-1,3′-indol]-2′(1′H)-one;
1′-[1-(3-chlorophenyl)-2-hydroxy-3-(methylamino)propyl]-6′-fluorospiro[cyclohexane-1,3′-indol]-2′(1′H)-one;
1-[1-(3-chloro-5-fluorophenyl)-2-hydroxy-3-(methylamino)propyl]-7-fluoro-3,3-dimethyl-1,3-dihydro-2H-indol-2-one;
1-(3-chloro-5-fluorophenyl)-1-(2,3-dihydro-1H-indol-1-yl)-3-(methylamino)propan-2-ol;
1-(3-chloro-5-fluorophenyl)-1-(7-fluoro-3,3-dimethyl-2,3-dihydro-1H-indol-1-yl)-3-(methylamino)propan-2-ol;
1-(3-chloro-5-fluorophenyl)-1-(3,3-dimethyl-2,3-dihydro-1H-indol-1-yl)-3-(methylamino)propan-2-ol;
7′-fluoro-1′-[1-(3-fluorophenyl)-2-hydroxy-3-(methylamino)propyl]spiro[cyclobutane-1,3′-indol]-2′(1′H)-one;
7′-fluoro-1′-[1-(3-fluorophenyl)-2-hydroxy-3-(methylamino)propyl]spiro[cyclopentane-1,3′-indol]-2′(1′H)-one;
6-fluoro-1-[1-(3-fluorophenyl)-2-hydroxy-3-(methylamino)propyl]-3,3-dimethyl-1,3-dihydro-2H-indol-2-one;
1-(7-fluoro-2,3-dihydro-1H-indol-1-yl)-3-(methylamino)-1-phenylpropan-2-ol;
4-fluoro-3-[1-(3-fluorophenyl)-2-hydroxy-3-(methylamino)propyl]-1-phenyl-1,3-dihydro-2H-benzimidazol-2-one;
4-fluoro-1-(3-fluorophenyl)-3-[1-(3-fluorophenyl)-2-hydroxy-3-(methylamino)propyl]-1,3-dihydro-2H-benzimidazol-2-one;
1-[3-amino-1-(3,5-difluorophenyl)-2-hydroxypropyl]-7-fluoro-3,3-dimethyl-1,3-dihydro-2H-indol-2-one; and
pharmaceutically acceptable salts thereof.

56. A compound according to claim 54 or 55,
wherein said pharmaceutically acceptable salt is hydrochloride.

57. A pharmaceutical composition, comprising:
a. at least one compound according to claim 1, 22, 36, 54, or 55, or a pharmaceutically acceptable salt thereof; and
b. at least one pharmaceutically acceptable carrier.

58. A method for treating or preventing a condition ameliorated by monoamine reuptake in a subject in need thereof, comprising the step of:
administering to said subject an effective amount of a compound according to claim 1, 22, 36, 54, or 55, or pharmaceutically acceptable salt thereof.

59. A method according to claim 58,
wherein said condition ameliorated by monoamine reuptake is selected from the group consisting of vasomotor symptoms, sexual dysfunction, gastrointestinal and genitourinary disorders, chronic fatigue syndrome, fibromylagia syndrome, nervous system disorders, and combinations thereof.

60. A method according to claim 59,
wherein said condition ameliorated by monoamine reuptake is selected from the group consisting of major depressive disorder, vasomotor symptoms, stress and urge urinary incontinence, fibromyalgia, pain, diabetic neuropathy, and combinations thereof.

61. A method for treating or preventing at least one vasomotor symptom in a subject in need thereof, comprising the step of:
administering to said subject an effective amount of a compound according to claim 1, 22, 36, 54, or 55, or pharmaceutically acceptable salt thereof.

62. A method according to claim 61,
wherein said vasomotor symptom is hot flush.

63. A method according to claim 62,
wherein said subject is human.

64. A method according to claim 63,
wherein said human is a female.

65. A method according to claim 64,
wherein said female is pre-menopausal.

66. A method according to claim 64,
wherein said female is peri-menopausal.

67. A method according to claim 64,
wherein said female is post-menopausal.

68. A method according to claim 63,
wherein said human is a male.

69. A method according to claim 68,
wherein said male is naturally, chemically or surgically andropausal.

70. A method for treating or preventing at least one depression disorder in a subject in need thereof, comprising the step of:
administering to said subject an effective amount of a compound according to claim 1, 22, 36, 54, or 55, or pharmaceutically acceptable salt thereof.

71. A method according to claim 70,
wherein said depression disorder is major depressive disorder, anxiety, sleep disturbance, or social phobia.

72. A method according to claim 71,
wherein said subject is human.

73. A method for treating or preventing at least one sexual dysfunction in a subject in need thereof, comprising the step of:
administering to said subject an effective amount of a compound according to claim 1, 22, 36, 54, or 55, or pharmaceutically acceptable salt thereof.

74. A method according to claim 73,
wherein said sexual dysfunction is desire-related or arousal-related.

75. A method according to claim 73,
wherein said subject is human.

76. A method for treating or preventing pain in a subject in need thereof, comprising the step of:
administering to said subject an effective amount of a compound according to claim 1, 22, 36, 54, or 55, or pharmaceutically acceptable salt thereof.

77. A method according to claim 76,
wherein said pain is acute centralized pain, acute peripheral pain, or a combination thereof.

78. A method according to claim 76,
wherein said pain is chronic centralized pain, chronic peripheral pain, or a combination thereof.

79. A method according to claim 76,
wherein said pain is neuropathic pain, visceral pain, musculoskeletal pain, bony pain, cancer pain, inflammatory pain, or a combination thereof.

80. A method according to claim 79,
wherein said neuropathic pain is associated with diabetes, post traumatic pain of amputation, lower back pain, cancer, chemical injury, toxins, major surgery, peripheral nerve damage due to traumatic injury compression, post-herpetic neuralgia, trigeminal neuralgia, lumbar or cervical radiculopathies, fibromyalgia, glossopharyngeal neuralgia, reflex sympathetic dystrophy, casualgia, thalamic syndrome, nerve root avulsion, reflex sympathetic dystrophy or post thoracotomy pain, nutritional deficiencies, viral infection, bacterial infection, metastatic infiltration, adiposis dolorosa, burns, central pain conditions related to thalamic conditions, and combinations thereof.

81. A method according to claim 79,
wherein said visceral pain is associated with ulcerative colitis, irritable bowel syndrome, irritable bladder, Crohn's disease, rheumatologic (arthralgias), tumors, gastritis, pancreatitis, infections of the organs, biliary tract disorders, and combinations thereof.

82. A method according to claim 76,
wherein said pain is female-specific pain.

83. A method according to claim 82,
wherein said subject is human.

84. A method for treating or preventing gastrointestinal or genitourinary disorder in a subject in need thereof, comprising the step of:
administering to said subject an effective amount of a compound according to claim 1, 22, 36, 54, or 55, or pharmaceutically acceptable salt thereof.

85. A method according to claim 84,
wherein said disorder is stress incontinence or urge urinary incontinence.

86. A method according to claim 84,
wherein said subject is human.

87. A method for treating or preventing chronic fatigue syndrome in a subject in need thereof, comprising the step of:
administering to said subject an effective amount of a compound according to claim 1, 22, 36, 54, or 55, or pharmaceutically acceptable salt thereof.

88. A method according to claim 87,
wherein said subject is human.

89. A method for treating or preventing fibromylagia syndrome in a subject in need thereof, comprising the step of:
administering to said subject an effective amount of a compound according to claim 1, 22, 36, 54, or 55, or pharmaceutically acceptable salt thereof.

90. A method according to claim 89,
wherein said subject is human.

91. A method for treating or preventing schizophrenia in a subject in need thereof, comprising the step of:
administering to said subject an effective amount of a compound according to claim 1, 22, 36, 54, or 55, or pharmaceutically acceptable salt thereof.

92. A method according to claim 91,
wherein said subject is human.
